SEQUENCE LISTING (190 sequences)

SEQ ID NO: 1 - protein, 629 aa, Artificial Sequence
  Description of Artificial Sequence Synthetic polypeptide
METDTLLLWVLLLWVPGSTGAGGSDYKDDDDKGGSVDVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLGGGGSGGGGSGGGGSGGGGSQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSGSTSGSGKPGSGEGSTKGDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 2 - protein, 740 aa, Artificial Sequence
  Description of Artificial Sequence Synthetic polypeptide
METDTLLLWVLLLWVPGSTGAGGSDYKDDDDKGGSVDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSGGGGSGGGGSGGGGSQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSGSTSGSGKPGSGEGSTKGDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 3 - protein, 740 aa, Artificial Sequence
  Description of Artificial Sequence Synthetic polypeptide
METDTLLLWVLLLWVPGSTGAGGSDYKDDDDKGGSVDIVLTQSPASLAVSLGERATINCRASESVSVIGAHLIHWYQQKPGQPPKLLIYLASNLETGVPARFSGSGSGTDFTLTISSLQAEDAAIYSCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSGGGGSGGGGSGGGGSGGGGSQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSGSTSGSGKPGSGEGSTKGDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 4 - protein, 740 aa, Artificial Sequence
  Description of Artificial Sequence Synthetic polypeptide
METDTLLLWVLLLWVPGSTGAGGSDYKDDDDKGGSVDIVMTQSQRFMTTSVGDRVSVTCKASQSVDSNVAWYQQKPRQSPKALIFSASLRFSGVPARFTGSGSGTDFTLTISNLQSEDLAEYFCQQYNNYPLTFGAGTKLELKRGSTSGSGKPGSGEGSTKGQVQLQQSGGGLVQPGGSLKLSCAASGIDFSRYWMSWVRRAPGKGLEWIGEINPDSSTINYAPSLKDKFIISRDNAKNTLYLQMSKVRSEDTALYYCASLYYDYGDAMDYWGQGTSVTVSSGGGGSGGGGSGGGGSGGGGSQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSGSTSGSGKPGSGEGSTKGDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 5 - protein, 740 aa, Artificial Sequence
  Description of Artificial Sequence Synthetic polypeptide
METDTLLLWVLLLWVPGSTGAGGSDYKDDDDKGGSVDIVMTQSPATLSVSVGDEVTLTCKASQSVDSNVAWYQQKPGQAPKLLIYSASLRFSGVPARFSGSGSGTDFTLTISSLQSEDFAVYYCQQYNNYPLTFGAGTKLELKRGSTSGSGKPGSGEGSTKGEVQLVESGGGLVQPGGSLRLSCAASGFTFSRYWMSWVRQAPGKGLEWVGEINPDSSTINYAPSLKGRFTISRDNAKNTLYLQMNSLRAEDTAVYYCASLYYDYGDAMDYWGQGTLVTVSSGGGGSGGGGSGGGGSGGGGSQVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSGSTSGSGKPGSGEGSTKGDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 6 - protein, 629 aa, Artificial Sequence
  Description of Artificial Sequence Synthetic polypeptide
METDTLLLWVLLLWVPGSTGAGGSDYKDDDDKGGSVDEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKGGGGSGGGGSGGGGSGGGGSVLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKLESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 741 aa, Artificial Sequence
  Description of Artificial Sequence Synthetic polypeptide
METDTLLLWVLLLWVPGSTGAGGSDYKDDDDKGGSVDEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKGGGGSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 741 aa, Artificial Sequence
  Description of Artificial Sequence Synthetic polypeptide
METDTLLLWVLLLWVPGSTGAGGSDYKDDDDKGGSVDEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKGGGGSGGGGSGGGGSGGGGSDIVLTQSPASLAVSLGERATINCRASESVSVIGAHLIHWYQQKPGQPPKLLIYLASNLETGVPARFSGSGSGTDFTLTISSLQAEDAAIYSCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - protein, 741 aa, Artificial Sequence
  Description of Artificial Sequence Synthetic polypeptide
METDTLLLWVLLLWVPGSTGAGGSDYKDDDDKGGSVDEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKGGGGSGGGGSGGGGSGGGGSDIVMTQSQRFMTTSVGDRVSVTCKASQSVDSNVAWYQQKPRQSPKALIFSASLRFSGVPARFTGSGSGTDFTLTISNLQSEDLAEYFCQQYNNYPLTFGAGTKLELKRGSTSGSGKPGSGEGSTKGQVQLQQSGGGLVQPGGSLKLSCAASGIDFSRYWMSWVRRAPGKGLEWIGEINPDSSTINYAPSLKDKFIISRDNAKNTLYLQMSKVRSEDTALYYCASLYYDYGDAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 741 aa, Artificial Sequence
  Description of Artificial Sequence Synthetic polypeptide
METDTLLLWVLLLWVPGSTGAGGSDYKDDDDKGGSVDEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKGGGGSGGGGSGGGGSGGGGSDIVMTQSPATLSVSVGDEVTLTCKASQSVDSNVAWYQQKPGQAPKLLIYSASLRFSGVPARFSGSGSGTDFTLTISSLQSEDFAVYYCQQYNNYPLTFGAGTKLELKRGSTSGSGKPGSGEGSTKGEVQLVESGGGLVQPGGSLRLSCAASGFTFSRYWMSWVRQAPGKGLEWVGEINPDSSTINYAPSLKGRFTISRDNAKNTLYLQMNSLRAEDTAVYYCASLYYDYGDAMDYWGQGTLVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - protein, 958 aa, Artificial Sequence
  Description of Artificial Sequence Synthetic polypeptide
METDTLLLWVLLLWVPGSTGAGGSDYKDDDDKGGSVDEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKGGGGSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 740 aa, Artificial Sequence
  Description of Artificial Sequence Synthetic polypeptide
METDTLLLWVLLLWVPGSTGAGGSDYKDDDDKGGSVDIVLTQSPASLAVSLGERATINCRASESVSVIGAHLIHWYQQKPGQPPKLLIYLASNLETGVPARFSGSGSGTDFTLTISSLQAEDAAIYSCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSGGGGSGGGGSGGGGSGGGGSEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 13 - protein, 957 aa, Artificial Sequence
  Description of Artificial Sequence Synthetic polypeptide
METDTLLLWVLLLWVPGSTGAGGSDYKDDDDKGGSVDIVLTQSPASLAVSLGERATINCRASESVSVIGAHLIHWYQQKPGQPPKLLIYLASNLETGVPARFSGSGSGTDFTLTISSLQAEDAAIYSCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSSGGGGSGGGGSGGGGSGGGGSEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 14 - protein, 5 aa, Artificial Sequence
  Description of Artificial Sequence Synthetic peptide
DYSIN

SEQ ID NO: 15 - protein, 6 aa, Artificial Sequence
  Description of Artificial Sequence Synthetic peptide
NTETRE

SEQ ID NO: 16 - protein, 8 aa, Artificial Sequence
  Description of Artificial Sequence Synthetic peptide
DYSYAMDY

SEQ ID NO: 17 - protein, 117 aa, Artificial Sequence
  Description of Artificial Sequence Synthetic polypeptide
QIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 18 - protein, 11 aa, Artificial Sequence
  Description of Artificial Sequence Synthetic peptide
SESVSVIGAHL

SEQ ID NO: 19 - protein, 3 aa, Artificial Sequence
  Description of Artificial Sequence Synthetic peptide
LAS

SEQ ID NO: 20 - protein, 6 aa, Artificial Sequence
  Description of Artificial Sequence Synthetic peptide
SRIFPR

SEQ ID NO: 21 - protein, 111 aa, Artificial Sequence
  Description of Artificial Sequence Synthetic polypeptide
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIK

SEQ ID NO: 22 - protein, 246 aa, Artificial Sequence
  Description of Artificial Sequence Synthetic polypeptide
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 23 - protein, 117 aa, Artificial Sequence
  Description of Artificial Sequence Synthetic polypeptide
QVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 24 - protein, 111 aa, Artificial Sequence
  Description of Artificial Sequence Synthetic polypeptide
DIVLTQSPASLAVSLGERATINCRASESVSVIGAHLIHWYQQKPGQPPKLLIYLASNLETGVPARFSGSGSGTDFTLTISSLQAEDAAIYSCLQSRIFPRTFGQGTKLEIK

SEQ ID NO: 25 - protein, 246 aa, Artificial Sequence
  Description of Artificial Sequence Synthetic polypeptide
DIVLTQSPASLAVSLGERATINCRASESVSVIGAHLIHWYQQKPGQPPKLLIYLASNLETGVPARFSGSGSGTDFTLTISSLQAEDAAIYSCLQSRIFPRTFGQGTKLEIKGSTSGSGKPGSGEGSTKGQVQLVQSGSELKKPGASVKVSCKASGYTFTDYSINWVRQAPGQGLEWMGWINTETREPAYAYDFRGRFVFSLDTSVSTAYLQISSLKAEDTAVYYCARDYSYAMDYWGQGTLVTVSS

SEQ ID NO: 26 - protein, 5 aa, Artificial Sequence
  Description of Artificial Sequence Synthetic peptide
RYWMS

SEQ ID NO: 27 - protein, 16 aa, Artificial Sequence
  Description of Artificial Sequence Synthetic peptide
EINPDSSTINYAPSLK

SEQ ID NO: 28 - protein, 12 aa, Artificial Sequence
  Description of Artificial Sequence Synthetic peptide
LYYDYGDAMDYW

SEQ ID NO: 29 - protein, 120 aa, Artificial Sequence
  Description of Artificial Sequence Synthetic polypeptide
QVQLQQSGGGLVQPGGSLKLSCAASGIDFSRYWMSWVRRAPGKGLEWIGEINPDSSTINYAPSLKDKFIISRDNAKNTLYLQMSKVRSEDTALYYCASLYYDYGDAMDYWGQGTSVTVSS

SEQ ID NO: 30 - protein, 11 aa, Artificial Sequence
  Description of Artificial Sequence Synthetic peptide
KASQSVDSNVA

SEQ ID NO: 31 - protein, 7 aa, Artificial Sequence
  Description of Artificial Sequence Synthetic peptide
SASLRFS

SEQ ID NO: 32 - protein, 11 aa, Artificial Sequence
  Description of Artificial Sequence Synthetic peptide
QQYNNYPLTFG

SEQ ID NO: 33 - protein, 108 aa, Artificial Sequence
  Description of Artificial Sequence Synthetic polypeptide
DIVMTQSQRFMTTSVGDRVSVTCKASQSVDSNVAWYQQKPRQSPKALIFSASLRFSGVPARFTGSGSGTDFTLTISNLQSEDLAEYFCQQYNNYPLTFGAGTKLELKR

SEQ ID NO: 34 - protein, 246 aa, Artificial Sequence
  Description of Artificial Sequence Synthetic polypeptide
DIVMTQSQRFMTTSVGDRVSVTCKASQSVDSNVAWYQQKPRQSPKALIFSASLRFSGVPARFTGSGSGTDFTLTISNLQSEDLAEYFCQQYNNYPLTFGAGTKLELKRGSTSGSGKPGSGEGSTKGQVQLQQSGGGLVQPGGSLKLSCAASGIDFSRYWMSWVRRAPGKGLEWIGEINPDSSTINYAPSLKDKFIISRDNAKNTLYLQMSKVRSEDTALYYCASLYYDYGDAMDYWGQGTSVTVSS

SEQ ID NO: 35 - protein, 120 aa, Artificial Sequence
  Description of Artificial Sequence Synthetic polypeptide
EVQLVESGGGLVQPGGSLRLSCAASGFTFSRYWMSWVRQAPGKGLEWVGEINPDSSTINYAPSLKGRFTISRDNAKNTLYLQMNSLRAEDTAVYYCASLYYDYGDAMDYWGQGTLVTVSS

SEQ ID NO: 36 - protein, 108 aa, Artificial Sequence
  Description of Artificial Sequence Synthetic polypeptide
DIVMTQSPATLSVSVGDEVTLTCKASQSVDSNVAWYQQKPGQAPKLLIYSASLRFSGVPARFSGSGSGTDFTLTISSLQSEDFAVYYCQQYNNYPLTFGAGTKLELKR

SEQ ID NO: 37 - protein, 246 aa, Artificial Sequence
  Description of Artificial Sequence Synthetic polypeptide
DIVMTQSPATLSVSVGDEVTLTCKASQSVDSNVAWYQQKPGQAPKLLIYSASLRFSGVPARFSGSGSGTDFTLTISSLQSEDFAVYYCQQYNNYPLTFGAGTKLELKRGSTSGSGKPGSGEGSTKGEVQLVESGGGLVQPGGSLRLSCAASGFTFSRYWMSWVRQAPGKGLEWVGEINPDSSTINYAPSLKGRFTISRDNAKNTLYLQMNSLRAEDTAVYYCASLYYDYGDAMDYWGQGTLVTVSS

SEQ ID NO: 38 - protein, 134 aa, Artificial Sequence
  Description of Artificial Sequence Synthetic polypeptide
VLHLVPINATSKDDSDVTEVMWQPALRRGRGLQAQGYGVRIQDAGVYLLYSQVLFQDVTFTMGQVVSREGQGRQETLFRCIRSMPSHPDRAYNSCYSAGVFHLHQGDILSVIIPRARAKLNLSPHGTFLGFVKL

SEQ ID NO: 39 - protein, 5 aa, Artificial Sequence
  Description of Artificial Sequence Synthetic peptide
TYWMN

SEQ ID NO: 40 - protein, 13 aa, Artificial Sequence
  Description of Artificial Sequence Synthetic peptide
MIHPSDSETRLNQ

SEQ ID NO: 41 - protein, 11 aa, Artificial Sequence
  Description of Artificial Sequence Synthetic peptide
STMIATRAMDY

SEQ ID NO: 42 - protein, 119 aa, Artificial Sequence
  Description of Artificial Sequence Synthetic polypeptide
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVS

SEQ ID NO: 43 - protein, 11 aa, Artificial Sequence
  Description of Artificial Sequence Synthetic peptide
KASQDVITGVA

SEQ ID NO: 44 - protein, 7 aa, Artificial Sequence
  Description of Artificial Sequence Synthetic peptide
SASYRYT

SEQ ID NO: 45 - protein, 9 aa, Artificial Sequence
  Description of Artificial Sequence Synthetic peptide
QQHYSTPLT

SEQ ID NO: 46 - protein, 107 aa, Artificial Sequence
  Description of Artificial Sequence Synthetic polypeptide
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 47 - protein, 244 aa, Artificial Sequence
  Description of Artificial Sequence Synthetic polypeptide
QVQLQQPGAELVRPGASVKLSCKASGYSFTTYWMNWVKQRPGQGLEWIGMIHPSDSETRLNQKFKDKATLTVDKSSSTAYMQLSSPTSEDSAVYYCARSTMIATRAMDYWGQGTSVTVSGSTSGSGKPGSGEGSTKGDIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 48 - protein, 5 aa, Artificial Sequence
  Description of Artificial Sequence Synthetic peptide
RYWMS

SEQ ID NO: 49 - protein, 17 aa, Artificial Sequence
  Description of Artificial Sequence Synthetic peptide
EINPDSSTINYAPSLKD

SEQ ID NO: 50 - protein, 10 aa, Artificial Sequence
  Description of Artificial Sequence Synthetic peptide
PDGNYWYFDV

SEQ ID NO: 51 - protein, 119 aa, Artificial Sequence
  Description of Artificial Sequence Synthetic polypeptide
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSS

SEQ ID NO: 52 - protein, 11 aa, Artificial Sequence
  Description of Artificial Sequence Synthetic peptide
KASQDVGIAVA

SEQ ID NO: 53 - protein, 7 aa, Artificial Sequence
  Description of Artificial Sequence Synthetic peptide
WASTRHT

SEQ ID NO: 54 - protein, 9 aa, Artificial Sequence
  Description of Artificial Sequence Synthetic peptide
QQYSSYPYT

SEQ ID NO: 55 - protein, 107 aa, Artificial Sequence
  Description of Artificial Sequence Synthetic polypeptide
DIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIK

SEQ ID NO: 56 - protein, 244 aa, Artificial Sequence
  Description of Artificial Sequence Synthetic polypeptide
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIK

SEQ ID NO: 57 - protein, 15 aa, Artificial Sequence
  Description of Artificial Sequence Synthetic peptide
RASESVTILGSHLIH

SEQ ID NO: 58 - protein, 7 aa, Artificial Sequence
  Description of Artificial Sequence Synthetic peptide
LASNVQT

SEQ ID NO: 59 - protein, 9 aa, Artificial Sequence
  Description of Artificial Sequence Synthetic peptide
LQSRTIPRT

SEQ ID NO: 60 - protein, 15 aa, Artificial Sequence
  Description of Artificial Sequence Synthetic peptide
RASESVSVIGAHLIH

SEQ ID NO: 61 - protein, 7 aa, Artificial Sequence
  Description of Artificial Sequence Synthetic peptide
LASNLET

SEQ ID NO: 62 - protein, 9 aa, Artificial Sequence
  Description of Artificial Sequence Synthetic peptide
LQSRIFPRT

SEQ ID NO: 63 - protein, 15 aa, Artificial Sequence
  Description of Artificial Sequence Synthetic peptide
RASESVTILGSHLIY

SEQ ID NO: 64 - protein, 8 aa, Artificial Sequence
  Description of Artificial Sequence Synthetic peptide
YLASNLET

SEQ ID NO: 65 - protein, 5 aa, Artificial Sequence
  Description of Artificial Sequence Synthetic peptide
DYWMS

SEQ ID NO: 66 - protein, 17 aa, Artificial Sequence
  Description of Artificial Sequence Synthetic peptide
EINPDSSTINYAPSLKG

SEQ ID NO: 67 - protein, 17 aa, Artificial Sequence
  Description of Artificial Sequence Synthetic peptide
EINPDSSTINYAPSLKD

SEQ ID NO: 68 - protein, 13 aa, Artificial Sequence
  Description of Artificial Sequence Synthetic peptide
LYYDYGDAMDYWG

SEQ ID NO: 69 - protein, 13 aa, Artificial Sequence
  Description of Artificial Sequence Synthetic peptide
SLYYDYGDAMDYW

SEQ ID NO: 70 - protein, 7 aa, Artificial Sequence
  Description of Artificial Sequence Synthetic peptide
SDDLRFS

SEQ ID NO: 71 - protein, 17 aa, Artificial Sequence
  Description of Artificial Sequence Synthetic peptide
MIHPSDSETRLNQKFKD

SEQ ID NO: 72 - protein, 17 aa, Artificial Sequence
  Description of Artificial Sequence Synthetic peptide
EINPDSSTINYTPSLKD

SEQ ID NO: 73 - protein, 12 aa, Artificial Sequence
  Description of Artificial Sequence Synthetic peptide
ESKYGPPCPPCP

SEQ ID NO: 74 - protein, 110 aa, Artificial Sequence
  Description of Artificial Sequence Synthetic polypeptide
APEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAK

SEQ ID NO: 75 - protein, 107 aa, Artificial Sequence
  Description of Artificial Sequence Synthetic polypeptide
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 76 - protein, 28 aa, Artificial Sequence
  Description of Artificial Sequence Synthetic peptide
MFWVLVVVGGVLACYSLLVTVAFIIFWV

SEQ ID NO: 77 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 78 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 17 aa, Artificial Sequence
  Description of Artificial Sequence Synthetic peptide
WINTETREPAYAYDFRG

SEQ ID NO: 80 - protein, 7 aa, Artificial Sequence
  Description of Artificial Sequence Synthetic peptide
GYTFTDY

SEQ ID NO: 81 - protein, 10 aa, Artificial Sequence
  Description of Artificial Sequence Synthetic peptide
GYTFTDYSIN

SEQ ID NO: 82 - protein, 11 aa, Artificial Sequence
  Description of Artificial Sequence Synthetic peptide
SESVTILGSHL

SEQ ID NO: 83 - protein, 6 aa, Artificial Sequence
  Description of Artificial Sequence Synthetic peptide
SRTIPR

SEQ ID NO: 84 - protein, 5 aa, Artificial Sequence
  Description of Artificial Sequence Synthetic peptide
DKTHT

SEQ ID NO: 85 - protein, 4 aa, Artificial Sequence
  Description of Artificial Sequence Synthetic peptide
CPPC

SEQ ID NO: 86 - protein, 15 aa, Artificial Sequence
  Description of Artificial Sequence Synthetic peptide
CPEPKSCDTPPPCPR

SEQ ID NO: 87 - protein, 12 aa, Artificial Sequence
  Description of Artificial Sequence Synthetic peptide
ELKTPLGDTTHT

SEQ ID NO: 88 - protein, 10 aa, Artificial Sequence
  Description of Artificial Sequence Synthetic peptide
KSCDKTHTCP

SEQ ID NO: 89 - protein, 7 aa, Artificial Sequence
  Description of Artificial Sequence Synthetic peptide
KCCVDCP

SEQ ID NO: 90 - protein, 7 aa, Artificial Sequence
  Description of Artificial Sequence Synthetic peptide
KYGPPCP

SEQ ID NO: 91 - protein, 15 aa, Homo sapiens
EPKSCDKTHTCPPCP

SEQ ID NO: 92 - protein, 12 aa, Homo sapiens
ERKCCVECPPCP

SEQ ID NO: 93 - protein, 12 aa, Artificial Sequence
  Description of Artificial Sequence Synthetic peptide
SPNMVPHAHHAQ

SEQ ID NO: 94 - protein, 12 aa, Artificial Sequence
  Description of Artificial Sequence Synthetic peptide
ESKYGPPCPSCP

SEQ ID NO: 95 - protein, 15 aa, Artificial Sequence
  Description of Artificial Sequence Synthetic peptide
EPKSCDKTYTCPPCP

SEQ ID NO: 96 - protein, 45 aa, Artificial Sequence
  Description of Artificial Sequence Synthetic polypeptide
TTTPAPRPPTPAPTIASQPLSLRPEACRPAAGGAVHTRGLDFACD

SEQ ID NO: 97 - protein, 110 aa, Artificial Sequence; identical to SEQ ID NO: 74
  Description of Artificial Sequence Synthetic polypeptide

SEQ ID NO: 98 - protein, 107 aa, Artificial Sequence; identical to SEQ ID NO: 75
  Description of Artificial Sequence Synthetic polypeptide

SEQ ID NO: 99 - protein, 28 aa, Artificial Sequence
  Description of Artificial Sequence Synthetic peptide
MFWVLVVVGGVLACYSLLVTVAFIIFWV

SEQ ID NO: 100 - protein, 23 aa, Artificial Sequence
  Description of Artificial Sequence Synthetic peptide
LGLLVAGVLVLLVSLGVAIHLCC

SEQ ID NO: 101 - protein, 25 aa, Artificial Sequence
  Description of Artificial Sequence Synthetic peptide
ALIVLGGVAGLLLFIGLGIFFCVRC

SEQ ID NO: 102 - protein, 23 aa, Artificial Sequence
  Description of Artificial Sequence Synthetic peptide
LCYLLDGILFIYGVILTALFLRV

SEQ ID NO: 103 - protein, 26 aa, Artificial Sequence
  Description of Artificial Sequence Synthetic peptide
WVLVVVGGVLACYSLLVTVAFIIFWV

SEQ ID NO: 104 - protein, 26 aa, Artificial Sequence
  Description of Artificial Sequence Synthetic peptide
VAAILGLGLVLGLLGPLAILLALYLL

SEQ ID NO: 105 - protein, 24 aa, Artificial Sequence
  Description of Artificial Sequence Synthetic peptide
ALPAALAVISFLLGLGLGVACVLA

SEQ ID NO: 106 - protein, 113 aa, Artificial Sequence
  Description of Artificial Sequence Synthetic polypeptide
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 107 - protein, 112 aa, Artificial Sequence
  Description of Artificial Sequence Synthetic polypeptide
MGGLEPCSRLLLLPLLLAVSGLRPVQAQAQSDCSCSTVSPGVLAGIVMGDLVLTVLIALAVYFLGRLVPRGRGAAEATRKQRITETESPYQELQGQRSDVYSDLNTQRPYYK

SEQ ID NO: 108 - protein, 102 aa, Artificial Sequence
  Description of Artificial Sequence Synthetic polypeptide
MGGLEPCSRLLLLPLLLAVSDCSCSTVSPGVLAGIVMGDLVLTVLIALAVYFLGRLVPRGRGAAEAATRKQRITETESPYQELQGQRSDVYSDLNTQRPYYK

SEQ ID NO: 109 - protein, 101 aa, Artificial Sequence
  Description of Artificial Sequence Synthetic polypeptide
MGGLEPCSRLLLLPLLLAVSDCSCSTVSPGVLAGIVMGDLVLTVLIALAVYFLGRLVPRGRGAAEATRKQRITETESPYQELQGQRSDVYSDLNTQRPYYK

SEQ ID NO: 110 - protein, 21 aa, Artificial Sequence
  Description of Artificial Sequence Synthetic peptide
ESPYQELQGQRSDVYSDLNTQ

SEQ ID NO: 111 - protein, 86 aa, Artificial Sequence
  Description of Artificial Sequence Synthetic polypeptide
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 112 - protein, 21 aa, Artificial Sequence
  Description of Artificial Sequence Synthetic peptide
DGVYTGLSTRNQETYETLKHE

SEQ ID NO: 113 - protein, 171 aa, Artificial Sequence
  Description of Artificial Sequence Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRMCQSCVELDPATVAGIIVTDVIATLLLALGVFCFAGHETGRLSGAADTQALLRNDQVYQPLRDRDDAQYSHLGGNWARNK

SEQ ID NO: 114 - protein, 127 aa, Artificial Sequence
  Description of Artificial Sequence Synthetic polypeptide
MEHSTFLSGLVLATLLSQVSPFKIPIEELEDRVFVNCNTSITWVEGTVGTLLSDITRLDLGKRILDPRGIYRCNGTDIYKDKESTVQVHYRTADTQALLRNDQVYQPLRDRDDAQYSHLGGNWARNK

SEQ ID NO: 115 - protein, 21 aa, Artificial Sequence
  Description of Artificial Sequence Synthetic peptide
DQVYQPLRDRDDAQYSHLGGN

SEQ ID NO: 116 - protein, 207 aa, Artificial Sequence
  Description of Artificial Sequence Synthetic polypeptide
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 117 - protein, 21 aa, Artificial Sequence
  Description of Artificial Sequence Synthetic peptide
NPDYEPIRKGQRDLYSGLNQR

SEQ ID NO: 118 - protein, 182 aa, Artificial Sequence
  Description of Artificial Sequence Synthetic polypeptide
MEQGKGLAVLILAIILLQGTLAQSIKGNHLVKVYDYQEDGSVLLTCDAEAKNITWFKDGKMIGFLTEDKKKWNLGSNAKDPRGMYQCKGSQNKSKPLQVYYRMCQNCIELNAATISGFLFAEIVSIFVLAVGVYFIAGQDGVRQSRASDKQTLLPNDQLYQPLKDREDDQYSHLQGNQLRRN

SEQ ID NO: 119 - protein, 21 aa, Artificial Sequence
  Description of Artificial Sequence Synthetic peptide
DQLYQPLKDREDDQYSHLQGN

SEQ ID NO: 120 - protein, 163 aa, Artificial Sequence
  Description of Artificial Sequence Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 121 - protein, 164 aa, Artificial Sequence
  Description of Artificial Sequence Synthetic polypeptide
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 122 - protein, 112 aa, Artificial Sequence; identical to SEQ ID NO: 78
  Description of Artificial Sequence Synthetic polypeptide

SEQ ID NO: 123 - protein, 21 aa, Artificial Sequence
  Description of Artificial Sequence Synthetic peptide
NQLYNELNLGRREEYDVLDKR

SEQ ID NO: 124 - protein, 22 aa, Artificial Sequence
  Description of Artificial Sequence Synthetic peptide
EGLYNELQKDKMAEAYSEIGMK

SEQ ID NO: 125 - protein, 21 aa, Artificial Sequence
  Description of Artificial Sequence Synthetic peptide
DGLYQGLSTATKDTYDALHMQ

SEQ ID NO: 126 - protein, 226 aa, Artificial Sequence
  Description of Artificial Sequence Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNGTLIIQNVNKSHGGIYVCRVQEGNESYQQSCGTYLRVRQPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 127 - protein, 188 aa, Artificial Sequence
  Description of Artificial Sequence Synthetic polypeptide
MPGGPGVLQALPATIFLLFLLSAVYLGPGCQALWMHKVPASLMVSLGEDAHFQCPHNSSNNANVTWWRVLHGNYTWPPEFLGPGEDPNEPPPRPFLDMGEGTKNRIITAEGIILLFCAVVPGTLLLFRKRWQNEKLGLDAGDEYEDENLYEGLNLDDCSMYEDISRGLQGTYQDVGSLNIGDVQLEKP

SEQ ID NO: 128 - protein, 21 aa, Artificial Sequence
  Description of Artificial Sequence Synthetic peptide
ENLYEGLNLDDCSMYEDISRG

SEQ ID NO: 129 - protein, 20 aa, Artificial Sequence
  Description of Artificial Sequence Synthetic peptide
METDTLLLWVLLLWVPGSTG

SEQ ID NO: 130 - protein, 17 aa, Homo sapiens
ELKTPLGDTTHTCPRCP

SEQ ID NO: 131 - protein, 68 aa, Artificial Sequence
  Description of Artificial Sequence Synthetic polypeptide
FWVLVVVGGVLACYSLLVTVAFIIFWVRSKRSRLLHSDYMNMTPRRPGPTRKHYQPYAPPRDFAAYRS

SEQ ID NO: 132 - protein, 68 aa, Artificial Sequence; identical to SEQ ID NO: 131
  Description of Artificial Sequence Synthetic polypeptide

SEQ ID NO: 133 - protein, 619 aa, Artificial Sequence
  Description of Artificial Sequence Synthetic polypeptide
MPDPAAHLPFFYGSISRAEAEEHLKLAGMADGLFLLRQCLRSLGGYVLSLVHDVRFHHFPIERQLNGTYAIAGGKAHCGPAELCEFYSRDPDGLPCNLRKPCNRPSGLEPQPGVFDCLRDAMVRDYVRQTWKLEGEALEQAIISQAPQVEKLIATTAHERMPWYHSSLTREEAERKLYSGAQTDGKFLLRPRKEQGTYALSLIYGKTVYHYLISQDKAGKYCIPEGTKFDTLWQLVEYLKLKADGLIYCLKEACPNSSASNASGAAAPTLPAHPSTLTHPQRRIDTLNSDGYTPEPARITSPDKPRPMPMDTSVYESPYSDPEELKDKKLFLKRDNLLIADIELGCGNFGSVRQGVYRMRKKQIDVAIKVLKQGTEKADTEEMMREAQIMHQLDNPYIVRLIGVCQAEALMLVMEMAGGGPLHKFLVGKREEIPVSNVAELLHQVSMGMKYLEEKNFVHRDLAARNVLLVNRHYAKISDFGLSKALGADDSYYTARSAGKWPLKWYAPECINFRKFSSRSDVWSYGVTMWEALSYGQKPYKKMKGPEVMAFIEQGKRMECPPECPPELYALMSDCWIYKWEDRPDFLTVEQRMRACYYSLASKVEGPPGSTQKAEAACA

SEQ ID NO: 134 - protein, 44 aa, Artificial Sequence
  Description of Artificial Sequence Synthetic polypeptide
FWVRSKRSRLLHSDYMNMTPRRPGPTRKHYQPYAPPRDFAAYRS

SEQ ID NO: 135 - protein, 35 aa, Artificial Sequence
  Description of Artificial Sequence Synthetic polypeptide
TKKKYSSSVHDPNGEYMFMRAVNTAKKSRLTDVTL

SEQ ID NO: 136 - protein, 37 aa, Artificial Sequence
  Description of Artificial Sequence Synthetic polypeptide
RRDQRLPPDAHKPPGGGSFRTPIQEEQADAHSTLAKI

SEQ ID NO: 137 - protein, 114 aa, Artificial Sequence
  Description of Artificial Sequence Synthetic polypeptide
CCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 138 - protein, 49 aa, Artificial Sequence
  Description of Artificial Sequence Synthetic polypeptide
HQRRKYRSNKGESPVEPAEPCRYSCPREEEGSTIPIQEDYRKPEPACSP

SEQ ID NO: 139 - protein, 187 aa, Artificial Sequence
  Description of Artificial Sequence Synthetic polypeptide
RRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 140 - protein, 54 aa, Artificial Sequence
  Description of Artificial Sequence Synthetic polypeptide
HIWQLRSQCMWPRETQLLLEVPPSTEDARSCQFPEEERGERSAEEKGRLGDLWV

SEQ ID NO: 141 - protein, 60 aa, Artificial Sequence
  Description of Artificial Sequence Synthetic polypeptide
CVKRRKPRGDVVKVIVSVQRKRQEAEGEATVIEALQAPPDVTTVAVEETIPSFTGRSPNH

SEQ ID NO: 142 - protein, 9 aa, Artificial Sequence
  Description of Artificial Sequence Synthetic peptide
YPYDVPDYA

SEQ ID NO: 143 - protein, 8 aa, Artificial Sequence
  Description of Artificial Sequence Synthetic peptide
DYKDDDDK

SEQ ID NO: 144 - protein, 10 aa, Artificial Sequence
  Description of Artificial Sequence Synthetic peptide
EQKLISEEDL

SEQ ID NO: 145 - protein, 5 aa, Artificial Sequence
  Description of Artificial Sequence Synthetic peptide
GSGGS

SEQ ID NO: 146 - protein, 4 aa, Artificial Sequence
  Description of Artificial Sequence Synthetic peptide
GGGS

SEQ ID NO: 147 - protein, 4 aa, Artificial Sequence
  Description of Artificial Sequence Synthetic peptide
GGSG

SEQ ID NO: 148 - protein, 5 aa, Artificial Sequence
  Description of Artificial Sequence Synthetic peptide
GGSGG

SEQ ID NO: 149 - protein, 5 aa, Artificial Sequence
  Description of Artificial Sequence Synthetic peptide
GSGSG

SEQ ID NO: 150 - protein, 5 aa, Artificial Sequence
  Description of Artificial Sequence Synthetic peptide
GSGGG

SEQ ID NO: 151 - protein, 5 aa, Artificial Sequence
  Description of Artificial Sequence Synthetic peptide
GGGSG

SEQ ID NO: 152 - protein, 5 aa, Artificial Sequence
  Description of Artificial Sequence Synthetic peptide
GSSSG

SEQ ID NO: 153 - DNA, 738 bp, Artificial Sequence
  Description of Artificial Sequence Synthetic polynucleotide
gacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgatgtggccgtgtactactgcctgcagagccggaccatcccccggacctttggcggaggcaccaaactggaaatcaagggctccacttctggctccggcaaacctggttctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgcgatggactactggggccagggcacctcagtcaccgtctcctca

SEQ ID NO: 154 - DNA, 738 bp, Artificial Sequence
  Description of Artificial Sequence Synthetic polynucleotide
gacatcgtgctgacccagagccccgccagcctggccgtgtctctgggcgagagagccaccatcaactgccgggccagcgagagcgtgtccgtgatcggcgctcacctgatccactggtatcagcagaagcccggccagccccccaagctgctgatctacctagccagcaatctggagaccggcgtgcccgccagattcagcggcagcggcagcggcaccgacttcaccctgaccatctcctctctgcaggccgaagatgcagccatctactcctgcctgcagagccggatcttcccccggacctttggccagggcaccaaactggaaatcaagggctccacttctggctccggcaaacctggttctggcgagggcagcacaaagggacaggtgcagctggtgcagagcggctctgagctgaagaaacccggcgccagcgtgaaggtgagctgcaaggcctccggctacaccttcaccgactacagcatcaactgggtgagacaggcccctggccagggcctggagtggatgggctggatcaacaccgagacaagagagcccgcctacgcctacgacttccggggcagattcgtcttcagcctggacaccagcgtcagcaccgcctacctgcagatctcttccctgaaggccgaggacaccgccgtctactattgcgcccgggactacagctacgcgatggactactggggccagggcaccctggtcaccgtctcctca

SEQ ID NO: 155 - DNA, 738 bp, Artificial Sequence
  Description of Artificial Sequence Synthetic polynucleotide
gacattgtgatgactcagtctcaaagattcatgaccacatcagtaggagacagggtcagcgtcacctgcaaggccagtcagagtgtggatagtaatgtagcctggtatcaacagaaacctcggcaatctcctaaagcactgattttctcggcatccctccggttcagtggagtccctgctcgcttcacaggcagtggatctgggacagatttcactctcaccatcagcaatctgcagtctgaagacttggcagagtatttctgtcaacaatataacaactatcctctcacgttcggtgctgggaccaagctggagctgaaacgtggctccacttctggctccggcaaacctggttctggcgagggcagcacaaagggacaggtgcagctgcagcagtctggaggtggcctggtgcagcctggaggatccctgaaactctcctgtgcagcctcaggaatcgattttagtagatactggatgagttgggttcggcgggctccagggaaaggactagaatggattggagaaattaatccagatagcagtacaataaactatgcaccatctctaaaggataaattcatcatctccagagacaacgccaaaaatacgttgtacctgcaaatgagcaaagtgcgctctgaggacacagccctttattactgtgcaagtctctactatgattacggggatgctatggactactggggtcaaggaacctcagtcaccgtctcctca

SEQ ID NO: 156 - DNA, 738 bp, Artificial Sequence
  Description of Artificial Sequence Synthetic polynucleotide
gacattgtgatgactcagtctcccgccaccctgagcgtgtcagtaggagacgaggtcaccctcacctgcaaggccagtcagagtgtggatagtaatgtagcctggtatcaacagaaacctgggcaagctcctaaactgctgatttactcggcatccctccggttcagtggagtccctgctcgcttcagcggcagtggatctgggacagatttcactctcaccatcagctctctgcagtctgaagacttcgcagtgtattactgtcaacaatataacaactatcctctcacgttcggtgctgggaccaagctggagctgaaacgtggctccacttctggctccggcaaacctggttctggcgagggcagcacaaagggagaggtgcagctggtcgaatctggaggtggcctggtgcagcctggaggatccctgaggctctcctgtgcagcctcaggatttacctttagtagatactggatgagttgggttcggcaggctccagggaaaggactagaatgggtgggagaaattaatccagatagcagtacaataaactatgcaccatctctaaagggcagattcaccatctccagagacaacgccaaaaatacgttgtacctgcaaatgaacagcctgcgcgctgaggacacagccgtgtattactgtgcaagtctctactatgattacggggatgctatggactactggggtcaaggaaccctcgtcaccgtctcctca

SEQ ID NO: 157 - DNA, 402 bp, Artificial Sequence
  Description of Artificial Sequence Synthetic polynucleotide
gtgctgcacctggtgcccatcaacgccaccagcaaggacgactctgatgtgaccgaggtgatgtggcagccagccctgagacggggcagaggcctgcaggcccagggctacggcgtgagaatccaggacgctggcgtgtacctgctgtactcccaggtgctgttccaggacgtgaccttcacaatgggccaggtggtgagccgggagggccagggcagacaggagaccctgttccggtgcattcgcagcatgcccagccaccccgacagagcctacaacagctgctacagcgctggcgtgtttcacctgcaccagggcgacatcctgagcgtgatcatccccagagccagagccaagctgaacctgtccccccacggcacctttctgggcttcgtgaagctg

SEQ ID NO: 158 - DNA, 732 bp, Artificial Sequence
  Description of Artificial Sequence Synthetic polynucleotide
caggtccaactgcagcagcctggggctgagctggtgaggcctggagcttcagtgaagctgtcctgcaaggcttcggggtactccttcaccacctactggatgaactgggtgaagcagaggcctggacaaggccttgagtggattggcatgattcatccttccgatagtgaaactaggttaaatcagaagttcaaggacaaggccacattgactgtagacaaatcctccagcacagcctacatgcaactcagcagcccgacatctgaggactctgcggtctattactgtgcacgttctactatgattgcgacgagggctatggactactggggtcaaggaacctcagtcaccgtctccgggtcaacttcaggctctgggaaaccaggcagcggtgagggttcaaccaagggtgacattgtgatgacccagtctcagaaatccatgtccacatcagtaggagacagggtcagcatcacctgcaaggccagtcaggatgttattactggtgtagcctggtatcaacagaaaccagggcaatctcctaaattactgatttactcggcatcctaccggtacactggagtccctgatcgcttcactggcagtggatctgggacggatttcactttcaccatcagcaatgtgcaggctgaagacctggcagtttattactgtcagcaacattatagtactcctctcactttcggtgctgggaccaagctggagctgaaa

SEQ ID NO: 159 - DNA, 732 bp, Artificial Sequence
  Description of Artificial Sequence Synthetic polynucleotide
gaggtacaattggtggagtctggaggcggtctcgttcaaccagggggcagcctcagactttcctgcgcggcaagtgggtttgacttctctcgatattggatgtcatgggtcaggcaggcaccggggaaaggtcttgagtggataggtgagattaaccctgactctagtaccatcaattacgctcccagcttgaaagataaattcataatttcacgagacaacgccaaaaacagtttgtacctgcaaatgaatagcttgagggcggaggatacggccgtttattactgtgctaggcccgacggtaactactggtattttgatgtatggggtcaaggcactctggtgactgtatcctctggcagcaccagcggctccggcaagcctggctctggcgagggcagcacaaagggagacatacagatgacgcagtccccttcatcactctctgcgagcgttggtgacagggtgactatcacatgcaaagcaagccaagatgtcggtatagccgttgcatggtatcagcagaaaccagggaaggtcccaaaactccttatatattgggcgagcacacgccacactggtgtccctgataggcctagtggtagtggcagtggaacggatttcacccttactatatccagtttgcaacctgaggatgtggccacgtattattgtcagcagtacagctcatatccttacacctttggtcaaggaactaaggtggaaattaag

SEQ ID NO: 160 - DNA, 36 bp, Artificial Sequence
  Description of Artificial Sequence Synthetic oligonucleotide
gaatctaagtacggaccgccctgccccccttgccct

SEQ ID NO: 161 - DNA, 330 bp, Artificial Sequence
  Description of Artificial Sequence Synthetic polynucleotide
gcccccgagttcgaaggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaag

SEQ ID NO: 162 - DNA, 321 bp, Artificial Sequence
  Description of Artificial Sequence Synthetic polynucleotide
ggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaag

SEQ ID NO: 163 - DNA, 84 bp, Artificial Sequence
  Description of Artificial Sequence Synthetic oligonucleotide
atgttctgggtgctggtggtggtcggaggcgtgctggcctgctacagcctgctggtcaccgtggccttcatcatcttttgggtg

SEQ ID NO: 164 - DNA, 126 bp, Artificial Sequence
  Description of Artificial Sequence Synthetic polynucleotide
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 165 - DNA, 336 bp, Artificial Sequence
  Description of Artificial Sequence Synthetic polynucleotide
cgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 166 - DNA, 2031 bp, Artificial Sequence
  Description of Artificial Sequence Synthetic polynucleotide
atggagacagacacactcctgctatgggtgctgctgctctgggttccaggttccacaggcgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgctgatccagctcgccagcaatgtgcagaccggcgtgcccgccagattcagcggcagcggcagcagaaccgacttcaccctgaccatcgaccccgtggaagaggacgatgtggccgtgtactactgcctgcagagccggaccatcccccggacctttggcggaggcaccaaactggaaatcaagggctccacttctggctccggcaaacctggttctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgcgatggactactggggccagggcacctcagtcaccgtctcctcagaatctaagtacggaccgccctgccccccttgccctgcccccgagttcgaaggcggacccagcgtgttcctgttcccccccaagcccaaggacaccctgatgatcagccggacccccgaggtgacctgcgtggtggtggacgtgagccaggaagatcccgaggtccagttcaattggtacgtggacggcgtggaagtgcacaacgccaagaccaagcccagagaggaacagttccagagcacctaccgggtggtgtctgtgctgaccgtgctgcaccaggactggctgaacggcaaagaatacaagtgcaaggtgtccaacaagggcctgcccagcagcatcgaaaagaccatcagcaaggccaagggccagcctcgcgagccccaggtgtacaccctgcctccctcccaggaagagatgaccaagaaccaggtgtccctgacctgcctggtgaagggcttctaccccagcgacatcgccgtggagtgggagagcaacggccagcctgagaacaactacaagaccacccctcccgtgctggacagcgacggcagcttcttcctgtacagccggctgaccgtggacaagagccggtggcaggaaggcaacgtctttagctgcagcgtgatgcacgaggccctgcacaaccactacacccagaagagcctgagcctgtccctgggcaagatgttctgggtgctggtggtggtgggcggggtgctggcctgctacagcctgctggtgacagtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 167 - DNA, 2172 bp, Artificial Sequence
  Description of Artificial Sequence Synthetic polynucleotide
atggagacagacacactcctgctatgggtgctgctgctctgggttccaggttccacaggcgaggtacaattggtggagtctggaggcggtctcgttcaaccagggggcagcctcagactttcctgcgcggcaagtgggtttgacttctctcgatattggatgtcatgggtcaggcaggcaccggggaaaggtcttgagtggataggtgagattaaccctgactctagtaccatcaattacgctcccagcttgaaagataaattcataatttcacgagacaacgccaaaaacagtttgtacctgcaaatgaatagcttgagggcggaggatacggccgtttattactgtgctaggcccgacggtaactactggtattttgatgtatggggtcaaggcactctggtgactgtatcctctggcagcaccagcggctccggcaagcctggctctggcgagggcagcacaaagggagacatacagatgacgcagtccccttcatcactctctgcgagcgttggtgacagggtgactatcacatgcaaagcaagccaagatgtcggtatagccgttgcatggtatcagcagaaaccagggaaggtcccaaaactccttatatattgggcgagcacacgccacactggtgtccctgataggcctagtggtagtggcagtggaacggatttcacccttactatatccagtttgcaacctgaggatgtggccacgtattattgtcagcagtacagctcatatccttacacctttggtcaaggaactaaggtggaaattaagggtggaggcggcagtggcggaggtgggtccggagggggcggtagcggtggcgggggatctgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgcttatccagctcgccagcaatgtgcagaccggcgtgcccgccagattcagcggcagcggcagcagaaccgacttcaccctgaccatcgaccccgtggaagaggacgatgtggccgtgtactactgcctgcagagccggaccatcccccggacctttggcggaggcaccaaactggaaatcaagggctccacttctggctccggcaaacctggttctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgcgatggactactggggccagggcacctcagtcaccgtctcctcagaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 168 - DNA, 2493 bp, Artificial Sequence
  Description of Artificial Sequence Synthetic polynucleotide
atggagacagacacactcctgctatgggtgctgctgctctgggttccaggttccacaggcgaggtacaattggtggagtctggaggcggtctcgttcaaccagggggcagcctcagactttcctgcgcggcaagtgggtttgacttctctcgatattggatgtcatgggtcaggcaggcaccggggaaaggtcttgagtggataggtgagattaaccctgactctagtaccatcaattacgctcccagcttgaaagataaattcataatttcacgagacaacgccaaaaacagtttgtacctgcaaatgaatagcttgagggcggaggatacggccgtttattactgtgctaggcccgacggtaactactggtattttgatgtatggggtcaaggcactctggtgactgtatcctctggcagcaccagcggctccggcaagcctggctctggcgagggcagcacaaagggagacatacagatgacgcagtccccttcatcactctctgcgagcgttggtgacagggtgactatcacatgcaaagcaagccaagatgtcggtatagccgttgcatggtatcagcagaaaccagggaaggtcccaaaactccttatatattgggcgagcacacgccacactggtgtccctgataggcctagtggtagtggcagtggaacggatttcacccttactatatccagtttgcaacctgaggatgtggccacgtattattgtcagcagtacagctcatatccttacacctttggtcaaggaactaaggtggaaattaagggtggaggcggcagtggcggaggtgggtccggagggggcggtagcggtggcgggggatctgacatcgtgctgacccagagcccccccagcctggccatgtctctgggcaagagagccaccatcagctgccgggccagcgagagcgtgaccatcctgggcagccacctgatccactggtatcagcagaagcccggccagccccccaccctgcttatccagctcgccagcaatgtgcagaccggcgtgcccgccagattcagcggcagcggcagcagaaccgacttcaccctgaccatcgaccccgtggaagaggacgatgtggccgtgtactactgcctgcagagccggaccatcccccggacctttggcggaggcaccaaactggaaatcaagggctccacttctggctccggcaaacctggttctggcgagggcagcacaaagggacagattcagctggtgcagagcggccctgagctgaagaaacccggcgagacagtgaagatcagctgcaaggcctccggctacaccttcaccgactacagcatcaactgggtgaaaagagcccctggcaagggcctgaagtggatgggctggatcaacaccgagacaagagagcccgcctacgcctacgacttccggggcagattcgccttcagcctggaaaccagcgccagcaccgcctacctgcagatcaacaacctgaagtacgaggacaccgccacctacttttgcgccctggactacagctacgcgatggactactggggccagggcacctcagtcaccgtctcctcagaatctaagtacggaccgccctgccccccttgccctggccagcctagagaaccccaggtgtacaccctgcctcccagccaggaagagatgaccaagaaccaggtgtccctgacctgcctggtcaaaggcttctaccccagcgatatcgccgtggaatgggagagcaacggccagcccgagaacaactacaagaccaccccccctgtgctggacagcgacggcagcttcttcctgtactcccggctgaccgtggacaagagccggtggcaggaaggcaacgtcttcagctgcagcgtgatgcacgaggccctgcacaaccactacacccagaagtccctgagcctgagcctgggcaagatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 169 - protein, 677 aa, Artificial Sequence
  Description of Artificial Sequence Synthetic polypeptide
METDTLLLWVLLLWVPGSTGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 724 aa, Artificial Sequence
  Description of Artificial Sequence Synthetic polypeptide
METDTLLLWVLLLWVPGSTGEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKGGGGSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - protein, 831 aa, Artificial Sequence
  Description of Artificial Sequence Synthetic polypeptide
METDTLLLWVLLLWVPGSTGEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKGGGGSGGGGSGGGGSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSESKYGPPCPPCPGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 172 - protein, 229 aa, Artificial Sequence
  Description of Artificial Sequence Synthetic polypeptide
ESKYGPPCPPCPAPEFEGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 173 - protein, 18 aa, Artificial Sequence
  Description of Artificial Sequence Synthetic peptide
GSTSGSGKPGSGEGSTKG

SEQ ID NO: 174 - protein, 482 aa, Artificial Sequence
  Description of Artificial Sequence Synthetic polypeptide
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGGGGSDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGSTSGSGKPGSGEGSTKGQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGGGGSDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIK

SEQ ID NO: 175 - protein, 482 aa, Artificial Sequence
  Description of Artificial Sequence Synthetic polypeptide
EVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSSGSTSGSGKPGSGEGSTKGDIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGGGGSDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIK

SEQ ID NO: 176 - protein, 482 aa, Artificial Sequence
  Description of Artificial Sequence Synthetic polypeptide
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGGGGSEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGSTSGSGKPGSGEGSTKGDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 177 - protein, 482 aa, Artificial Sequence
  Description of Artificial Sequence Synthetic polypeptide
DIVLTQSPPSLAMSLGKRATISCRASESVTILGSHLIHWYQQKPGQPPTLLIQLASNVQTGVPARFSGSGSRTDFTLTIDPVEEDDVAVYYCLQSRTIPRTFGGGTKLEIKGGGGSDIQMTQSPSSLSASVGDRVTITCKASQDVGIAVAWYQQKPGKVPKLLIYWASTRHTGVPDRPSGSGSGTDFTLTISSLQPEDVATYYCQQYSSYPYTFGQGTKVEIKGSTSGSGKPGSGEGSTKGEVQLVESGGGLVQPGGSLRLSCAASGFDFSRYWMSWVRQAPGKGLEWIGEINPDSSTINYAPSLKDKFIISRDNAKNSLYLQMNSLRAEDTAVYYCARPDGNYWYFDVWGQGTLVTVSSGGGGSQIQLVQSGPELKKPGETVKISCKASGYTFTDYSINWVKRAPGKGLKWMGWINTETREPAYAYDFRGRFAFSLETSASTAYLQINNLKYEDTATYFCALDYSYAMDYWGQGTSVTVSS

SEQ ID NO: 178 - protein, 5 aa, Artificial Sequence
  Description of Artificial Sequence Synthetic peptide
GGGGS

SEQ ID NO: 179 - protein, 30 aa, Artificial Sequence
  Description of Artificial Sequence Synthetic polypeptide
  MISC_FEATURE (1)..(30): This sequence may encompass 1-6 “Gly Gly Gly Gly Ser” repeating units
GGGGSGGGGSGGGGSGGGGSGGGGSGGGGS

SEQ ID NO: 180 - protein, 30 aa, Artificial Sequence
  Description of Artificial Sequence Synthetic polypeptide
  MISC_FEATURE (1)..(30): This sequence may encompass 1-6 “Glu Ala Ala Ala Lys” repeating units
EAAAKEAAAKEAAAKEAAAKEAAAKEAAAK

SEQ ID NO: 181 - protein, 20 aa, Artificial Sequence
  Description of Artificial Sequence Synthetic peptide
GGGGSGGGGSGGGGSGGGGS

SEQ ID NO: 182 - protein, 5 aa, Artificial Sequence
  Description of Artificial Sequence Synthetic peptide
GGGGS

SEQ ID NO: 183 - protein, 5 aa, Artificial Sequence
  Description of Artificial Sequence Synthetic peptide
EAAAK

SEQ ID NO: 184 - protein, 9 aa, Unknown
  Description of Unknown “LAGLIDADG” family motif peptide
LAGLIDADG

SEQ ID NO: 185 - DNA, 321 bp, Artificial Sequence
  Description of Artificial Sequence Synthetic polynucleotide
gacattgtgatgactcagtctcaaagattcatgaccacatcagtaggagacagggtcagcgtcacctgcaaggccagtcagagtgtggatagtaatgtagcctggtatcaacagaaacctcggcaatctcctaaagcactgattttctcggcatccctccggttcagtggagtccctgctcgcttcacaggcagtggatctgggacagatttcactctcaccatcagcaatctgcagtctgaagacttggcagagtatttctgtcaacaatataacaactatcctctcacgttcggtgctgggaccaagctggagctgaaa

SEQ ID NO: 186 - DNA, 321 bp, Artificial Sequence
  Description of Artificial Sequence Synthetic polynucleotide
gacattgtgatgacccagtctcagaaatccatgtccacatcagtaggagacagggtcagcatcacctgcaaggccagtcaggatgttattactggtgtagcctggtatcaacagaaaccagggcaatctcctaaattactgatttactcggcatcctaccggtacactggagtccctgatcgcttcactggcagtggatctgggacggatttcactttcaccatcagcaatgtgcaggctgaagacctggcagtttattactgtcagcaacattatagtactcctctcactttcggtgctgggaccaagctggagctgaaa

SEQ ID NO: 187 - DNA, 363 bp, Homo sapiens
gctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctctaacatgtcagcgttattgtaatgcaagtaagtaatattgcttgaacgattattcattggtgtgaactattctgtctatatggactgcttattcagagaatcaacataatgggcatgatggtgagttttcttgaatcaaaaagagaaaggaagcaaggcagtgatt

SEQ ID NO: 188 - DNA, 365 bp, Artificial Sequence
  Description of Artificial Sequence Synthetic polynucleotide
gctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatactcctcctagctaacatgtcagcgttattgtaatgcaagtaagtaatattgcttgaacgattattcattggtgtgaactattctgtctatatggactgcttattcagagaatcaacataatgggcatgatggtgagttttcttgaatcaaaaagagaaaggaagcaaggcagtgatt

SEQ ID NO: 189 - DNA, 363 bp, Homo sapiens; identical to SEQ ID NO: 187

SEQ ID NO: 190 - DNA, 351 bp, Artificial Sequence
  Description of Artificial Sequence Synthetic polynucleotide
gctcttgctgcatttgctctggaattcttgtagagatattacttgtccttccaggctgttctttctgtagctcccttgttttctttttgtgatcatgttgcagatggctgggcagtgctcccaaaatgaatattttgacagtttgttgcatgcttgcataccttgtcaacttcgatgttcttctaatacatgtcagcgttattgtaatgcaagtaagtaatattgcttgaacgattattcattggtgtgaactattctgtctatatggactgcttattcagagaatcaacataatgggcatgatggtgagttttcttgaatcaaaaagagaaaggaagcaaggcagtgatt

(19) United States
(12) Patent Application Publication
(10) Pub. No.: US 2021/0230289 A1
(43) Pub. Date: Jul. 29, 2021

(54) SINGLE-CHAIN BISPECIFIC CHIMERIC ANTIGEN RECEPTORS FOR THE TREATMENT OF CANCER

(71) Applicant: The Regents of the University of California, Oakland, CA (US)

(72) Inventors: Yvonne Yu-hsuan CHEN, Los Angeles, CA (US); Eugenia ZAH, Los Angeles, CA (US)

(21) Appl. No.: 17/251,287

(22) PCT Filed: Jun. 12, 2019
(86) PCT No.: PCT/US2019/036731

§ 371 Date: Dec. 11, 2020

Related U.S. Application Data
(60) Provisional application No. 62/684,107, filed on Jun. 12, 2018.
(60) Provisional application No. 62/684,315, filed on Jun. 13, 2018.

(51) Int. Cl. C07K 16/28; C07K 14/705; C07K 14/725; C12N 15/86; C12N 5/0783; A61P 35/00; A61K 35/17; A61K 38/17

(52) U.S. Cl. CPC C07K 16/2878; C07K 16/2803; C07K 14/70521; C07K 14/7051; C07K 14/70578; C12N 15/86; A61K 2039/505; A61P 35/00; A61K 35/17; C07K 16/2818; A61K 38/1774; A61K 38/177; C07K 2317/31; C12N 5/0636

(57) ABSTRACT

Methods and compositions are provided concerning a bispecific chimeric antigen receptor (CAR) targeting BCMA and CS1, particularly in multiple myeloma patients. The bispecific CAR has advantages over dual or separate BCMA and CS1 CAR molecules. In some embodiments, there is a bispecific chimeric antigen receptor comprising a) a bispecific extracellular binding domain comprising both i) a BCMA-binding region and ii) a CSI-binding region; b) a single transmembrane domain; and, c) a single cytoplasmic region comprising a primary intracellular signaling domain.

### CROSS-REFERENCE TO RELATED APPLICATIONS

This application claims the benefit of U.S. Provisional Applications No. 62/684,107, filed Jun. 12, 2018, and No. 62/684,315, filed Jun. 13, 2018, the contents of which applications are incorporated into the present application by reference in their entirety.

### BACKGROUND

Multiple myeloma (MM) is a cancer of plasma cells that accounts for over 30,000 new cancer diagnoses each year (American Cancer Society). Despite the use of therapeutics ranging from monoclonal antibodies to proteasome inhibitors, MM is currently incurable regardless of the patients' age and pre-diagnosis health status. B-cell maturation antigen (BCMA) and CS1 (also known as signaling lymphocytic activation molecule family member 7, or SLAMF7) are two surface antigens found on MM cells. Several clinical trials have shown that T cells expressing chimeric antigen receptors (CARs) that target BCMA can achieve complete remission in the treatment of MM (Ali et al., 2016; Cohen et al., 2016; Berdeja et al., 2017). However, relapse is common, including the outgrowth of both BCMA+ and BCMA− tumor cells (Ali et al., 2016; Cohen et al., 2016; Berdeja et al., 2017). Relapse with BCMA+ tumor suggests insufficient efficacy of the anti-BCMA CAR-T cells, and the outgrowth of BCMA− tumor indicates susceptibility to antigen escape (i.e., loss of the tumor antigen targeted by the therapeutic T cells). CARs targeting CS1 have also been evaluated in both T cells and natural killer (NK) cells for the treatment of MM in pre-clinical settings, but no clinical data have been reported. Furthermore, CS1 is expressed on the surface of T cells, and the expression of highly potent anti-CS1 CARs has been shown to cause T cell fratricide (Gogishvili et al., 2017). This behavior necessitates fine-tuning of CAR signaling in response to CS1 in order to achieve therapeutic efficacy against tumor cells without eliminating T cells.

Due to the high incidence of relapse, there is a need in the art for more effective therapies for treating multiple myeloma.

### SUMMARY OF THE DISCLOSURE

The current disclosure fulfills the aforementioned need in the art by providing single-chain chimeric antigen receptors (CARs) that simultaneously target BCMA and CS1 for the treatment of cancer. Accordingly, certain aspects of the disclosure relate to treating multiple myeloma, which is particularly difficult. Further embodiments relate to monospecific BCMA CAR polypeptides. Compositions and methods concerning polypeptides that are bispecific chimeric antigen receptors binding both BCMA and CS1 through a single CAR polypeptide are provided as a solution for treating cancer, particularly multiple myeloma. Embodiments include bispecific BCMA/CS1 CAR and/or monospecific BCMA CAR polypeptides, nucleic acids encoding bispecific BCMA/CS1 and/or monospecific BCMA CAR polypeptides, vectors comprising nucleic acids encoding bispecific BCMA/CS1 and/or monospecific BCMA CAR polypeptides, cells containing nucleic acids or vectors encoding bispecific BCMA/CS1 and/or monospecific BCMA CAR polypeptides, cells expressing bispecific BCMA/CS1 or monospecific BCMA CAR polypeptides on their surface, pharmaceutical compositions comprising cells expressing bispecific BCMA/CS1 or monospecific BCMA CAR polypeptides on their surface, methods of making bispecific BCMA/CS1 or monospecific BCMA CAR polypeptides and cells capable of expressing bispecific BCMA/CS1 or monospecific BCMA CAR polypeptides, methods of making T cells and natural killer cells expressing bispecific BCMA/CS1 or monospecific BCMA CAR polypeptides, and methods of treating a patient with compositions encoding, expressing, and/or comprising bispecific BCMA/CS1 or monospecific BCMA CAR polypeptides and expressing bispecific BCMA/CS1 or monospecific BCMA CAR polypeptides.

The bispecific BCMA/CS1 CAR polypeptides provided herein are CAR polypeptides that target both BCMA and CS1 with the functional domains of a single CAR. This is distinct from a polypeptide that comprises one CAR targeting BCMA and another separate CAR targeting CS1, which is a dual CAR polypeptide that may or may not be separated into two distinct CAR polypeptides by cleaving an amino acid linker between the two distinct CAR polypeptides that are capable of each acting as a CAR (one targeting BCMA and the other targeting CS1). The bispecific BCMA/CS1 CAR described herein has superior properties over dual CARs, such as the prevention of antigen escape and other advantages, as described herein.

The CAR molecules discussed herein have the three main regions of a CAR molecule, which are an extracellular domain that binds to one or more target molecule(s), a cytoplasmic region that contains a primary intracellular signaling domain, and a transmembrane region between the extracellular domain and the cytoplasmic domain. Some CAR molecules have a spacer that is between the extracellular domain and the transmembrane domain. Furthermore, one or more linkers may be included in CAR molecules between or within one or more regions, such as between different binding regions within the extracellular domain or within a binding region, such as between the variable region of a light chain (VH) and the variable region of a heavy chain (VL). Any embodiment regarding a specific region may be implemented with respect to any other specific region disclosed herein. Specific regions that can be implemented with any other specific region include, but are not limited to, the following: extracellular domain; BCMA-binding region; BCMA/CS1 loop; BCMA-specific scFv; complementarity-determining region 1 (CDR1), complementarity-determining region 2 (CDR2), and/or complementarity-determining region 3 (CDR3) of an anti-BCMA antibody heavy chain; CDR1, CDR2, and/or CDR3 of an anti-BCMA antibody light chain; VL from an anti-BCMA antibody; VH from an anti-BCMA antibody; murine or humanized c11D5.3 scFv; CDR1, CDR2, and/or CDR3 of murine or humanized c11D5.3 scFv or antibody heavy chain; CDR1, CDR2, and/or CDR3 of murine or humanized c11D5.3 scFv or antibody light chain; VL from murine or humanized c1D5.3 scFv or antibody; VH from murine or humanized c11D5.3 scFv or antibody; murine or humanized J22.9-xi scFv; CDR1, CDR2, and/or CDR3 of murine or humanized J22.9-xi scFv or antibody heavy chain; CDR1, CDR2, and/or CDR3 of murine or humanized J22.9-xi scFv or antibody light chain; VL from murine or humanized J22.9-xi scFv or antibody; VH from murine or humanized J22.9-xi scFv or antibody; APRIL fragment or dAPRIL region; CS1-binding region; CS1-specific scFv; CDR1, CDR2, and/or CDR3 of an anti-CS1 antibody heavy chain; CDR1, CDR2, and/or CDR3 of an anti-CS1 antibody light chain; VL from an anti-CS1 antibody; VH from an anti-CS1 antibody; Luc90 scFv; CDR1, CDR2, and/or CDR3 of Luc90 scFv or antibody heavy chain; CDR1, CDR2, and/or CDR3 of Luc90 scFv or antibody light chain; VL from Luc90 scFv or antibody; VH from Luc90 scFv or antibody; huLuc63 scFv; CDR1, CDR2, and/or CDR3 of huLuc63 scFv or antibody heavy chain; CDR1, CDR2, and/or CDR3 of huLuc63 scFv or antibody light chain; VL from huLuc63 scFv or antibody; VH from huLuc63 scFv or antibody; linker, extracellular spacer, transmembrane domain, cytoplasmic domain; intracellular signaling domain; primary intracellular signaling domain; costimulatory domain; tag; detection peptide, and leader peptide. Any of these regions may be immediately adjacent either on the N-terminal side or the C-terminal side of another region depending on its function but it is also contemplated that there may be intervening amino acids between contiguous regions that are at least or at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 amino acids in length (or any range derivable therein).

Further aspects relate to a chimeric antigen receptor (CAR) comprising: a) an extracellular binding domain comprising a BCMA-binding region and an extracellular spacer of SEQ ID NO:172 or 73; b) a single transmembrane domain; and, c) a single cytoplasmic region comprising a primary intracellular signaling domain. Yet further aspects relate to a chimeric antigen receptor (CAR) comprising: a) an extracellular binding domain comprising a BCMA scFv of SEQ ID NO:22 or 25 and an extracellular spacer of SEQ ID NO:172; b) a single transmembrane domain of SEQ ID NO:76; and, c) a cytoplasmic region comprising a costimulatory domain of SEQ ID NO:77 and a primary intracellular signaling domain of SEQ ID NO:78.

Method aspects of the disclosure relate to the use of the CAR molecules, compositions, and cells of the disclosure for the treatment of cancer. In some embodiments, the cancer comprises a blood cancer. In some embodiments, the cancer comprises a BCMA+ cancer, wherein a BCMA+ cancer is one that comprises BCMA+ cells or comprises at least 0.5, 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, or 90% BCMA+ cancer cells in a population of tumor cells. In some embodiments, the cancer comprises multiple myeloma.

Throughout the disclosure, the references made to c11D5.3 may refer to the murine or humanized c11D5.3 antibody or antibody derivative or domain, such as scFv, CDR, or variable region. Similarly, references made to J22.9xi may refer to the murine or humanized J22.9xi antibody or antibody derivative or domain, such as scFv, CDR, or variable region.

The CAR polypeptides of the current disclosure may have a region, domain, linker, spacer, or other portion thereof that comprises or consists of an amino acid sequence that is at least, at most, or exactly 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical (or any range derivable therein) to all or a portion of the amino acid sequences described herein. In certain embodiments, a CAR polypeptide comprises or consists of an amino acid sequence that is, is at least, is at most, or exactly 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% identical (or any range derivable therein) to any one of SEQ ID NOs:1-13 or 169-171.

In some embodiments, there is a bispecific chimeric antigen receptor comprising a) a bispecific extracellular binding domain comprising i) one or more BCMA-binding regions, and ii) one or more CS1-binding regions separated by one or more linkers; b) a single transmembrane domain; and, c) a single cytoplasmic region comprising a primary intracellular signaling domain. In some embodiments, the bispecific extracellular binding domain a) comprises a BCMA/CS1 loop. In some embodiments, the bispecific extracellular binding domain a) comprises i) a BCMA-binding region, that is a single-chain variable fragment (scFv) or a binding region of a proliferation-inducing ligand (dAPRIL) and ii) a CS1-specific scFv separated by a linker. The bispecific CARs disclosed herein are understood to have a single extracellular domain that is capable of binding BCMA and CS1, a transmembrane domain, and an intracellular domain.

In particular embodiments, there is a bispecific chimeric antigen receptor comprising: a) a bispecific extracellular binding domain comprising both i) a BCMA-binding region that is a single-chain variable fragment (scFv) or a binding region of A PRoliferation-Inducing Ligand (APRIL) and ii) a CS1-specific scFv separated by a linker; b) a single transmembrane domain; and, c) a single cytoplasmic region comprising a primary intracellular signaling domain.

In additional embodiments, there is a bispecific chimeric antigen receptor (CAR) comprising: i) a bispecific extracellular binding domain comprising one or more BCMA− binding regions and one or more CS1-binding regions separated by one or more linkers; wherein the BCMA-binding regions comprise CDR1, CDR2, and CDR3 from the heavy and light chains of the C11D5.3 or J22.9-xi antibody and wherein the CS1-binding regions comprise CDR1, CDR2, and CDR3 from the heavy and light chains of the Luc90 or huLuc63 antibody and wherein the linker comprises G4S; ii) a hinge spacer between 8-300 amino acids in length; iii) one CD28 transmembrane domain; and, iv) one cytoplasmic region comprising 4-1BB co-stimulatory domain and CD3zeta intracellular signaling domain.

Some aspects concern a bispecific chimeric antigen receptor (CAR) comprising: i) a bispecific extracellular binding domain comprising a BCMA-binding region comprising a binding region of dAPRIL and a CS1-specific scFv separated by a linker; wherein the CS1-specific scFv comprises CDR1, CDR2, and CDR3 from the heavy and light chains of the Luc90 or huLuc63 antibody and wherein the linker comprises (G4S)ₙ and n is 1, 2, 3, 4, 5, or 6; ii) a hinge spacer between 8-300 amino acids in length; iii) one CD28 transmembrane domain; and, iv) one cytoplasmic region comprising 4-1BB co-stimulatory domain and CD3zeta intracellular signaling domain.

The extracellular binding domain for a CAR has a polypeptide region that binds BCMA (BCMA-binding region) and another polypeptide region that binds CS1 (CS1-binding region). It is contemplated that in some embodiments, the CS1 binding region is membrane proximal, meaning its amino acid sequence is closer to the amino acid sequence of the transmembrane domain than the amino acid sequence of the BCMA binding region, which would then be considered membrane distal. In other embodiments, the BCMA-binding region is membrane proximal and the CS1-binding region is membrane distal.

The BCMA-binding region and the CS1-binding region have a linker separating them in certain embodiments. In certain embodiments, the BCMA-binding region comprises a BCMA-binding scFv and the CS1-binding region comprises a CS1-binding scFv. In some additional or alternative embodiments, the BCMA-binding scFv and the CS1-binding scFv each comprise a VH and VL. In some embodiments, the VH and VL of the BCMA-binding region and/or the VH and VL of the CS1-binding region are separated by a linker. The order of the variable regions can be VH-VL, while in other embodiments it is VL-VH. It is contemplated that a polypeptide may comprise multiple linkers such as 1, 2, 3, 4, 5 or more linkers. The linker is, is at least, or is at most 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 amino acids (or any range derivable therein) in length. In certain embodiments, the linker is 4-40 amino acids in length. It is contemplated that a linker may separate any domain/region in the CAR polypeptides described herein. In some embodiments, the linker is composed of only glycine and serine residues (a glycine-serine linker). In some embodiments, the linker comprises or consists of (G4S)ₙ, wherein n is 1, 2, 3, 4, 5, or 6. In other embodiments, the linker is (EAAAK)ₙ, wherein n is 1, 2, 3, 4, 5, or 6.

It is contemplated that the BCMA-binding region and/or CS1-binding region of the disclosure may be an scFv, a BCMA/CS1 loop, a single-antibody domain (e.g., a nanobody VH), or a diabody. In some embodiments, the BCMA-binding region and/or the CS1 binding region comprises an scFv. In some embodiments, the BCMA and CS1 binding regions comprise a BCMA/CS1 loop. In some embodiments, the BCMA-binding region and/or the CS1 binding region comprises a nanobody.

In some embodiments, the bispecific CAR comprises a BCMA/CS1 loop. In specific embodiments, the BCMA/CS1 loop comprises, from amino to carboxy end or from carboxy to amino end, a CS1 VL, a BCMA VH, a BCMA VL, and a CS1 VH. In specific embodiments, the BCMA/CS1 loop comprises, from amino to carboxy end or from carboxy to amino end, a CS1 VH, a BCMA VH, a BCMA VL, and a CS1 VL. In specific embodiments, the BCMA/CS1 loop comprises, from amino to carboxy end or from carboxy to amino end, a CS1 VL, a BCMA VL, a BCMA VH, and a CS1 VH. In specific embodiments, the BCMA/CS1 loop comprises, from amino to carboxy end or from carboxy to amino end, a CS1 VH, a BCMA VL, a BCMA VH, and a CS1 VL. In specific embodiments, the BCMA/CS1 loop comprises, from amino to carboxy end or from carboxy to amino end, a BCMA VL, a CS1 VH, a CS1 VL, and a BCMA VH. In specific embodiments, the BCMA/CS1 loop comprises, amino to carboxy end or from carboxy to amino end, a BCMA VH, a CS1 VH, a CS1 VL, and a BCMA VL. In specific embodiments, the BCMA/CS1 loop comprises, amino to carboxy end or from carboxy to amino end, a BCMA VL, a CS1 VL, a CS1 VH, and a BCMA VH. In specific embodiments, the BCMA/CS1 loop comprises, from amino to carboxy end or from carboxy to amino end, a BCMA VH, a CS1 VL, a CS1 VH, and a BCMA VL. Each VH and VL region may be separated from another VH and VL region by at least one or more linkers. In some embodiments, the linker comprises (G4S)₄. In some embodiments, the linker comprises SEQ ID NO:173. Also included in the embodiments of the disclosure are nucleic acids encoding the BCMA/CS1 loop molecules and/or loop CAR molecules as well as compositions and cells comprising the BCMA/CS1 loop CAR polypeptides or nucleic acids.

In some embodiments, a BCMA-binding region comprises all or part of the antigen binding portion of an anti-BCMA antibody. Many embodiments will comprise an anti-BCMA monoclonal antibody. In some embodiments, a BCMA-binding region comprises all or part of a variable heavy chain and/or a variable light chain from an anti-BCMA antibody. In certain embodiments, a BCMA-binding region comprises a BCMA-specific scFv.

“Single-chain Fv” or “scFv” antibody fragments comprise at least a portion of the VH and VL domains of an antibody, such as the CDRs of each, wherein these domains are present in a single polypeptide chain. It is contemplated that an scFv includes a CDR1, CDR2, and/or CDR3 of a heavy chain variable region and a CDR1, CDR2, and/or CDR3 of a light chain variable region in some embodiments. It is further contemplated that a CDR1, CDR2, or CDR3 may comprise or consist of a sequence set forth in a SEQ ID NO provided herein as CDR1, CDR2, or CDR3, respectively. A CDR may also comprise 1, 2, 3, 4, 5, 6, 7, 8, 9, 10 or more contiguous amino acid residues (or any range derivable therein) flanking one or both sides of a particular CDR sequence; therefore, there may be one or more additional amino acids at the N-terminal or C-terminal end of a particular CDR sequence, such as those shown in Tables 2-5. Any light-chain CDR1 (LCDR1) shown in Tables 2-5 may be substituted for any other LCDR1 shown in Tables 2-5 for the same binding target. The same is true for LCDR2, LCDR3, heavy-chain CDR1 (HCDR1), HCDR2, and HCDR3 shown in Tables 2-5.

It is also contemplated that an scFv may comprise more than the CDRs of a light chain variable region and/or a heavy chain region. In some embodiments, all or part of a light chain variable region and/or all or part of a heavy chain variable region is included in an scFv that is part of a binding domain. In some embodiments, the order is VH-VL, while in other embodiments, the order is VL-VH. Moreover, a VH, VL, VH-VL, or VL-VH sequence provided herein may have 1, 2, 3, 4, 5, 6, 7, 8, 9, 10 or more additions, deletions, and/or substitutions, particularly if such changes do not alter the CDRs of the light and heavy variable chain regions.

A “BCMA-specific scFv” comprises at least a portion of the VH and VL domains, such as the CDRs, of an antibody that binds BCMA. In some embodiments, a CAR comprises a BCMA-specific scFv comprising either i) CDR1, CDR2, and/or CDR3 of both the heavy and light variable chains from murine or humanized c11D5.3 antibody or murine or humanized J229-xi antibody or ii) the heavy and light chain variable regions from murine or humanized c11D5.3 antibody or murine or humanized J229-xi antibody.

In particular embodiments, the BCMA-binding regions comprise heavy-chain CDR1 (SEQ ID NO:14), CDR2 (SEQ ID NO:15), and CDR3 (SEQ ID NO:16) from anti-BCMA antibody c11D5.3. In additional or alternative embodiments, the BCMA-binding regions comprise light-chain CDR1 (SEQ ID NO:18), CDR2 (SEQ ID NO:19), and CDR3 (SEQ ID NO:20) from anti-BCMA antibody c11D5.3. In certain embodiments, the BCMA− binding regions comprise heavy-chain CDR1 (SEQ ID NO:14), CDR2 (SEQ ID NO:15), and CDR3 (SEQ ID NO:16) and light-chain CDR1 (SEQ ID NO:18), CDR2 (SEQ ID NO:19), and CDR3 (SEQ ID NO:20) from anti-BCMA antibody c11D5.3. In further embodiments, the BCMA-binding regions comprise a heavy-chain variable region from murine anti-BCMA antibody c11D5.3 (SEQ ID NO:17). In other embodiments, the BCMA-binding regions comprise a light-chain variable region from murine anti-BCMA antibody c11D5.3 (SEQ ID NO:21). Some CAR molecules have a BCMA-binding region that comprises a variable region comprising SEQ ID NO:22, which is VL-VH of murine anti-BCMA antibody c11D5.3.

In certain embodiments, the BCMA-binding regions comprise a humanized variable region from an anti-BCMA antibody. In particular embodiments a heavy-chain variable region and/or a light-chain variable region from anti-BCMA antibody is/are humanized, such as the murine anti-BCMA antibody c11D5.3. In some embodiments, a humanized heavy-chain variable region comprises the amino acid sequence of SEQ ID NO:23, which is a humanized c11D5.3 VH. In other embodiments, a humanized light-chain variable region of an anti-BCMA antibody comprises the amino acid sequence of SEQ ID NO:24, which is a humanized c11D5.3 VL. In further embodiments, the BCMA-binding regions comprise a humanized variable heavy chain and humanized variable light chain from murine anti-BCMA antibody c11D5.3. In specific embodiments, the BCMA-binding regions comprise the amino acid sequence of the variable regions of the humanized heavy and light chains of c11D5.3 (SEQ ID NO:25).

Other BCMA-binding regions used in CAR molecules are based on the J22.9-xi antibody. In some embodiments, the BCMA-binding regions comprise heavy-chain CDR1 (SEQ ID NO:26), CDR2 (SEQ ID NO:27), and CDR3 (SEQ ID NO:28) from anti-BCMA antibody J22.9-xi. Additionally or alternatively, the BCMA-binding regions comprise light-chain CDR1 (SEQ ID NO:30), CDR2 (SEQ ID NO:31), and CDR3 (SEQ ID NO:32) from anti-BCMA antibody J22.9-xi. In some embodiments, the BCMA-binding regions comprise heavy-chain CDR1 (SEQ ID NO:26), CDR2 (SEQ ID NO:27), and CDR3 (SEQ ID NO:28) and light-chain CDR1 (SEQ ID NO:30), CDR2 (SEQ ID NO:31), and CDR3 (SEQ ID NO:32) from anti-BCMA antibody J22.9-xi. In further embodiments, the BCMA-binding regions comprise a heavy-chain variable region from murine anti-BCMA antibody J22.9-xi (SEQ ID NO:29). In other embodiments, the BCMA-binding regions comprise a light-chain variable region from murine anti-BCMA antibody J22.9-xi (SEQ ID NO:33). In particular embodiments, the BCMA-binding regions comprise a variable region comprising SEQ ID NO:34, which is the VL-VH region of murine anti-BCMA antibody J22.9-xi.

In certain embodiments, a BCMA-binding region comprises a BCMA-specific scFv or loop that comprises a humanized heavy-chain and/or light chain variable region from humanized anti-BCMA antibody J22.9-xi. In some embodiments, a humanized heavy-chain variable region comprises the nucleic-acid sequence of SEQ ID NO:35 (J22.9-xi human VH). In some embodiments, a humanized light-chain variable region comprises the nucleic-acid sequence of SEQ ID NO:36 (J22.9-xi human VL). In further embodiments, the BCMA− binding regions comprise a humanized variable heavy chain and humanized variable light chain from anti-BCMA antibody J22.9-xi murine. In a specific embodiment, the BCMA− binding regions comprise the amino acid sequence of SEQ ID NO:37, which is a humanized VL-VH region of J22.9-xi).

In some cases, a BCMA-binding region includes all or part of a BCMA ligand and not a portion of an anti-BCMA antibody. In certain embodiments, the BCMA ligand is A PRoliferation Inducing Ligand (APRIL). APRIL binds both BCMA and transmembrane activator and cyclophilin ligand (TACI). As a result, using an APRIL-based CAR may provide a safeguard against antigen escape because it would bind to the tumor cell by TACI even if BCMA has been lost. In particular embodiments, a BCMA-binding region contains a derivative APRIL (“dAPRIL”), which comprises a part of APRIL that is involved in BCMA binding. In some embodiments, dAPRIL is a full-length APRIL molecule from which the N-terminal proteoglycan binding domain has been removed. This proteoglycan binding region is not essential for BCMA or TACI-binding and has been shown to promote APRIL-induced tumor-cell proliferation. In particular embodiments, dAPRIL is, is at least, or is at most 70, 75, 76, 77, 78, 79, 80, 81, 82, 83, 84, 85, 86, 87, 88, 89, 90, 91, 92, 93, 94, 95, 96, 97, 98, 99, or 100% (or any range derivable therein) to SEQ ID NO:38 or contains 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120 contiguous amino acids of SEQ ID NO:38. It is contemplated that there may be up to 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more amino acid substitutions, insertions, and/or deletions with respect to SEQ ID NO:38. In certain embodiments, a substitution is a conservative substitution based on the BLOSUM62 matrix of amino acids scoring 0 or higher. In some embodiments dAPRIL comprises or consists of SEQ ID NO:38.

It is contemplated that the BCMA-binding region of a CAR molecule may have an amino acid sequence that has, has at least or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, or contiguous amino acid deletions with respect to any of SEQ ID NOs:14-38, 57-70, or 79-83. Alternatively, the BCMA-binding region of a CAR molecule may have an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOs: 14-38, 57-70, or 79-83. Moreover, in some embodiments, the BCMA-binding region comprises an amino acid region of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 in any of SEQ ID NOs: 14-38, 57-70, or 79-83 (where position 1 is at the N-terminus of the SEQ ID NO).

A “CS1-specific scFv” comprises at least a portion of the VH and VL domains, such as the CDRs, of an antibody that binds CS1. In some embodiments, a bispecific CAR comprises a CS1-specific scFv comprising either i) CDR1, CDR2, and/or CDR3 of both the heavy and light variable chains from the Luc90 antibody or the huLuc63 antibody or ii) the heavy and light chain variable regions from the Luc90 antibody or huLuc63 antibody.

In particular embodiments, the CS1-binding regions comprise heavy-chain CDR1 (SEQ ID NO:39), CDR2 (SEQ ID NO:40), and CDR3 (SEQ ID NO:41) from murine anti-CS1 antibody Luc90. In additional or alternative embodiments, the CS1-binding regions comprise light-chain CDR1 (SEQ ID NO:43), CDR2 (SEQ ID NO:44), and CDR3 (SEQ ID NO:45) from murine anti-CS1 antibody Luc90. In certain embodiments, the CS1-binding regions comprise heavy-chain CDR1 (SEQ ID NO:39), CDR2 (SEQ ID NO:40), and CDR3 (SEQ ID NO:41) and light-chain CDR1 (SEQ ID NO:43), CDR2 (SEQ ID NO:44), and CDR3 (SEQ ID NO:45) from murine anti-CS1 antibody Luc90. In further embodiments, the CS1-binding regions comprise a heavy-chain variable region from murine anti-CS1 antibody Luc90 (SEQ ID NO:42). In other embodiments, the CS1-binding regions comprise a light-chain variable region from murine anti-CS1 antibody Luc90 (SEQ ID NO:46). Some bispecific CAR molecules have one or more CS1-binding regions comprising a variable region comprising SEQ ID NO:47, which is VH-VL of murine anti-CS1 antibody Luc90. It is contemplated that Luc90 may be humanized. In some embodiments, the CS1-binding regions comprise the CDRs of Luc90 but other parts of the variable region for the heavy and light chains are humanized.

In further embodiments, the CS1-binding regions comprise heavy-chain CDR1 (SEQ ID NO:48), CDR2 (SEQ ID NO:49), and CDR3 (SEQ ID NO:50) from anti-CS1 antibody huLuc63 (humanized Luc63 antibody). In additional or alternative embodiments, the CS1-binding regions comprise light-chain CDR1 (SEQ ID NO:52), CDR2 (SEQ ID NO:53), and CDR3 (SEQ ID NO:54) from anti-CS1 antibody huLuc63. In certain embodiments, the CS1-binding regions comprise heavy-chain CDR1 (SEQ ID NO:48), CDR2 (SEQ ID NO:49), and CDR3 (SEQ ID NO:50) and light-chain CDR1 (SEQ ID NO:52), CDR2 (SEQ ID NO:53), and CDR3 (SEQ ID NO:54) from anti-CS1 antibody huLuc63. In further embodiments, the CS1-binding regions comprise a heavy-chain variable region from anti-CS1 antibody huLuc63 (SEQ ID NO:51). In other embodiments, the CS1-binding regions comprise a light-chain variable region from anti-CS1 antibody Luc90 (SEQ ID NO:55). Some CAR molecules have a CS1-binding region(s) comprising a variable region comprising SEQ ID NO:56, which is VH-VL of anti-CS1 antibody huLuc63.

It is contemplated that the CS1-binding region(s) of a CAR molecule may have an amino acid sequence that has, has at least or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, or contiguous amino acid deletions with respect to any of SEQ ID NOs:39-56, 71, or 72. Alternatively, the CS1-binding region of a bispecific CAR molecule may have an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOs:39-56, 71, or 72. Moreover, in some embodiments, the CS1-binding region comprises an amino acid region of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 in any of SEQ ID NOS:39-56, 71, or 72 (where position 1 is at the N-terminus of the SEQ ID NO).

It is contemplated that a nucleic acid molecule of the disclosure, such as those described in SEQ ID NOS:153-168 and other nucleic acids encoding any of the following as described herein: extracellular domain; BCMA-binding region; BCMA-specific scFv; complementarity-determining region 1 (CDR1), complementarity-determining region 2 (CDR2), and/or complementarity-determining region 3 (CDR3) of an anti-BCMA antibody heavy chain; CDR1, CDR2, and/or CDR3 of an anti-BCMA antibody light chain; VL from an anti-BCMA antibody; VH from an anti-BCMA antibody; murine or humanized c11D5.3 scFv or antibody; CDR1, CDR2, and/or CDR3 of murine or humanized c11D5.3 scFv or antibody heavy chain; CDR1, CDR2, and/or CDR3 of murine or humanized c11D5.3 scFv or antibody light chain; VL from murine or humanized c11D5.3 antibody or scFv; VH from murine or humanized c11D5.3 antibody or scFv; murine or humanized J22.9-xi scFv; CDR1, CDR2, and/or CDR3 of murine or humanized J22.9-xi scFv or antibody heavy chain; CDR1, CDR2, and/or CDR3 of murine or humanized J22.9-xi scFv or antibody light chain; VL from murine or humanized J22.9-xi scFv or antibody; VH from murine or humanized J22.9-xi antibody or scFv; APRIL fragment or dAPRIL region; CS1-binding region; CS1-specific scFv; CDR1, CDR2, and/or CDR3 of an anti-CS1 antibody heavy chain; CDR1, CDR2, and/or CDR3 of an anti-CS1 antibody light chain; VL from an anti-CS1 antibody; VH from an anti-CS1 antibody; Luc90 scFv; CDR1, CDR2, and/or CDR3 of Luc90 scFv or antibody heavy chain; CDR1, CDR2, and/or CDR3 of Luc90 scFv or antibody light chain; VL from Luc90 scFv or antibody; VH from Luc90 scFv or antibody; huLuc63 scFv; CDR1, CDR2, and/or CDR3 of huLuc63 scFv or antibody heavy chain; CDR1, CDR2, and/or CDR3 of huLuc63 scFv or antibody light chain; VL from huLuc63 scFv or antibody; VH from huLuc63 scFv or antibody; linker, extracellular spacer, transmembrane domain, cytoplasmic domain; intracellular signaling domain; primary intracellular signaling domain; costimulatory domain; tag; detection peptide, or leader peptide has a nucleic acid sequence that has, has at least or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more substitutions, contiguous nucleic acid additions, and/or contiguous nucleic acid deletions (or any range derivable therein) with respect to any of SEQ ID NOs: 153-168. Alternatively, the nucleic acid molecules of the disclosure may have a nucleic acid sequence that comprises or consists of a nucleic acid that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOs:153-168. Moreover, in some embodiments, the nucleic acid molecule of the disclosure comprises a nucleic acid of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more contiguous nucleic acids (or any range derivable therein) starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 in any of SEQ ID NOs:153-168 (where position 1 is at the 5′ of the SEQ ID NO). Furthermore, it is also comtemplated that the nucleic acids of the disclosure may be codon optimized. In some embodiments, a nucleic acid molecule of the disclosure may encode for a polypeptide that has an amino acid sequence that has, has at least, or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, and/or contiguous amino acid deletions (or any range derivable therein) with respect to any of the peptide and polypeptides shown in Tables 1-7 or polypeptides described herein. Further embodiments provide for nucleic acid molecules of the disclosure that encode for a polypeptide having an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of the peptide and polypeptides shown in Tables 1-7 or polypeptides described herein. Moreover, in some embodiments, the nucleic acid of the disclosure encodes for an amino acid of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more contiguous amino acids (or any range derivable therein) starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, or 195 in any of the peptide and polypeptides shown in Tables 1-7 or polypeptides described herein (where position 1 is at the N-terminus of the SEQ ID NO).

Different combinations of particular BCMA-binding regions coupled with specific CS1-binding regions are contemplated. In some CAR molecules, the BCMA-binding region comprises dAPRIL or comprises CDR1, CDR2, and/or CDR3 of both the variable heavy and light chains or the heavy and light chain variable regions from c11D5.3, and/or J229-xi scFv or antibody; and the CS1-binding region comprises CDR1, CDR2, and/or CDR3 of both the variable heavy and light chains or the heavy and light chain variable regions from hu (humanized) Luc63 and/or Luc90 scFv or antibody.

In specific embodiments, the BCMA-binding region comprises dAPRIL and the CS1-binding region comprises CDR1, CDR2, and/or CDR3 of both the variable heavy and light chains or the heavy and light chain variable regions from huLuc63 antibody. In other embodiments, the BCMA-binding region comprises dAPRIL and the CS1-binding region comprises CDR1, CDR2, and/or CDR3 of both the variable heavy and light chains or the heavy and light chain variable regions from Luc90 antibody. In further embodiments, the BCMA-binding region comprises an scFv or loop comprising CDR1, CDR2, and/or CDR3 of both the variable heavy and light chains or the heavy and light chain variable regions from c11D5.3 antibody and the CS1-binding region comprises an scFv or loop comprising CDR1, CDR2, and/or CDR3 of both the variable heavy and light chains or the heavy and light chain variable regions from huLuc63 antibody. In other embodiments, the BCMA-binding region comprises CDR1, CDR2, and/or CDR3 of both the variable heavy and light chains or the heavy and light chain variable regions from c11D5.3 antibody and the CS1-binding region comprises CDR1, CDR2, and/or CDR3 of both the variable heavy and light chains or the heavy and light chain variable regions from Luc90 antibody. In further embodiments, the BCMA-binding region comprises CDR1, CDR2, and/or CDR3 of both the variable heavy and light chains or humanized heavy and humanized light chain variable regions based on c11D5.3 antibody and the CS1-binding region comprises CDR1, CDR2, and/or CDR3 of both the variable heavy and light chains or the heavy and light chain variable regions from huLuc63 antibody. In other embodiments, the BCMA-binding region comprises CDR1, CDR2, and/or CDR3 of both the variable heavy and light chains or humanized heavy and humanized light chain variable regions based on c11D5.3 antibody and the CS1-binding region comprises CDR1, CDR2, and/or CDR3 of both the variable heavy and light chains or the heavy and light chain variable regions from Luc90 antibody.

In further embodiments, the BCMA-binding region comprises CDR1, CDR2, and/or CDR3 of both the variable heavy and light chains or the heavy and light chain variable regions from murine or humanized J22.9.xi antibody and the CS1-binding region comprises CDR1, CDR2, and/or CDR3 of both the variable heavy and light chains or the heavy and light chain variable regions from huLuc63 antibody. In other embodiments, the BCMA− binding region comprises CDR1, CDR2, and/or CDR3 of both the variable heavy and light chains or the heavy and light chain variable regions from murine or humanized J22.9.xi antibody and the CS1-binding region comprises CDR1, CDR2, and/or CDR3 of both the variable heavy and light chains or the heavy and light chain variable regions from Luc90 antibody. In further embodiments, the BCMA-binding region comprises CDR1, CDR2, and/or CDR3 of both the variable heavy and light chains or humanized heavy and humanized light chain variable regions based on murine or humanized J22.9.xi antibody and the CS1-binding region comprises CDR1, CDR2, and/or CDR3 of both the variable heavy and light chains or the heavy and light chain variable regions from huLuc63 antibody. In other embodiments, the BCMA-binding region comprises CDR1, CDR2, and/or CDR3 of both the variable heavy and light chains or humanized heavy and humanized light chain variable regions based on murine or humanized J22.9.xi antibody and the CS1-binding region comprises CDR1, CDR2, and/or CDR3 of both the variable heavy and light chains or the heavy and light chain variable regions from Luc90 antibody.

Some CAR molecules comprise an extracellular spacer. The extracellular spacer is between the transmembrane domain and the extracellular domain. In some embodiments, the extracellular spacer comprises a hinge region. In some embodiments, the hinge is the hinge region of an IgG molecule. In some embodiments, the hinge is a hinge region known in the art or described herein. In some embodiments, the extracellular spacer comprises or further comprises a CH2CH3 region of an IgG molecule. In some embodiments, the extracellular spacer comprises one or more of a hinge region, CH1, CH2, and CH3 region. In some embodiments, the extracellular spacer comprises or consists of the IgG4 hinge. In some embodiments, the extracellular spacer comprises or consists of the IgG4 hinge and CH3. In some embodiments, the extracellular spacer comprises or consists of the IgG4 hinge, a CH2 region, and a CH3 region. In some embodiments, the extracellular spacer is derived from a hinge, CH1, CH2, and/or CH3 region or other region of an IgG1, IgG2, IgG3, IgG4, IgA1, IgA2, IgD, IgE, or IgM from human, mouse, rat, dog, donkey, goat, or rabbit. In some embodiments, the extracellular spacer comprises the hinge and CH2CH3 region of an IgG molecule. In some embodiments, the CH2CH3 region of an IgG molecule has additional L235E/N297Q or L235D/N297Q mutations to prevent Fc receptor binding. In some embodiments, the peptide spacer consists of the hinge region of an IgG molecule. In some embodiments, the peptide spacer is less than 30, 20, 15, 10, 9, 8, 7, 6, 5, or 4 amino acids. A spacer is, is at least, or is at most 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 441, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000 amino acids in length (and any range derivable therein). In certain embodiments, the extracellular spacer is between 8 and 1000 amino acids in length, between 8 and 500 amino acids in length, between 100-300 amino acids in length, or fewer than 100 amino acids in length. In specific embodiments, the extracellular spacer is an IgG4 hinge, a CD8a hinge, an IgG1 hinge, or a CD34 hinge. In additional embodiments, the extracellular spacer further comprises a CH1, CH2 and/or a CH3 domain (though CD8a hinge does not have CH2 and CH3 domains, so it may be excluded as further comprising one of these domains).

CAR molecules have a transmembrane domain between the extracellular domain and the cytoplasmic region (also referred to as an intracellular domain). Embodiments include a transmembrane domain that is an alpha or beta chain of the T cell receptor, CD28, CD3 epsilon, CD45, CD4, CD5, CD8, CD9, CD16, CD22, CD33, CD37, CD64, CD80, CD86, CD123, CD134, CD137 or CD154 transmembrane domain. In particular embodiments, the transmembrane domain is a CD28 transmembrane domain.

CAR molecules have a cytoplasmic region that mediates internal cell signaling. In particular embodiments, this is accomplished with the signaling domain from CD3ζ (zeta), which acts as a primary or main intracellular signaling domain. The bispecific CAR molecules provided herein have a single cytoplasmic region for both targeting regions (BCMA and CS1), as opposed to each targeting region having its own cytoplasmic region. A cytoplasmic region includes 1, 2, or 3 costimulatory domains in further embodiments. In particular embodiments, a cytoplasmic region comprises two costimulatory domains. In certain embodiments, a costimulatory domain is 4-1BB (CD137), CD28, IL-15Rα, OX40, CD2, CD27, CDS, ICAM-1, LFA-1 (CD11a/CD18), ICOS (CD278) though other costimulatory domains may also be included. In certain embodiments, the costimulatory domain is 4-1BB.

In further embodiments, a CAR molecule also comprises a tag that can be used to sort and/or identify the CAR molecule in a host cell. In some embodiments, the tag is further defined as a therapeutic control. In some embodiments, the tag or therapeutic control is less than a full-length polypeptide and is truncated. For instance, to remove one or more functional domains from the tag. In certain embodiments, the truncated protein is EGFR (EGFRt), which can be used to detect expression of the CAR. In other embodiments, the tag is colorimetric or fluorescent.

In some embodiments, there is a bispecific chimeric antigen receptor (CAR) comprising: i) a bispecific extracellular binding domain comprising a BCMA single-chain variable fragment (scFv) and a CS1-specific scFv separated by a linker; wherein the BCMA− specific scFv comprises CDR1, CDR2, and CDR3 from the heavy and light chains of the c11D5.3 or J22.9-xi antibody and wherein the CS1-specific scFv comprises CDR1, CDR2, and CDR3 from the heavy and light chains of the Luc90 or huLuc63 antibody and wherein the linker comprises G4S; ii) a hinge spacer between 8-300 amino acids in length; iii) one CD28 transmembrane domain; and, iv) one cytoplasmic region comprising 4-1BB co-stimulatory domain and CD3zeta intracellular signaling domain. In certain embodiments, the BCMA-specific scFv is membrane proximal, while in other embodiments, the BCMA− specific scFv is membrane distal.

In other embodiments, there is a bispecific chimeric antigen receptor (CAR) comprising: i) a bispecific extracellular binding domain comprising a BCMA-binding region comprising dAPRIL and a CS1-binding region separated by a linker; wherein the CS1-binding region comprises CDR1, CDR2, and CDR3 from the heavy and light variable chains of the Luc90 or huLuc63 antibody and wherein the linker comprises G4S; ii) a hinge spacer between 8-300 amino acids in length; iii) one CD28 transmembrane domain; and, iv) one cytoplasmic region comprising 4-1BB co-stimulatory domain and CD3zeta intracellular signaling domain. In some embodiments, dAPRIL is membrane proximal, while in other embodiments, dAPRIL is membrane distal. In specific embodiments, dAPRIL comprises or consists of the amino acid sequence of SEQ ID NO:38.

In particular embodiments, there is a bispecific chimeric antigen receptor (CAR) comprising in order from the amino to carboxy end of the CAR: i) a bispecific extracellular binding domain comprising a BCMA-specific scFv or loop comprising SEQ ID NO:25; a (G4S)₄ linker; and a CS1-specific scFv or loop comprising SEQ ID NO:56; ii) a hinge spacer comprising a IgG4 hinge with CH2 and/or CH3 regions and wherein the hinge spacer is between 100-250 amino acids in length; iii) one CD28 transmembrane domain; and, iv) one cytoplasmic region comprising 4-1BB co-stimulatory domain and CD3zeta intracellular signaling domain.

In further embodiments, there is a bispecific chimeric antigen receptor (CAR) comprising in order from the amino to carboxy end of the CAR: i) a bispecific extracellular binding domain comprising a CS1-specific scFv or loop comprising SEQ ID NO:56; a (G4S)₄ linker; and a BCMA-specific scFv or loop comprising SEQ ID NO:25; ii) a hinge spacer comprising a IgG4 hinge and wherein the spacer is between 4-50 amino acids in length; iii) one CD28 transmembrane domain; and, iv) one cytoplasmic region comprising 4-1BB co-stimulatory domain and CD3zeta intracellular signaling domain.

In a specific embodiment, there is a bispecific chimeric antigen receptor (CAR) comprising in order from the amino to carboxy end of the CAR: i) a bispecific extracellular binding domain comprising a BCMA-specific scFv or loop comprising SEQ ID NO:25; a (G4S)₄ linker; and a CS1-specific scFv or loop comprising SEQ ID NO:47; ii) a hinge spacer comprising a IgG4 hinge and wherein the spacer is between 4-50 amino acids in length; iii) one CD28 transmembrane domain; and, iv) one cytoplasmic region comprising 4-1BB co-stimulatory domain and CD3zeta intracellular signaling domain.

In some specific embodiments, the CAR comprises or consists of SEQ ID NO:1. In some specific embodiments, the CAR comprises or consists of SEQ ID NO:2. In some specific embodiments, the CAR comprises or consists of SEQ ID NO:3. In some specific embodiments, the CAR comprises or consists of SEQ ID NO:4. In some specific embodiments, the CAR comprises or consists of SEQ ID NO:5. In some specific embodiments, the CAR comprises or consists of SEQ ID NO:6. In some specific embodiments, the CAR comprises or consists of SEQ ID NO:7. In some specific embodiments, the CAR comprises or consists of SEQ ID NO:8. In some specific embodiments, the CAR comprises or consists of SEQ ID NO:9. In some specific embodiments, the CAR comprises or consists of SEQ ID NO:10. In some specific embodiments, the CAR comprises or consists of SEQ ID NO:11. In some specific embodiments, the CAR comprises or consists of SEQ ID NO:12. In some specific embodiments, the CAR comprises or consists of SEQ ID NO:13. In some specific embodiments, the CAR comprises or consists of SEQ ID NO:169. In some specific embodiments, the CAR comprises or consists of SEQ ID NO:170. In some specific embodiments, the CAR comprises or consists of SEQ ID NO:171.

In certain embodiments, polypeptides described throughout this disclosure are isolated, meaning it is not found in the cellular milieu. In some cases, they are purified, which means it is mostly if not completely separated from polypeptides having a different amino acid sequence and/or chemical formula. The polypeptides of the current disclosure may have one or more of an extracellular binding domain, a BCMA-binding region, a CS1-binding region, a proliferation-inducing agent binding region, a CS1-specific scFv, BCMA/CS1 loop a transmembrane domain, a cytoplasmic region, a linker, a BCMA-specific scFv, a heavy chain CDR1, CDR2, and/or CDR3, a light chain CDR1, CDR2, and/or CDR3, a dAPRIL fragment, an extracellular spacer, and a costimulatory domain that has at least, at most, or exactly 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identity (or any range derivable therein) to all or a portion of the amino acid sequences described herein.

In certain embodiments, polypeptides described throughout this disclosure are isolated, meaning they are not found in the cellular milieu. In some cases, they are purified, which means it is mostly if not completely separated from polypeptides having a different amino acid sequence and/or chemical formula.

Nucleic acids comprising a sequence that encodes the chimeric antigen receptors disclosed herein, and portions thereof, are provided in embodiments. A nucleic acid may comprise RNA or DNA. In certain embodiments, the nucleic acid is an expression construct. In some embodiments, the expression construct is a vector. In certain embodiments, the vector is a viral vector. The viral vector is a retroviral vector or derived from a retrovirus in particular embodiments. In some embodiments, the retroviral vector comprises a lentiviral vector or is derived from a lentivirus. It is noted that a viral vector is an integrating nucleic acid in certain embodiments. Additionally, a nucleic acid may be a molecule involved in gene editing such that a nucleic acid (such as a guide RNA) encoding a CAR is used to incorporate a CAR-coding sequence into a particular locus of the genome, such as the TRAC gene. This involves a gene editing system such as CRISPR/Cas9 in some embodiments. A nucleic acid, polynucleotide, or polynucleotide region (or a polypeptide or polypeptide region) has a certain percentage (for example, 60%, 65%, 70%, 75%, 80%, 85%, 90%, 95%, 98% or 99%—or any range derivable therein) of “sequence identity” or “homology” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. It is contemplated that a nucleic acid may have such sequence identity or homology to any nucleic acid SEQ ID NO provided herein.

In other embodiments, there is a cell or a population of cells comprising a nucleic acid that encodes all or part of any CAR discussed herein. In certain embodiments, a cell or population of cells contains within its genome a sequence encoding any of the CAR polypeptides described herein. This includes, but is not limited to, a lentivirus or retrovirus that has integrated into the cell's genome. In some embodiments, a cell or population of cells expresses all or part of any CAR discussed herein, including, but not limited to those with the amino acid sequence of any of SEQ ID NO: 1-13 or 169-171. Progeny (F1, F2, and beyond) of cells in which a nucleic acid encoding a CAR polypeptide was introduced are included in the cells or populations of cells disclosed herein. In some embodiments, a cell or population of cells is a T cell, a natural killer (NK) cell, a natural killer T cell (NKT), an invariant natural killer T cell (iNKT), stem cell, lymphoid progenitor cell, peripheral blood mononuclear cell (PBMC), bone marrow cell, fetal liver cell, embryonic stem cell, cord blood cell, induced pluripotent stem cell (iPS cell). Specific embodiments concern a cell that is a T cell or an NK cell. In some embodiments, T cell comprises a naïve memory T cell. In some embodiments, the naïve memory T cell comprises a CD4+ or CD8+ T cell. In some embodiments, the cells are a population of cells comprising both CD4+ and CD8+ T cells. In some embodiments, the cells are a population of cells comprising naïve memory T cells comprising CD4+ and CD8+ T cells. In some embodiments, the T cell comprises a T cell from a population of CD14 depleted, CD25 depleted, and/or CD62L enriched PBMCs. In embodiments involving a population of cells, the population is about, is at least about, or is at most about 10², 10⁴, 10⁴, 10⁵, 10⁶, 10⁷, 108, 10⁹, 10¹⁰, 10¹¹, 10¹² cells (or any range derivable therein. In certain embodiments, there are about 10³-10⁸ cells. In certain embodiments, cells are autologous with respect to a patient who will receive them. In other embodiments, cells are not autologous and may be allogenic.

In some aspects, the disclosure relates to a cell comprising one or more polypeptides described herein. In some embodiments, the cell is an immune cell. In some embodiments, the cell is a progenitor cell or stem cell. In some embodiments, the progenitor or stem cell is in vitro differentiated into an immune cell. In some embodiments, the cell is a T cell. In some embodiments, the cell is a CD4+ or CD8+ T cell. In some embodiments, the cell is a natural killer cell. In some embodiments, the cell is ex vivo. The term immune cells includes cells of the immune system that are involved in defending the body against both infectious disease and foreign materials. Immune cells may include, for example, neutrophils, eosinophils, basophils, natural killer cells, lymphocytes such as B cells and T cells, and monocytes. T cells may include, for example, CD4+, CD8+, T helper cells, cytotoxic T cells, 76 T cells, regulatory T cells, suppressor T cells, and natural killer T cells. In a specific embodiment, the T cell is a regulatory T cell.

Also included as an embodiment is a composition comprising the population of cells, wherein the composition is a pharmaceutically acceptable formulation.

Methods of making and using the chimeric antigen receptors, nucleic acids encoding such CARs, and cells and compositions containing these CARs are also provided. Methods include methods for making a cell that expresses a CAR, for treating a patient with cancer, for treating a patient with multiple myeloma, for developing a T cell or an NK cell that expresses a CAR, for expressing a bispecific BCMA/CS1 or monospecific BCMA CAR molecule; for generating a bispecific BCMA/CS1 or monospecific BCMA CAR molecule; and for targeting cells expressing BCMA and CS1.

Steps of methods include 1, 2, 3, 4, 5, 6, 7, 8, 9, 10 or more of the following steps: cloning regions of a BCMA/CS1 bispecific or BCMA monospecific CAR; introducing into a cell a nucleic acid that encodes a BCMA/CS1 bispecific or BCMA monospecific CAR; editing the genome of a cell to express a BCMA/CS1 bispecific or BCMA monospecific CAR; infecting a cell with a viral vector encoding a BCMA/CS1 bispecific or BCMA monospecific CAR; transfecting a cell with a guide RNA (gRNA) for editing a genome to express a BCMA/CS1 bispecific or BCMA monospecific CAR; culturing a cell or a population of cells; expanding a cell or a population of cells; differentiating a cell or a population of cells into a cell with one or more T cell or NK cell properties; culturing a cell with serum-free medium; culturing a cell under conditions to produce a T cell or NK cell; purifying cells that express BCMA/CS1 bispecific or BCMA monospecific CARs; administering cells expressing a BCMA/CS1 bispecific or BCMA monospecific CAR to a patient; obtaining cells from a patient; isolating cells from a patient; selecting cells that express a BCMA/CS1 bispecific or BCMA monospecific CAR; isolating cells using a sortable tag; detecting a tag associated with a BCMA/CS1 bispecific or BCMA monospecific CAR; measuring a tag associated with a BCMA/CS1 bispecific or BCMA monospecific CAR; or administering other cancer therapy to a patient in addition to administering cells that express BCMA/CS1 bispecific or BCMA monospecific CAR molecules.

In certain embodiments, there are methods of making a cell that expresses a chimeric antigen receptor comprising introducing into a cell a nucleic acid encoding one of the CAR molecules discussed herein or a nucleic acid that allows gene editing of the cell's genome to express one of the CAR molecules discussed herein. In certain embodiments, a cell is infected with a lentivirus encoding the CAR. In other embodiments a cell is a T cell, a natural killer (NK) cell, a natural killer T cell (NKT), an invariant natural killer T cell (iNKT), stem cell, lymphoid progenitor cell, peripheral blood mononuclear cell (PBMC), bone marrow cell, fetal liver cell, embryonic stem cell, cord blood cell, induced pluripotent stem cell (iPS cell). In cases where a cell is not yet a T cell or NK cell, a method may also include culturing the cell under conditions that promote the differentiation of the cell into a T cell or an NK cell. In additional embodiments, methods include culturing the cell under conditions to expand the cell before and/or after introducing the nucleic acid into the cell. In some embodiments, cells are cultured with serum-free medium.

Additional methods concern treating a patient with cancer comprising administering to the patient an effective amount of the composition comprising a cell population expressing a BCMA/CS1 bispecific or BCMA monospecific CAR or a cell population comprising a nucleic acid encoding a BCMA/CS1 bispecific or BCMA monospecific CAR. In some embodiments, the patient has a myeloma or lymphoma. In particular embodiments, a patient has multiple myeloma. In additional embodiments, a patient has relapsed multiple myeloma. Further embodiments include a step of administering an additional therapy to the patient. Further embodiments include a step of administering chemotherapy and/or radiation to the patient. In some embodiments, the additional therapy comprises an immunotherapy. In some embodiments, the additional therapy comprises an additional therapy described herein. In some embodiments, the immunotherapy comprises immune checkpoint inhibitor therapy. In some embodiments, the immunotherapy comprises an immunotherapy described herein. In some embodiments, the immune checkpoint inhibitor therapy comprises a PD-1 inhibitor. In some embodiments, the immune checkpoint inhibitor therapy comprises one or more inhibitors of one or more immune checkpoint proteins described herein.

In some embodiments, there are methods of treating a patient with multiple myeloma comprising administering to the patient a composition of the disclosure. Further embodiments relate to methods of treating a patient with multiple myeloma comprising administering to the patient a composition comprising a population of cells expressing a chimeric antigen receptor (CAR) comprising: a) a bispecific extracellular binding domain comprising i) a dAPRIL fragment or a BCMA single-chain variable fragment (scFv) and ii) a CS1-specific scFv separated by a linker; wherein the BCMA-specific scFv comprises CDR1, CDR2, and CDR3 from the heavy and light chains of C11D5.3 or J22.9-xi and wherein the CS1-specific scFv comprises CDR1, CDR2, and CDR3 from the heavy and light chains of Luc90 or huLuc63 and wherein the linker comprises G4S; b) a hinge spacer between 8-300 amino acids in length; c) one CD28 transmembrane domain; and, d) one cytoplasmic region comprising 4-1BB co-stimulatory domain and CD3zeta intracellular signaling domain. In some embodiments, cells are autologous.

In specific embodiments there are methods of treating a patient with multiple myeloma comprising administering to the patient a composition comprising a population of cells expressing a bispecific chimeric antigen receptor (CAR) comprising in order from the amino to carboxy end of the CAR: i) a bispecific extracellular binding domain comprising a BCMA-specific scFv comprising SEQ ID NO:24; a (G4S)₄ linker; and a CS1-specific scFv comprising SEQ ID NO:55 ii) a hinge spacer comprising a IgG4 hinge, CH2, and CH3 region and wherein the spacer is between 200-250 amino acids in length; iii) one CD28 transmembrane domain; and, iv) one cytoplasmic region comprising 4-1BB co-stimulatory domain and CD3zeta intracellular signaling domain.

In other embodiments, there are methods of treating a patient with multiple myeloma comprising administering to the patient a composition comprising a population of cells expressing a bispecific chimeric antigen receptor (CAR) comprising in order from the amino to carboxy end of the CAR: i) a bispecific extracellular binding domain comprising a CS1-specific scFv comprising SEQ ID NO:55; a (G4S)₄ linker; and a BCMA-specific scFv comprising SEQ ID NO:21; ii) a hinge spacer comprising a IgG4 hinge and CH2 region and wherein the spacer is between 100-150 amino acids in length; iii) one CD28 transmembrane domain; and, iv) one cytoplasmic region comprising 4-1BB co-stimulatory domain and CD3zeta intracellular signaling domain.

Some embodiments involve methods of treating a patient with multiple myeloma comprising administering to the patient a composition comprising a population of cells expressing a bispecific chimeric antigen receptor (CAR) comprising in order from the amino to carboxy end of the CAR: i) a bispecific extracellular binding domain comprising a BCMA− specific scFv comprising SEQ ID NO:25; a (G4S)₄ linker; and a CS1-specific scFv comprising SEQ ID NO:56 ii) a hinge spacer comprising a IgG4 hinge, CH2, and CH3 region and wherein the spacer is between 200-250 amino acids in length; iii) one CD28 transmembrane domain; and, iv) one cytoplasmic region comprising 4-1BB co-stimulatory domain and CD3zeta intracellular signaling domain.

In further embodiments, there are methods of treating a patient with multiple myeloma comprising administering to the patient a composition comprising a population of cells expressing a bispecific chimeric antigen receptor (CAR) comprising in order from the amino to carboxy end of the CAR: i) a bispecific extracellular binding domain comprising a CS1-specific scFv comprising SEQ ID NO:56; a (G4S)₄ linker; and a and BCMA-specific scFv comprising SEQ ID NO:25; ii) a hinge spacer comprising a IgG4 hinge and wherein the spacer is between 4-50 amino acids in length; iii) one CD28 transmembrane domain; and, iv) one cytoplasmic region comprising 4-1BB co-stimulatory domain and CD3zeta intracellular signaling domain.

In certain embodiments there are methods of treating a patient with multiple myeloma comprising administering to the patient a composition comprising a population of cells expressing a bispecific chimeric antigen receptor (CAR) comprising in order from the amino to carboxy end of the CAR: i) a bispecific extracellular binding domain comprising a BCMA-specific scFv comprising SEQ ID NO:25; a (G4S)₄ linker; and a CS1-specific scFv comprising SEQ ID NO:47 ii) a hinge spacer comprising a IgG4 hinge and wherein the spacer is between 4-50 amino acids in length; iii) one CD28 transmembrane domain; and, iv) one cytoplasmic region comprising 4-1BB co-stimulatory domain and CD3zeta intracellular signaling domain.

Further embodiments concern methods of treating a patient with multiple myeloma comprising administering to the patient a composition comprising a population of cells expressing a bispecific chimeric antigen receptor (CAR) comprising in order from the amino to carboxy end of the CAR: i) a bispecific extracellular binding domain comprising a BCMA− specific scFv comprising SEQ ID NO:24; a (G4S)₄ linker; and a CS1-specific scFv comprising SEQ ID NO:55; ii) a hinge spacer comprising a IgG4 hinge, CH2, and CH3 region and wherein the spacer is between 200-250 amino acids in length; iii) one CD28 transmembrane domain; and, iv) one cytoplasmic region comprising 4-1BB co-stimulatory domain and CD3zeta intracellular signaling domain.

Further aspects of the disclosure relate to methods for making the polypeptides of the disclosure comprising expressing a nucleotide encoding the polypeptide in a cell. Further aspects relate to cultured cells, frozen cells, suspended cells, or adhered cells comprising a CAR polypeptide described herein.

Aspects of the disclosure relate to a method for treating a disease or pathological condition comprising administering a cell of the disclosure to a patient. In some embodiments, the patient is a human patient.

In certain embodiments, there are methods of treating a patient with multiple myeloma comprising administering to the patient a composition comprising a population of cells expressing a bispecific chimeric antigen receptor (CAR) comprising in order from the amino to carboxy end of the CAR: i) a bispecific extracellular binding domain comprising a BCMA-specific scFv comprising SEQ ID NO:25; a (G4S)₄ linker; and a CS1-specific scFv comprising SEQ ID NO:56; ii) a hinge spacer comprising a IgG4 hinge, CH2, and CH3 region and wherein the spacer is between 200-250 amino acids in length; iii) one CD28 transmembrane domain; and, iv) one cytoplasmic region comprising 4-1BB co-stimulatory domain and CD3zeta intracellular signaling domain.

In other embodiments, there are methods of treating a patient with multiple myeloma comprising administering to the patient a composition comprising a population of cells expressing a bispecific chimeric antigen receptor (CAR) comprising in order from the amino to carboxy end of the CAR: i) a bispecific extracellular binding domain comprising a CS1-specific scFv comprising SEQ ID NO:56; a (G4S)₄ linker; a BCMA-specific scFv comprising SEQ ID NO:25; ii) a hinge spacer comprising a IgG4 hinge and wherein the spacer is between 4-50 amino acids in length; iii) one CD28 transmembrane domain; and, iv) one cytoplasmic region comprising 4-1BB co-stimulatory domain and CD3zeta intracellular signaling domain.

Additional embodiments concern methods of treating a patient with multiple myeloma comprising administering to the patient a composition comprising a population of cells expressing a bispecific chimeric antigen receptor (CAR) comprising in order from the amino to carboxy end of the CAR: i) a bispecific extracellular binding domain comprising a BCMA-specific scFv comprising SEQ ID NO:25; a (G4S)₄ linker; and a CS1-specific scFv comprising SEQ ID NO:47; ii) a hinge spacer comprising a IgG4 hinge and wherein the spacer is between 4-50 amino acids in length; iii) one CD28 transmembrane domain; and, iv) one cytoplasmic region comprising 4-1BB co-stimulatory domain and CD3zeta intracellular signaling domain.

In some embodiments, the method further comprises contacting the cells with feeder cells. In some embodiments, the feeder cells are irradiated. Feeder cells or support cells can include, for example, fibroblasts, mouse embryonic fibroblasts, JK1 cells, SNL 76/7 cells, human fetal skin cells, human fibroblasts, and human foreskin fibroblasts.

In some embodiments, the method excludes contacting T cells with feeder cells. In some cases, the excluded feeder cells are from a different animal species as the T cells.

In one embodiment of the methods described herein, the subject or patient to be treated is a human subject. The terms “individual,” “subject,” “host,” and “patient,” used interchangeably herein, refer to a mammal, including, but not limited to, murines (e.g., rats, mice), lagomorphs (e.g., rabbits), non-human primates, humans, canines, felines, ungulates (e.g., equines, bovines, ovines, porcines, caprines), etc.

When referring to peptides and polypeptides herein, the sequences and structures are written and interpreted as proceeding from the N-terminus to the C-terminus, which is standard practice in the art.

Any embodiment disclosed herein can be implemented or combined with any other embodiment disclosed herein, including aspects of embodiments for regions or domains of a CAR molecule can be combined and/or substituted and any and all other regions or domains. Moreover, any CAR molecule described herein can be implemented in the context of any method described herein. Similarly, aspects of any method embodiment can be combined and/or substituted with any other method embodiment disclosed herein. Moreover, any method disclosed herein may be recited in the form of “use of a composition” for achieving the method. It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention. Throughout this application, the term “about” is used to indicate that a value includes the standard deviation of error for the device or method being employed to determine the value.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-C. Rational design of BCMA/CS1 bispecific CARs for adoptive T cell therapy. (A) Schematic of adoptive T cell therapy (ACT). In ACT, primary human T cells are isolated from patients, expanded ex vivo, and then reinfused back into the patient as a cancer treatment. The T cells may be tumor-infiltrating lymphocytes (TILs; i.e., T cells that already express receptors that enable them to target cancer cells), or the T cells may be genetically modified to express either tumor-targeting T cell receptors (TCRs) or chimeric antigen receptors (CARs). (B) Bispecific CARs are single-chain proteins consisting of two antibody-derived single-chain variable fragments (scFvs) linked in series via a peptide linker to an IgG4 hinge extracellular spacer, CD28 transmembrane domain, 4-1BB co-stimulatory domain and CD3zeta chain. A panel of CAR variants was constructed from two CS1-binding scFvs and three BCMA-specific binding domains. Murine and humanized versions of BCMA-binding scFvs (c1D5.3 and J22.9-xi) were evaluated. (C) Known binding-epitope locations of scFvs in established CS1 CARs. Using modular DNA assembly, bispecific CARs were constructed where huLuc63 scFv was fixed in the membrane distal position and luc90 was fixed at the membrane proximal position plus a BCMA-binding domain in the membrane-distal position.

FIG. 2. Generation and screening of bispecific CAR-T cells. CAR-T cells are generated in a 14-day cycle. Starting on day 9 post-stimulation, CAR-T cells are characterized for anti-tumor function in various assays.

FIG. 3. Primary T cells express different bispecific CARs with varying efficiencies. An initial panel of 10 bispecific CARs and the corresponding single-input CARs were generated by isothermal DNA assembly. CARs were tagged with an N-terminal FLAG tag, and FLAG and expression levels were quantified by surface antibody staining followed by flow cytometry. The J22.9-xi-Luc90 and huJ22.9-xi-Luc90 CARs were subsequently eliminated from the panel due to poor expression.

FIG. 4A-C. Bispecific CAR panel demonstrates varying performance levels against BCMA+ and CS1+ targets. (A) Cell-lysis activity of single-input and bispecific CAR-T cells against K562 targets engineered to express either BCMA or CS1. Cells were seeded at an effector-to-target (E:T) ratio of 2:1, where effector-cell seeding was based on CAR+ T cell count. The fraction of viable K562 cells left after a 20-hour coincubation was quantified by fluorescence imaging of target cells using IncuCyte. All bispecific CARs in this panel contain a short extracellular spacer (IgG4 hinge) that connects the membrane-proximal scFv to the transmembrane domain. The data represented by the bars above each construct correspond to BCMA+K562 (left bar above each construct) and CS1+K562 (right bar above each construct). (B) Proliferation of single-input and bispecific BCMA/CS1 CAR-T cells upon antigen stimulation. CAR-T cells were stained with CellTrace violet (CTV) dye before being coincubated with parental (BCMA−/CS1-), BCMA+, or CS1+ target cells at a 2:1 E:T ratio. CTV median fluorescence intensity (MFI) was quantified by flow cytometry after a 5-day co-incubation. CARs containing huLuc63 paired with dAPRIL, J22.9-xi, and huJ22.9-xi were subsequently eliminated from the panel based on poor cytotoxicity and/or T cell proliferation. The data represented by the bars above each construct correspond to (from left to right) Parental K562, BCMA+K562, and CS1+K562. (C) Cytotoxicity of reduced bispecific CAR-T cell panel upon repeated antigen challenge. CAR-T cells were coincubated with K562 target cells at a 1:1 E:T ratio and re-challenged every 2 days with fresh target cells. Viable target cell count was quantified 2 days after each target cell addition by flow cytometry. ‘C#’ denotes the challenge number and ‘D#’ denotes the number of days post challenge. The data represented by the bars above each construct correspond to (from left to right) C1D2 (BCMA+/CS1+K562); C2D2 (BCMA+/CS1+K562); C3D2 (BCMA+K562); C4D2 (CS1+K562). (D) Characterization of the remaining K562 target cell populations after 4 challenges from part C reveals differing antigen preference among the panel of bispecific CARs. Values shown are the means of triplicates, with error bars indicating +1 standard deviation (SD). Data shown are representative of results from 3 independent experiments performed with cells from 3 different healthy donors. Based on these results, c11D5.3-Luc90, huc11D5.3-Luc90, and huLuc63-c11D5.3 were selected for further evaluation and optimization.

FIG. 5. c11D5.3 scFv shows superior function when separated from the cell membrane by a long extracellular spacer. Cytotoxicity of single-input CAR-T cell panel upon repeated antigen challenge. CAR-T cells were coincubated with target cells at a 1:1 E:T ratio and re-challenged every 2 days with fresh target cells. Viable target cell count was quantified 2 days after each target cell addition by flow cytometry. ‘C#’ denotes the challenge number and ‘D#’ denotes the number of days post challenge. Values shown are the mean of triplicates with error bars indicating +1 SD. Based on these results, attempts were made to further improve the huLuc63-c11D5.3 CAR by either moving the c11D5.3 scFv to the membrane-distal position or increasing the length of the extracellular spacer that connects the membrane-proximal scFv to the transmembrane domain. The data represented by the bars above each construct correspond to (from left to right) C1D2 (BCMA+/CS1+K562); C2D2 (BCMA+/CS1+K562); C3D2 (BCMA+ or CS1+K562); C4D2 (BCMA+ or CS1+K562).

FIG. 6A-C. Modified huluc63-c11D5.3 CARs exhibit improved BCMA-targeting but remain inferior to top-performing candidates. (A) Target cell lysis by T cells expressing modified bispecific CAR panel following 24-hour coincubation with K562 target cells. The data represented by the bars above each construct correspond to (from left to right) Parent K562; BCMA+K562; CS1+K562; and BCMA+/CS1+K562. (B) Proliferation of T cells expressing modified bispecific CAR panel following coincubation with K562 target cells. Samples in A and B were prepared and analyzed as described in FIG. 3. The data represented by the bars above each construct correspond to (from left to right) Parent K562; BCMA+K562; CS1+K562; and BCMA+/CS1+K562. (C) Cytotoxicity of single-input, bispecific, or dual-CAR T cells upon repeated antigen challenge. CAR-T cells were coincubated with wildtype (BCMA+/CS1+) MM.1S target cells at a 1:2 E:T ratio and re-challenged every 2 days with fresh target cells. The number of viable MM.1S target cells remaining 2 days after the 5th round of antigen stimulation was quantified by flow cytometry. Values shown are the means of triplicates with error bars indicating +1 SD.

FIG. 7A-D. Top-performing BCMA/CS1 bispecific CAR-T cells outperform T cells co-expressing BCMA and CS1 CARs. (A) Schematic of bispecific vs. dual CARs. (B) Expression of single-input, bispecific, or dual CARs in primary T cells. Single-input and bispecific CARs were each tagged with an N-terminal FLAG tag. In dual-CAR constructs, the CS1 CAR was N-terminally tagged with a FLAG tag while the BCMA CAR was N-terminally tagged with a HA tag. CAR surface expression levels were quantified by surface antibody staining of FLAG and HA tags followed by flow cytometry. Results indicate that single-chain, bispecific CARs have superior surface expression compared to co-expression of two full-length, single-input CARs. (C) Target cell lysis by T cells expressing single-input, bispecific, or dual CARs challenged with wildtype (BCMA+/CS1+) or CRISPR/Cas9-modified BCMA−/CS1+ and BCMA+/CS1− MM.1S myeloma cells. Cells were seeded at an E:T ratio of 2:1, and remaining viable target cell count after a 24-hour coincubation was quantified by fluorescence imaging on IncuCyte. The data represented by the bars above each construct correspond to (from left to right) BCMA+/CS1− MM.1S; BCMA−/CS1+MM.1S; and BCMA+/CS1+MM.1S (D) Proliferation of CAR-T cells co-incubated with parental K562, wildtype (BCMA+/CS1+) MM.1S, or CRISPR/Cas9-modified BCMA−/CS1+ and BCMA+/CS1− MM.1S myeloma cells. The data represented by the bars above each construct correspond to (from left to right) Parent K562; BCMA+/CS1− MM.1S; BCMA−/CS1+MM.1S; and BCMA+/CS1+MM.1S. Compared to bispecific CAR-T cells, dual CAR-T cells exhibit poor antigen-specific proliferation.

FIG. 8. K562 cells express higher BCMA and CS1 levels than MM.1S cells. Parental K562 (BCMA−/CS1−) cells were transduced to express a transgenic copy of BCMA, CS1, or both BCMA and CS1. MM.1S cells naturally express BCMA and CS1. MM.1S cells were modified using CRISPR/Cas9 to generate single-antigen cells lines (BCMA+/CS1− and BCMA−/CS1+). BCMA and CS1 antigen expression levels of target cells were quantified by surface antibody staining followed by flow cytometry.

FIG. 9A-E. BCMA/CS1 bispecific CARs demonstrate tumor killing in vitro and in vivo. (A) Naïve memory (NM) CAR-T cells exhibit superior in vitro killing compared to CD3+ and CD8+ CAR-T cells. Target cell lysis by NM, CD3+, or CD8+ CAR-T cells coincubated with wildtype (BCMA+/CS1+) or CRISPR/Cas9-modified BCMA−/CS1+ and BCMA+/CS1− MM.1S myeloma cells. NM cells were CD14- and CD25-depleted and enriched for CD62L expression. Cells were seeded at a 2:1 E:T ratio, and remaining viable target cell count after a 24-hour coincubation was quantified by fluorescence imaging on IncuCyte. The data represented by the bars above each construct corresponds to (from left to right) BCMA+/CS1− MM.1S; BCMA−/CS1+MM.1S; BCMA+/CS1+MM.1S. P values were calculated by paired two-tailed Student's t-test; n.s. not statistically significant (p>0.05); * p<0.05; ** p<0.01. (B) Schematic of in vivo multiple myeloma model. NOD/scid/γ−/− (NSG) mice were injected with wildtype MM.1S tumor cells stably expressing firefly luciferase. On day 6, mice were treated with a tail-vein injection of CAR-T cells. Tumor progression was tracked by bioluminescence imaging. Mice were re-dosed 21 days after initial tumor injection. (C) Mice engrafted with MM.1S xenografts and treated with EGFRt CAR-T cells succumbed to tumor burden and were sacrificed on day 40. Mice engrafted with MM.1S cells and treated with bispecific (c1D5.3-Luc90 Short) CAR-T cells initially exhibited tumor regression but eventually relapsed. Following T cell re-dose, tumor clearance was observed in one mouse. (D) Survival curve of mice shown in B. (E) At the time of sacrifice, tumors were recovered from mice engrafted with MM.1S cells and treated with bispecific CAR-T cells. Data shown were collected from mouse #1. Analysis of BCMA and CS1 antigen expression on tumors indicate that antigen expression was retained in relapsed tumors. Failure to clear tumors was attributable to tumor-model aggressiveness.

FIG. 10A-B. Anti-CS1 CAR-T cells exhibit signs of fratricide but do not impede cell expansion and manufacturing. (A) To evaluate CAR-T cell fratricide, bispecific and single-input CAR-T cells were coincubated with untransduced (UT) T cells at a 2:1 ratio. UT T cells were stained with CTV to distinguish them from T cells that were exposed to lentivirus but not transduced. After 24 hours, viable UT T cell count was quantified by flow cytometry. Values shown are the means of triplicates with error bars indicating +1 SD. (B) Proliferation of T cells expressing the top-performing BCMA/CS1 bispecific CARs and corresponding single-input CARs 10 days post activation by CD3/CD28 Dynabeads. Fold-proliferation is defined as the number of CAR+ T cells on day 10 normalized to the number of T cells transduced on day 2 post-activation. Values, averages, and error bars indicating ±1 SD from 5 donors are shown. No statistically significant difference in proliferation was observed across the CAR-T cell panel.

FIG. 11A-C. High-throughput generation and screening of BCMA/CS1 OR-gate CARs. a. A vertically integrated design process for CAR-T cell therapy development. b. Schematic of a single-chain bispecific (OR-gate) CAR, which contains two ligand-binding domains connected in tandem. A panel of CAR variants was constructed from two CS1-binding scFvs and three BCMA-specific binding domains. Both murine and humanized versions of BCMA-binding scFvs (c11D5.3 and J22.9-xi) were evaluated. The CS1-binding huLuc63 and Luc90 scFvs were fixed at the membrane-distal and membrane-proximal positions, respectively, based on binding-epitope analysis. c. Methodology for producing and screening bispecific CARs. CAR-T cells were generated in a 14-day cycle. Starting on day 9 post-stimulation, CAR-T cells were characterized for anti-tumor function in various assays, which could last up to two weeks.

FIG. 12A-D. OR-gate CAR panel demonstrates varying performance levels against BCMA⁺ and CS1⁺ targets. a. Cell-lysis activity of single-input and bispecific CD8+ CAR-T cells against K562 targets engineered to express either BCMA or CS1. Cells were seeded at an effector-to-target (E:T) ratio of 2:1, where effector-cell seeding was based on CAR+ T cell count. The fraction of viable K562 cells left after a 20-hour coincubation was quantified by fluorescence imaging of target cells using IncuCyte. All bispecific CARs in this panel contained a short extracellular spacer. b. Proliferation of single-input and bispecific BCMA/CS1 CD8+ CAR-T cells upon antigen stimulation. CAR-T cells were stained with CellTrace Violet (CTV) dye. CTV median fluorescence intensity (MFI) was quantified by flow cytometry after a 5-day coincubation with parental (BCMA⁻/CS1⁻), BCMA⁺, CS1⁺, or BCMA⁺/CS1⁺ K562 target cells at a 2:1 E:T ratio. CARs containing huLuc63 paired with dAPRIL, J22.9-xi, and huJ22.9-xi were subsequently eliminated from the panel based on poor cytotoxicity and/or T cell proliferation. c. Cytotoxicity of reduced bispecific CAR-T cell panel upon repeated antigen challenge. CD8⁺ CAR-T cells were coincubated with K562 target cells at a 1:1 E:T ratio and re-challenged every 2 days with fresh target cells. Viable target cell count was quantified by flow cytometry 2 days after each targeT cell addition. ‘C#’ denotes the challenge number and ‘D#’ denotes the number of days post challenge. d. Characterization of the remaining K562 targeT cell populations after 4 challenges from part C reveals differing antigen preference among the panel of bispecific CARs. Values shown are the means of triplicates, with error bars indicating +1 standard deviation (SD).

FIG. 13A-D. OR-gate CAR-T cells outperform T cells co-expressing individual BCMA and CS1 CARs. a. Single-input and bispecific CARs were tagged with an N-terminal FLAG tag. In DualCAR constructs, the CS1 CAR was N-terminally tagged with a FLAG tag while the BCMA CAR was N-terminally tagged with a HA tag. b. CAR surface expression levels were quantified by surface antibody staining of FLAG and HA tags followed by flow cytometry. c. Target cell lysis by naïve/memory (NM) T cells expressing single-input, bispecific, or dual CARs challenged with wildtype (BCMA+/CS1+) or CRISPR/Cas9-modified BCMA−/CS1+ and BCMA+/CS1− MM.1S myeloma cells. Cells were seeded at a 2:1 E:T ratio, and viable target cell count after a 24-hour coincubation was quantified by fluorescence imaging on IncuCyte. d. NM CAR-T cell proliferation following a 5-day coincubation with MM.1S or K562 target cells. Values shown are the means of triplicates with error bars indicating +1 SD. P values were calculated by paired two-tailed Student's t-test; n.s. not statistically significant (p>0.05); * p<0.05; ** p<0.01. Data shown are representative of results from 3 independent experiments performed with cells from 3 different healthy donors.

FIG. 14A-E. Naïve/memory (NM) CAR-T cells exhibit superior in vitro and in vivo anti-tumor killing compared to CD3⁺ and CD8⁺ CAR-T cells. a. Interferon (IFN)-γ production of CD8⁺ or NM single-input and bispecific CAR-T cells following 24-hour coincubation with wildtype (BCMA⁺/CS1⁺) or CRISPR/Cas9-modified BCMA⁻/CS1⁺ and BCMA⁺/CS1⁻ MM.1S cells at a 2:1 E:T ratio. TNF and IL-2 production followed similar trends (FIG. 23). b. Target cell lysis by NM or CD8⁺ single-input and bispecific CAR-T cells following repeated antigen challenge. Cells were seeded at an E:T ratio of 1:2. Number of remaining BCMA⁺/CS1⁺ MM.1S target cells was quantified after 4 rounds of antigen challenge. c. Proliferation of NM or CD8⁺ single-input and OR-gate CAR-T cells following a 5-day co-incubation with target cells. d. Evaluation of NM, CD8⁺, and CD3⁺ huc11D5.3-Luc90 CAR-T cells in vivo. NSG mice were engrafted with 2×10⁶ WT MM.1S cells, and 0.5×10⁶ T cells of the indicated subtype and CAR expression were injected 6 days post tumor injection. Tumor progression was tracked by bioluminescence imaging. e. Survival curve of mice shown in (d). Experiment was terminated on day 108. At the time of sacrifice, the last mouse in the NM/OR-gate CAR treatment group still maintained tumor clearance.

FIG. 15A-D. BCMA/CS1 OR-gate CAR-T cells prevent antigen escape in vivo. a. Evaluation of single-input and bispecific CAR-T cells in vivo. Mice were engrafted with a mixture of 1.5×10⁶ MM.1S cells containing a 1:1:1 ratio of BCMA⁺/CS1⁻, BCMA⁻/CS1⁺, and BCMA⁺/CS1⁺ cells. Tumor-bearing animals were treated with 1.5×10⁶ EGFRt- or CAR-expressing T cells on day 5 (5 days after tumor injection) and re-dosed with 1.5×10⁶ EGFRt- or CAR-expressing T cells on day 13. Six mice were included in each initial treatment group but only 5 mice in the huLuc63-c11D5.3 group were re-dosed due to limited T cell availability. Tumor progression was monitored by bioluminescence imaging. b. Survival of mice shown in (a). Statistical difference (depicted) between survival of huLuc63-c11D5.3-treatment group compared with other treatment groups was determined using log-rank analysis; n.s. not statistically significant (p>0.05); * p<0.05; ** p<0.01. c. BCMA/CS1 antigen expression on tumors harvested from mice treated with CS1 single-input Luc90 Short, huLuc63 Long, or BCMA single-input c11D5.3 Long CAR-T cells. d. Antigen expression pattern on tumor cells recovered at the time of animal sacrifice. Each data point corresponds to an individual tumor sample recovered that included more than 100 tumor cells as detected by flow cytometry; each mouse generally contained multiple tumors at the time of sacrifice.

FIG. 16A-B Combination therapy of BCMA/CS1 OR-gate CAR-T cells with anti-PD-1 antibody increases anti-tumor efficacy and durability of response in vivo. a. Mice were engrafted with 1.5×10⁶ WT MM.1S cells. Tumor-bearing animals were treated with 1.5×10⁶ EGFRt- or CAR-expressing T cells on day 8 (8 days after tumor injection) and re-dosed with 1.5×10⁶ EGFRt- or CAR-expressing T cells on day 16. Tumor progression was monitored by bioluminescence imaging and shown for individual animals in each test group (n=6). b. PD-1 expression on T cells persisting in mice at time of animal sacrifice. c. Survival of mice shown in (a).

FIG. 17A-B Rational design of BCMA/CS1 OR-gate CARs and single-input CARs. a. OR-gate CARs were constructed based on the binding-epitope location of CS1-specific scFvs. b. Schematic of single-chain bispecific CARs and c. single-input CARs. CARs were tagged with an N-terminal FLAG tag and fused via a self-cleaving T2A peptide to truncated epidermal growth factor receptor (EGFRt), which serves as a transduction marker.

FIG. 18A-B. Bispecific CARs present on primary T cell surface with varying efficiencies. a. FLAG-tagged CAR expression levels were quantified by surface antibody staining followed by flow cytometry. b. Alignment of J22.9-xi V_{L} and Luc90 V_{L} nucleotide sequences.

FIG. 19. C11D5.3 scFv shows superior function when separated from the cell membrane by a long extracellular spacer in the CAR context. Cytotoxicity of single-input CAR-T cells upon repeated antigen challenge. CD8⁺ CAR-T cells were coincubated with the indicated target cells at a 1:1 E:T ratio and re-challenged every 2 days with fresh target cells. Viable target cell count was quantified by flow cytometry 2 days after each target cell addition. ‘C#’ denotes the challenge number and ‘D#’ denotes the number of days post challenge. Values shown are the mean of triplicates with error bars indicating +1 standard deviation (SD).

FIG. 20A-B. Schematic of additional OR-gate CAR and DualCAR constructs. a. The huLuc63-c11D5.3 Short CAR was modified in an attempt to improve BCMA targeting by replacing the short spacer with a long spacer and/or placing the c11D5.3 scFv in the membrane-distal position. b. DualCAR constructs were constructed where the CS1 CAR was N-terminally tagged with a FLAG tag while the BCMA CAR was N-terminally tagged with a HA tag. The top-performing single-input BCMA CAR identified from previous assays was selected for DualCAR comparison.

FIG. 21A-C. Modified huluc63-c11D5.3 BCMA/CS1 CARs exhibit improved BCMA-targeting but remains inferior to top-performing candidates. a. Cytokine production of BCMA+K562 cells (top) and CS1+K562 cells (bottom), b. target cell lysis, and c. proliferation by CD8⁺ T cells expressing the modified CAR panel following 24-hour coincubation with BCMA⁺ or CS1⁺ K562 cells. P values were calculated by paired two-tailed Student's t-test; n.s. not statistically significant (p>0.05); * p<0.05; ** p<0.01.

FIG. 22A-E. CS1-specific OR-gate CAR-T cells expand efficiently ex vivo. a. CS1 is expressed at higher levels on CD8⁺ T cells than on CD4⁺ T cells. CS1 expression on CD4⁺ and CD8⁺ T cells from the same donor was quantified by surface antibody staining of CS1 by flow cytometry. b. Fratricide by single-input or bispecific CAR-T cells coincubated with CTV-stained, untransduced (UT) CD8⁺ T cells. Cells were seeded at a 2:1, [CAR-T cells]:[UT-T cell] ratio, and viable CTV-stained UT-T cells were quantified by flow cytometry after a 24-hour coincubation. Percent lysis of UT CD8⁺ T cells was normalized to cells lysed in the EGFRt control group. Values, means, and error bars indicating ±1 SD from 4 donors are shown. No statistically significant difference in proliferation was observed across the CAR-T cell panel. c. Proliferation of T cells expressing the top-two BCMA/CS1 bispecific CARs or corresponding single-input CARs 10 days post activation by CD3/CD28 Dynabeads. Fold-proliferation is defined as the number of CAR+ T cells on day 10 normalized to the number of T cells on the day of transduction (i.e., 2 days post activation). Values, means, and error bars indicating ±1 SD from 5 donors are shown. No statistically significant difference in proliferation was observed across the CAR-T cell panel. d. Target cell lysis by NM single-input and bispecific CARs following repeated antigen challenge. Cells were seeded at a 1:2 E:T ratio. The number of viable BCMA⁺/CS1⁺ MM.1S target cells remaining 2 days after the fifth challenge (i.e., on C5D2) was quantified by flow cytometry. The means of triplicate samples are shown with error bars indicating +1 SD. e. CAR-T cell proliferation over 5 rounds of repeated antigen challenge. Number of CAR-T cells remaining 2 days after each round of antigen challenge was quantified by flow cytometry. Statistics for C5D2 are depicted. P values were calculated by paired two-tailed Student's t-test; n.s. not statistically significant (p>0.05); * p<0.05; ** p<0.01.

FIG. 23. NM CAR-T cells show higher cytokine production levels than CD8⁺ T cells. IL-2 and TNF production of CD8⁺ vs. NM CAR-T cells coincubated with different target cells.

FIG. 24A-B. Naïve/memory CAR-T cells successfully cleared tumors in one mouse and relapsed tumor cells retained antigen expression. a. Tumor progression in mice described in FIG. 4d following T cell re-dose. Tumor signal was monitored by bioluminescence imaging. b. BCMA and CS1 expression on tumor cells isolated from relapsed animals. Data shown are tumors isolated from mouse 1 treated with NM CAR-T cells (sacrificed on Day 60).

FIG. 25. Mice were injected with BCMA⁺/CS1⁻, BCMA⁻/CS1⁺, and BCMA⁺/CS1⁺ cells mixed at a 1:1:1 ratio

FIG. 26A-B. Tumor progression in CAR-treated mice. a. Average tumor radiance in mice treated with single-input CAR-T cells. b. Tumor radiance in individual mice from all treatment groups. One mouse in the huLuc63-c11D5.3 treatment group exhibited complete tumor clearance, and no tumor cells were detected at the time of conclusion of the study.

FIG. 27A-C. Residual tumors recovered from animals treated with OR-gate CAR-T cells contained mutations in the CRISPR-targeted BCMA site, indicating origin from the engineered BCMA⁻/CS1⁺ MM.1S cell line. Two tumor samples (87.2% and 88.9% BCMA⁻/CS1⁺, respectively) recovered from two different animals in the huLuc63-c11D5.3 Short CAR-T cell-treated group shown in FIG. 15 were analyzed by amplicon sequencing. Full sequencing data are shown in Supplementary Data Set 1. The most dominant BCMA sequence in each tumor sample (accounting for 99.5% and 99.7% of all reads in each sample, respectively) is shown in alignment with the WT BCMA sequence within the sequenced amplicon. Highlights mark the PAM and guide RNA sequences used in CRISPR/Cas9-mediated gene editing in order to generate the BCMA⁻/CS1⁺ MM.1S line used in the animal study.

FIG. 28A-B. Kill rate constant of top-performing OR-gate CARs. Killing of MM.1S tumor cells by OR-gate CAR-T cells was evaluated over a period of 24 hours by IncuCyte. Kill rate constant with standard error (SE) was determined by fitting the data to a log-linear model in R. The mean % tumor cells remaining across time from triplicate samples are shown, with the model overlaid and error bars representing ±1 SD. Shading indicates the 95% confidence interval of the model's fit.

FIG. 29A-C. Recovered tumor cells remain targetable by OR-gate CAR-T cells, and BCMA and CS1 expression pattern does not affect in vitro MM.1S growth rate. a. Tumor cells recovered from OR-gate CAR-T cell-treated mice remain susceptible to OR-gate CAR-T cell-mediated killing. MM.1S tumor cells recovered from mice #5 and #6 in the huLuc63-c11D5.3 Short CAR-T cell-treated group shown in FIG. 15 were sorted to purity based on EGFP expression, expanded, and subjected to co-incubation with huLuc63-c11D5.3 Short CAR-T cells at multiple E:T ratios. BCMA⁻/CS1⁺ MM.1S cells and a mixture of 1:1:1 of BCMA⁺/CS1⁺, BCMA⁻/CS1⁺, and BCMA⁺/CS1⁻ MM.1S cells were included as controls. Results indicate the tumor cells remain vulnerable to killing by the OR-gate CAR-T cells. The mean % tumor cells remaining across time from triplicate samples are shown as described in FIG. 28. b. Kill rates (±SE) of the data shown in (a) were determined by fitting the data to a log-linear model in R. c. BCMA⁺/CS1⁻, BCMA⁻/CS1⁺, and BCMA⁺/CS1+MM.1S cells were combined at equal ratios (1:1:1) and co-cultured for 35 days. The % of each cell type quantified throughout the co-culture period showed equal growth rates for the three MM.1S cell lines. Values shown are the means of triplicates with error bars indicating +1 SD.

FIG. 30A-F. Lentivirally transduced CAR-T cells outperform HDR-modified CAR-T cells in vitro. a. Schematic of an AAV vector encoding homology arms (left and right, LHA and RHA) flanking an integration cassette consisting of a splice-acceptor (SA) site, a T2A sequence, and the FLAG-tagged huLuc63-c11D5.3 OR-gate CAR. b. Representative TCR α/β and FLAG-tag flow plots 11 days post RNP nucleofection and AAV transduction. c. HDR-modified OR-gate CAR-T cells exhibit relatively poor viability 11 days post RNP nucleofection/AAV transduction. d-f. Upon repeated antigen challenge with WT MM.1S cells, HDR-modified OR-gate CAR-T cells exhibit d. inferior cytotoxicity, e. weaker antigen-stimulated T cell proliferation, and f. stronger and longer-lasting exhaustion-marker (PD-1 and LAG-3) expression than lentivirally transduced OR-gate CAR-T cells. Values shown are the mean of triplicates with error bars indicating ±1 SD. Results shown are representative of cells generated from two healthy donors.

FIG. 31A-C. CAR-T cells achieve long-term persistent in vivo but upregulate PD-1 expression. Tumor mass, bone marrow, and cardiac blood samples recovered from animals in the study shown in FIG. 15 at the time of sacrifice were analyzed for (a) the presence of cells expressing human CD45, (b) the frequency of EGFRt+ (and thus CAR+) cells among huCD45+ cells, and (c) the frequency of PD-1+ cells among huCD45+ cells.

FIG. 32. BCMA/CS1 OR-Gate CAR-T cells can eradicate solid tumor nodules in vivo. NSG mice were engrafted with MM.1S tumors and treated with BCMA/CS1 OR-gate CAR-T cells as described in FIG. 16. Two animals in the group treated with huLuc63-c11D5.3 Short CAR-T cells without anti-PD-1 developed palpable solid tumor nodules in the chest and flank, but the tumors eventually regressed to below detection levels and the animals have remained viable in a tumor-free state for >3 weeks at the time of this writing. Bioluminescence imaging of the animals in dorsal and ventral views are shown.

FIG. 33. Percent survival of single-input BCMA-CAR T therapy compared to controls and bispecific CAR T therapy.

### DESCRIPTION OF ILLUSTRATIVE EMBODIMENTS

Multiple myeloma (MM) is an incurable disease affecting plasma cells, which are B cells that play a critical role in the humoral immune response. The adoptive transfer of chimeric antigen receptor (CAR)-T cells targeting B-cell maturation antigen (BCMA) has achieved up to 100% response rate in the treatment of MM patients. However, following initial response to therapy, progression of tumors with downregulated BCMA expression has been observed, suggesting antigen escape as a critical limitation of the treatment. Another potential target for MM is CS1 (SLAMF7), which is highly expressed on MM. While CS1 CARs have demonstrated efficacy against myeloma, CS1 CAR-T cells may be susceptible to fratricide since CS1 is also expressed at high levels in activated T cells. To address these limitations, the inventors constructed a panel of single-chain bispecific CARs for the treatment of MM. Using high-throughput characterization methods, the inventors identified BCMA/CS1 CAR-T cells that effectively target both BCMA and CS1 while retaining robust capacity for ex vivo expansion. In addition, BCMA/CS1 CAR-T cells could effectively control tumor growth in established MM xenografts in vivo. Overall, the BCMA/CS1 bispecific CAR presents a promising treatment approach to prevent antigen escape in CAR-T cell therapy against MM.

### I. DEFINITIONS

The peptides of the disclosure relate to peptides comprising CARs or chimeric antigen receptors. CARs are engineered receptors, which graft an arbitrary specificity onto an immune effector cell. Typically, these receptors are used to graft the specificity of a monoclonal antibody onto a T cell. The receptors are called chimeric because they are composed of parts from different sources.

The terms “protein,” “polypeptide,” and “peptide” are used interchangeably herein when referring to a gene product.

“Homology,” or “identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 60% identity, less than 50% identity, less than 40% identity, less than 30% identity, or less than 25% identity, with one of the sequences of the current disclosure.

The terms “amino portion,” “N-terminus,” “amino terminus,” and the like as used herein are used to refer to order of the regions of the polypeptide. Furthermore, when something is N-terminal to a region it is not necessarily at the terminus (or end) of the entire polypeptide, but just at the N-terminus of the region or domain. Similarly, the terms “carboxy portion,” “C-terminus,” “carboxy terminus,” and the like as used herein is used to refer to order of the regions of the polypeptide, and when something is C-terminal to a region it is not necessarily at the terminus (or end) of the entire polypeptide, but just at the C-terminus of the region or domain.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

The term “xeno-free (XF)” or “animal component-free (ACF)” or “animal free,” when used in relation to a medium, an extracellular matrix, or a culture condition, refers to a medium, an extracellular matrix, or a culture condition which is essentially free from heterogeneous animal-derived components. For culturing human cells, any proteins of a non-human animal, such as mouse, would be xeno components. In certain aspects, the xeno-free matrix may be essentially free of any non-human animal-derived components, therefore excluding mouse feeder cells or Matrigel™. Matrigel™ is a solubilized basement membrane preparation extracted from the Engelbreth-Holm-Swarm (EHS) mouse sarcoma, a tumor rich in extracellular matrix proteins to include laminin (a major component), collagen IV, heparin sulfate proteoglycans, and entactin/nidogen. In some embodiments, the compositions described herein or cells of the disclosure are cultured in and/or prepared in/with xeno-free or animal component-free or animal free medium.

Cells are “substantially free” of certain reagents or elements, such as serum, signaling inhibitors, animal components or feeder cells, exogenous genetic elements or vector elements, as used herein, when they have less than 10% of the element(s), and are “essentially free” of certain reagents or elements when they have less than 1% of the element(s). However, even more desirable are cell populations wherein less than 0.5% or less than 0.1% of the total cell population comprise exogenous genetic elements or vector elements.

A culture, matrix or medium are “essentially free” of certain reagents or elements, such as serum, signaling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium. The serum-free medium may be essentially free of serum.

A “gene,” “polynucleotide,” “coding region,” “sequence,” “segment,” “fragment,” or “transgene” which “encodes” a particular protein, is a nucleic acid molecule which is transcribed and optionally also translated into a gene product, e.g., a polypeptide, in vitro or in vivo when placed under the control of appropriate regulatory sequences. The coding region may be present in either a cDNA, genomic DNA, or RNA form. When present in a DNA form, the nucleic acid molecule may be single-stranded (i.e., the sense strand) or double-stranded. The boundaries of a coding region are determined by a start codon at the 5′ (amino) terminus and a translation stop codon at the 3′ (carboxy) terminus. A gene can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the gene sequence.

The term “cell” is herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.). The term BCMA/CS1 loop refers to an extracellular region in which one scFv (either BCMA or CS1) is nested in between the VL and VH of the other scFv. Exemplary embodiments are described herein and exemplified by SEQ ID NOS:174-177.

As used herein, the term “stem cell” refers to a cell capable of self-replication and pluripotency or multipotency. Typically, stem cells can regenerate an injured tissue. Stem cells herein may be, but are not limited to, embryonic stem (ES) cells, induced pluripotent stem cells or tissue stem cells (also called tissue-specific stem cell, or somatic stem cell).

“Embryonic stem (ES) cells” are pluripotent stem cells derived from early embryos. An ES cell was first established in 1981, which has also been applied to production of knockout mice since 1989. In 1998, a human ES cell was established, which is currently becoming available for regenerative medicine.

Unlike ES cells, tissue stem cells have a limited differentiation potential. Tissue stem cells are present at particular locations in tissues and have an undifferentiated intracellular structure. Therefore, the pluripotency of tissue stem cells is typically low. Tissue stem cells have a higher nucleus/cytoplasm ratio and have few intracellular organelles. Most tissue stem cells have low pluripotency, a long cell cycle, and proliferative ability beyond the life of the individual. Tissue stem cells are separated into categories, based on the sites from which the cells are derived, such as the dermal system, the digestive system, the bone marrow system, the nervous system, and the like. Tissue stem cells in the dermal system include epidermal stem cells, hair follicle stem cells, and the like. Tissue stem cells in the digestive system include pancreatic (common) stem cells, liver stem cells, and the like. Tissue stem cells in the bone marrow system include hematopoietic stem cells, mesenchymal stem cells, and the like. Tissue stem cells in the nervous system include neural stem cells, retinal stem cells, and the like.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as fibroblast, a hematopoietic cell, a myocyte, a neuron, an epidermal cell, or the like, by introducing certain factors, referred to as reprogramming factors.

“Pluripotency” refers to a stem cell that has the potential to differentiate into all cells constituting one or more tissues or organs, or particularly, any of the three germ layers: endoderm (interior stomach lining, gastrointestinal tract, the lungs), mesoderm (muscle, bone, blood, urogenital), or ectoderm (epidermal tissues and nervous system). “Pluripotent stem cells” used herein refer to cells that can differentiate into cells derived from any of the three germ layers, for example, direct descendants of totipotent cells or induced pluripotent cells.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

In some embodiments, the methods are useful for reducing the size and/or cell number of a tumor. In some embodiments, the method of the disclosure are useful for inhibiting the growth of tumors, such as solid tumors, in a subject.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The term functional antibody fragment includes antigen binding fragments of antibodies, including e.g., Fab′, F(ab′)₂, Fab, Fv, rlgG, and scFv fragments. The term scFv refers to a single chain Fv antibody in which the variable domains of the heavy chain and of the light chain of a traditional two chain antibody have been joined to form one chain.

A “signal peptide” refers to a peptide sequence that directs the transport and localization of the protein within a cell, e.g. to a certain cell organelle (such as the endoplasmic reticulum) and/or the cell surface. A signal peptide directs the nascent protein into the endoplasmic reticulum. This is essential if the receptor is to be glycosylated and anchored in the cell membrane. Generally, the signal peptide natively attached to the amino-terminal most component is used (e.g. in an scFv with orientation light chain-linker-heavy chain, the native signal of the light-chain is used). In some embodiments the signal peptide is SEQ ID NO:18.

In some embodiments, the signal peptide is cleaved after passage of the endoplasmic reticulum (ER), i.e. is a cleavable signal peptide. In some embodiments, a restriction site is at the carboxy end of the signal peptide to facilitate cleavage.

The use of a single chain variable fragment (scFv) is of particular interest. scFvs are recombinant molecules in which the variable regions of light and heavy immunoglobulin chains encoding antigen-binding domains are engineered into a single polypeptide. Generally, the V_{H} and V_{L} sequences are joined by a linker sequence. See, for example, Ahmad (2012) Clinical and Developmental Immunology Article ID 980250, herein specifically incorporated by reference. Described herein are BCMA-specific scFv molecules that comprise the variable regions of light and heavy immunoglobulin chains encoding BCMA-binding domains that are engineered into a single polypeptide. Similarly, the CS1-specific scFv molecules described herein comprise the variable regions of light and heavy immunoglobulin chains encoding CS1-binding domains that are engineered into a single polypeptide.

As used herein, the term “binding affinity” refers to the equilibrium constant for the reversible binding of two agents and is expressed as a dissociation constant (Kd). Binding affinity can be at least 1-fold greater, at least 2-fold greater, at least 3-fold greater, at least 4-fold greater, at least 5-fold greater, at least 6-fold greater, at least 7-fold greater, at least 8-fold greater, at least 9-fold greater, at least 10-fold greater, at least 20-fold greater, at least 30-fold greater, at least 40-fold greater, at least 50-fold greater, at least 60-fold greater, at least 70-fold greater, at least 80-fold greater, at least 90-fold greater, at least 100-fold greater, or at least 1000-fold greater, or more (or any derivable range therein), than the binding affinity of an antibody for unrelated amino acid sequences. As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding” refers to a direct association between two molecules, due to, for example, covalent, electrostatic, hydrophobic, and ionic and/or hydrogen-bond interactions, including interactions such as salt bridges and water bridges.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

As used herein the specification, “a” or “an” may mean one or more. As used herein in the claim(s), when used in conjunction with the word “comprising”, the words “a” or “an” may mean one or more than one. Any embodiment set forth using the term “comprising” can also be implemented with respect to the terms “consisting of” or “consisting essentially of.” The phrase “consisting of” excludes any element, step, or component not specified. The phrase “consisting essentially of” limits the scope of described subject matter to the specified molecules or sequences or steps and those that do not materially affect its basic and novel characteristics.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

### II. POLYPEPTIDES

A. Antigen Binding Regions

The antigen-binding region may be a single-chain variable fragment (scFv) derived from a BCMA and/or CS1 antibody or a binding region of a BCMA ligand, such as A PRoliferation-Inducing Ligand (APRIL). “Single-chain Fv” or “scFv” antibody fragments comprise the V_{H} and V_{L} domains of an antibody, wherein these domains are present in a single polypeptide chain. In some embodiments, the antigen-binding domain further comprises a peptide linker between the VH and VL domains, which may facilitate the scFv forming the desired structure for antigen binding.

The variable regions of the antigen-binding domains of the polypeptides of the disclosure can be modified by mutating amino acid residues within the VH and/or VL CDR 1, CDR 2 and/or CDR 3 regions to improve one or more binding properties (e.g., affinity) of the antibody. The term “CDR” refers to a complementarity-determining region that is based on a part of the variable chains in immunoglobulins (antibodies) and T cell receptors, generated by B cells and T cells respectively, where these molecules bind to their specific antigen. Since most sequence variation associated with immunoglobulins and T cell receptors is found in the CDRs, these regions are sometimes referred to as hypervariable regions. Mutations may be introduced by site-directed mutagenesis or PCR-mediated mutagenesis and the effect on antibody binding, or other functional property of interest, can be evaluated in appropriate in vitro or in vivo assays. Preferably conservative modifications are introduced and typically no more than one, two, three, four or five residues within a CDR region are altered. The mutations may be amino acid substitutions, additions or deletions.

Framework modifications can be made to the antibodies to decrease immunogenicity, for example, by “backmutating” one or more framework residues to the corresponding germline sequence.

It is also contemplated that the antigen binding domain may be multi-specific or multivalent by multimerizing the antigen binding domain with VH and VL region pairs that bind either the same antigen (multi-valent) or a different antigen (multi-specific).

The binding affinity of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M. In some embodiments, the K_{D} of the antigen binding region, such as the variable regions (heavy chain and/or light chain variable region), or of the CDRs may be at least 10⁻⁵M, 10⁻⁶M, 10⁻⁷M, 10⁻⁸M, 10⁻⁹M, 10⁻¹⁰M, 10⁻¹¹M, 10⁻¹²M, or 10⁻¹³M (or any derivable range therein). In some embodiments, the binding affinity of the BCMA binding region for the BCMA antigen is greater than the binding affinity for the CS1 binding region for the CS1 antigen. In some embodiments, the binding affinity of the CS1 binding region for the CS1 antigen is greater than the binding affinity for the BCMA binding region for the BCMA antigen. In some embodiments, the binding affinity of the BCMA binding region for the BCMA antigen is at least 1.5, 2, 2.5, 3, 3.5, 4, 4.5, 5, 5.5, 6, 6.5, 7, 7.5, 8, 8.5, 9, 9.5, or 10 times greater (or any derivable ranger therein) than the binding affinity of the CS1 binding region for the CS1 antigen. In some embodiments, the binding affinity of the CS1 binding region for the CS1 antigen is at least 1.2, 1.3, 1.4, 1.5, 1.6, 1.7, 1.8, 1.9, 2, 2.1, 2.2, 2.3, 2.4, 2.5, 3, 3.5, 4, 4.5, 5, 5.5, 6, 6.5, 7, 7.5, 8, 8.5, 9, 9.5, or 10 times greater (or any derivable ranger therein) than the binding affinity of the BCMA binding region for the BCMA antigen.

Binding affinity, K_{A}, or K_{D} can be determined by methods known in the art such as by surface plasmon resonance (SRP)-based biosensors, by kinetic exclusion assay (KinExA), by optical scanner for microarray detection based on polarization-modulated oblique-incidence reflectivity difference (OI-RD), or by ELISA.

In some embodiments, the BCMA and/or CS1-binding region is humanized. In some embodiments, the polypeptide comprising the humanized binding region has equal, better, or at least 80, 81, 82, 83, 84, 85, 86, 87, 88, 89, 90, 91, 92, 93, 94, 95, 96, 97, 98, 99, 100, 101, 102, 103, 104, 104, 106, 106, 108, 109, 110, 115, or 120% binding affinity or expression level in host cells, compared to a polypeptide comprising a non-humanized binding region, such as a binding region from a mouse.

In some embodiments, the framework regions, such as FR1, FR2, FR3, and/or FR4 of a human framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a mouse framework.

In some embodiments, the framework regions, such as FR1, FR2, FR3, and/or FR4 of a mouse framework can each or collectively have at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 (or any derivable range therein) amino acid substitutions, contiguous amino acid additions, or contiguous amino acid deletions with respect to a human framework.

The substitution may be at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 of FR1, FR2, FR3, or FR4 of a heavy or light chain variable region.

B. Extracellular Spacer

An extracellular spacer may link the antigen-binding domain to the transmembrane domain. It should be flexible enough to allow the antigen-binding domain to orient in different directions to facilitate antigen binding. In one embodiment, the spacer is the hinge region from IgG. Alternatives include the CH2CH3 region of immunoglobulin and portions of CD3. In some embodiments, the CH2CH3 region may have L235E/N297Q or L235D/N297Q modifications, or at least 75%, at least 80%, at least 85%, at least 90%, at least 95%, at least 98%, or 100% amino acid sequence identity of the CH2CH3 region. In some embodiments, the spacer is from IgG4.

As used herein, the term “hinge” refers to a flexible polypeptide connector region (also referred to herein as “hinge region” or “spacer”) providing structural flexibility and spacing to flanking polypeptide regions and can consist of natural or synthetic polypeptides. A “hinge” derived from an immunoglobulin (e.g., IgG1) is generally defined as stretching from Glu216 to Pro230 of human IgG1 (Burton (1985) Molec. Immunol., 22: 161-206). Hinge regions of other IgG isotypes may be aligned with the IgG1 sequence by placing the first and last cysteine residues forming inter-heavy chain disulfide (S—S) bonds in the same positions. The hinge region may be of natural occurrence or non-natural occurrence, including but not limited to an altered hinge region as described in U.S. Pat. No. 5,677,425. The hinge region can include a complete hinge region derived from an antibody of a different class or subclass from that of the CH1 domain. The term “hinge” can also include regions derived from CD8 and other receptors that provide a similar function in providing flexibility and spacing to flanking regions.

The extracellular spacer can have a length of at least, at most, or exactly 4, 5, 6, 7, 8, 9, 10, 12, 15, 16, 17, 18, 19, 20, 20, 25, 30, 35, 40, 45, 50, 75, 100, 110, 119, 120, 130, 140, 150, 160, 170, 180, 190, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, 290, 300, 325, 350, or 400 amino acids (or any derivable range therein). In some embodiments, the extracellular spacer consists of or comprises a hinge region from an immunoglobulin (e.g. IgG). Immunoglobulin hinge region amino acid sequences are known in the art; see, e.g., Tan et al. (1990) Proc. Natl. Acad. Sci. USA 87: 162; and Huck et al. (1986) Nucl. Acids Res.

The length of an extracellular spacer may have effects on the CAR's signaling activity and/or the CAR-T cells' expansion properties in response to antigen-stimulated CAR signaling. In some embodiments, a shorter spacer such as less than 50, 45, 40, 30, 35, 30, 25, 20, 15, 14, 13, 12, 11, or 10 amino acids. In some embodiments, a longer spacer, such as one that is at least 50, 60, 70, 80, 90, 100, 110, 120, 130, 140, 150, 200, 201, 202, 203, 204, 205, 206, 207, 208, 209, 210, 211, 212, 213, 214, 215, 216, 217, 218, 219, 220, 225, 226, 227, 228, 229, 230, 231, 232, 233, 234, 235, 236, 237, 238, 239, 240, 241, 242, 243, 244, 245, 246, 247, 248, 249, 250, 260, 270, 280, or 290 amino acids may have the advantage of increased expansion in vivo or in vitro.

As non-limiting examples, an immunoglobulin hinge region can include one of the following amino acid sequences: DKTHT (SEQ ID NO:84); CPPC (SEQ ID NO:85); CPEPKSCDTPPPCPR (SEQ ID NO:86); ELKTPLGDTTHT (SEQ ID NO:87); KSCDKTHTCP (SEQ ID NO:88); KCCVDCP (SEQ ID NO:89); KYGPPCP (SEQ ID NO:90); EPKSCDKTHTCPPCP (SEQ ID NO:91—human IgG1 hinge); ERKCCVECPPCP (SEQ ID NO:92—human IgG2 hinge); ELKTPLGDTTHTCPRCP (SEQ ID NO:130—human IgG3 hinge); SPNMVPHAHHAQ (SEQ ID NO:93); ESKYGPPCPPCP (SEQ ID NO:73) or ESKYGPPCPSCP (SEQ ID NO:94) (human IgG4 hinge-based) and the like. In some embodiments, the hinge is SEQ ID NO:73 or SEQ ID NO:94. In some embodiments, the hinge is SEQ ID NO:73.

The extracellular spacer can comprise an amino acid sequence of a human IgG1, IgG2, IgG3, or IgG4, hinge region. The extracellular spacer may also include one or more amino acid substitutions and/or insertions and/or deletions compared to a wild-type (naturally-occurring) hinge region. For example, His229 of human IgG1 hinge can be substituted with Tyr, so that the hinge region comprises the sequence EPKSCDKTYTCPPCP (SEQ ID NO:95).

The extracellular spacer can comprise an amino acid sequence derived from human CD8; e.g., the hinge region can comprise the amino acid sequence: TTTPAPRPPTPAPTIASQPLSLRPEACRPAAGGAVHTRGLDFACD (SEQ ID NO:96), or a variant thereof.

The extracellular spacer may comprise or further comprise a CH2 region. An exemplary CH2 region is APEFEGGPSVFLFPPKPKDTLMISRTPEVTCVVVDVSQEDPEVQFNWYVDGVEVHNA KTKPREEQFQSTYRVVSVLTVLHQDWLNGKEYKCKVSNKGLPSSIEKTISKAK (SEQ ID NO:97). The extracellular spacer may comprise or further comprise a CH3 region. An exemplary CH3 region is

(SEQ ID NO: 98)
GQPREPQVYTLPPSQEEMTKNQVSLTCLVKGFYPSDIAVEW
ESNGQPENNYKTTPPVLDSDGSFFLYSRLTVDKSRWQEGNV
FSCSVMHEALHNHYTQKSLSLSLGK.

When the extracellular spacer comprises multiple parts, such as a hinge, CH2, and/or CH3, there may be anywhere from 0-50 amino acids in between the various parts. For example, there may be at least, at most, or exactly 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 45, or 50 amino acids (or any derivable range therein) between the hinge and the CH2 or CH3 region or between the CH2 and CH3 region when both are present. In some embodiments, the extracellular spacer consists essentially of a hinge, CH2, and/or CH3 region, meaning that the hinge, CH2, and/or CH3 region is the only identifiable region present and all other domains or regions are excluded, but further amino acids not part of an identifiable region may be present.

C. Transmembrane Domain

The transmembrane domain is a hydrophobic alpha helix that spans the membrane. Different transmembrane domains may result in different receptor stability.

The transmembrane domain is interposed between the extracellular spacer and the cytoplasmic region. In some embodiments, the transmembrane domain is interposed between the extracellular spacer and one or more costimulatory regions. In some embodiments, a linker is between the transmembrane domain and the one or more costimulatory regions.

Any transmembrane domain that provides for insertion of a polypeptide into the cell membrane of a eukaryotic (e.g., mammalian) cell may be suitable for use. As one non-limiting example, the transmembrane sequence MFWVLVVVGGVLACYSLLVTVAFIIFWV (SEQ ID NO:99), which is CD28-derived can be used. In some embodiments, the transmembrane domain is CD8 beta derived: LGLLVAGVLVLLVSLGVAIHLCC (SEQ ID NO:100); CD4 derived: ALIVLGGVAGLLLFIGLGIFFCVRC (SEQ ID NO:101); CD3 zeta derived: LCYLLDGILFIYGVILTALFLRV (SEQ ID NO:102); CD28 derived: WVLVVVGGVLACYSLLVTVAFIIFWV (SEQ ID NO:103); CD134 (OX40) derived: VAAILGLGLVLGLLGPLAILLALYLL (SEQ ID NO:104); or CD7 derived: ALPAALAVISFLLGLGLGVACVLA (SEQ ID NO:105). In some embodiments, the transmembrane domain is derived from CD28, CD8, CD4, CD3-zeta, CD134, or CD7.

D. Cytoplasmic Region

After antigen recognition, receptors cluster and a signal is transmitted to the cell through the cytoplasmic region. In some embodiments, the costimulatory domains described herein are part of the cytoplasmic region.

Cytoplasmic regions and/or costimulatory regions suitable for use in the polypeptides of the disclosure include any desired signaling domain that provides a distinct and detectable signal (e.g., increased production of one or more cytokines by the cell; change in transcription of a target gene; change in activity of a protein; change in cell behavior, e.g., cell death; cellular proliferation; cellular differentiation; cell survival; modulation of cellular signaling responses; etc.) in response to activation by way of binding of the antigen to the antigen binding domain. In some embodiments, the cytoplasmic region includes at least one (e.g., one, two, three, four, five, six, etc.) ITAM motif as described herein. In some embodiments, the cytoplasmic region includes DAP10/CD28 type signaling chains.

Cytoplasmic regions suitable for use in the polypeptides of the disclosure include immunoreceptor tyrosine-based activation motif (ITAM)-containing intracellular signaling polypeptides. An ITAM motif is YX₁X₂(L/I), where X₁ and X₂ are independently any amino acid. In some cases, the cytoplasmic region comprises 1, 2, 3, 4, or 5 ITAM motifs. In some cases, an ITAM motif is repeated twice in an endodomain, where the first and second instances of the ITAM motif are separated from one another by 6 to 8 amino acids, e.g., (YX₁X₂(L/I))(X3)ₙ(YX₁X₂(L/I)), where n is an integer from 6 to 8, and each of the 6-8 X₃ can be any amino acid.

A suitable cytoplasmic region may be an ITAM motif-containing portion that is derived from a polypeptide that contains an ITAM motif. For example, a suitable cytoplasmic region can be an ITAM motif-containing domain from any ITAM motif-containing protein. Thus, a suitable endodomain need not contain the entire sequence of the entire protein from which it is derived. Examples of suitable ITAM motif-containing polypeptides include, but are not limited to: DAP12, DAP10, FCER1G (Fc epsilon receptor I gamma chain); CD3D (CD3 delta); CD3E (CD3 epsilon); CD3G (CD3 gamma); CD3-zeta; and CD79A (antigen receptor complex-associated protein alpha chain).

In some cases, the cytoplasmic region is derived from DAP12 (also known as TYROBP; TYRO protein tyrosine kinase binding protein; KARAP; PLOSL; DN AX-activation protein 12; KAR-associated protein; TYRO protein tyrosine kinase-binding protein; killer activating receptor associated protein; killer-activating receptor-associated protein; etc.). For example, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 106)
MGGLEPCSRLLLLPLLLAVSGLRPVQAQ
AQSDCSCSTVSPGVLAGIVMGDLVLTVL
IALAVYFLGRLVPRGRGAAEAATRKORI
TETESPYOELOGORSDVYSDLNTQRPYY
K;
(SEQ ID NO: 107)
MGGLEPCSRLLLLPLLLAVSGLRPVQAQ
AQSDCSCSTVSPGVLAGIVMGDLVLTVL
IALAVYFLGRLVPRGRGAAEATRKORIT
ETESPYOELOGORSDVYSDLNTQRPYYK;
(SEQ ID NO: 108)
MGGLEPCSRLLLLPLLLAVSDCSCSTVS
PGVLAGIVMGDLVLTVLIALAVYFLGRL
VPRGRGAAEAATRKORITETESPYOELO
GORSDVYSDLNTQRPYYK;
or
(SEQ ID NO: 109)
MGGLEPCSRLLLLPLLLAVSDCSCSTV
SPGVLAGIVMGDLVLTVLIALAVYFLG
RLVPRGRGAAEATRKORITETESPYOE
LOGORSDVYSDLNTQRPYYK.

In some embodiments, a suitable cytoplasmic region can comprise an ITAM motif-containing portion of the full length DAP12 amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 110)
ESPYOELOGORSDVYSDLNTO.

In some embodiments, the cytoplasmic region is derived from FCER1G (also known as FCRG; Fc epsilon receptor I gamma chain; Fc receptor gamma-chain; fc-epsilon R1-gamma; fcRgamma; fceRI gamma; high affinity immunoglobulin epsilon receptor subunit gamma; immunoglobulin E receptor, high affinity, gamma chain; etc.). For example, a suitable endodomain polypeptide can comprise an amino acid sequence having at least 75%, at least 80%, at least 85%, at least 90%, at least 95%, at least 98%, or 100% amino acid sequence identity to

(SEQ ID NO: 111)
MIPAVVLLLLLLVEQAAALGEPQLCYILDA
ILFLYGIVLTLLYCRLKIQVRKAAITSYEK
SDGVYTGLSTRNQETYETLKHEKPPQ.

In some embodiments, a suitable cytoplasmic region can comprise an ITAM motif-containing portion of the full length FCER1G amino acid sequence. Thus, a suitable endodomain polypeptide can comprise an amino acid sequence having at least 75%, at least 80%, at least 85%, at least 90%, at least 95%, at least 98%, or 100%, amino acid sequence identity to

(SEQ ID NO: 112)
DGVYTGLSTRNOETYETLKHE.

In some embodiments, the cytoplasmic region is derived from T cell surface glycoprotein CD3 delta chain (also known as CD3D; CD3-DELTA; T3D; CD3 antigen, delta subunit; CD3 delta; CD3δ; CD3d antigen, delta polypeptide (TiT3 complex); OKT3, delta chain; T cell receptor T3 delta chain; T cell surface glycoprotein CD3 delta chain; etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 170 aa, of either of the following amino acid sequences (2 isoforms):

(SEQ ID NO: 113)
MEHSTFLSGLVLATLLSQVSPFKIPIEELEDRVFVNCNTSITWVE
GTVGTLLSDITRLDLGKRILDPRGIYRCNGTDIYKDKESTVQVHY
RMCQSCVELDPATVAGIIVTDVIATLLLALGVFCFAGHETGRLSG
AADTOALLRNDOVYOPLRDRDDAOYSHLGGNWARNK
or
(SEQ ID NO: 114)
MEHSTFLSGLVLATLLSQVSPFKIPIEELEDRVFVNCNTSITWVE
GTVGTLLSDITRLDLGKRILDPRGIYRCNGTDIYKDKESTVOVHY
RTADTOALLRNDOVYOPLRDRDDAQYSHLGGNWARNK.

In some embodiments, a suitable cytoplasmic region can comprise an ITAM motif-containing portion of the full length CD3 delta amino acid sequence. Thus, a suitable endodomain polypeptide can comprise an amino acid sequence having at least 75%, at least 80%, at least 85%, at least 90%, at least 95%, at least 98%, or 100%, amino acid sequence identity to DOVYOPLRDRDDAOYSHLGGN (SEQ ID NO:115).

In some embodiments, the cytoplasmic region is derived from T cell surface glycoprotein CD3 epsilon chain (also known as CD3e, CD3ε; T cell surface antigen T3/Leu-4 epsilon chain, T cell surface glycoprotein CD3 epsilon chain, AI504783, CD3, CD3epsilon, T3e, etc.). For example, a suitable endodomain polypeptide can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 205 aa, of the following amino acid sequence:

(SEQ ID NO: 116)
MQSGTHWRVLGLCLLSVGVWGQDGNEEMGGITQTPYKVSISGTTV
ILTCPQYPGSEILWQHNDKNIGGDEDDKNIGSDEDHLSLKEFSEL
EQSGYYVCYPRGSKPEDANFYLYLRARVCENCMEMDVMSVATIVI
VDICITGGLLLLVYYWSKNRKAKAKPVTRGAGAGGRQRGQNKERP
PPVPNPDYEPIRKGQRDLYSGLNQRRI.

In some embodiments, a suitable cytoplasmic region can comprise an ITAM motif-containing portion of the full length CD3 epsilon amino acid sequence. Thus, a suitable endodomain polypeptide can comprise an amino acid sequence having at least 75%, at least 80%, at least 85%, at least 90%, at least 95%, at least 98%, or 100%, amino acid sequence identity to NPDYEPIRKGQRDLYSGLNQR (SEQ ID NO:117).

In some embodiments, the cytoplasmic region is derived from T cell surface glycoprotein CD3 gamma chain (also known as CD3G, CD37, T cell receptor T3 gamma chain, CD3-GAMMA, T3G, gamma polypeptide (TiT3 complex), etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 180 aa, of the following amino acid sequence:

(SEQ ID NO: 118)
MEQGKGLAVLILAIILLQGTLAQSIKGNHLVKVYDYQEDGSVLLT
CDAEAKNITWFKDGKMIGFLTEDKKKWNLGSNAKDPRGMYQCKGS
QNKSKPLQVYYRMCQNCIELNAATISGFLFAEIVSIFVLAVGVYF
IAGODGVROSRASDKOTLLPNDOLYOPLKDREDDQYSHLQGNQLR
RN.

In some embodiments, a suitable cytoplasmic region can comprise an ITAM motif-containing portion of the full length CD3 gamma amino acid sequence. Thus, a suitable cytoplasmic region can comprise an amino acid sequence having at least 75%, at least 80%, at least 85%, at least 90%, at least 95%, at least 98%, or 100%, amino acid sequence identity to DOLYOPLKDREDDOYSHLOGN (SEQ ID NO:119).

In some embodiments, the cytoplasmic region is derived from T cell surface glycoprotein CD3 zeta chain (also known as CD3Z, CD3ζ, T cell receptor T3 zeta chain, CD247, CD3-ZETA, CD3H, CD3Q, T3Z, TCRZ, etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 140 aa, from about 140 aa to about 150 aa, or from about 150 aa to about 160 aa, of either of the following amino acid sequences (2 isoforms):

(SEQ ID NO: 120)
MKWKALFTAAILQAQLPITEAQSFGLLDPKLCYLLDGILFIYGVIL
TALFLRVKFSRSADAPAYQQGQNQLYNELNLGRREEYDVLDKRRGR
DPEMGGKPRRKNPQEGLYNELQKDKMAEAYSEIGMKGERRRGKGHD
GLYQGLSTATKDTYDALHMQALPPR
or
(SEQ ID NO: 121)
MKWKALFTAAILQAQLPITEAQSFGLLDPKLCYLLDGILFIYGVIL
TALFLRVKFSRSADAPAYQQGQNQLYNELNLGRREEYDVLDKRRGR
DPEMGGKPQRRKNPQEGLYNELQKDKMAEAYSEIGMKGERRRGKGH
DGLYQGLSTATKDTYDALHMQALPPR.

In some embodiments, the cytoplasmic region comprises

(SEQ ID NO: 78)
RVKFSRSADAPAYQQGQNQLYNELNLGRREEYDVLDKR
RGRDPEMGGKPRRKNPQEGLYNELQKDKMAEAYSEIGM
KGERRRGKGHDGLYQGLSTATKDTYDALHMQALPPR.

In some embodiments, a suitable cytoplasmic region can comprise an ITAM motif-containing portion of the full length CD3 zeta amino acid sequence. Thus, a suitable cytoplasmic region can comprise an amino acid sequence having at least 75%, at least 80%, at least 85%, at least 90%, at least 95%, at least 98%, or 100%, amino acid sequence identity to any of the following amino acid sequences:

(SEQ ID NO: 122)
RVKFSRSADAPAYOQGONOLYNELNLGRREEYD
VLDKRRGRDPEMGGKPRRKNPOEGLYNELOKDK
MAEAYSEIGMKGERRRGKGHDGLYOGLSTATKD
TYDALHMQALPPR;
(SEQ ID NO: 123)
NOLYNELNLGRREEYDVLDKR;
(SEQ ID NO: 124)
EGLYNELQKDKMAEAYSEIGMK;
or
(SEQ ID NO: 125)
DGLYOGLSTATKDTYDALHMO.

In some embodiments, the cytoplasmic region is derived from CD79A (also known as B-cell antigen receptor complex-associated protein alpha chain; CD79a antigen (immunoglobulin-associated alpha); MB-1 membrane glycoprotein; ig-alpha; membrane-bound immunoglobulin-associated protein; surface IgM-associated protein; etc.). For example, a suitable cytoplasmic region can comprise an amino acid sequence having at least 75%, at least 80%, at least 85%, at least 90%, at least 95%, at least 98%, or 100%, amino acid sequence identity to a contiguous stretch of from about 100 amino acids to about 110 amino acids (aa), from about 110 aa to about 115 aa, from about 115 aa to about 120 aa, from about 120 aa to about 130 aa, from about 130 aa to about 150 aa, from about 150 aa to about 200 aa, or from about 200 aa to about 220 aa, of either of the following amino acid sequences (2 isoforms)

(SEQ ID NO: 126)
MPGGPGVLQALPATIFLLFLLSAVYLGPGCQALWMHKVPASLM
VSLGEDAHFQCPHNSSNNANVTWWRVLHGNYTWPPEFLGPGED
PNGTLIIQNVNKSHGGIYVCRVQEGNESYQQSCGTYLRVRQPP
PRPFLDMGEGTKNRIITAEGIILLFCAVVPGTLLLFRKRWONE
KLGLDAGDEYEDENLYEGLNLDDCSMYEDISRGLOGTYQDVGS
LNIGDVQLEKP;
or
(SEQ ID NO: 127)
MPGGPGVLQALPATIFLLFLLSAVYLGPGCQALWMHKVPASLMV
SLGEDAHFQCPHNSSNNANVTWWRVLHGNYTWPPEFLGPGEDPN
EPPPRPFLDMGEGTKNRIITAEGIILLFCAVVPGTLLLFRKRWQ
NEKLGLDAGDEYEDENLYEGLNLDDCSMYEDISRGLQGTYQDVG
SLNIGDVQLEKP.

In some embodiments, a suitable cytoplasmic region can comprise an ITAM motif-containing portion of the full length CD79A amino acid sequence. Thus, a suitable cytoplasmic region can comprise an amino acid sequence having at least 75%, at least 80%, at least 85%, at least 90%, at least 95%, at least 98%, or 100%, amino acid sequence identity to the following amino acid sequence: ENLYEGLNLDDCSMYEDISRG (SEQ ID NO:128).

In some embodiments, suitable cytoplasmic regions can comprise a DAP10/CD28 type signaling chain. An example of a CD28 signaling chain is the amino acid sequence FWVLVVVGGVLACYSLLVTVAFIIFWVRSKRSRLLHSDYMNMTPRRPGPTRKHYQP YAPPRDFAAYRS (SEQ ID NO:131). In some embodiments, a suitable endodomain comprises an amino acid sequence having at least 85%, at least 90%, at least 95%, at least 98%, or at least 99%, amino acid sequence identity to the entire length of the amino acid sequence

(SEQ ID NO: 132)
FWVLVVVGGVLACYSLLVTVAFIIFWVRS
KRSRLLHSDYMNMTPRRPGPTRKHYQPYA
PPRDFAAYRS.

Further cytoplasmic regions suitable for use in the polypeptides of the disclosure include a ZAP70 polypeptide, e.g., a polypeptide comprising an amino acid sequence having at least 85%, at least 90%, at least 95%, at least 98%, at least 99%, or 100%, amino acid sequence identity to a contiguous stretch of from about 300 amino acids to about 400 amino acids, from about 400 amino acids to about 500 amino acids, or from about 500 amino acids to 619 amino acids, of the following amino acid sequence:

(SEQ ID NO: 133)
MPDPAAHLPFFYGSISRAEAEEHLKLAGMADGLFLLRQCLRSLGGY
VLSLVHDVRFHHFPIERQLNGTYAIAGGKAHCGPAELCEFYSRDPD
GLPCNLRKPCNRPSGLEPQPGVFDCLRDAMVRDYVRQTWKLEGEAL
EQAIISQAPQVEKLIATTAHERMPWYHSSLTREEAERKLYSGAQTD
GKFLLRPRKEQGTYALSLIYGKTVYHYLISQDKAGKYCIPEGTKFD
TLWQLVEYLKLKADGLIYCLKEACPNSSASNASGAAAPTLPAHPST
LTHPQRRIDTLNSDGYTPEPARITSPDKPRPMPMDTSVYESPYSDP
EELKDKKLFLKRDNLLIADIELGCGNFGSVRQGVYRMRKKQIDVAI
KVLKQGTEKADTEEMMREAQIMHQLDNPYIVRLIGVCQAEALMLVM
EMAGGGPLHKFLVGKREEIPVSNVAELLHQVSMGMKYLEEKNFVHR
DLAARNVLLVNRHYAKISDFGLSKALGADDSYYTARSAGKWPLKWY
APECINFRKFSSRSDVWSYGVTMWEALSYGQKPYKKMKGPEVMAFI
EQGKRMECPPECPPELYALMSDCWIYKWEDRPDFLTVEQRMRACYY
SLASKVEGPPGSTQKAEAACA.

a. Costimulatory Region

Non-limiting examples of suitable costimulatory regions, such as those included in the cytoplasmic region, include, but are not limited to, polypeptides from 4-1BB (CD137), CD28, ICOS, OX-40, BTLA, CD27, CD30, GITR, and HVEM.

A co-stimulatory region may have a length of at least, at most, or exactly 20, 25, 30, 35, 40, 50, 60, 70, 80, 90, 100, 150, 200, or 300 amino acids or any range derivable therein. In some embodiments, the costimulatory region is derived from an intracellular portion of the transmembrane protein 4-1BB (also known as TNFRSF9; CD137; CDw137; ILA; etc.). For example, a suitable costimulatory region can comprise an amino acid sequence having at least 75%, at least 80%, at least 85%, at least 90%, at least 95%, at least 98%, or 100% amino acid sequence identity to

(SEQ ID NO: 77)
KRGRKKLLYIFKQPFMRPVQTTQEEDGCSCRFPEEEEGGCEL.

In some embodiments, the costimulatory region is derived from an intracellular portion of the transmembrane protein CD28 (also known as Tp44). For example, a suitable costimulatory region can comprise an amino acid sequence having at least 75%, at least 80%, at least 85%, at least 90%, at least 95%, at least 98%, or 100% amino acid sequence identity to FWVRSKRSRLLHSDYMNMTPRRPGPTRKHYQPYAPPRDFAAYRS (SEQ ID NO:134).

In some embodiments, the costimulatory region is derived from an intracellular portion of the transmembrane protein ICOS (also known as AILIM, CD278, and CVID1). For example, a suitable costimulatory region can comprise an amino acid sequence having at least 75%, at least 80%, at least 85%, at least 90%, at least 95%, at least 98%, or 100% amino acid sequence identity to TKKKYSSSVHDPNGEYMFMRAVNTAKKSRLTDVTL (SEQ ID NO:135).

In some embodiments, the costimulatory region is derived from an intracellular portion of the transmembrane protein OX-40 (also known as TNFRSF4, RP5-902P8.3, ACT35, CD134, OX40, TXGP1L). For example, a suitable co-stimulatory region can comprise an amino acid sequence having at least 75%, at least 80%, at least 85%, at least 90%, at least 95%, at least 98%, or 100% amino acid sequence identity to

(SEQ ID NO: 136)
RRDQRLPPDAHKPPGGGSFRTPIQEEQADAHSTLAKI.

In some embodiments, the costimulatory region is derived from an intracellular portion of the transmembrane protein BTLA (also known as BTLA1 and CD272). For example, a suitable costimulatory region can comprise an amino acid sequence having at least 75%, at least 80%, at least 85%, at least 90%, at least 95%, at least 98%, or 100% amino acid sequence identity to

(SEQ ID NO: 137)
CCLRRHQGKQNELSDTAGREINLVDAHLK
SEQTEASTRQNSQVLLSETGIYDNDPDLC
FRMQEGSEVYSNPCLEENKPGIVYASLNH
SVIGPNSRLARNVKEAPTEYASICVRS.

In some embodiments, the costimulatory region is derived from an intracellular portion of the transmembrane protein CD27 (also known as S 152, T14, TNFRSF7, and Tp55). For example, a suitable costimulatory region can comprise an amino acid sequence having at least 75%, at least 80%, at least 85%, at least 90%, at least 95%, at least 98%, or 100% amino acid sequence identity to

(SEQ ID NO: 138)
HQRRKYRSNKGESPVEPAEPCRYSCPR
EEEGSTIPIQEDYRKPEPACSP.

In some embodiments, the costimulatory region is derived from an intracellular portion of the transmembrane protein CD30 (also known as TNFRSF8, D1S166E, and Ki-1). For example, a suitable costimulatory region can comprise an amino acid sequence having at least 75%, at least 80%, at least 85%, at least 90%, at least 95%, at least 98%, or 100% amino acid sequence identity to

(SEQ ID NO: 139)
RRACRKRIRQKLHLCYPVQTSQPKLELV
DSRPRRSSTQLRSGASVTEPVAEERGLM
SQPLMETCHSVGAAYLESLPLQDASPAG
GPSSPRDLPEPRVSTEHTNNKIEKIYIM
KADTVIVGTVKAELPEGRGLAGPAEPEL
EEELEADHTPHYPEQETEPPLGSCSDVM
LSVEEEGKEDPLPTAASGK.

In some embodiments, the costimulatory region is derived from an intracellular portion of the transmembrane protein GITR (also known as TNFRSF18, RP5-902P8.2, AITR, CD357, and GITR-D). For example, a suitable co-stimulatory region can comprise an amino acid sequence having at least 75%, at least 80%, at least 85%, at least 90%, at least 95% at least 98% or 100% amino acid sequence identity to

(SEQ ID NO: 140)
HIWQLRSQCMWPRETQLLLEVPPSTEDAR
SCQFPEEERGERSAEEKGRLGDLWV.

In some embodiments, the costimulatory region derived from an intracellular portion of the transmembrane protein HVEM (also known as TNFRSF14, RP3-395M20.6, ATAR, CD270, HVEA, HVEM, LIGHTR, and TR2). For example, a suitable costimulatory region can comprise an amino acid sequence having at least 75%, at least 80%, at least 85%, at least 90%, at least 95%, at least 98%, or 100% amino acid sequence identity to

(SEQ ID NO: 141)
CVKRRKPRGDVVKVIVSVQRKRQEAEGEAT
VIEALQAPPDVTTVAVEET1PSFTGRSPNH.

E. Detection Peptides

In some embodiments, the polypeptides described herein may further comprise a detection peptide. Suitable detection peptides include hemagglutinin (HA; e.g., YPYDVPDYA (SEQ ID NO:142); FLAG (e.g., DYKDDDDK (SEQ ID NO:143); c-myc (e.g., EQKLISEEDL; SEQ ID NO:144), and the like. Other suitable detection peptides are known in the art.

F. Peptide Linkers

In some embodiments, the polypeptides of the disclosure include peptide linkers (sometimes referred to as a linker). A peptide linker may be used to separate any of the peptide domain/regions described herein. As an example, a linker may be between the signal peptide and the antigen binding domain, between the VH and VL of the antigen binding domain, between the antigen binding domain and the peptide spacer, between the peptide spacer and the transmembrane domain, flanking the costimulatory region or on the N- or C-region of the costimulatory region, and/or between the transmembrane domain and the endodomain. The peptide linker may have any of a variety of amino acid sequences. Domains and regions can be joined by a peptide linker that is generally of a flexible nature, although other chemical linkages are not excluded. A linker can be a peptide of between about 6 and about 40 amino acids in length, or between about 6 and about 25 amino acids in length. These linkers can be produced by using synthetic, linker-encoding oligonucleotides to couple the proteins.

Peptide linkers with a degree of flexibility can be used. The peptide linkers may have virtually any amino acid sequence, bearing in mind that suitable peptide linkers will have a sequence that results in a generally flexible peptide. The use of small amino acids, such as glycine and alanine, are of use in creating a flexible peptide. The creation of such sequences is routine to those of skill in the art.

Suitable linkers can be readily selected and can be of any suitable length, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Suitable linkers can be readily selected and can be of any of a suitable of different lengths, such as from 1 amino acid (e.g., Gly) to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids.

Exemplary flexible linkers include glycine polymers (G)ₙ, glycine-serine polymers (including, for example, (GS)ₙ, (GSGGS)ₙ (SEQ ID NO:145), (G4S)ₙ and (GGGS)ₙ (SEQ ID NO:146), where n is an integer of at least one. In some embodiments, n is at least, at most, or exactly 1, 2, 3, 4, 5, 6, 7, 8, 9, or 10 (or any derivable range therein). Glycine-alanine polymers, alanine-serine polymers, and other flexible linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains. Exemplary spacers can comprise amino acid sequences including, but not limited to, GGSG (SEQ ID NO:147), GGSGG (SEQ ID NO:148), GSGSG (SEQ ID NO:149), GSGGG (SEQ ID NO:150), GGGSG (SEQ ID NO:151), GSSSG (SEQ ID NO:152), and the like.

In further embodiments, the linker comprises (EAAAK)ₙ, wherein n is an integer of at least one. In some embodiments, n is at least, at most, or exactly 1, 2, 3, 4, 5, 6, 7, 8, 9, or 10 (or any derivable range therein).

G. Therapeutic Controls

In some embodiments of the methods and compositions described herein, the CAR molecule is co-expressed with a therapeutic control.

Therapeutic controls regulate cell proliferation, facilitate cell selection (for example selecting cells which express the chimeric antigen receptors of the invention) or a combination thereof. In one embodiment, regulating cell proliferation comprises up-regulating cell proliferation to promote cell propagation. In another embodiment, regulating cell proliferation comprises down-regulating cell proliferation so as to reduce or inhibit cell propagation. In some embodiments, the agents that serve as therapeutic controls may promote enrichment of cells which express the chimeric antigen receptors which may result in a therapeutic advantage. In some embodiments, agents which serve as therapeutic controls may biochemically interact with additional compositions so as to regulate the functioning of the therapeutic controls. For example, EGFRt (a therapeutic control) may biochemically interact with cetuximab so as to regulate the function of EGFRt in selection, tracking, cell ablation or a combination thereof.

Exemplary therapeutic controls include truncated epidermal growth factor receptor (EGFRt), chimeric cytokine receptors (CCR) and/or dihydroxyfolate receptor (DHFR) (e.g., mutant DHFR). The polynucleotides encoding the CAR and the therapeutic control(s) may be linked via IRES sequences or via polynucleotide sequences encoding cleavable linkers. The CARs of the invention are constructed so that they may be expressed in cells, which in turn proliferate in response to the presence of at least one molecule that interacts with at least one antigen-specific targeting region, for instance, an antigen. In further embodiments, the therapeutic control comprises a cell-surface protein wherein the protein lacks intracellular signaling domains. It is contemplated that any cell surface protein lacking intracellular signaling or modified (e.g. by truncation) to lack intracellular signaling may be used. Further examples of a therapeutic control include truncated LNGFR, truncated CD19 etc. . . . wherein the truncated proteins lack intracellular signaling domains.

“Co-express” as used herein refers to simultaneous expression of two or more genes. Genes may be nucleic acids encoding, for example, a single protein or a chimeric protein as a single polypeptide chain. For example, the CARs of the disclosure may be co-expressed with a therapeutic control (for example truncated epidermal growth factor (EGFRt)), wherein the CAR is encoded by a first polynucleotide chain and the therapeutic control is encoded by a second polynucleotide chain. In an embodiment, the first and second polynucleotide chains are linked by a nucleic acid sequence that encodes a cleavable linker The polynucleotides encoding the CAR and the therapeutic control system may be linked by IRES sequences. Alternately, the CAR and the therapeutic control are encoded by two different polynucleotides that are not linked via a linker but are instead encoded by, for example, two different vectors. Further, the CARs of the disclosure may be co-expressed with a therapeutic control and CCR, a therapeutic control and DHFR (for example mutant DHFR) or a therapeutic control and CCR and DHFR (for example mutant DHFR). The CAR, therapeutic control and CCR may be co-expressed and encoded by first, second and third polynucleotide sequences, respectively, wherein the first, second and third polynucleotide sequences are linked via IRES sequences or sequences encoding cleavable linkers. Alternately, these sequences are not linked via linkers but instead are encoded via, for example, separate vectors. The CAR, therapeutic control and DHFR (for example mutant DHFR) may be co-expressed and encoded by first, second and fourth polynucleotide sequences, respectively, wherein the first, second and fourth polynucleotide sequences are linked via IRES sequences or via sequences encoding cleavable linkers. Alternately, these sequences are not linked via linkers but instead encoded via, for example, separate vectors. The CAR, therapeutic control, CCR and DHFR (for example mutant DHFR) may be co-expressed and encoded by first, second, third and fourth polynucleotide sequences, respectively, wherein the first, second, third and fourth polynucleotide sequences are linked via IRES sequences or sequences encoding cleavable linkers. Alternately, these sequences are not linked via linkers but instead are encoded via, for example, separate vectors. If the aforementioned sequences are encoded by separate vectors, these vectors may be simultaneously or sequentially transfected.

Further aspects of the therapeutic controls, CAR molecules, and methods of use for the compositions of the disclosure can be found in U.S. Pat. No. 9,447,194, which is herein incorporated by reference for all purposes.

H. Additional Modifications and Polypeptide Embodiments

Additionally, the polypeptides of the disclosure may be chemically modified. Glycosylation of the polypeptides can be altered, for example, by modifying one or more sites of glycosylation within the polypeptide sequence to increase the affinity of the polypeptide for antigen (U.S. Pat. Nos. 5,714,350 and 6,350,861).

It is contemplated that a region or fragment of a polypeptide of the disclosure may have an amino acid sequence that has, has at least or has at most 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200 or more amino acid substitutions, contiguous amino acid additions, or contiguous amino acid deletions with respect to any of SEQ ID NOs:1-152, or 169-177. Alternatively, a region or fragment of a polypeptide of the disclosure may have an amino acid sequence that comprises or consists of an amino acid sequence that is, is at least, or is at most 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% (or any range derivable therein) identical to any of SEQ ID NOs:1-152, or 169-177. Moreover, in some embodiments, a region or fragment comprises an amino acid region of 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 or more contiguous amino acids starting at position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500 in any of SEQ ID NOs:1-152, or 169-177 (where position 1 is at the N-terminus of the SEQ ID NO). The polypeptides of the disclosure may include 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 or more variant amino acids or nucleic acid substitutions or be at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% similar, identical, or homologous with at least, or at most 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000, 1500, or 2000 or more contiguous amino acids or nucleic acids, or any range derivable therein, of any of SEQ ID NOs:1-152, or 169-177.

The polypeptides of the disclosure may include at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, or 615 substitutions (or any range derivable therein).

The substitution may be at amino acid position 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 650, 700, 750, 800, 850, 900, 1000, 1500, or 2000 of any of SEQ ID NOs:1-152, or 169-177 (or any derivable range therein).

The polypeptides described herein may be of a fixed length of at least, at most, or exactly 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 300, 400, 500, 550, 1000 or more amino acids (or any derivable range therein).

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

Proteins may be recombinant, or synthesized in vitro. Alternatively, a non-recombinant or recombinant protein may be isolated from bacteria. It is also contemplated that bacteria containing such a variant may be implemented in compositions and methods. Consequently, a protein need not be isolated.

The term “functionally equivalent codon” is used herein to refer to codons that encode the same amino acid, such as the six codons for arginine or serine, and also refers to codons that encode biologically equivalent amino acids.

It also will be understood that amino acid and nucleic acid sequences may include additional residues, such as additional N- or C-terminal amino acids, or 5′ or 3′ sequences, respectively, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences that may, for example, include various non-coding sequences flanking either of the 5′ or 3′ portions of the coding region.

The following is a discussion based upon changing of the amino acids of a protein to create an equivalent, or even an improved, second-generation molecule. For example, certain amino acids may be substituted for other amino acids in a protein structure without appreciable loss of interactive binding capacity. Structures such as, for example, an enzymatic catalytic domain or interaction components may have amino acid substituted to maintain such function. Since it is the interactive capacity and nature of a protein that defines that protein's biological functional activity, certain amino acid substitutions can be made in a protein sequence, and in its underlying DNA coding sequence, and nevertheless produce a protein with like properties. It is thus contemplated by the inventors that various changes may be made in the DNA sequences of genes without appreciable loss of their biological utility or activity.

In other embodiments, alteration of the function of a polypeptide is intended by introducing one or more substitutions. For example, certain amino acids may be substituted for other amino acids in a protein structure with the intent to modify the interactive binding capacity of interaction components. Structures such as, for example, protein interaction domains, nucleic acid interaction domains, and catalytic sites may have amino acids substituted to alter such function. Since it is the interactive capacity and nature of a protein that defines that protein's biological functional activity, certain amino acid substitutions can be made in a protein sequence, and in its underlying DNA coding sequence, and nevertheless produce a protein with different properties. It is thus contemplated by the inventors that various changes may be made in the DNA sequences of genes with appreciable alteration of their biological utility or activity.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. It is understood that an amino acid can be substituted for another having a similar hydrophilicity value and still produce a biologically equivalent and immunologically equivalent protein.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known and include: arginine and lysine; glutamate and aspartate; serine and threonine; glutamine and asparagine; and valine, leucine and isoleucine.

In specific embodiments, all or part of proteins described herein can also be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Alternatively, recombinant DNA technology may be employed wherein a nucleotide sequence that encodes a peptide or polypeptide is inserted into an expression vector, transformed or transfected into an appropriate host cell and cultivated under conditions suitable for expression.

One embodiment includes the use of gene transfer to cells, including microorganisms, for the production and/or presentation of proteins. The gene for the protein of interest may be transferred into appropriate host cells followed by culture of cells under the appropriate conditions. A nucleic acid encoding virtually any polypeptide may be employed. The generation of recombinant expression vectors, and the elements included therein, are discussed herein. Alternatively, the protein to be produced may be an endogenous protein normally synthesized by the cell used for protein production.

### III. CELLS

Certain embodiments relate to cells comprising polypeptides or nucleic acids of the disclosure. In some embodiments the cell is an immune cell or a T cell. “T cell” includes all types of immune cells expressing CD3 including T-helper cells, invariant natural killer T (iNKT) cells, cytotoxic T cells, T-regulatory cells (Treg) gamma-delta T cells, natural-killer (NK) cells, and neutrophils. The T cell may refer to a CD4+ or CD8+ T cell.

Suitable mammalian cells include primary cells and immortalized cell lines. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), human embryonic kidney (HEK) 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RATI cells, mouse L cells (ATCC No. CCLI.3), HLHepG2 cells, Hut-78, Jurkat, HL-60, NK cell lines (e.g., NKL, NK92, and YTS), and the like.

In some instances, the cell is not an immortalized cell line, but is instead a cell (e.g., a primary cell) obtained from an individual. For example, in some cases, the cell is an immune cell obtained from an individual. As an example, the cell is a T lymphocyte obtained from an individual. As another example, the cell is a cytotoxic cell obtained from an individual. As another example, the cell is a stem cell or progenitor cell obtained from an individual.

### IV. METHODS FOR MODIFYING GENOMIC DNA

In certain embodiments, the genomic DNA is modified either to include additional mutations, insertions, or deletions, or to integrate certain molecular constructs of the disclosure so that the constructs are expressed from the genomic DNA. In some embodiments, a nucleic acid encoding a polypeptide of the disclosure is integrated into the genomic DNA of a cell. In some embodiments, the integration is targeted integration. In some embodiments, targeted integration is achieved through the use of a DNA digesting agent/polynucleotide modification enzyme, such as a site-specific recombinase and/or a targeting endonuclease. The term “DNA digesting agent” refers to an agent that is capable of cleaving bonds (i.e. phosphodiester bonds) between the nucleotide subunits of nucleic acids. One specific target is the TRAC (T cell receptor alpha constant) locus. For instance, cells would first be electroporated with a ribonucleoprotein (RNP) complex consisting of Cas9 protein complexed with a single-guide RNA (sgRNA) targeting the TRAC (T cell receptor alpha constant) locus. Fifteen minutes post electroporation, the cells would be treated with AAV6 carrying the HDR template that encodes for the CAR.

Therefore, one aspect, the current disclosure includes targeted integration. One way of achieving this is through the use of an exogenous nucleic acid sequence (i.e., a landing pad) comprising at least one recognition sequence for at least one polynucleotide modification enzyme, such as a site-specific recombinase and/or a targeting endonuclease. Site-specific recombinases are well known in the art, and may be generally referred to as invertases, resolvases, or integrases. Non-limiting examples of site-specific recombinases may include lambda integrase, Cre recombinase, FLP recombinase, gamma-delta resolvase, Tn3 resolvase, (DC31 integrase, Bxbl-integrase, and R4 integrase. Site-specific recombinases recognize specific recognition sequences (or recognition sites) or variants thereof, all of which are well known in the art. For example, Cre recombinases recognize LoxP sites and FLP recombinases recognize FRT sites.

Contemplated targeting endonucleases include zinc finger nucleases (ZFNs), meganucleases, transcription activator-like effector nucleases (TALENs), CRIPSR/Cas-like endonucleases, I-Tevl nucleases or related monomeric hybrids, or artificial targeted DNA double strand break inducing agents. Exemplary targeting endonucleases is further described below. For example, typically, a zinc finger nuclease comprises a DNA binding domain (i.e., zinc finger) and a cleavage domain (i.e., nuclease), both of which are described below. Also included in the definition of polynucleotide modification enzymes are any other useful fusion proteins known to those of skill in the art, such as may comprise a DNA binding domain and a nuclease.

A landing pad sequence is a nucleotide sequence comprising at least one recognition sequence that is selectively bound and modified by a specific polynucleotide modification enzyme such as a site-specific recombinase and/or a targeting endonuclease. In general, the recognition sequence(s) in the landing pad sequence does not exist endogenously in the genome of the cell to be modified. For example, where the cell to be modified is a CHO cell, the recognition sequence in the landing pad sequence is not present in the endogenous CHO genome. The rate of targeted integration may be improved by selecting a recognition sequence for a high efficiency nucleotide modifying enzyme that does not exist endogenously within the genome of the targeted cell. Selection of a recognition sequence that does not exist endogenously also reduces potential off-target integration. In other aspects, use of a recognition sequence that is native in the cell to be modified may be desirable. For example, where multiple recognition sequences are employed in the landing pad sequence, one or more may be exogenous, and one or more may be native.

One of ordinary skill in the art can readily determine sequences bound and cut by site-specific recombinases and/or targeting endonucleases.

Multiple recognition sequences may be present in a single landing pad, allowing the landing pad to be targeted sequentially by two or more polynucleotide modification enzymes such that two or more unique nucleic acids (comprising, among other things, receptor genes and/or inducible reporters) can be inserted. Alternatively, the presence of multiple recognition sequences in the landing pad, allows multiple copies of the same nucleic acid to be inserted into the landing pad. When two nucleic acids are targeted to a single landing pad, the landing pad includes a first recognition sequence for a first polynucleotide modification enzyme (such as a first ZFN pair), and a second recognition sequence for a second polynucleotide modification enzyme (such as a second ZFN pair). Alternatively, or additionally, individual landing pads comprising one or more recognition sequences may be integrated at multiple locations. Increased protein expression may be observed in cells transformed with multiple copies of a payload Alternatively, multiple gene products may be expressed simultaneously when multiple unique nucleic acid sequences comprising different expression cassettes are inserted, whether in the same or a different landing pad. Regardless of the number and type of nucleic acid, when the targeting endonuclease is a ZFN, exemplary ZFN pairs include hSIRT, hRSK4, and hAAVS1, with accompanying recognition sequences.

Generally speaking, a landing pad used to facilitate targeted integration may comprise at least one recognition sequence. For example, a landing pad may comprise at least one, at least two, at least three, at least four, at least five, at least six, at least seven, at least eight, at least nine, or at least ten or more recognition sequences. In embodiments comprising more than one recognition sequence, the recognition sequences may be unique from one another (i.e. recognized by different polynucleotide modification enzymes), the same repeated sequence, or a combination of repeated and unique sequences.

One of ordinary skill in the art will readily understand that an exogenous nucleic acid used as a landing pad may also include other sequences in addition to the recognition sequence(s). For example, it may be expedient to include one or more sequences encoding selectable or screenable genes as described herein, such as antibiotic resistance genes, metabolic selection markers, or fluorescence proteins. Use of other supplemental sequences such as transcription regulatory and control elements (i.e., promoters, partial promoters, promoter traps, start codons, enhancers, introns, insulators and other expression elements) can also be present.

In addition to selection of an appropriate recognition sequence(s), selection of a targeting endonuclease with a high cutting efficiency also improves the rate of targeted integration of the landing pad(s). Cutting efficiency of targeting endonucleases can be determined using methods well-known in the art including, for example, using assays such as a CEL-1 assay or direct sequencing of insertions/deletions (Indels) in PCR amplicons.

The type of targeting endonuclease used in the methods and cells disclosed herein can and will vary. The targeting endonuclease may be a naturally-occurring protein or an engineered protein. One example of a targeting endonuclease is a zinc-finger nuclease, which is discussed in further detail below.

Another example of a targeting endonuclease that can be used is an RNA-guided endonuclease comprising at least one nuclear localization signal, which permits entry of the endonuclease into the nuclei of eukaryotic cells. The RNA-guided endonuclease also comprises at least one nuclease domain and at least one domain that interacts with a guiding RNA. An RNA-guided endonuclease is directed to a specific chromosomal sequence by a guiding RNA such that the RNA-guided endonuclease cleaves the specific chromosomal sequence. Since the guiding RNA provides the specificity for the targeted cleavage, the endonuclease of the RNA-guided endonuclease is universal and may be used with different guiding RNAs to cleave different target chromosomal sequences. Discussed in further detail below are exemplary RNA-guided endonuclease proteins. For example, the RNA-guided endonuclease can be a CRISPR/Cas protein or a CRISPR/Cas-like fusion protein, an RNA-guided endonuclease derived from a clustered regularly interspersed short palindromic repeats (CRISPR)/CRISPR-associated (Cas) system.

The targeting endonuclease can also be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named “LAGLIDADG” has become a valuable tool for the study of genomes and genome engineering. Meganucleases may be targeted to specific chromosomal sequence by modifying their recognition sequence using techniques well known to those skilled in the art. See, for example, Epinat et al., 2003, Nuc. Acid Res., 31(11):2952-62 and Stoddard, 2005, Quarterly Review of Biophysics, pp. 1-47.

Yet another example of a targeting endonuclease that can be used is a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. See, e.g., Sanjana et al., 2012, Nature Protocols 7(1):171-192; Bogdanove A J, Voytas D F., 2011, Science, 333(6051):1843-6; Bradley P, Bogdanove A J, Stoddard B L., 2013, Curr Opin Struct Biol., 23(1):93-9.

Another exemplary targeting endonuclease is a site-specific nuclease. In particular, the site-specific nuclease may be a “rare-cutter” endonuclease whose recognition sequence occurs rarely in a genome. Preferably, the recognition sequence of the site-specific nuclease occurs only once in a genome. Alternatively, the targeting nuclease may be an artificial targeted DNA double strand break inducing agent.

In some embodiments, targeted integrated can be achieved through the use of an integrase. For example, The phiC31 integrase is a sequence-specific recombinase encoded within the genome of the bacteriophage phiC31. The phiC31 integrase mediates recombination between two 34 base pair sequences termed attachment sites (att), one found in the phage and the other in the bacterial host. This serine integrase has been show to function efficiently in many different cell types including mammalian cells. In the presence of phiC31 integrase, an attB-containing donor plasmid can be unidirectional integrated into a target genome through recombination at sites with sequence similarity to the native attP site (termed pseudo-attP sites). phiC31 integrase can integrate a plasmid of any size, as a single copy, and requires no cofactors. The integrated transgenes are stably expressed and heritable.

In one embodiment, genomic integration of polynucleotides of the disclosure is achieved through the use of a transposase. For example, a synthetic DNA transposon (e.g. “Sleeping Beauty” transposon system) designed to introduce precisely defined DNA sequences into the chromosome of vertebrate animals can be used. The Sleeping Beauty transposon system is composed of a Sleeping Beauty (SB) transposase and a transposon that was designed to insert specific sequences of DNA into genomes of vertebrate animals. DNA transposons translocate from one DNA site to another in a simple, cut-and-paste manner. Transposition is a precise process in which a defined DNA segment is excised from one DNA molecule and moved to another site in the same or different DNA molecule or genome.

As do all other Tc1/mariner-type transposases, SB transposase inserts a transposon into a TA dinucleotide base pair in a recipient DNA sequence. The insertion site can be elsewhere in the same DNA molecule, or in another DNA molecule (or chromosome). In mammalian genomes, including humans, there are approximately 200 million TA sites. The TA insertion site is duplicated in the process of transposon integration. This duplication of the TA sequence is a hallmark of transposition and used to ascertain the mechanism in some experiments. The transposase can be encoded either within the transposon or the transposase can be supplied by another source, in which case the transposon becomes a non-autonomous element. Non-autonomous transposons are most useful as genetic tools because after insertion they cannot independently continue to excise and re-insert. All of the DNA transposons identified in the human genome and other mammalian genomes are non-autonomous because even though they contain transposase genes, the genes are non-functional and unable to generate a transposase that can mobilize the transposon.

### V. METHODS

Aspects of the current disclosure relate to methods for treating cancer, such as multiple myeloma. In further embodiments, the CAR molecules described herein may be used for stimulating an immune response. The immune response stimulation may be done in vitro, in vivo, or ex vivo. In some embodiments, the CAR molecules described herein are for preventing relapse. The method generally involves genetically modifying a mammalian cell with an expression vector, or an RNA (e.g., in vitro transcribed RNA), comprising nucleotide sequences encoding a polypeptide of the disclosure or directly transferring the polypeptide to the cell. The cell can be an immune cell (e.g., a T lymphocyte or NK cell), a stem cell, a progenitor cell, etc. In some embodiments, the cell is a cell described herein.

In some embodiments, the genetic modification is carried out ex vivo. For example, a T lymphocyte, a stem cell, or an NK cell (or cell described herein) is obtained from an individual; and the cell obtained from the individual is genetically modified to express a polypeptide of the disclosure. In some cases, the genetically modified cell is activated ex vivo. In other cases, the genetically modified cell is introduced into an individual (e.g., the individual from whom the cell was obtained); and the genetically modified cell is activated in vivo.

In some embodiments, the methods relate to administration of the cells or peptides described herein for the treatment of a cancer or administration to a person with a cancer. In some embodiments, the cancer is multiple myeloma. In some embodiments, the cancer is a B-cell cancer. In some embodiments the cancer is diffuse large B-cell lymphoma, follicular lymphoma, marginal zone B-cell lymphoma, mucosa-associated lymphatic tissue lymphoma, small lymphocytic lymphoma (also known as chronic lymphocytic leukemia, CLL), mantle cell lymphoma, primary mediastinal (thymic) large B cell lymphoma, T cell/histiocyte-rich large B-cell lymphoma, primary cutaneous diffuse large B-cell lymphoma, EBV positive diffuse large B-cell lymphoma, burkitt's lymphoma, lymphoplasmacytic lymphoma, nodal marginal zone B cell lymphoma, splenic marginal zone lymphoma, intravascular large B-cell lymphoma, primary effusion lymphoma, lymphomatoid granulomatosis, central nervous system lymphoma, ALK-positive large B-cell lymphoma, plasmablastic lymphoma, or large B-cell lymphoma. In some embodiments, the cancer comprises a blood cancer. In some embodiments, the blood cancer comprises myeloma, leukemia, lymphoma, Non-Hodgkin lymphoma, Hodgkin lymphoma, a myeloid neoplasm, a lymphoid neoplasm, acute lymphoblastic leukemia (ALL), acute myelogenous leukemia (AML), chronic lymphocytic leukemia (CLL), chronic myelogenous leukemia (CML), acute monocytic leukemia (AMoL), chronic myeloid leukaemia, BCR-ABL1-positive, chronic neutrophilic leukaemia, polycythaemia vera, primary myelofibrosis, essential thrombocythaemia, chronic eosinophilic leukaemia, NOS, myeloproliferative neoplasm, cutaneous mastocytosis, indolent systemic mastocytosis, systemic mastocytosis with an associated haematological neoplasm, aggressive systemic mastocytosis, mast cell leukaemia, mast cell sarcoma, myeloid/lymphoid neoplasms with PDGFRA rearrangement, myeloid/lymphoid neoplasms with PDGFRB rearrangement, myeloid/lymphoid neoplasms with FGFR1 rearrangement, myeloid/lymphoid neoplasms with PCM1-JAK2, chronic myelomonocytic leukaemia, atypical chronic myeloid leukaemia, BCR-ABL1-negative, juvenile myelomonocytic leukaemia, myelodysplastic/myeloproliferative neoplasm with ring sideroblasts and thrombocytosis, myelodysplastic/myeloproliferative neoplasm, myelodysplastic syndrome with single lineage dysplasia, myelodysplastic syndrome with ring sideroblasts and single lineage dysplasia, myelodysplastic syndrome with ring sideroblasts and multilineage dysplasia, myelodysplastic syndrome with multilineage dysplasia, myelodysplastic syndrome with excess blasts, myelodysplastic syndrome with isolated del(5q), myelodysplastic syndrome, unclassifiable, refractory cytopenia of childhood, acute myeloid leukaemia with germline CEBPA mutation, myeloid neoplasms with germline DDX41 mutation, myeloid neoplasms with germline RUNX1 mutation, myeloid neoplasms with germline ANKRD26 mutation, myeloid neoplasms with germline ETV6 mutation, myeloid neoplasms with germline GATA2 mutation, AML with t(8;21)(q22;q22.1) RUNX1-RUNXiT1; AML with inv(16)(p13.1q22) or t(16;16)(p13.1;q22) CBFB-MYH11; acute promyelocytic leukaemia with PML-RARA, AML with t(9;11)(p21.3;q23.3) KMT2A-MLLT3; AML with t(6;9)(p23;q34.1) DEK-NUP214; AML with inv(3)(q21.3q26.2) or t(3;3)(q21.3;q26.2) GATA2, MECOM; AML (megakaryoblastic) with t(1;22)(p13.3;q13.1) RBM15-MKL1; AML with BCR-ABL1; AML with mutated NPM1; AML with biallelic mutation of CEBPA; AML with mutated RUNX1; AML with myelodysplasia-related changes; Therapy-related myeloid neoplasms; AML with minimal differentiation; AML without maturation; AML with maturation; acute myelomonocytic leukaemia, acute monoblastic and monocytic leukaemia, pure erythroid leukaemia, acute megakaryoblastic leukaemia, acute basophilic leukaemia, acute panmyelosis with myelofibrosis, myeloid sarcoma, myeloid proliferations associated with Down syndrome, blastic plasmacytoid dendritic cell neoplasm, acute undifferentiated leukaemia, mixed-phenotype acute leukaemia with t(9;22)(q34.1;q11.2) BCR-ABL1; mixed-phenotype acute leukaemia with t(v;11q23.3) KMT2A-rearranged; mixed-phenotype acute leukaemia, B/myeloid; mixed-phenotype acute leukaemia, T/myeloid; mixed-phenotype acute leukaemia, rare types; acute leukaemias of ambiguous lineage, B-lymphoblastic leukaemia/lymphoma, B-lymphoblastic leukaemia/lymphoma with t(9;22)(q34.1;q11.2) BCR-ABL1; B-lymphoblastic leukaemia/lymphoma with t(v;11q23.3) KMT2A-rearranged; B-lymphoblastic leukaemia/lymphoma with t(12;21)(p13.2;q22.1) ETV6-RUNX1; B-lymphoblastic leukaemia/lymphoma with hyperdiploidy; B-lymphoblastic leukaemia/lymphoma with hypodiploidy (hypodiploid ALL); B-lymphoblastic leukaemia/lymphoma with t(5;14)(q31.1;q32.1) IGH/IL3; B-lymphoblastic leukaemia/lymphoma with t(1;19)(q23;p13.3) TCF3-PBX1; B-lymphoblastic leukaemia/lymphoma, BCR-DBL 1-like; B-lymphoblastic leukaemia/lymphoma with iAMP21; T-lymphoblastic leukaemia/lymphoma; Early T-cell precursor lymphoblastic leukaemia; NK-lymphoblastic leukaemia/lymphoma; chronic lymphocytic leukaemia (CLL)/small lymphocytic lymphoma; monoclonal B-cell lymphocytosis, CLL-type; monoclonal B-cell lymphocytosis, non-CLL-type; B-cell prolymphocytic leukaemia; splenic marginal zone lymphoma, hairy cell leukaemia, splenic diffuse red pulp small B-cell lymphoma, hairy cell leukaemia variant, Waldentrom macroglobulinemia, IgM monoclonal gammopathy, mu heavy chain disease, gamma heavy chain disease, alpha heavy chain disease, plasma cell neoplasms, extranodal marginal zone lymphoma of mucosa-associated lymphoid tissue (MALT lymphoma), nodal marginal zone lymphoma, follicular lymphoma, paediatric-type follicular lymphoma, large B-cell lymphoma with IRF4 rearrangement, primary cutaneous follicle centre lymphoma, mantle cell lymphoma, diffuse large B-cell lymphoma (DLBCL), T-cell/histiocyte-rich large B-cell lymphoma, primary DLBCL of the CNS, primary cutaneous DLBCL, EBV-positive DLBCL, EBV-positive mucocutaneous ulcer, DLBCL associated with chronic inflammation, lymphomatoid granulomatosis, grade 1,2, lymphomatoid granulomatosis, grade 3, primary mediastinal (thymic) large B-cell lymphoma, intravascular large B-cell lymphoma, ALK-positive large B-cell lymphoma, plasmablastic lymphoma, primary effusion lymphoma, multicentric Castleman disease, HHV8-positive DLBCL, HHV8-positive germinotropic lymphoproliferative disorder, Burkitt lymphoma, Burkitt-like lymphoma with 11q aberration, high-grade B-cell lymphoma, B-cell lymphoma, unclassifiable, with features intermediate between DLBCL and classic Hodgkin lymphoma, and histiocytic and dendritic cell neoplasms.

### VI. ADDITIONAL THERAPIES

A. Immunotherapy

In some embodiments, the methods comprise administration of a cancer immunotherapy. Cancer immunotherapy (sometimes called immuno-oncology, abbreviated IO) is the use of the immune system to treat cancer. Immunotherapies can be categorized as active, passive or hybrid (active and passive). These approaches exploit the fact that cancer cells often have molecules on their surface that can be detected by the immune system, known as tumor-associated antigens (TAAs); they are often proteins or other macromolecules (e.g. carbohydrates). Active immunotherapy directs the immune system to attack tumor cells by targeting TAAs. Passive immunotherapies enhance existing anti-tumor responses and include the use of monoclonal antibodies, lymphocytes and cytokines. Immunotherapies useful in the methods of the disclosure are described below.

1. Checkpoint Inhibitors and Combination Treatment

Embodiments of the disclosure may include administration of immune checkpoint inhibitors (also referred to as checkpoint inhibitor therapy), which are further described below. The checkpoint inhibitor therapy may be a monotherapy, targeting only one cellular checkpoint proteins or may be combination therapy that targets at least two cellular checkpoint proteins. For example, the checkpoint inhibitor monotherapy may comprise one of: a PD-1, PD-L1, or PD-L2 inhibitor or may comprise one of a CTLA-4, B7-1, or B7-2 inhibitor. The checkpoint inhibitor combination therapy may comprise one of: a PD-1, PD-L1, or PD-L2 inhibitor and, in combination, may further comprise one of a CTLA-4, B7-1, or B7-2 inhibitor. The combination of inhibitors in combination therapy need not be in the same composition, but can be administered either at the same time, at substantially the same time, or in a dosing regimen that includes periodic administration of both of the inhibitors, wherein the period may be a time period described herein.

a. PD-1, PD-L1, and PD-L2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PD-L1 on epithelial cells and tumor cells. PD-L2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PD-L1 activity.

Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2.

In some embodiments, the PD-1 inhibitor is a molecule that inhibits the binding of PD-1 to its ligand binding partners. In a specific aspect, the PD-1 ligand binding partners are PD-L1 and/or PD-L2. In another embodiment, a PD-L1 inhibitor is a molecule that inhibits the binding of PD-L1 to its binding partners. In a specific aspect, PD-L1 binding partners are PD-1 and/or B7-1. In another embodiment, the PD-L2 inhibitor is a molecule that inhibits the binding of PD-L2 to its binding partners. In a specific aspect, a PD-L2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-L1 inhibitor comprises AMP-224. Nivolumab, also known as MDX-1106-04, MDX-1106, ONO-4538, BMS-936558, and OPDIVO, is an anti-PD-1 antibody described in WO2006/121168. Pembrolizumab, also known as MK-3475, Merck 3475, lambrolizumab, KEYTRUDA, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PD-L2-Fc fusion soluble receptor described in WO2010/027827 and WO2011/066342. Additional PD-1 inhibitors include MEDIO680, also known as AMP-514, and REGN2810.

In some embodiments, the immune checkpoint inhibitor is a PD-L1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PD-L2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the V_{H} region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PD-L1, or PD-L2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

b. CTLA-4, B7-1, and B7-2 Inhibitors

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA-4 is a member of the immunoglobulin superfamily that is expressed on the surface of Helper T cells and transmits an inhibitory signal to T cells. CTLA-4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or V_{H} and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WO01/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. Inhibition of Co-Stimulatory Molecules

In some embodiments, the immunotherapy comprises an inhibitor of a co-stimulatory molecule. In some embodiments, the inhibitor comprises an inhibitor of B7-1 (CD80), B7-2 (CD86), CD28, ICOS, OX40 (TNFRSF4), 4-1BB (CD137; TNFRSF9), CD40L (CD40LG), GITR (TNFRSF18), and combinations thereof. Inhibitors include inhibitory antibodies, polypeptides, compounds, and nucleic acids.

3. Dendritic Cell Therapy

Dendritic cell therapy provokes anti-tumor responses by causing dendritic cells to present tumor antigens to lymphocytes, which activates them, priming them to kill other cells that present the antigen. Dendritic cells are antigen presenting cells (APCs) in the mammalian immune system. In cancer treatment, they aid cancer antigen targeting. One example of cellular cancer therapy based on dendritic cells is sipuleucel-T.

One method of inducing dendritic cells to present tumor antigens is by vaccination with autologous tumor lysates or short peptides (small parts of protein that correspond to the protein antigens on cancer cells). These peptides are often given in combination with adjuvants (highly immunogenic substances) to increase the immune and anti-tumor responses. Other adjuvants include proteins or other chemicals that attract and/or activate dendritic cells, such as granulocyte macrophage colony-stimulating factor (GM-CSF).

Dendritic cells can also be activated in vivo by making tumor cells express GM-CSF. This can be achieved by either genetically engineering tumor cells to produce GM-CSF or by infecting tumor cells with an oncolytic virus that expresses GM-CSF.

Another strategy is to remove dendritic cells from the blood of a patient and activate them outside the body. The dendritic cells are activated in the presence of tumor antigens, which may be a single tumor-specific peptide/protein or a tumor cell lysate (a solution of broken down tumor cells). These cells (with optional adjuvants) are infused and provoke an immune response.

Dendritic cell therapies include the use of antibodies that bind to receptors on the surface of dendritic cells. Antigens can be added to the antibody and can induce the dendritic cells to mature and provide immunity to the tumor.

4. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

5. Adoptive T-Cell Therapy

Adoptive T cell therapy is a form of passive immunization by the transfusion of T-cells (adoptive cell transfer). They are found in blood and tissue and usually activate when they find foreign pathogens. Specifically, they activate when the T-cell's surface receptors encounter cells that display parts of foreign proteins on their surface antigens. These can be either infected cells, or antigen presenting cells (APCs). They are found in normal tissue and in tumor tissue, where they are known as tumor infiltrating lymphocytes (TILs). They are activated by the presence of APCs such as dendritic cells that present tumor antigens. Although these cells can attack the tumor, the environment within the tumor is highly immunosuppressive, preventing immune-mediated tumor death.

Multiple ways of producing and obtaining tumor targeted T-cells have been developed. T-cells specific to a tumor antigen can be removed from a tumor sample (TILs) or filtered from blood. Subsequent activation and culturing is performed ex vivo, with the results reinfused. Activation can take place through gene therapy, or by exposing the T cells to tumor antigens.

It is contemplated that a cancer treatment may exclude any of the cancer treatments described herein. Furthermore, embodiments of the disclosure include patients that have been previously treated for a therapy described herein, are currently being treated for a therapy described herein, or have not been treated for a therapy described herein. In some embodiments, the patient is one that has been determined to be resistant to a therapy described herein. In some embodiments, the patient is one that has been determined to be sensitive to a therapy described herein.

B. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

C. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

D. Neoantigens

In some embodiments, the additional therapy comprises neoantigen administration. Many tumors express mutations. These mutations potentially create new targetable antigens (neoantigens) for use in T cell immunotherapy. The presence of CD8⁺ T cells in cancer lesions, as identified using RNA sequencing data, is higher in tumors with a high mutational burden. The level of transcripts associated with cytolytic activity of natural killer cells and T cells positively correlates with mutational load in many human tumors.

E. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN₂), cyclophosphamide and/or ifosfamide, melphalan (L-sarcolysin), and chlorambucil. Cyclophosphamide (CYTOXAN) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m2. Further, 5-FU dosing schedules may be for a variety of time periods, for example up to six weeks, or as determined by one of ordinary skill in the art to which this disclosure pertains.

Gemcitabine diphosphate (GEMZAR, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

F. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

G. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

H. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

### VII. PHARMACEUTICAL COMPOSITIONS

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most about or at least about 3, 4, 5, 6, 7, 8, 9, 10 or more. The administrations may range from 2-day to 12-week intervals, more usually from one to two week intervals. The course of the administrations may be followed by assays for alloreactive immune responses and T cell activity.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, subcutaneous, or even intraperitoneal routes. Typically, such compositions can be prepared as injectables, either as liquid solutions or suspensions and the preparations can also be emulsified.

The pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (i.e. cells of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

In certain embodiments, the effective dose of the pharmaceutical composition is one which can provide a blood level of about 1 μM to 150 μM. In another embodiment, the effective dose provides a blood level of about 4 μM to 100 μM.; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VIII. SEQUENCES

The amino acid sequence of exemplary CAR molecules useful in the methods and compositions of the disclosure are shown below.

TABLE 1
CARs

| | | SEQ ID |
|---|---|---|
| Name | Sequence | NO |
| dAPRIL- | METDTLLLWVLLLWVPGSTGAGGSDYKDDDDKGGSVDVLHLVPINATS | 1 |
| Luc90 | KDDSDVTEVMWQPALRRGRGLQAQGYGVRIQDAGVYLLYSQVLFQDV | |
| Short | TFTMGQVVSREGQGRQETLFRCIRSMPSHPDRAYNSCYSAGVFHLHQG | |
| | DILSVIIPRARAKLNLSPHGTFLGFVKLGGGGSGGGGSGGGGSGGGGSQ | |
| | VQLQQPGAELVRPGASVKLSCKASGYSFTTYWMNWVKQRPGQGLEWI | |
| | GMIHPSDSETRLNQKFKDKATLTVDKSSSTAYMQLSSPTSEDSAVYYCA | |
| | RSTMIATRAMDYWGQGTSVTVSGSTSGSGKPGSGEGSTKGDIVMTQSQ | |
| | KSMSTSVGDRVSITCKASQDVITGVAWYQQKPGQSPKLLIYSASYRYTG | |
| | VPDRFTGSGSGTDFTFTISNVQAEDLAVYYCQQHYSTPLTFGAGTKLEL | |
| | KESKYGPPCPPCPMFWVLVVVGGVLACYSLLVTVAFIIFWVKRGRKKLL | |
| | YIFKQPFMRPVQTTQEEDGCSCRFPEEEEGGCELRVKFSRSADAPAYQQ | |
| | GQNQLYNELNLGRREEYDVLDKRRGRDPEMGGKPRRKNPQEGLYNEL | |
| | QKDKMAEAYSEIGMKGERRRGKGHDGLYQGLSTATKDTYDALHMQAL | |
| | PPR | |
| c11D5.3- | METDTLLLWVLLLWVPGSTGAGGSDYKDDDDKGGSVDIVLTQSPPSLA | 2 |
| Luc90 | MSLGKRATISCRASESVTILGSHLIHWYQQKPGQPPTLLIQLASNVQTGV | |
| Short | PARFSGSGSRTDFTLTIDPVEEDDVAVYYCLQSRTIPRTFGGGTKLEIKGS | |
| | TSGSGKPGSGEGSTKGQIQLVQSGPELKKPGETVKISCKASGYTFTDYSI | |
| | NWVKRAPGKGLKWMGWINTETREPAYAYDFRGRFAFSLETSASTAYLQ | |
| | INNLKYEDTATYFCALDYSYAMDYWGQGTSVTVSSGGGGSGGGGSGG | |
| | GGSGGGGSQVQLQQPGAELVRPGASVKLSCKASGYSFTTYWMNWVKQ | |
| | RPGQGLEWIGMIHPSDSETRLNQKFKDKATLTVDKSSSTAYMQLSSPTSE | |
| | DSAVYYCARSTMIATRAMDYWGQGTSVTVSGSTSGSGKPGSGEGSTKG | |
| | DIVMTQSQKSMSTSVGDRVSITCKASQDVITGVAWYQQKPGQSPKLLIY | |
| | SASYRYTGVPDRFTGSGSGTDFTFTISNVQAEDLAVYYCQQHYSTPLTFG | |
| | AGTKLELKESKYGPPCPPCPMFWVLVVVGGVLACYSLLVTVAFIIFWVK | |
| | RGRKKLLYIFKQPFMRPVQTTQEEDGCSCRFPEEEEGGCELRVKFSRSAD | |
| | APAYQQGQNQLYNELNLGRREEYDVLDKRRGRDPEMGGKPRRKNPQE | |
| | GLYNELQKDKMAEAYSEIGMKGERRRGKGHDGLYQGLSTATKDTYDA | |
| | LHMQALPPR | |
| huC11D5. | METDTLLLWVLLLWVPGSTGAGGSDYKDDDDKGGSVDIVLTQSPASLA | 3 |
| 3-Luc90 | VSLGERATINCRASESVSVIGAHLIHWYQQKPGQPPKLLIYLASNLETGV | |
| Short | PARFSGSGSGTDFTLTISSLQAEDAAIYSCLQSRIFPRTFGQGTKLEIKG | |
| | STSGSGKPGSGEGSTKGQVQLVQSGSELKKPGASVKVSCKASGYTFTDYSI | |
| | NWVRQAPGQGLEWMGWINTETREPAYAYDFRGRFVFSLDTSVSTAYLQ | |
| | ISSLKAEDTAVYYCARDYSYAMDYWGQGTLVTVSSGGGGSGGGGSGG | |
| | GGSGGGGSQVQLQQPGAELVRPGASVKLSCKASGYSFTTYWMNWVKQ | |
| | RPGQGLEWIGMIHPSDSETRLNQKFKDKATLTVDKSSSTAYMQLSSPTSE | |
| | DSAVYYCARSTMIATRAMDYWGQGTSVTVSGSTSGSGKPGSGEGSTKG | |
| | DIVMTQSQKSMSTSVGDRVSITCKASQDVITGVAWYQQKPGQSPKLLIY | |
| | SASYRYTGVPDRFTGSGSGTDFTFTISNVQAEDLAVYYCQQHYSTPLTFG | |
| | AGTKLELKESKYGPPCPPCPMFWVLVVVGGVLACYSLLVTVAFIIFWVK | |
| | RGRKKLLYIFKQPFMRPVQTTQEEDGCSCRFPEEEEGGCELRVKFSRSAD | |
| | APAYQQGQNQLYNELNLGRREEYDVLDKRRGRDPEMGGKPRRKNPQE | |
| | GLYNELQKDKMAEAYSEIGMKGERRRGKGHDGLYQGLSTATKDTYDA | |
| | LHMQALPPR | |
| J22.9-xi- | METDTLLLWVLLLWVPGSTGAGGSDYKDDDDKGGSVDIVMTQSQRFM | 4 |
| Luc90 | TTSVGDRVSVTCKASQSVDSNVAWYQQKPRQSPKALIFSASLRFSGVPA | |
| Short | RFTGSGSGTDFTLTISNLQSEDLAEYFCQQYNNYPLTFGAGTKLELKRGS | |
| | TSGSGKPGSGEGSTKGQVQLQQSGGGLVQPGGSLKLSCAASGIDFSRYW | |
| | MSWVRRAPGKGLEWIGEINPDSSTINYAPSLKDKFIISRDNAKNTLYLQM | |
| | SKVRSEDTALYYCASLYYDYGDAMDYWGQGTSVTVSSGGGGSGGGGS | |
| | GGGGSGGGGSQVQLQQPGAELVRPGASVKLSCKASGYSFTTYWMNWV | |
| | KQRPGQGLEWIGMIHPSDSETRLNQKFKDKATLTVDKSSSTAYMQLSSP | |
| | TSEDSAVYYCARSTMIATRAMDYWGQGTSVTVSGSTSGSGKPGSGEGS | |
| | TKGDIVMTQSQKSMSTSVGDRVSITCKASQDVITGVAWYQQKPGQSPK | |
| | LLIYSASYRYTGVPDRFTGSGSGTDFTFTISNVQAEDLAVYYCQQHYSTP | |
| | LTFGAGTKLELKESKYGPPCPPCPMFWVLVVVGGVLACYSLLVTVAFIIF | |
| | WVKRGRKKLLYIFKQPFMRPVQTTQEEDGCSCRFPEEEEGGCELRVKFS | |
| | RSADAPAYQQGQNQLYNELNLGRREEYDVLDKRRGRDPEMGGKPRRK | |
| | NPQEGLYNELQKDKMAEAYSEIGMKGERRRGKGHDGLYQGLSTATKD | |
| | TYDALHMQALPPR | |
| huJ22.9- | METDTLLLWVLLLWVPGSTGAGGSDYKDDDDKGGSVDIVMTQSPATLS | 5 |
| xi-Luc90 | VSVGDEVTLTCKASQSVDSNVAWYQQKPGQAPKLLIYSASLRFSGVPAR | |
| Short | FSGSGSGTDFTLTISSLQSEDFAVYYCQQYNNYPLTFGAGTKLELKRGST | |
| | SGSGKPGSGEGSTKGEVQLVESGGGLVQPGGSLRLSCAASGFTFSRYWM | |
| | SWVRQAPGKGLEWVGEINPDSSTINYAPSLKGRFTISRDNAKNTLYLQM | |
| | NSLRAEDTAVYYCASLYYDYGDAMDYWGQGTLVTVSSGGGGSGGGGS | |
| | GGGGSGGGGSQVQLQQPGAELVRPGASVKLSCKASGYSFTTYWMNWV | |
| | KQRPGQGLEWIGMIHPSDSETRLNQKFKDKATLTVDKSSSTAYMQLSSP | |
| | TSEDSAVYYCARSTMIATRAMDYWGQGTSVTVSGSTSGSGKPGSGEGS | |
| | TKGDIVMTQSQKSMSTSVGDRVSITCKASQDVITGVAWYQQKPGQSPK | |
| | LLIYSASYRYTGVPDRFTGSGSGTDFTFTISNVQAEDLAVYYCQQHYSTP | |
| | LTFGAGTKLELKESKYGPPCPPCPMFWVLVVVGGVLACYSLLVTVAFIIF | |
| | WVKRGRKKLLYIFKQPFMRPVQTTQEEDGCSCRFPEEEEGGCELRVKFS | |
| | RSADAPAYQQGQNQLYNELNLGRREEYDVLDKRRGRDPEMGGKPRRK | |
| | NPQEGLYNELQKDKMAEAYSEIGMKGERRRGKGHDGLYQGLSTATKD | |
| | TYDALHMQALPPR | |
| huLuc63- | METDTLLLWVLLLWVPGSTGAGGSDYKDDDDKGGSVDEVQLVESGGG | 6 |
| dAPRIL | LVQPGGSLRLSCAASGFDFSRYWMSWVRQAPGKGLEWIGEINPDSSTIN | |
| Short | YAPSLKDKFIISRDNAKNSLYLQMNSLRAEDTAVYYCARPDGNYWYFD | |
| | VWGQGTLVTVSSGSTSGSGKPGSGEGSTKGDIQMTQSPSSLSASVGDRV | |
| | TITCKASQDVGIAVAWYQQKPGKVPKLLIYWASTRHTGVPDRPSGSGSG | |
| | TDFTLTISSLQPEDVATYYCQQYSSYPYTFGQGTKVEIKGGGGSGGGGS | |
| | GGGGSGGGGSVLHLVPINATSKDDSDVTEVMWQPALRRGRGLQAQGY | |
| | GVRIQDAGVYLLYSQVLFQDVTFTMGQVVSREGQGRQETLFRCIRSMPS | |
| | HPDRAYNSCYSAGVFHLHQGDILSVIIPRARAKLNLSPHGTFLGFVKLES | |
| | KYGPPCPPCPMFWVLVVVGGVLACYSLLVTVAFIIFWVKRGRKKLLYIF | |
| | KQPFMRPVQTTQEEDGCSCRFPEEEEGGCELRVKFSRSADAPAYQQGQN | |
| | QLYNELNLGRREEYDVLDKRRGRDPEMGGKPRRKNPQEGLYNELQKD | |
| | KMAEAYSEIGMKGERRRGKGHDGLYQGLSTATKDTYDALHMQALPPR | |
| huLuc63- | METDTLLLWVLLLWVPGSTGAGGSDYKDDDDKGGSVDEVQLVESGGG | 7 |
| c11D5.3 | LVQPGGSLRLSCAASGFDFSRYWMSWVRQAPGKGLEWIGEINPDSSTIN | |
| Short | YAPSLKDKFIISRDNAKNSLYLQMNSLRAEDTAVYYCARPDGNYWYFD | |
| | VWGQGTLVTVSSGSTSGSGKPGSGEGSTKGDIQMTQSPSSLSASVGDRV | |
| | TITCKASQDVGIAVAWYQQKPGKVPKLLIYWASTRHTGVPDRPSGSGSG | |
| | TDFTLTISSLQPEDVATYYCQQYSSYPYTFGQGTKVEIKGGGGSGGGGS | |
| | GGGGSGGGGSDIVLTQSPPSLAMSLGKRATISCRASESVTILGSHLIHWY | |
| | QQKPGQPPTLLIQLASNVQTGVPARFSGSGSRTDFTLTIDPVEEDDVAVY | |
| | YCLQSRTIPRTFGGGTKLEIKGSTSGSGKPGSGEGSTKGQIQLVQSGPE | |
| | LKKPGETVKISCKASGYTFTDYSINWVKRAPGKGLKWMGWINTETREPAY | |
| | AYDFRGRFAFSLETSASTAYLQINNLKYEDTATYFCALDYSYAMDYWG | |
| | QGTSVTVSSESKYGPPCPPCPMFWVLVVVGGVLACYSLLVTVAFIIFWV | |
| | KRGRKKLLYIFKQPFMRPVQTTQEEDGCSCRFPEEEEGGCELRVKFSRSA | |
| | DAPAYQQGQNQLYNELNLGRREEYDVLDKRRGRDPEMGGKPRRKNPQ | |
| | EGLYNELQKDKMAEAYSEIGMKGERRRGKGHDGLYQGLSTATKDTYD | |
| | ALHMQALPPR | |
| huLuc63- | METDTLLLWVLLLWVPGSTGAGGSDYKDDDDKGGSVDEVQLVESGGG | 8 |
| huc11D5.3 | LVQPGGSLRLSCAASGFDFSRYWMSWVRQAPGKGLEWIGEINPDSSTIN | |
| Short | YAPSLKDKFIISRDNAKNSLYLQMNSLRAEDTAVYYCARPDGNYWYFD | |
| | VWGQGTLVTVSSGSTSGSGKPGSGEGSTKGDIQMTQSPSSLSASVGDRV | |
| | TITCKASQDVGIAVAWYQQKPGKVPKLLIYWASTRHTGVPDRPSGSGSG | |
| | TDFTLTISSLQPEDVATYYCQQYSSYPYTFGQGTKVEIKGGGGSGGGGS | |
| | GGGGSGGGGSDIVLTQSPASLAVSLGERATINCRASESVSVIGAHLIHWY | |
| | QQKPGQPPKLLIYLASNLETGVPARFSGSGSGTDFTLTISSLQAEDAAIYS | |
| | CLQSRIFPRTFGQGTKLEIKGSTSGSGKPGSGEGSTKGQVQLVQSGSELK | |
| | KPGASVKVSCKASGYTFTDYSINWVRQAPGQGLEWMGWINTETREPAY | |
| | AYDFRGRFVFSLDTSVSTAYLQISSLKAEDTAVYYCARDYSYAMDYWG | |
| | QGTLVTVSSESKYGPPCPPCPMFWVLVVVGGVLACYSLLVTVAFIIFWV | |
| | KRGRKKLLYIFKQPFMRPVQTTQEEDGCSCRFPEEEEGGCELRVKFSRSA | |
| | DAPAYQQGQNQLYNELNLGRREEYDVLDKRRGRDPEMGGKPRRKNPQ | |
| | EGLYNELQKDKMAEAYSEIGMKGERRRGKGHDGLYQGLSTATKDTYD | |
| | ALHMQALPPR | |
| huLuc63- | METDTLLLWVLLLWVPGSTGAGGSDYKDDDDKGGSVDEVQLVESGGG | 9 |
| J22.9-xi | LVQPGGSLRLSCAASGFDFSRYWMSWVRQAPGKGLEWIGEINPDSSTIN | |
| Short | YAPSLKDKFIISRDNAKNSLYLQMNSLRAEDTAVYYCARPDGNYWYFD | |
| | VWGQGTLVTVSSGSTSGSGKPGSGEGSTKGDIQMTQSPSSLSASVGDRV | |
| | TITCKASQDVGIAVAWYQQKPGKVPKLLIYWASTRHTGVPDRPSGSGSG | |
| | TDFTLTISSLQPEDVATYYCQQYSSYPYTFGQGTKVEIKGGGGSGGGGS | |
| | GGGGSGGGGSDIVMTQSQRFMTTSVGDRVSVTCKASQSVDSNVAWYQ | |
| | QKPRQSPKALIFSASLRFSGVPARFTGSGSGTDFTLTISNLQSEDLAEYFC | |
| | QQYNNYPLTFGAGTKLELKRGSTSGSGKPGSGEGSTKGQVQLQQSGGG | |
| | LVQPGGSLKLSCAASGIDFSRYWMSWVRRAPGKGLEWIGEINPDSSTIN | |
| | YAPSLKDKFIISRDNAKNTLYLQMSKVRSEDTALYYCASLYYDYGDAM | |
| | DYWGQGTSVTVSSESKYGPPCPPCPMFWVLVVVGGVLACYSLLVTVAF | |
| | IIFWVKRGRKKLLYIFKQPFMRPVQTTQEEDGCSCRFPEEEEGGCELRVK | |
| | FSRSADAPAYQQGQNQLYNELNLGRREEYDVLDKRRGRDPEMGGKPR | |
| | RKNPQEGLYNELQKDKMAEAYSEIGMKGERRRGKGHDGLYQGLSTAT | |
| | KDTYDALHMQALPPR | |
| huLuc63- | METDTLLLWVLLLWVPGSTGAGGSDYKDDDDKGGSVDEVQLVESGGG | 10 |
| huJ22.9-xi | LVQPGGSLRLSCAASGFDFSRYWMSWVRQAPGKGLEWIGEINPDSSTIN | |
| Short | YAPSLKDKFIISRDNAKNSLYLQMNSLRAEDTAVYYCARPDGNYWYFD | |
| | VWGQGTLVTVSSGSTSGSGKPGSGEGSTKGDIQMTQSPSSLSASVGDRV | |
| | TITCKASQDVGIAVAWYQQKPGKVPKLLIYWASTRHTGVPDRPSGSGSG | |
| | TDFTLTISSLQPEDVATYYCQQYSSYPYTFGQGTKVEIKGGGGSGGGGS | |
| | GGGGSGGGGSDIVMTQSPATLSVSVGDEVTLTCKASQSVDSNVAWYQQ | |
| | KPGQAPKLLIYSASLRFSGVPARFSGSGSGTDFTLTISSLQSEDFAVYYCQ | |
| | QYNNYPLTFGAGTKLELKRGSTSGSGKPGSGEGSTKGEVQLVESGGGLV | |
| | QPGGSLRLSCAASGFTFSRYWMSWVRQAPGKGLEWVGEINPDSSTINYA | |
| | PSLKGRFTISRDNAKNTLYLQMNSLRAEDTAVYYCASLYYDYGDAMDY | |
| | WGQGTLVTVSSESKYGPPCPPCPMFWVLVVVGGVLACYSLLVTVAFIIF | |
| | WVKRGRKKLLYIFKQPFMRPVQTTQEEDGCSCRFPEEEEGGCELRVKFS | |
| | RSADAPAYQQGQNQLYNELNLGRREEYDVLDKRRGRDPEMGGKPRRK | |
| | NPQEGLYNELQKDKMAEAYSEIGMKGERRRGKGHDGLYQGLSTATKD | |
| | TYDALHMQALPPR | |
| huLuc63- | METDTLLLWVLLLWVPGSTGAGGSDYKDDDDKGGSVDEVQLVESGGG | 11 |
| c11D5.3 | LVQPGGSLRLSCAASGFDFSRYWMSWVRQAPGKGLEWIGEINPDSSTIN | |
| Long | YAPSLKDKFIISRDNAKNSLYLQMNSLRAEDTAVYYCARPDGNYWYFD | |
| | VWGQGTLVTVSSGSTSGSGKPGSGEGSTKGDIQMTQSPSSLSASVGDRV | |
| | TITCKASQDVGIAVAWYQQKPGKVPKLLIYWASTRHTGVPDRPSGSGSG | |
| | TDFTLTISSLQPEDVATYYCQQYSSYPYTFGQGTKVEIKGGGGSGGGGS | |
| | GGGGSGGGGSDIVLTQSPPSLAMSLGKRATISCRASESVTILGSHLIHWY | |
| | QQKPGQPPTLLIQLASNVQTGVPARFSGSGSRTDFTLTIDPVEEDDVAVY | |
| | YCLQSRTIPRTFGGGTKLEIKGSTSGSGKPGSGEGSTKGQIQLVQSGPELK | |
| | KPGETVKISCKASGYTFTDYSINWVKRAPGKGLKWMGWINTETREPAY | |
| | AYDFRGRFAFSLETSASTAYLQINNLKYEDTATYFCALDYSYAMDYWG | |
| | QGTSVTVSSESKYGPPCPPCPAPEFEGGPSVFLFPPKPKDTLMISRTPEVT | |
| | CVVVDVSQEDPEVQFNWYVDGVEVHNAKTKPREEQFQSTYRVVSVLT | |
| | VLHQDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDGSFF | |
| | LYSRLTVDKSRWQEGNVFSCSVMHEALHNHYTQKSLSLSLGKMFWVL | |
| | VVVGGVLACYSLLVTVAFIIFWVKRGRKKLLYIFKQPFMRPVQTTQEED | |
| | GCSCRFPEEEEGGCELRVKFSRSADAPAYQQGQNQLYNELNLGRREEYD | |
| | VLDKRRGRDPEMGGKPRRKNPQEGLYNELQKDKMAEAYSEIGMKGER | |
| | RRGKGHDGLYQGLSTATKDTYDALHMQALPPR | |
| huc11D5.3- | METDTLLLWVLLLWVPGSTGAGGSDYKDDDDKGGSVDIVLTQSPASLA | 12 |
| huLuc63 | VSLGERATINCRASESVSVIGAHLIHWYQQKPGQPPKLLIYLASNLETGV | |
| Short | PARFSGSGSGTDFTLTISSLQAEDAAIYSCLQSRIFPRTFGQGTKLEIKG | |
| | STSGSGKPGSGEGSTKGQVQLVQSGSELKKPGASVKVSCKASGYTFTDYSI | |
| | NWVRQAPGQGLEWMGWINTETREPAYAYDFRGRFVFSLDTSVSTAYLQ | |
| | ISSLKAEDTAVYYCARDYSYAMDYWGQGTLVTVSSGGGGSGGGGSGG | |
| | GGSGGGGSEVQLVESGGGLVQPGGSLRLSCAASGFDFSRYWMSWVRQ | |
| | APGKGLEWIGEINPDSSTINYAPSLKDKFIISRDNAKNSLYLQMNSLRAE | |
| | DTAVYYCARPDGNYWYFDVWGQGTLVTVSSGSTSGSGKPGSGEGSTK | |
| | GDIQMTQSPSSLSASVGDRVTITCKASQDVGIAVAWYQQKPGKVPKLLI | |
| | YWASTRHTGVPDRPSGSGSGTDFTLTISSLQPEDVATYYCQQYSSYPYTF | |
| | GQGTKVEIKESKYGPPCPPCPMFWVLVVVGGVLACYSLLVTVAFIIFWV | |
| | KRGRKKLLYIFKQPFMRPVQTTQEEDGCSCRFPEEEEGGCELRVKFSRSA | |
| | DAPAYQQGQNQLYNELNLGRREEYDVLDKRRGRDPEMGGKPRRKNPQ | |
| | EGLYNELQKDKMAEAYSEIGMKGERRRGKGHDGLYQGLSTATKDTYD | |
| | ALHMQALPPR | |
| huc11D5.3- | METDTLLLWVLLLWVPGSTGAGGSDYKDDDDKGGSVDIVLTQSPASLA | 13 |
| huLuc63 | VSLGERATINCRASESVSVIGAHLIHWYQQKPGQPPKLLIYLASNLETGV | |
| Long | PARFSGSGSGTDFTLTISSLQAEDAAIYSCLQSRIFPRTFGQGTKLEIKG | |
| | STSGSGKPGSGEGSTKGQVQLVQSGSELKKPGASVKVSCKASGYTFTDYSI | |
| | NWVRQAPGQGLEWMGWINTETREPAYAYDFRGRFVFSLDTSVSTAYLQ | |
| | ISSLKAEDTAVYYCARDYSYAMDYWGQGTLVTVSSGGGGSGGGGSGG | |
| | GGSGGGGSEVQLVESGGGLVQPGGSLRLSCAASGFDFSRYWMSWVRQ | |
| | APGKGLEWIGEINPDSSTINYAPSLKDKFIISRDNAKNSLYLQMNSLRAE | |
| | DTAVYYCARPDGNYWYFDVWGQGTLVTVSSGSTSGSGKPGSGEGSTK | |
| | GDIQMTQSPSSLSASVGDRVTITCKASQDVGIAVAWYQQKPGKVPKLLI | |
| | YWASTRHTGVPDRPSGSGSGTDFTLTISSLQPEDVATYYCQQYSSYPYTF | |
| | GQGTKVEIKESKYGPPCPPCPAPEFEGGPSVFLFPPKPKDTLMISRTPEVT | |
| | CVVVDVSQEDPEVQFNWYVDGVEVHNAKTKPREEQFQSTYRVVSVLT | |
| | VLHQDWLNGKEYKCKVSNKGLPSSIEKTISKAKGQPREPQVYTLPPSQE | |
| | EMTKNQVSLTCLVKGFYPSDIAVEWESNGQPENNYKTTPPVLDSDGSFF | |
| | LYSRLTVDKSRWQEGNVFSCSVMHEALHNHYTQKSLSLSLGKMFWVL | |
| | VVVGGVLACYSLLVTVAFIIFWVKRGRKKLLYIFKQPFMRPVQTTQEED | |
| | GCSCRFPEEEEGGCELRVKFSRSADAPAYQQGQNQLYNELNLGRREEYD | |
| | VLDKRRGRDPEMGGKPRRKNPQEGLYNELQKDKMAEAYSEIGMKGER | |
| | RRGKGHDGLYQGLSTATKDTYDALHMQALPPR | |

Exemplary CDR embodiments of the BCMA binding region (C11D5.3 antibody) include the following:

TABLE 2
CDRs of C11D3.5 BCMA binding regions.

| | HCDR1 | HCDR2 | HCDR3 |
|---|---|---|---|
| C11D5.3 VH | DYSIN | NTETRE | DYSY |
| | (SEQ ID | (SEQ ID | AMDY |
| | NO: 14) | NO: 15) | (SEQ ID |
| | | | NO: 16) |
| | DYSIN | WINTETR | DYSY |
| | (SEQ ID | EPAYAYD | AMDY |
| | NO: 14) | ERG | (SEQ ID |
| | | (SEQ ID | NO: 16) |
| | | NO: 79) | |
| | GYTFTDY | NTETRE | DYSY |
| | (SEQ ID | (SEQ ID | AMDY |
| | NO: 80) | NO: 15) | (SEQ ID |
| | | | NO: 16) |
| | GYTFT | WINTETR | DYSY |
| | DYSIN | EPAYAYD | AMDY |
| | (SEQ ID | ERG | (SEQ ID |
| | NO: 81) | (SEQ ID | NO: 16) |
| | | NO: 79) | |
| | LCDR1 | LCDR2 | LCDR3 |
| C11D5.3 VL | SESVTI | LAS | SRTIPR |
| (murine) | LGSHL | (SEQ ID | (SEQ ID |
| | (SEQ ID | NO: 19) | NO: 83) |
| | NO: 82) | | |
| C11D5.3 VL | SESVS | LAS | SRIFPR |
| (humanized) | VIGAHL | (SEQ ID | (SEQ ID |
| | (SEQ ID | NO: 19) | NO: 20) |
| | NO: 18) | | |
| C11D5.3 VL | RASES | LASNVQT | LQSRT |
| | VTILG | (SEQ ID | IPRT |
| | SHLIH | NO: 58) | (SEQ ID |
| | (SEQ ID | | NO: 59) |
| | NO: 57) | | |
| | RASES | LASNLET | LQSRI |
| | VSVIG | (SEQ ID | FPRT |
| | AHL1H | NO: 61) | (SEQ ID |
| | (SEQ ID | | NO: 62) |
| | NO: 60) | | |
| | RASESV | LASNVQT | LQSRT |
| | SVIGAH | (SEQ ID | IPRT |
| | LIH | NO: 58) | (SEQ ID |
| | (SEQ ID | | NO: 59) |
| | NO: 60) | | |
| | RASESVT | LASNVQT | LQSRT |
| | ILGSHL | (SEQ ID | IPRT |
| | IY | NO: 58) | (SEQ ID |
| | (SEQ ID | | NO: 59) |
| | NO: 63) | | |
| | RASESVT | LASNVQT | LQSRT |
| | ILGSHL | (SEQ ID | IPRT |
| | IH | NO: 58) | (SEQ ID |
| | (SEQ ID | | NO: 59) |
| | NO: 57) | | |
| | RASESVS | YLASN | LQSRI |
| | VIGAHL | LET | FPRT |
| | IH | (SEQ ID | (SEQ ID |
| | (SEQ ID | NO: 64) | NO: 62) |
| | NO: 60) | | |

Exemplary CDR embodiments of the BCMA binding region (J22.9-xi antibody) include the following:

TABLE 3
CDRs of J22.9-xi BCMA binding regions.

| | HCDR1 | HCDR2 | HCDR3 |
|---|---|---|---|
| J22.9-xi-VH | RYWMS | EINPDSS | LYYDYG |
| | (SEQ ID | TINYAPS | DAMDYW |
| | NO: 26) | LK | (SEQ ID |
| | | (SEQ ID | NO: 28) |
| | | NO: 27) | |
| | DYWMS | EINPDSS | LYYDYG |
| | (SEQ ID | TINYAPS | DAMDYWG |
| | NO: 65) | LKG | (SEQ ID |
| | | (SEQ ID | NO: 68) |
| | | NO: 66) | |
| | DYWMS | EINPDS | SLYYDY |
| | (SEQ ID | STINYA | GDAMDYW |
| | NO: 65) | PSLKG | (SEQ ID |
| | | (SEQ ID | NO: 69) |
| | | NO: 66) | |
| | RYWMS | EINPDSS | LYYDYG |
| | (SEQ ID | TINYAPS | DAMDYW |
| | NO: 26) | LKD | (SEQ ID |
| | | (SEQ ID | NO: 28) |
| | | NO: 67) | |
| | RYWMS | EINPDSS | LYYDYG |
| | (SEQ ID | TINYAPS | DAMDYW |
| | NO: 26) | LKG | (SEQ ID |
| | | (SEQ ID | NO: 28) |
| | | NO: 66) | |
| | LCDR1 | LCDR2 | LCDR3 |
| J22.9-xi-VL | KASQSV | SASLRFS | QQYNNY |
| | DSNVA | (SEQ ID | PLTFG |
| | (SEQ ID | NO: 31) | (SEQ ID |
| | NO: 30) | | NO: 32) |
| | KASQSV | SDDLRFS | QQYNNY |
| | DSNVA | (SEQ ID | PLTFG |
| | (SEQ ID | NO: 70) | (SEQ ID |
| | NO: 30) | | NO: 32) |

Exemplary CDR embodiments of the CS1 binding region (from the Luc90 antibody) include the following:

TABLE 4
CDRs of Luc90 CS1 binding regions.

| | HCDR1 | HCDR2 | HCDR3 |
|---|---|---|---|
| Murine | TYWMN | MIHPSDS | STMIAT |
| Luc90- | (SEQ ID | ETRLNQ | RAMDY |
| VH | NO: 39) | (SEQ ID | (SEQ ID |
| | | NO: 40) | NO: 41) |
| | TYWMN | MIHPSDSE | STMIAT |
| | (SEQ ID | TRLNQKFKD | RAMDY |
| | NO: 39) | (SEQ ID | (SEQ ID |
| | | NO: 71) | NO: 41) |
| | LCDR1 | LCDR2 | LCDR3 |
| Murine | KASQDVI | SASYRYT | QQHYS |
| Luc90- | TGVA | (SEQ ID | TPLT |
| VL | (SEQ ID | NO: 44) | (SEQ ID |
| | NO: 43) | | NO: 45) |

Exemplary CDR embodiments of the CS1 binding region (from the Luc63 antibody) include the following:

TABLE 5
CDRs of Luc63 CS1 binding regions.

| | HCDR1 | HCDR2 | HCDR3 |
|---|---|---|---|
| huLuc63- | RYWMS | EINPDS | PDGNYW |
| (Humanized | (SEQ ID | STINYA | YFDV |
| Luc63) | NO: 48) | PSLKD | (SEQ ID |
| VH | | (SEQ ID | NO: 50) |
| | | NO: 49) | |
| | RYWMS | EINPDSS | PDGNYW |
| | (SEQ ID | TINYTPS | YFDV |
| | NO: 48) | LKD | (SEQ ID |
| | | (SEQ ID | NO: 50) |
| | | NO: 72) | |
| | LCDR1 | LCDR2 | LCDR3 |
| huLuc63- | KASQDV | WASTRHT | QQYSSY |
| VL | GIAVA | (SEQ ID | PYT |
| | (SEQ ID | NO: 53) | (SEQ ID |
| | NO: 52) | | NO: 54) |

Other polypeptides useful in the methods and compositions of the current disclosure are tabulated below:

TABLE 6
Polypeptide domains useful in
embodiments of the disclosure.

| | | |
|---|---|---|
| | | SEQ |
| | | ID |
| Description | Amino Acid Sequence | NO. |

BCMA-targeting domains

| | | |
|---|---|---|
| Murine | QIQLVQSGPELKKPGETVKISC | 17 |
| c11D5.3 | KASGYTHTDYSINWVKRAPGK | |
| VH | GLKWMGWINTETREPAYAYDFR | |
| | GRFAFSLETSASTAYLQINNLK | |
| | YEDTATYFCALDYSYAMDYWGQ | |
| | GTSVTVSS | |
| Murine | DIVLTQSPPSLAMSLGKRATIS | 21 |
| c11D5.3 | CRASESVTILGSHLIHWYQQKP | |
| VL | GQPPTLLIQLASNVQTGVPARF | |
| | SGSGSRTDFTLTIDPVEEDDVA | |
| | VYYCLQSRTIPRTFGGGTKLEI | |
| | K | |
| Murine | DIVLTQSPPSLAMSLGKRATIS | 22 |
| c11D5.3 | CRASESVTILGSHLIHWYQQKP | |
| scFv | GQPPTLLIQLASNVQTGVPARF | |
| (VL-VH) | SGSGSRTDFTLTIDPVEEDDVA | |
| | VYYCLQSRTIPRTFGGGTKLEI | |
| | KGSTSGSGKPGSGEGSTKGQIQ | |
| | LVQSGPELKKPGETVKISCKAS | |
| | GYTFTDYSINWVKRAPGKGLKW | |
| | MGWINTETREPAYAYDFRGRFA | |
| | FSLETSASTAYLQINNLKYEDT | |
| | ATYFCALDYSYAMDYWGQGTSV | |
| | TVSS | |
| Humanized | QVQLVQSGSELKKPGASVKVSC | 23 |
| c11D5.3 VH | KASGYTFTDYSINWVRQAPGQG | |
| | LEWMGWINTETREPAYAYDFRG | |
| | RFVFSLDTSVSTAYLQISSLKA | |
| | EDTAVYYCARDYSYAMDYWGQG | |
| | TLVTVSS | |
| Humanized | DIVLTQSPASLAVSLGERATIN | 24 |
| c11D5.3 VL | CRASESVSVIGAHLIHWYQQKP | |
| | GQPPKLLIYLASNLETGVPARF | |
| | SGSGSGTDFTLTISSLQAEDAA | |
| | IYSCLQSRIFPRTFGQGTKLEI | |
| | K | |
| Humanized | DIVLTQSPASLAVSLGERATIN | 25 |
| c11D5.3 | CRASESVSVIGAHLIHWYQQKP | |
| scFv | GQPPKLLIYLASNLETGVPARF | |
| (VL-VH) | SGSGSGTDFTLTISSLQAEDAA | |
| | IYSCLQSRIFPRTFGQGTKLEI | |
| | KGSTSGSGKPGSGEGSTKGQVQ | |
| | LVQSGSELKKPGASVKVSCKAS | |
| | GYTFTDYSINWVRQAPGQGLEW | |
| | MGWINTETREPAYAYDFRGRFV | |
| | FSLDTSVSTAYLQISSLKAEDT | |
| | AVYYCARDYSYAMDYWGQGTLV | |
| | TVSS | |
| murine | QVQLQQSGGGLVQPGGSLKLSC | 29 |
| J22.9-xi | AASGIDFSRYWMSWVRRAPGKG | |
| VH | LEWIGEINPDSSTINYAPSLKD | |
| | KFIISRDNAKNTLYLQMSKVRS | |
| | EDTALYYCASLYYDYGDAMDYW | |
| | GQGTSVTVSS | |
| murine | DIVMTQSQRFMTTSVGDRVSVT | 33 |
| J22.9-xi | CKASQSVDSNVAWYQQKPRQSP | |
| VL | KALIFSASLRFSGVPARFTGSG | |
| | SGTDFTLTISNLQSEDLAEYFC | |
| | QQYNNYPLTFGAGTKLELKR | |
| murine | DIVMTQSQRFMTTSVGDRVSVT | 34 |
| J22.9-xi | CKASQSVDSNVAWYQQKPRQSP | |
| scFv | KALIFSASLRFSGVPARFTGSG | |
| (VL-VH) | SGTDFTLTISNLQSEDLAEYFC | |
| | QQYNNYPLTFGAGTKLELKRGS | |
| | TSGSGKPGSGEGSTKGQVQLQQ | |
| | SGGGLVQPGGSLKLSCAASGID | |
| | FSRYWMSWVRRAPGKGLEWIGE | |
| | INPDSSTINYAPSLKDKFIISR | |
| | DNAKNTLYLQMSKVRSEDTALY | |
| | YCASLYYDYGDAMDYWGQGTSV | |
| | TVSS | |
| Humanized | EVQLVESGGGLVQPGGSLRLSC | 35 |
| J22.9-xi | AASGFTFSRYWMSWVRQAP | |
| VH | GKGLEWVGEINPDSSTINYAPS | |
| | LKGRFTISRDNAKNTLYLQMNS | |
| | LRAEDTAVYYCASLYYDYGDAM | |
| | DYWGQGTLVTVSS | |
| Humanized | DIVMTQSPATLSVSVGDEVTLT | 36 |
| J22.9-xi VL | CKASQSVDSNVAWYQQKPGQAP | |
| | KLLIYSASLRFSGVPARFSGSG | |
| | SGTDFTLTISSLQSEDFAVYYC | |
| | QQYNNYPLTFGAGTKLELKR | |
| Humanized | DIVMTQSPATLvSVSVGDEVTL | 37 |
| J22.9-xi | TCKASQSVDSNVAWYQQKPGQA | |
| scFv | PKLLIYSASLRFSGVPARFSGS | |
| (VL-VH) | GSGTDFTLTISSLQSEDFAVYY | |
| | CQQYNNYPLTFGAGTKLELKRG | |
| | STSGSGKPGSGEGSTKGEVQLV | |
| | ESGGGLVQPGGSLRLSCAASGF | |
| | TFSRYWMSWVRQAPGKGLEWVG | |
| | EINPDSSTINYAPSLKGRFTIS | |
| | RDNAKNTLYLQMNSLRAEDTAV | |
| | YYCASLYYDYGDAMDYWGQGTL | |
| | VTVSS | |
| dAPRIL | VLHLVPINATSKDDSDVTEVMW | 38 |
| | QPALRRGRGLQAQGYGVRIQDA | |
| | GVYLLYSQVLFQDVTFTMGQVV | |
| | SREGQGRQETLFRCIRSMPSHP | |
| | DRAYNSCYSAGVFHLHQGDILS | |
| | VIIPRARAKLNLSPHGTFLGFV | |
| | KL | |

CS1-targeting domains

| | | |
|---|---|---|
| Murine | QVQLQQPGAELVRPGASVKLSC | 42 |
| Luc90 | KASGYSFTTYWMNWVKQRPGQG | |
| VH | LEWIGMIHPSDSETRLNQKFKD | |
| | KATLTVDKSSSTAYMQLSSPTS | |
| | EDSAVYYCARSTMIATRAMDYW | |
| | GQGTSVTVS | |
| Murine | DIVMTQSQKSMSTSVGDRVSIT | 46 |
| Luc90 | CKASQDVITGVAWYQQKPGQSP | |
| VL | KLLIYSASYRYTGVPDRFTGSG | |
| | SGTDFTFTISNVQAEDLAVYYC | |
| | QQHYSTPLTFGAGTKLELK | |
| Murine | QVQLQQPGAELVRPGASVKLSC | 47 |
| Luc90 | KASGYSFTTYWMNWVKQRPGQG | |
| (VH-VL) | LEWIGMIHPSDSETRLNQKFKD | |
| | KATLTVDKSSSTAYMQLsspts | |
| | edsavyycarstmiatramdyw | |
| | gqgtsvtvsgstsgsGKPGSGE | |
| | GSTKGDIVMTQSQKSMSTSVGD | |
| | RVSITCKASQDVITGVAWYQQK | |
| | PGQSPKLLIYSASYRYTGVPDR | |
| | FTGSGSGTDFTFTISNVQAEDL | |
| | AVYYCQQHYSTPLTFGAGTKLE | |
| | LK | |
| huLuc63 VH | EVQLVESGGGLVQPGGSLRLSC | 51 |
| | AASGFDFSRYWMSWVRQAPGKG | |
| | LEWIGEINPDSSTINYAPSLKD | |
| | KFIISRDNAKNSLYLQMNSLRA | |
| | EDTAVYYCARPDGNYWYFDVWG | |
| | QGTLVTVSS | |
| huLuc63 VL | DIQMTQSPSSLSASVGDRVTIT | 55 |
| | CKASQDVGIAVAWYQQKPGKVP | |
| | KLLIYWASTRHTGVPDRPSGSG | |
| | SGTDFTLTISSLQPEDVATYYC | |
| | QQYSSYPYTFGQGTKVEIK | |
| huLuc63 | EVQLVESGGGLVQPGGSLRLSC | 56 |
| (VH-VL) | AASGFDFSRYWMSWVRQAPGKG | |
| | LFWIGFINPDSSTINYAPSLKD | |
| | KFIISRDNAKNSLYLQMNSLRA | |
| | FDTAVYYCARPDGNYWYFDVWG | |
| | QGTLVTVSSGvSTSGSGKPGSG | |
| | FGSTKGDIQMTQSPSSLSASVG | |
| | DRVTITCKASQDVGIAVAWYQQ | |
| | KPGKVPKLLIYWASTRHTGVPD | |
| | RPSGSGSGTDFTLTISSLQPFD | |
| | VATYYCQQYSSYPYTFGQGTKV | |
| | EIK | |

Extracellular spacer, transmembrane domain,
and intracellular signaling domains

| | | |
|---|---|---|
| IgG4 hinge- | FSKYGPPCPPCP | 73 |
| CH2(L235E, | APEFEGGPSVFLFPPKPKDTLM | 74 |
| N297Q)- | ISRTPEVTCVVVDVSQFDPFVQ | |
| | FNWYVDGVEVHNAKTKPRFFQF | |
| | QSTYRVVSVLTVLHQDWLNGKF | |
| | YKCKVSNKGLPSSIFKTISKAK | |
| CH3 | GQPRFPQVYTLPPSQFFMTKNQ | 75 |
| | VSLTCLVKGFYPSDIAVFWFSN | |
| | GQPFNNYKTTPPVLDSDGSFFL | |
| | YSRLTVDKSRWQFGNVFSCSVM | |
| | HFALHNHYTQKSLSLSLGK | |
| CD28trans- | MFWVLVVVGGVLACYSLLVTV | 76 |
| membrane | AFIIFWV | |
| 4-1BB- | KRGRKKLLYIFKQPFMRPVQTT | 77 |
| | QEEDGCSCRFPEEEEGGCEL | |
| zeta | RVKFSRSADAPAYQQGQNQLYN | 78 |
| | FLNLGRRFFYDVLDKRRGRDPF | |
| | MGGKPRRKNPQFGLYNFLQKDK | |
| | MAFAYSFIGMKGFRRRGKGHDG | |
| | LYQGLSTATKDTYDALHMQALP | |
| | PR | |

In some embodiments, the spacer comprises the IgG4 hinge-CH2 (L235E, N297Q)-CH3 of SEQ ID NO: 172: ESKYGPPCPPCPAPEFEGGPSVFLFPPKPKDTLMISRTPEVTCVVVDVSQEDPEVQFN WYVDGVEVHNAKTKPREEQFQSTYRVVSVLTVLHQDWLNGKEYKCKVSNKGLPSS IEKTISKAKGQPREPQVYTLPPSQEEMTKNQVSLTCLVKGFYPSDIAVEWESNGQPEN NYKTTPPVLDSDGSFFLYSRLTVDKSRWQEGNVFSCSVMHEALHNHYTQKSLSLSL GK. In some embodiments, the spacer comprises or consists of the IgG4 hinge of SEQ ID NO:73.

Exemplary nucleic acid sequences of domain and regions described herein are provided in the table below:

TABLE 7
Nucleic acid sequences of CAR components

| | | |
|---|---|---|
| | | SEQ ID |
| Description | Nucleic Acid Sequence | NO. |

BCMA-targeting domains

| | | |
|---|---|---|
| Murine | gacatcgtgctgacccagagccccccc | 153 |
| c11D5.3 | agcctggccatgtctctgggcaagaga | |
| scFv | gccaccatcagctgccgggccagcgag | |
| (VL-VH) | agcgtgaccatcctgggcagccacctg | |
| | atccactggtatcagcagaagcccggc | |
| | cagccccccaccctgctgatccagctc | |
| | gccagcaatgtgcagaccggcgtgccc | |
| | gccagattcagcggcagcggcagcaga | |
| | accgacttcaccctgaccatcgacccc | |
| | gtggaagaggacgatgtggccgtgtac | |
| | tactgcctgcagagccggaccatcccc | |
| | cggacctttggcggaggcaccaaactg | |
| | gaaatcaagggctccacttctggctcc | |
| | ggcaaacctggttctggcgagggcagc | |
| | acaaagggacagattcagctggtgcag | |
| | agcggccctgagctgaagaaacccggc | |
| | gagacagtgaagatcagctgcaaggcc | |
| | tccggctacaccttcaccgactacagc | |
| | atcaactgggtgaaaagagcccctggc | |
| | aagggcctgaagtggatgggctggatc | |
| | aacaccgagacaagagagcccgcctac | |
| | gcctacgacttccggggcagattcgcc | |
| | ttcagcctggaaaccagcgccagcacc | |
| | gcctacctgcagatcaacaacctgaag | |
| | tacgaggacaccgccacctacttttgc | |
| | gccctggactacagctacgcgatggac | |
| | tactggggccagggcacctcagtcacc | |
| | gtctcctca | |
| Humanized | gacatcgtgctgacccagagccccgcc | 154 |
| c11D5.3 | agcctggccgtgtctctgggcGagaga | |
| scFv | gccaccatcaactgccgggccagcgag | |
| (VL-VH) | agcgtgtccgtgatcggcgctcacctg | |
| | atccactggtatcagcagaagcccggc | |
| | cagccccccaagctgctgatctacctA | |
| | gccagcaatctggagaccggcgtgccc | |
| | gccagattcagcggcagcggcagcggc | |
| | accgacttcaccctgaccatctcctct | |
| | ctgcaggccgaagatgcagccatctac | |
| | tcctgcctgcagagccggatcttcccc | |
| | cggacctttggccagggcaccaaactg | |
| | gaaatcaagggctccacttctggctcc | |
| | ggcaaacctggttctggcgagggcagc | |
| | acaaagggacaggtgcagctggtgcag | |
| | agcggctctgagctgaagaaacccggc | |
| | gccagcgtgaaggtgagctgcaaggcc | |
| | tccggctacaccttcaccgactacagc | |
| | atcaactgggtgagacaggcccctggc | |
| | cagggcctggagtggatgggctggatc | |
| | aacaccgagacaagagagcccgcctac | |
| | gcctacgacttccggggcagattcgtc | |
| | ttcagcctggacaccagcgtcagcacc | |
| | gcctacctgcagatctcttccctgaag | |
| | gccgaggacaccgccgtctactattgc | |
| | gcccgggactacagctacgcgatggac | |
| | tactggggccagggcaccctggtcacc | |
| | gtctcctca | |
| murine | gacattgtgatgactcagtctcaaaga | 155 |
| J22.9-xi | ttcatgaccacatcagtaggagacagg | |
| scFv | gtcagcgtcacctgcaaggccagtcag | |
| (VL-VH) | agtgtggatagtaatgtagcctggtat | |
| | caacagaaacctcggcaatctcctaaa | |
| | gcactgattttctcggcatccctccgg | |
| | ttcagtggagtccctgctcgcttcaca | |
| | ggcagtggatctgggacagatttcact | |
| | ctcaccatcagcaatctgcagtctgaa | |
| | gacttggcagagtatttctgtcaacaa | |
| | tataacaactatcctctcacgttcggt | |
| | gctgggaccaagctggagctgaaacgt | |
| | ggctccacttctggctccggcaaacct | |
| | ggttctggcgagggcagcacaaaggga | |
| | caggtgcagctgcagcagtctggaggt | |
| | ggcctggtgcagcctggaggatccctg | |
| | aaactctcctgtgcagcctcaggaatc | |
| | gattttagtagatactggatgagttgg | |
| | gttcggcgggctccagggaaaggacta | |
| | gaatggattggagaaattaatccagat | |
| | agcagtacaataaactatgcaccatct | |
| | ctaaaggataaattcatcatctccaga | |
| | gacaacgccaaaaatacgttg | |
| | tacctgcaaatgagcaaagtgcgctct | |
| | gaggacacagccctttattactgtgca | |
| | agtctctactatgattacggggatgct | |
| | atggactactggggtcaaggaacctca | |
| | gtcaccgtctcctca | |
| Humanized | gacattgtgatgactcagtctcccgcc | 156 |
| J22.9- | accctgagcgtgtcagtaggagacgag | |
| xi scFv | gtcaccctcacctgcaaggccagtcag | |
| (VL-VH) | agtgtggatagtaatgtagcctggtat | |
| | caacagaaacctgggcaagctcctaaa | |
| | ctgctgatttactcggcatccctccgg | |
| | ttcagtggagtccctgctcgcttcagc | |
| | ggcagtggatctgggacagatttcact | |
| | ctcaccatcagctctctgcagtctgaa | |
| | gacttcgcagtgtattactgtcaacaa | |
| | tataacaactatcctctcacgttcggt | |
| | gctgggaccaagctggagctgaaacgt | |
| | ggctccacttctggctccggcaaacct | |
| | ggttctggcgagggcagcacaaaggga | |
| | gaggtgcagctggtcgaatctggaggt | |
| | ggcctggtgcagcctggaggatccctg | |
| | aggctctcctgtgcagcctcaggattt | |
| | acctttagtagatactggatgagttgg | |
| | gttcggcaggctccagggaaaggacta | |
| | gaatgggtgggagaaattaatccagat | |
| | agcagtacaataaactatgcaccatct | |
| | ctaaagggcagattcaccatctccaga | |
| | gacaacgccaaaaatacgttgtacctg | |
| | caaatgaacagcctgcgcgctgaggac | |
| | acagccgtgtattactgtgcaagtctc | |
| | tactatgattacggggatgctatggac | |
| | tactggggtcaaggaaccctcgtcacc | |
| | gtctcctca | |
| dAPRIL | gtgctgcacctggtgcccatcaacgcc | 157 |
| | accagcaaggacgactctgatgtgacc | |
| | gaggtgatgtggcagccagccctgaga | |
| | cggggcagaggcctgcaggcccagggc | |
| | tacggcgtgagaatccaggacgctggc | |
| | gtgtacctgctgtactcccaggtgctg | |
| | ttccaggacgtgaccttcacaatgggc | |
| | caggtggtgagccgggagggccagggc | |
| | agacaggagaccctgttccggtgcatt | |
| | cgcagcatgcccagccaccccgacaga | |
| | gcctacaacagctgctacagcgctggc | |
| | gtgtttcacctgcaccagggcgacatc | |
| | ctgagcgtgatcatccccagagccaga | |
| | gccaagctgaacctgtccccccacggc | |
| | acctttctgggcttcgtgaagctg | |

CS1-targeting domains

| | | |
|---|---|---|
| Murine | caggtccaactgcagcagcctggggct | 158 |
| Luc90 | gagctggtgaggcctggagcttcagtg | |
| (VH-VL) | aagctgtcctgcaaggcttcggggtac | |
| | tccttcaccacctactggatgaactgg | |
| | gtgaagcagaggcctggacaaggcctt | |
| | gagtggattggcatgattcatccttcc | |
| | gatagtgaaactaggttaaatcagaag | |
| | ttcaaggacaaggccacattgactgta | |
| | gacaaatcctccagcacagcctacatg | |
| | caactcagcagcccgacatctgaggac | |
| | tctgcggtctattactgtgcacgttct | |
| | actatgattgcgacgagggctatggac | |
| | tactggggtcaaggaacctcagtcacc | |
| | gtctccgggtcaacttcaggctctggg | |
| | aaaccaggcagcggtgagggttcaacc | |
| | aagggtgacattgtgatgacccagtct | |
| | cagaaatccatgtccacatcagtagga | |
| | gacagggtcagcatcacctgcaaggcc | |
| | agtcaggatgttattactggtgtagcc | |
| | tggtatcaacagaaaccagggcaatct | |
| | cctaaattactgatttactcggcatcc | |
| | taccggtacactggagtccctgatcgc | |
| | ttcactggcagtggatctgggacggat | |
| | ttcactttcaccatcagcaatgtgcag | |
| | gctgaagacctggcagtttattactgt | |
| | cagcaacattatagtactcctctcact | |
| | ttcggtgctgggaccaagctggagctg | |
| | aaa | |
| huLuc63 | gaggtacaattggtggagtctggaggc | 159 |
| (VH-VL) | ggtctcgttcaaccagggggcagcctc | |
| | agactttcctgcgcggcaagtgggttt | |
| | gacttctctcgatattggatgtcatgg | |
| | gtcaggcaggcaccggggaaaggtctt | |
| | gagtggataggtgagattaaccctgac | |
| | tctagtaccatcaattacgctcccagc | |
| | ttgaaagataaattcataatttcacga | |
| | gacaacgccaaaaacagtttgtacctg | |
| | caaatgaatagcttgagggcggaggat | |
| | acggccgtttattactgtgctaggccc | |
| | gacggtaactactggtattttgatgta | |
| | tggggtcaaggcactctggtgactgta | |
| | tcctctggcagcaccagcggctccggc | |
| | aagcctggctctggcgagggcagcaca | |
| | aagggagacatacagatgacgcagtcc | |
| | ccttcatcactctctgcgagcgttggt | |
| | gacagggtgactatcacatgcaaagca | |
| | agccaagatgtcggtatagccgttgca | |
| | tggtatcagcagaaaccagggaaggtc | |
| | ccaaaactccttatatattgggcgagc | |
| | acacgccacactggtgtccctgatagg | |
| | cctagtggtagtggcagtggaacggat | |
| | ttcacccttactatatccagtttgcaa | |
| | cctgaggatgtggccacgtattattgt | |
| | cagcagtacagctcatatccttacacc | |
| | tttggtcaaggaactaaggtggaaatt | |
| | aag | |

Extracellular spacer, transmembrane domain,
and intracellular signaling domains

| | | |
|---|---|---|
| IgG4 hinge- | gaatctaagtacggaccgccctgcccc | 160 |
| | ccttgccct | |
| CH2(L235E, | gcccccgagttcgaaggcggacccagc | 161 |
| N297Q)- | gtgttcctgttcccccccaagcccaag | |
| | gacaccctgatgatcagccggaccccc | |
| | gaggtgacctgcgtggtggtggacgtg | |
| | agccaggaagatcccgaggtccagttc | |
| | aattggtacgtggacggcgtggaagtg | |
| | cacaacgccaagaccaagcccagagag | |
| | gaacagttccagagcacctaccgggtg | |
| | gtgtctgtgctgaccgtgctgcaccag | |
| | gactggctgaacggcaaagaatacaag | |
| | tgcaaggtgtccaacaagggcctgccc | |
| | agcagcatcgaaaagaccatcagcaag | |
| | gccaag | |
| CH3 | ggccagcctcgcgagccccaggtgtac | 162 |
| | accctgcctccctcccaggaagagatg | |
| | accaagaaccaggtgtccctgacctgc | |
| | ctggtgaagggcttctaccccagcgac | |
| | atcgccgtggagtgggagagcaacggc | |
| | cagcctgagaacaactacaagaccacc | |
| | cctcccgtgctggacagcgacggcagc | |
| | ttcttcctgtacagccggctgaccgtg | |
| | gacaagagccggtggcaggaaggcaac | |
| | gtctttagctgcagcgtgatgcacgag | |
| | gccctgcacaaccactacacccagaag | |
| | agcctgagcctgtccctgggcaag | |
| CD28tm- | atgttctgggtgctggtggtggtcgga | 163 |
| | ggcgtgctggcctgctacagcctgctg | |
| | gtcaccgtggccttcatcatcttttgg | |
| | gtg | |
| 4-1BB- | aaacggggcagaaagaaactcctgtat | 164 |
| | atattcaaacaaccatttatgagacca | |
| | gtacaaactactcaagaggaagatggc | |
| | tgtagctgccgatttccagaagaagaa | |
| | gaaggaggatgtgaactg | |
| Zeta | cgggtgaagttcagcagaagcgccgac | 165 |
| | gcccctgcctaccagcagggccagaat | |
| | cagctgtacaacgagctgaacctgggc | |
| | agaagggaagagtacgacgtcctggat | |
| | aagcggagaggccgggaccctgagatg | |
| | ggcggcaagcctcggcggaagaacccc | |
| | caggaaggcctgtataacgaactgcag | |
| | aaagacaagatggccgaggcctacagc | |
| | gagatcggcatgaagggcgagcggagg | |
| | cggggcaagggccacgacggcctgtat | |
| | cagggcctgtccaccgccaccaaggat | |
| | acctacgacgccctgcacatgcaggcc | |
| | ctgcccccaagg | |

TABLE 8
Further exemplary CAR embodiments

| | | SEQ |
|---|---|---|
| | | ID |
| Description | SEQUENCE | NO: |
| C11D5.3 Long | ATGGAGACAGACACACTCCTGCTATGGGTGCTGCT | 166 |
| CAR: MKLEADER | GCTCTGGGTTCCAGGTTCCACAGGCgacatcgtgctgaccca | |
| (UPPERCASE)- | gagcccccccagcctggccatgtctctgggcaagagagccaccatcagctgccgg | |
| bcma | gccagcgagagcgtgaccatcctgggcagccacctgatccaclggtatcagcaga | |
| (c11d5.3) | agcccggccagccccccaccctgctgatccagctcgccagcaatgtgcagaccgg | |
| scfv | cgtgcccgccagattcagcggcagcggcagcagaaccgacttcaccctgaccatc | |
| (vl/vh) | gaccccgtggaagaggacgatgtggccgtgtactactgcctgcagagccggacca | |
| (lowercase)- | tcccccggacctttggcggaggcaccaaactggaaatcaagggctccacttctggc | |
| IGG4 L235E N297Q | tccggcaaacctggttctggcgagggcagcacaaagggacagattcagctggtgc | |
| (UPPERCASE | agagcggccctgagctgaagaaacccggcgagacagtgaagatcagctgcaagg | |
| ITALIC)- | cctccggctacaccttcaccgactacagcatcaactgggtgaaaagagcccctggc | |
| cd28tm- | aagggcctgaagtggatgggctggatcaacaccgagacaagagagcccgcctac | |
| (lowercase | gcctacgacttccggggcagattcgccttcagcctggaaaccagcgccagcaccg | |
| italic)-4- | cctacctgcagatcaacaacctgaagtacgaggacaccgccacctacttttgcgccc | |
| 1BB(UPPERCASE | tggactacagctacgcgatggactactggggccagggcacctcagtcaccgtctcc | |
| DOUBLE | tcaGAATCTAAGTACGGACCGCCCTGCCCCCCTTGCCC | |
| UNDERLINE)-zeta | TGCCCCCGAGTTCGAAGGCGGACCCAGCGTGTTCCT | |
| (lowercase | GTTCCCCCCCAAGCCCAAGGACACCCTGATGATCAGC | |
| double | CGGACCCCCGAGGTGACCTGCGTGGTGGTGGACGTG | |
| underline) | AGCCAGGAAGATCCCGAGGTCCAGTTCAATTGGTACG | |
| *Single | TGGACGGCGTGGAAGTGCACAACGCCAAGACCAAGC | |
| Underlined | CCAGAGAGGAACAGTTCCAGAGCACCTACCGGGTGG | |
| portion denotes | TGTCTGTGCTGACCGTGCTGCACCAGGACTGGCTGAA | |
| the | CGGCAAAGAATACAAGTGCAAGGTGTCCAACAAGGGC | |
| linker that | CTGCCCAGCAGCATCGAAAAGACCATCAGCAAGGCCA | |
| connects | AGGGCCAGCCTCGCGAGCCCCAGGTGTACACCCTGC | |
| the heavy and | CTCCCTCCCAGGAAGAGATGACCAAGAACCAGGTGTC | |
| light | CCTGACCTGCCTGGTGAAGGGCTTCTACCCCAGCGAC | |
| chains of | ATCGCCGTGGAGTGGGAGAGCAACGGCCAGCCTGAG | |
| the scFv. | AACAACTACAAGACCACCCCTCCCGTGCTGGACAGCG | |
| | ACGGCAGCTTCTTCCTGTACAGCCGGCTGACCGTGGA | |
| | CAAGAGCCGGTGGCAGGAAGGCAACGTCTTTAGCTG | |
| | CAGCGTGATGCACGAGGCCCTGCACAACCACTACACC | |
| | CAGAAGAGCCTGAGCCTGTCCCTGGGCAAGatgttctgggt | |
| | gctggtggtggtgggcggggtgctggcctgctacagcctgctggtgacagtggcc | |
| | ttcatcatcttttgggtgAAACGGGGCAGAAAGAAACTCCTG | |
| | TATATATTCAAACAACCATTTATGAGACCAGTACA | |
| | AACTACTCAAGAGGAAGATGGCTGTAGCTGCCGAT | |
| | TTCCAGAAGAAGAAGAAGGAGGATGTGAACTGcgg | |
| | gtgaagttcagcagaagcgccgacgcccctgcctaccagcagggccagaatcag | |
| | ctgtacaacgagctgaacctgggcagaagggaagagtacgacgtcctggataagc | |
| | ggagaggccgggaccctgagatgggcggcaagcctcggcggaagaaccccca | |
| | ggaaggcctgtataacgaactgcagaaagacaagatggccgaggcctacagcga | |
| | gatcggcatgaagggcgagcggaggcggggcaagggccacgacggcctgtatc | |
| | agggcctgtccaccgccaccaaggatacctacgacgccctgcacatgcaggccct | |
| | gcccccaagg | |
| c11D5.3 Long | METDTLLLWVLLLWVPGSTGdivltqsppslamslgkratiscras | 169 |
| CAR: MKLEADER | esvtilgshlihwyqqkpgqpptlliqlasnvqtgvparfsgsgsrtdftltidpvcc | |
| (UPPERCASE)- | ddvavyyclqsrtiprtfgggtkleikgstsgsgkpgsgegstkgqiqlvqsgpel | |
| bcma(c11d5.3)scfv | kkpgetvkisckasgytftdysinwvkrapgkglkwmgwintetrepayaydf | |
| (vl/vh)(lowercase)- | rgrfafsletsastaylqinnlkyedtatyfcaldysyamdywgqgtsvtvssESK | |
| IGG4 L235E N297Q | YGPPCPPCPAPEFEGGPSVFLFPPKPKDTLMISRTPEVT | |
| (UPPERCASE | CVVVDVSQEDPEVQFNWYVDGVEVHNAKTKPREEQFQ | |
| ITALIC)-cd28tm- | STYRVVSVLTVLHQDWLNGKEYKCKVSNKGLPSSIEKTIS | |
| (lowercase | KAKGQPREPQVYTLPPSQEEMTKNQVSLTCLVKGFYPS | |
| italic)-4-1BB | DIAVEWESNGQPENNYKTTPPVLDSDGSFFLYSRLTVDK | |
| (UPPERCASE | SRWQEGNVFSCSVMHEALHNHYTQKSLSLSLGKmfwvlvv | |
| DOUBLE UNDERLINE)- | vggvlacysllvtvafiifwvKRGRKKLLYIFKQPFMRPVQTTQE | |
| zeta | EDGCSCRFPEEEEGGCELrvkfsrsadapavaaganalvnelnlgrr | |
| (lowercase double | eeydyldkrrgrdpemggkprrknpqeglynelqkdkmaeayseigmkgerr | |
| underline) | rgkghdglyqglstatkdtydalhmqalppr | |
| *Single Underlined | | |
| portion denotes | | |
| the linker that | | |
| connects the | | |
| heavy and light | | |
| chains of the scFv. | | |
| huLuc63-(G4S)4- | ATGGAGACAGACACACTCCTGCTATGGGTGCTGCT | 167 |
| C11D5.3 | GCTCTGGGTTCCAGGTTCCACAGGCgaggtacaattggtgga | |
| Short | gtctggaggcggtctcgttcaaccagggggcagcctcagactttcctgcgcggcaa | |
| CAR: MKLEADER | gtgggtttgacttctctcgatattggatgtcatgggtcaggcaggcaccggggaaag | |
| (UPPERCASE)- | gtcttgagtggataggtgagattaaccctgactctagtaccatcaattacgctcccag | |
| cs1(huluc63)scfv | cttgaaagataaattcataatttcacgagacaacgccaaaaacagtttgtacctgcaa | |
| (vh/vl) | atgaatagcttgagggcggaggatacggccgtttattactgtgctaggcccgacggt | |
| (lowercase)- | aactactggtattttgatgtatggggtcaaggcactctggtgactgtatcctctggcag | |
| (G4S1)4-LINKER | caccagcggctccggcaagcctggctctggcgagggcagcacaaagggagacat | |
| (UPPERCASE ITALIC)- | acagatgacgcagtccccttcatcactctctgcgagcgttggtgacagggtgactat | |
| bcma(c11d5.3)scfv | cacatgcaaagcaagccaagatgtcggtatagccgttgcatggtatcagcagaaac | |
| (vl/vh) | cagggaaggtcccaaaactccttatatattgggcgagcacacgccacactggtgtc | |
| (lowercase | cctgataggcctagtggtagtggcagtggaacggatttcacccttactatatccagttt | |
| italic)-IGG4 HINGE | gcaacctgaggatgtggccacgtattattgtcagcagtacagctcatatccttacacct | |
| (UPPERCASE, | ttggtcaaggaactaaggtggaaattaagGGTGGAGGCGGCAGTGG | |
| DOUBLE | CGGAGGTGGGTCCGGAGGGGGCGGTAGCGGTGGCG | |
| UNDERLINE)- | GGGGATCTgacatcgtgctgacccagagcccccccagcctggccatgtctc | |
| cd28tm | tgggcaagagagccaccatcagctgccgggccagcgagagcgtgaccatcctg | |
| (lowercase, | ggcagccacctgatccactggtatcagcagaagcccggccagccccccaccct | |
| double underline)- | gcttatccagctcgccagcaatgtgcagaccggcgtgcccgccagattcagcgg | |
| 4-1BB | cagcggcagcagaaccgacttcaccctgaccatcgaccccgtggaagaggac | |
| (UPPERCASE, | gatgtggccgtgtactactgcctgcagagccggaccatcccccggacctttggcg | |
| ITALIC, DOUBLE | gaggcaccaaactggaaatcaagggctccacttctggctccggcaaacctggtt | |
| UNDERLINE)-zeta | ctggcgagggcagcacaaagggacagattcagctggtgcagagcggccctga | |
| (lowercase, | gctgaagaaacccggcgagacagtgaagatcagctgcaaggcctccggctac | |
| Italic, | accttcaccgactacagcatcaactgggtgaaaagagcccctggcaagggcct | |
| double underline) | gaagtggatgggctggatcaacaccgagacaagagagcccgcctacgcctac | |
| *single underlined | gacttccggggcagattcgccttcagcctggaaaccagcgccagcaccgcctac | |
| portion denotes the | ctgcagatcaacaacctgaagtacgaggacaccgccacctacttttgcgccctgg | |
| linker that connects | actacagctacgcgatggactactggggccagggcacctcagtcaccgtctcctc | |
| the heavy and light | aGAATCTAAGTACGGACCGCCCTGCCCCCCTTGCC | |
| chains of | CTatgttctgggtgctggtggtggtcggaggcgtgctggcctgctacagcctgctg | |
| a given | gtcaccgtggccttcatcatcttttgggtg [symbol] | |
| scFv. | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] | |
| huLuc63-(G4G)4- | METDTLLLWVLLLWVPGSTGevqlvesggglvqpggslrlscaa | 170 |
| C11D5.3 Short | sgfdfsrywmswvrqapgkglewigeinpdsstinyapslkdkfiisrdnakns | |
| CAR: MKLEADER | lylqmnslraedtavyycarpdgnywyfdvwgqgtlvtvssgstsgsgkpgsg | |
| (UPPERCASE)- | egstkgdiqmtqspsslsasvgdrvtitckasqdvgiavawyqqkpgkvpklli | |
| cs1(huluc63)scfv | ywastrhtgvpdrpsgsgsgtdftltisslqpedvatyycqqyssypytfgqgtkv | |
| (vh/vl) (lowercase)- | eikGGGGSGGGGSGGGGSGGGGSdivltqsppslamslgkratisc | |
| (G4S1)4-LINKER | rasesvtilgshlihwyqqkpgqpptlliqlasnvqtgvparfsgsgsrtdftltidpv | |
| (UPPERCASE | eeddvavvvclqsrtiprtfgggtkleikgstsgsgkpgsgegstkgqiqlvqsgpe | |
| ITALIC)- | Ikkpgetvkisckasgytftdysinwvkrapgkglkwmgwintetrepayaydfr | |
| bcma(c11d5.3) scfv | grfafslelsastaylqinnlkyedtatyfcaldysyamdywgqgtsvtvssESK | |
| (vl/vh) (lowercase | YGPPCPPCPmfwvlvvvggvlacvsllvtvafiifwv [symbol] | |
| Italic)-IGG4 HINGE | [symbol] [symbol] | |
| (UPPERCASE, | [symbol] [symbol] | |
| DOUBLE | [symbol] [symbol] | |
| UNDERLINE) | | |
| cd28tm (lowercase, | | |
| double underline)-4- | | |
| 1BB (UPPERCASE, | | |
| ITALIC, DOUBLE | | |
| UNDERLINE)-zeta | | |
| (lowercase, italic, | | |
| double underline) | | |
| *single underlined | | |
| portion denotes the | | |
| linker that connects | | |
| the heavy and light | | |
| chains of a given | | |
| scF | | |
| huLuc63-(G4S)4- | ATGGAGACAGACACACTCCTGCTATGGGTGCTGCT | 168 |
| c11D5.3 Medium | GCTCTGGGTTCCAGGTTCCACAGGCgaggtacaattggtgga | |
| CAR: MKLEADER | gtctggaggcggtctcgttcaaccagggggcagcctcagactttcctgcgcggcaa | |
| (UPPERCASE)- | gtgggtttgacttctctcgatattggatgtcatgggtcaggcaggcaccggggaaag | |
| cs1(huluc63)scfv | gtcttgagtggataggtgagattaaccctgactctagtaccatcaattacgctcccag | |
| (vh/vl) | cttgaaagataaattcataatttcacgagacaacgccaaaaacagtttgtacctgcaa | |
| (lowercase)- | atgaatagcttgagggcggaggatacggccgtttattactgtgctaggcccgacggt | |
| (G4S1)4-LlNKER | aactactggtattttgatgtatggggtcaaggcactctggtgactgtatcctctggcag | |
| (UPPERCASE ITALIC)- | caccagcggctccggcaagcctggctctggcgagggcagcacaaagggagacat | |
| bcma(c11d5.3) | acagatgacgcagtccccttcatcactctctgcgagcgttggtgacagggtgactat | |
| scfv (vl/vh) | cacatgcaaagcaagccaagatgtcggtatagccgttgcatggtatcagcagaaac | |
| (lowercase italic)- | cagggaaggtcccaaaactccttatatattgggcgagcacacgccacactggtgtc | |
| IGG4 HINGE, | cctgataggcctagtggtagtggcagtggaacggatttcacccttactatatccagttt | |
| (UPPERCASE, | gcaacctgaggatgtggccacgtattattgtcagcagtacagctcatatccttacacct | |
| DOUBLE UNDERLINE)- | ttggtcaaggaactaaggtggaaattaagGGTGGAGGCGGCAGTGG | |
| cd28tm (lowercase, | CGGAGGTGGGTCCGGAGGGGGCGGTAGCGGTGGCG | |
| double underline)- | GGGGATCTgacatcgtgctgacccagagcccccccagcctggccatgtctc | |
| 4-1BB (UPPERCASE, | tgggcaagagagccaccatcagctgccgggccagcgagagcgtgaccatcctg | |
| ITALIC, DOUBLE, | ggcagccacctgatccactggtatcagcagaagcccggccagccccccaccct | |
| UNDERLINE)-zeta | gcttatccagctcgccagcaatgtgcagaccggcgtgcccgccagattcagcgg | |
| (lowercase, italic, | cagcggcagcagaaccgacttcaccctgaccatcgaccccgtggaagaggac | |
| double underline) | gatgtggccgtgtactactgcctgcagagccggaccatcccccggacctttggcg | |
| *single underlined | gaggcaccaaactggaaatcaagggctccacttctggctccggcaaacctggtt | |
| portion denotes the | ctggcgagggcagcacaaagggacagattcagctggtgcagagcggccctga | |
| linker that | gctgaagaaacccggcgagacagtgaagatcagctgcaaggcctccggctac | |
| connects the | accttcaccgactacagcatcaactgggtgaaaagagcccctggcaagggcct | |
| heavy and light | gaagtggatgggctggatcaacaccgagacaagagagcccgcctacgcctac | |
| chains of a | gacttccggggcagattcgccttcagcctggaaaccagcgccagcaccgcctac | |
| given scFv. | ctgcagatcaacaacctgaagtacgaggacaccgccacctacttttgcgccctgg | |
| | actacagctacgcgatggactactggggccagggcacctcagtcaccgtctcctc | |
| | aGAATCTAAGTACGGACCGCCCTGCCCCCCTTGCC | |
| | CTGGCCAGCCTAGAGAACCCCAGGTGTACACCCTG | |
| | CCTCCCAGCCAGGAAGAGATGACCAAGAACCAGG | |
| | TGTCCCTGACCTGCCTGGTCAAAGGCTTCTACCCCA | |
| | GCGATATCGCCGTGGAATGGGAGAGCAACGGCCA | |
| | GCCCGAGAACAACTACAAGACCACCCCCCCTGTGC | |
| | TGGACAGCGACGGCAGCTTCTTCCTGTACTCCCGG | |
| | CTGACCGTGGACAAGAGCCGGTGGCAGGAAGGCA | |
| | ACGTCTTCAGCTGCAGCGTGATGCACGAGGCCCTG | |
| | CACAACCACTACACCCAGAAGTCCCTGAGCCTGAG | |
| | CCTGGGCAAGatgttctgggtgctggtggtggtcggaggcgtgctggcct | |
| | gctacagcctgctggtcaccgtggccttcatcatcttttgggtg [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] [symbol] | |
| | [symbol] | |
| huLuc63-(G4S)4- | METDTLLLWVLLLWVPGSTGevqlvesggglvqpggslrlscaa | 171 |
| c11D5.3 Medium | sgfdfsrywmswvrqapgkglewigeinpdsstinyapslkdkfiisrdnakns | |
| CAR: MKLEADER | lylqmnslraedtavyycarpdgnywyfdvwgqgtlvtvssgstsgsgkpgsg | |
| (UPPERCASE)- | egstkgdiqmtqspsslsasvgdrvtitckasqdvgiavawyqqkpgkvpklli | |
| cs1(huluc63) | ywastrhtgvpdrpsgsgsgtdftltisslqpedvatyycqqyssypytfgqgtkv | |
| scfv | eikGGGGSGGGGSGGGGSGGGGSdivltqsppslamslgkratisc | |
| (vh/vl) | rasesvtilgshlihwyqqkpgqpptlliqlasnvqtgvparfsgsgsrtdftltidpv | |
| (lowercase)- | eeddvavvvclqsrtiprtfgggtkleikgstsgsgkpgsgegstkgaialvasgpe | |
| (G4S1)4-LINKER | Ikkpgetvkisckasgytftdysinwvkrapgkglkwmgwintetrepayaydfr | |
| (UPPERCASE | grfafsletsastaylqinnlkyedtatyfcaldysyamdywgqgtsvtvssESK | |
| ITALIC)- | YGPPCPPCPGQPREPQVYTLPPSQEEMTKNQVSLTCL | |
| bcma(c11d5.3)scfv | VKGFYPSDTAVEWESNGQPENNYKTTPPVLDSDGSFF | |
| (vl/vh) | LYSRETVDKSRWQEGNVFSCSVMHEALHNHYTQKS | |
| (lowercase | LSLSLGKmfwvlvvvggvlacvsllvtvafiifwv [symbol] | |
| italic)- | [symbol] [symbol] | |
| IGG4 | [symbol] [symbol] | |
| HINGE | [symbol] [symbol] | |
| (UPPERCASE, | | |
| DOUBLE | | |
| UNDERLINE)- | | |
| cd28tm | | |
| (lowercase, | | |
| double | | |
| underline)-4- | | |
| 1BB (UPPERCASE, | | |
| ITALIC, DOUBLE | | |
| UNDERLINE)-zeta | | |
| (lowercase, | | |
| italic, | | |
| double | | |
| underline) | | |
| *single | | |
| underlined | | |
| portion denotes | | |
| the | | |
| linker that | | |
| connects | | |
| the heavy | | |
| and light | | |
| chains of a | | |
| given | | |
| scFv. | | |

Further CAR embodiments include the following:

BCMA/CS1 Loop CAR 1: MKleader-CS1(huLuc63)Vh-G4S-BCMA(C11D5.3)Vl-linker-BCMA(C11D5.3)Vh-G4S-CS1(huLuc63)Vl-IgG4 Hinge-CD28tm-4-1BB-Zeta

BCMA/CS1 Loop CAR 2: MKleader-CS1(huLuc63)Vh-G4S-BCMA(C11D5.3)Vh-linker-BCMA(C11D5.3)Vl-G4S-CS1(huLuc63)Vl-IgG4 Hinge-CD28tm-4-1BB-Zeta

BCMA/CS1 Loop CAR 3: MKleader-BCMA(C11D5.3)Vl-G4S-CS1(huLuc63)Vh-linker-CS1(huLuc63)Vl-G4S-BCMA(C11D5.3)Vh-IgG4 Hinge-CD28tm-4-1BB-Zeta

BCMA/CS1 Loop CAR 4: MKleader-BCMA(C11D5.3)Vl-G4S-CS1(huLuc63)Vl-linker-CS1(huLuc63)Vh-G4S-BCMA(C11D5.3)Vh-IgG4 Hinge-CD28tm-4-1BB-Zeta

BCMA/CS1 Loop CAR 5: MKleader-CS1(huLuc63)Vh-G4S-BCMA(C11D5.3)Vl-linker-BCMA(C11D5.3)Vh-G4S-CS1(huLuc63)Vl-IgG4 Hinge_CH3-CD28tm-4-1BB-Zeta

BCMA/CS1 Loop CAR 6: MKleader-CS1(huLuc63)Vh-G4S-BCMA(C11D5.3)Vh-linker-BCMA(C11D5.3)Vl-G4S-CS1(huLuc63)Vl-IgG4 Hinge_CH3-CD28tm-4-1BB-Zeta

BCMA/CS1 Loop CAR 7: MKleader-BCMA(C11D5.3)Vl-G4S-CS1(huLuc63)Vh-linker-CS1(huLuc63)Vl-G4S-BCMA(C11D5.3)Vh-IgG4 Hinge_CH3-CD28tm-4-1BB-Zeta

BCMA/CS1 Loop CAR 8: MKleader-BCMA(C11D5.3)Vl-G4S-CS1(huLuc63)Vl-linker-CS1(huLuc63)Vh-G4S-BCMA(C11D5.3)Vh-IgG4 Hinge_CH3-CD28tm-4-1BB-Zeta

BCMA/CS1 Loop CAR 9: MKleader-CS1(huLuc63)Vh-G4S-BCMA(C11D5.3)Vl-linker-BCMA(C11D5.3)Vh-G4S-CS1(huLuc63)Vl-IgG4 Hinge_CH2_CH3-CD28tm-4-1BB-Zeta

BCMA/CS1 Loop CAR 10: MKleader-CS1(huLuc63)Vh-G4S-BCMA(C11D5.3)Vh-linker-BCMA(C11D5.3)Vl-G4S-CS1(huLuc63)Vl-IgG4 Hinge_CH2_CH3-CD28tm-4-1BB-Zeta

BCMA/CS1 Loop CAR 11: MKleader-BCMA(C11D5.3)Vl-G4S-CS1(huLuc63)Vh-linker-CS1(huLuc63)Vl-G4S-BCMA(C11D5.3)Vh-IgG4 Hinge_CH2_CH3-CD28tm-4-1BB-Zeta

BCMA/CS1 Loop CAR 12: MKleader-BCMA(C11D5.3)Vl-G4S-CS 1(huLuc63)Vl-linker-CS1(huLuc63)Vh-G4S-BCMA(C11D5.3)Vh-IgG4 Hinge_CH2_CH3-CD28tm-4-1BB-Zeta.

In some embodiments, the CAR comprises a BCMA/CS1 Loop, as shown above. Exemplary BCMA/CS1 Loop sequences include the following:

| BCMA/CS1 Loop | Sequence |
|---|---|
| CS1(huLuc63) | EVQLVESGGGLVQPGGSLRLSCAASGFD |
| Vh-G4S- | FSRYWMSWVRQAPGKGLEWIGEINPDSS |
| BCMA(C11D5.3) | TINYAPSLKDKFIISRDNAKNSLYLQMN |
| Vl-linker- | SLRAEDTAVYYCARPDGNYWYFDVWGQG |
| BCMA(C11D5.3) | TLVTVSSGGGGSDIVLTQSPPSLAMSLG |
| Vh-G4S-CS1(huLuc63) | KRATISCRASESVTILGSHLIHWYQQKP |
| Vl (Loop CAR | GQPPTLLIQLASNVQTGVPARFSGSGSR |
| 1, 5, 9) | TDFTLTIDPVEEDDVAVYYCLQSRTIPR |
| | TFGGGTKLEIKGSTSGSGKPGSGEGSTK |
| | GQIQLVQSGPELKKPGETVKISCKASGY |
| | TFTDYSINWVKRAPGKGLKWMGWINTET |
| | REPAYAYDFRGRFAFSLETSASTAYLQI |
| | NNLKYEDTATYFCALDYSYAMDYWGQGT |
| | SVTVSSGGGGSDIQMTQSPSSLSASVGD |
| | RVTITCKASQDVGIAVAWYQQKPGKVPK |
| | LLIYWASTRHTGVPDRPSGSGSGTDFTL |
| | TISSLQPEDVATYYCQQYSSYPYTFGQG |
| | TKVEIK |
| | (SEQ ID NO: 174) |
| CS1(huLuc63) | EVQLVESGGGLVQPGGSLRLSCAASGFD |
| Vh-G4S- | FSRYWMSWVRQAPGKGLEWIGEINPDSS |
| BCMA(11D5.3)Vh- | TINYAPSLKDKFIISRDNAKNSLYLQMN |
| linker- | SLRAEDTAVYYCARPDGNYWYFDVWGQG |
| BCMA(C11D5.3)Vl- | TLVTVSSGGGGSQIQLVQSGPELKKPGE |
| G4S-CS1(huLuc63) | TVKISCKASGYTFTDYSINWVKRAPGKG |
| Vl (Loop CAR 2, | LKWMGWINTETREPAYAYDFRGRFAFSL |
| 6, 10) | ETSASTAYLQINNLKYEDTATYFCALDY |
| | SYAMDYWGQGTSVTVSSGSTSGSGKPGS |
| | GEGSTKGDIVLTQSPPSLAMSLGKRATI |
| | SCRASESVTILGSHLIHWYQQKPGQPPT |
| | LLIQLASNVQTGVPARFSGSGSRTDFTL |
| | TIDPVEEDDVAVYYCLQSRTIPRTFGGG |
| | TKLEIKGGGGSDIQMTQSPSSLSASVGD |
| | RVTITCKASQDVGIAVAWYQQKPGKVPK |
| | LLIYWASTRHTGVPDRPSGSGSGTDFTL |
| | TISSLQPEDVATYYCQQYSSYPYTFGQG |
| | TKVEIK |
| | (SEQ ID NO: 175) |
| BCMA(C11D5.3) | DIVLTQSPPSLAMSLGKRATISCRASES |
| Vl-G4S-CS1 | VTILGSHLIHWYQQKPGQPPTLLIQLAS |
| (huLuc63) | NVQTGVPARFSGSGSRTDFTLTIDPVEE |
| Vh-linker- | DDVAVYYCLQSRTIPRTFGGGTKLEIKG |
| CS1(huLuc63) | GGGSEVQLVESGGGLVQPGGSLRLSCAA |
| Vl-G4S- | SGFDFSRYWMSWVRQAPGKGLEWIGEIN |
| BCMA(C11D5.3) | PDSSTINYAPSLKDKFIISRDNAKNSLY |
| Vh | LQMNSLRAEDTAVYYCARPDGNYWYFDV |
| (Loop CAR | WGQGTLVTVSSGSTSGSGKPGSGEGSTK |
| 3, 7, 11) | GDIQMTQSPSSLSASVGDRVTITCKASQ |
| | DVGIAVAWYQQKPGKVPKLLIYWASTRH |
| | TGVPDRPSGSGSGTDFTLTISSLQPEDV |
| | ATYYCQQYSSYPYTFGQGTKVEIKGGGG |
| | SQIQLVQSGPELKKPGETVKISCKASGY |
| | TFTDYSINWVKRAPGKGLKWMGWINTET |
| | REPAYAYDFRGRFAFSLETSASTAYLQI |
| | NNLKYEDTATYFCALDYSYAMDYWGQGT |
| | SVTVSS |
| | (SEQ ID NO: 176) |
| BCMA(C11D5.3) | DIVLTQSPPSLAMSLGKRATISCRASES |
| Vl-G4S-CS1 | VTILGSHLIHWYQQKPGQPPTLLIQLAS |
| (huLuc63) | NVQTGVPARFSGSGSRTDFTLTIDPVEE |
| Vl-linker- | DDVAVYYCLQSRTIPRTFGGGTKLEIKG |
| CS1(huLuc63) | GGGSDIQMTQSPSSLSASVGDRVTITCK |
| Vh-G4S- | ASQDVGIAVAWYQQKPGKVPKLLIYWAS |
| BCMA(C11D5.3) | TRHTGVPDRPSGSGSGTDFTLTISSLQP |
| Vh | EDVATYYCQQYSSYPYTFGQGTKVEIKG |
| (Loop CAR 4, | STSGSGKPGSGEGSTKGEVQLVESGGGL |
| 8, 12) | VQPGGSLRLSCAASGFDFSRYWMSWVRQ |
| | APGKGLEWIGEINPDSSTINYAPSLKDK |
| | FIISRDNAKNSLYLQMNSLRAEDTAVYY |
| | CARPDGNYWYFDVWGQGTLVTVSSGGGG |
| | SQIQLVQSGPELKKPGETVKISCKASGY |
| | TFTDYSINWVKRAPGKGLKWMGWINTET |
| | REPAYAYDFRGRFAFSLETSASTAYLQI |
| | NNLKYEDTATYFCALDYSYAMDYWGQGT |
| | SVTVSS (SEQ ID NO: 177) |

“IgG4 CH2” in the above has the following nucleic acid and protein sequences:

(SEQ ID NO: 161)
gcccccgagttcgaaggcggacccagcgtgttcctgttccccc
ccaagcccaaggacaccctgatgatcagccggacccccgaggt
gacctgcgtggtggtggacgtgagccaggaagatcccgaggtc
cagttcaattggtacgtggacggcgtggaagtgcacaacgcca
agaccaagcccagagaggaacagttccagagcacctaccgggt
ggtgtctgtgctgaccgtgctgcaccaggactggctgaacggc
aaagaatacaagtgcaaggtgtccaacaagggcctgcccagca
gcatcgaaaagaccatcagcaaggccaag;
(SEQ ID NO: 74
APEFEGGPSVFLFPPKPKDTLMISRTPEVTCVVVDVSQEDPEV
QFNWYVDGVEVHNAKTKPREEQFQSTYRVVSVLTVLHQDWLNG
KEYKCKVSNKGLPSSIEKTIKAK.

In some embodiments, the CAR molecules of the disclosure comprise a leader sequence. In some embodiments, the leader sequence comprises METDTLLLWVLLLWVPGSTG (SEQ ID NO:129). In some embodiments, the CAR comprises a linker of SEQ ID NO:173: GSTSGSGKPGSGEGSTKG. In some embodiments, the linker of SEQ ID NO:173 is between a VH and VL region of a BCMA or CS1-binding region.

Further embodiments, including embodiments for VH regions, VL regions, CDRs, signaling domains, cytoplasmic regions, transmembrane domains, and linkers, for example are shown in WO2010104949, WO2013154760, WO2016014565, WO2014068079, WO2015052538, U.S. Pat. No. 7,709,610, WO2014055370, and WO2014179759, which are herein incorporated by reference for all purposes.

### IX. EXAMPLES

The following examples are included to demonstrate embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: High-Throughput Design and Characterization of BCMA/CS1 Bispecific Chimeric Antigen Receptors (CARs) for Multiple Myeloma

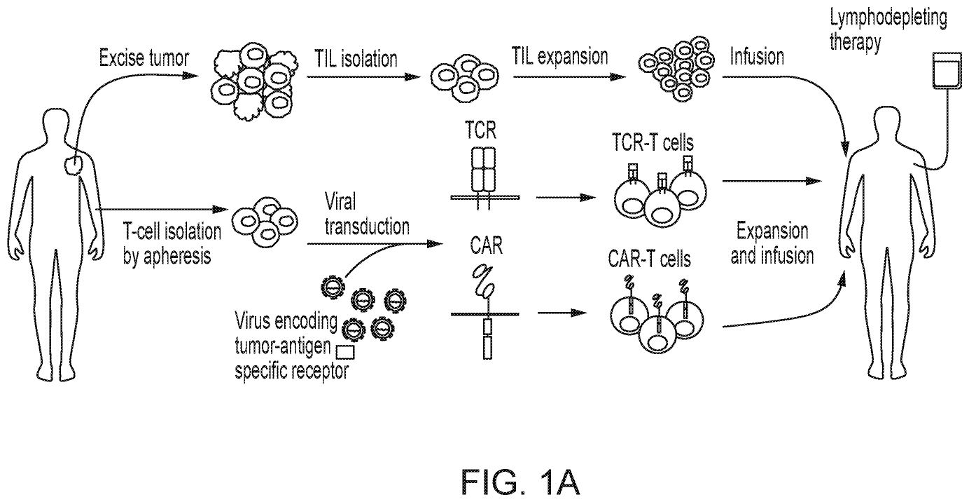

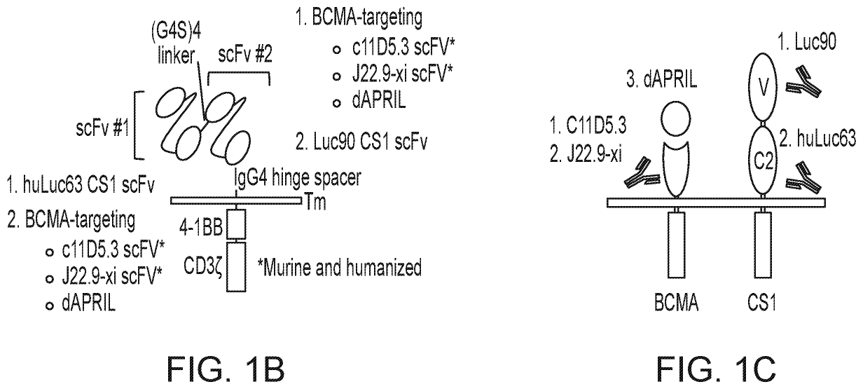

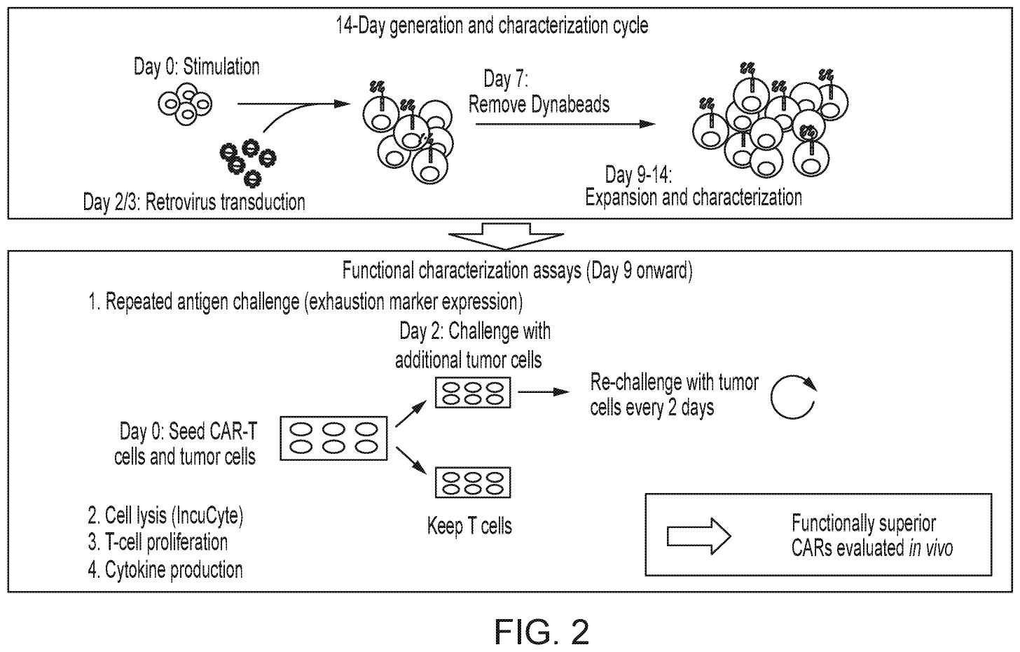

Multiple myeloma (MM) is an incurable disease affecting plasma cells, which are B cells that play a critical role in the humoral immune response. The adoptive transfer of chimeric antigen receptor (CAR)-T cells targeting B-cell maturation antigen (BCMA) has achieved up to 100% response rate in the treatment of MM patients. However, following initial response to therapy, progression of tumors with downregulated BCMA expression has been observed, suggesting antigen escape as a critical limitation of the treatment. Another potential target for MM is CS1 (SLAMF7), which is highly expressed on MM. While CS1 CARs have demonstrated efficacy against myeloma, CS1 CAR-T cells may be susceptible to fratricide since CS1 is also expressed at high levels in activated T cells. To address these limitations, the inventors constructed a panel of single-chain bispecific CARs for the treatment of MM. Using high-throughput characterization methods, the inventors identified BCMA/CS1 CAR-T cells that effectively target both BCMA and CS1 while retaining robust capacity for ex vivo expansion. In addition, BCMA/CS1 CAR-T cells could effectively control tumor growth in established MM xenografts in vivo. Overall, the BCMA/CS1 bispecific CAR presents a promising treatment approach to prevent antigen escape in CAR-T cell therapy against MM. In comparison to dual CARs (i.e., co-expressing two full-length CARs, each targeting a single antigen), the single-chain bispecific CAR-T cells developed here have a significantly smaller DNA footprint and can therefore be more efficiently transduced into T cells to meet manufacturing requirements for generating therapeutic T cells for adoptive T cell therapy. FIGS. 1-6 of the application and the figure descriptions for FIGS. 1-6 further describe the experimental data.

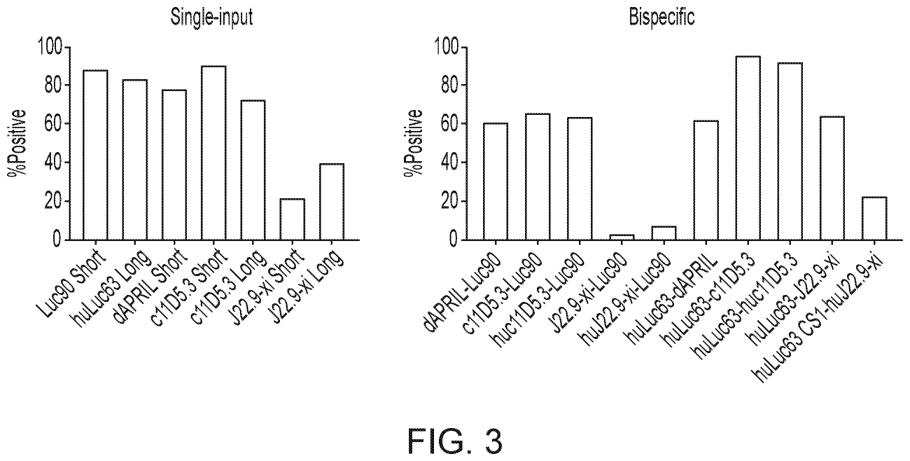

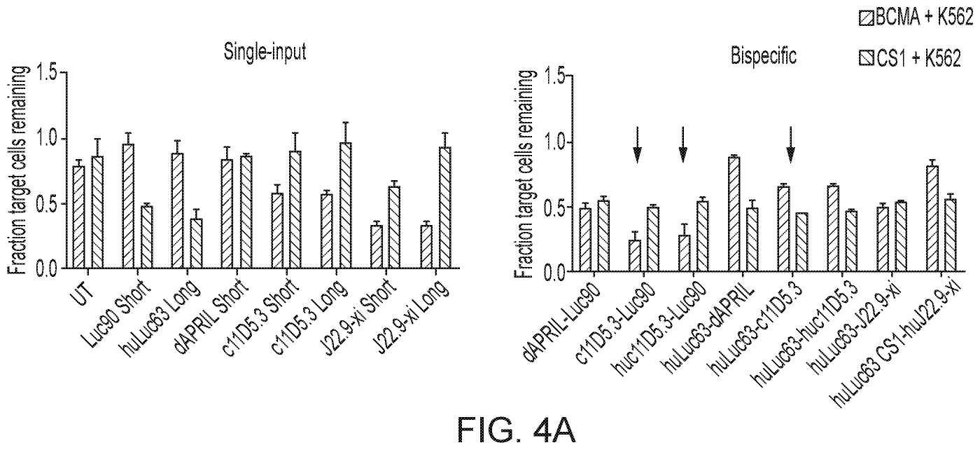

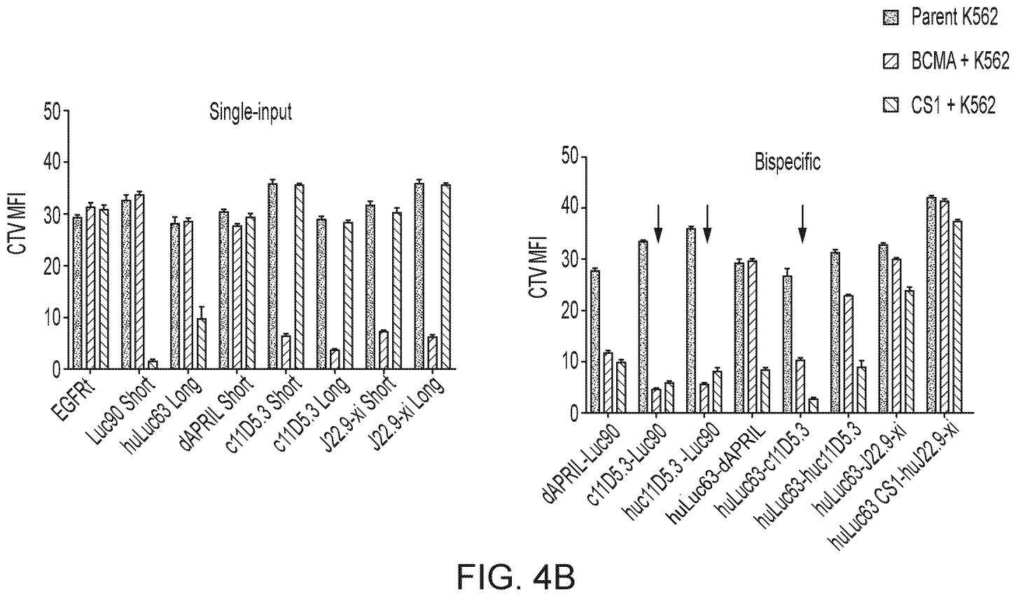

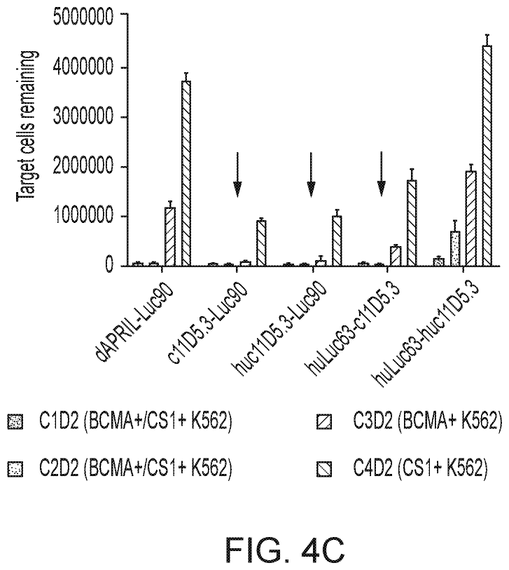

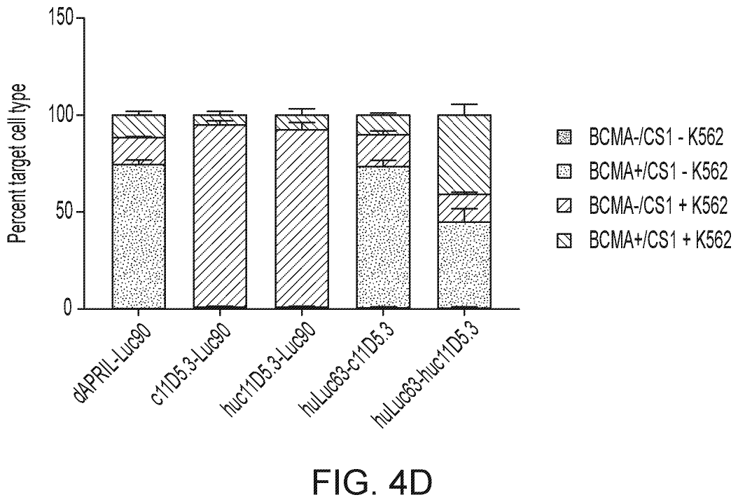

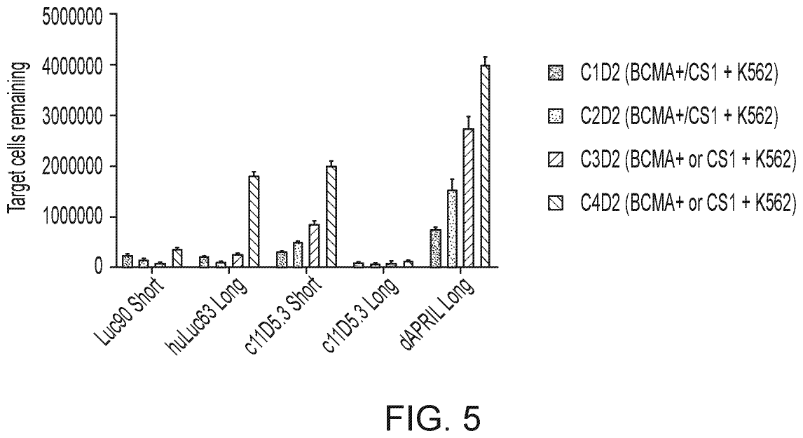

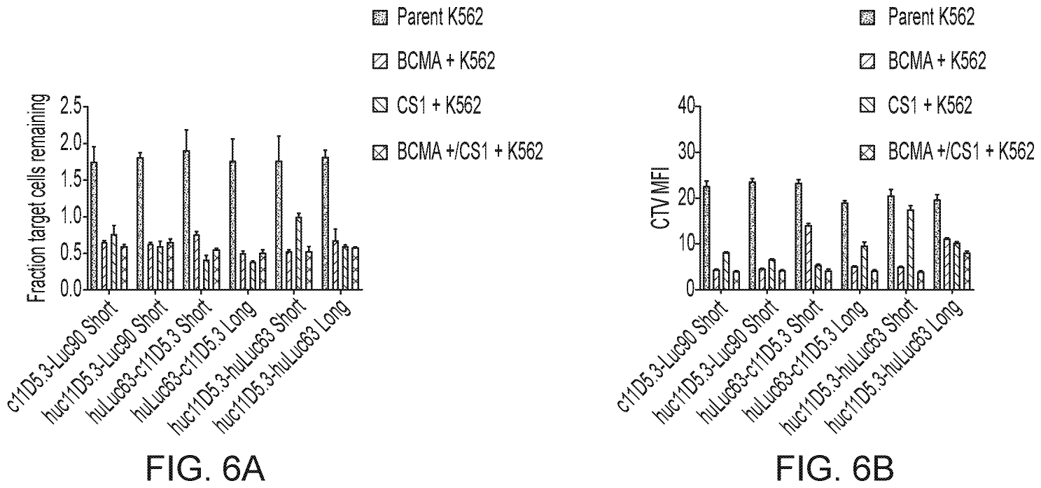

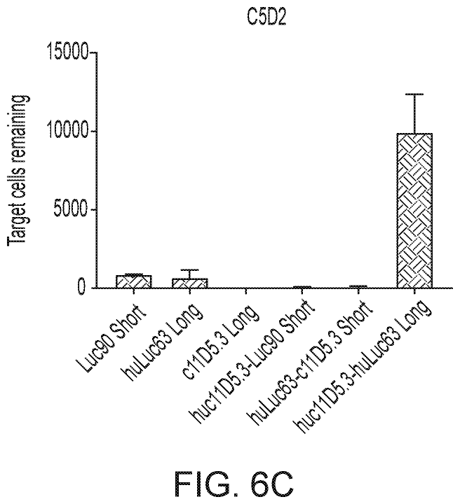

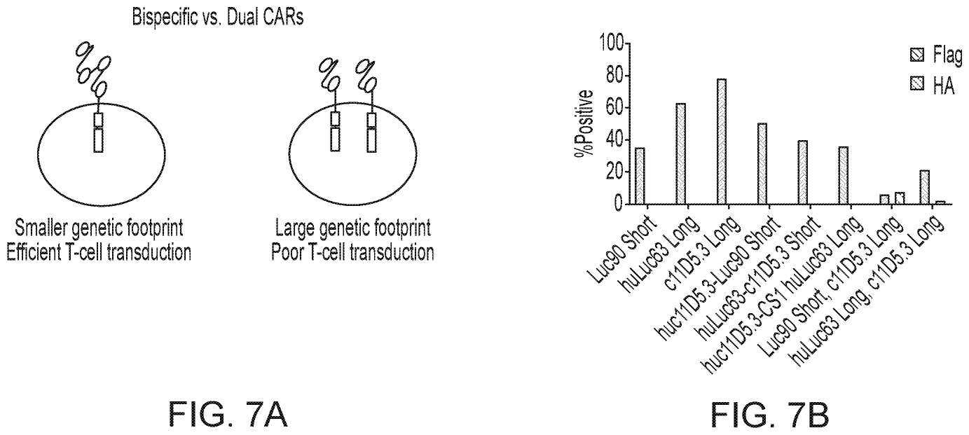

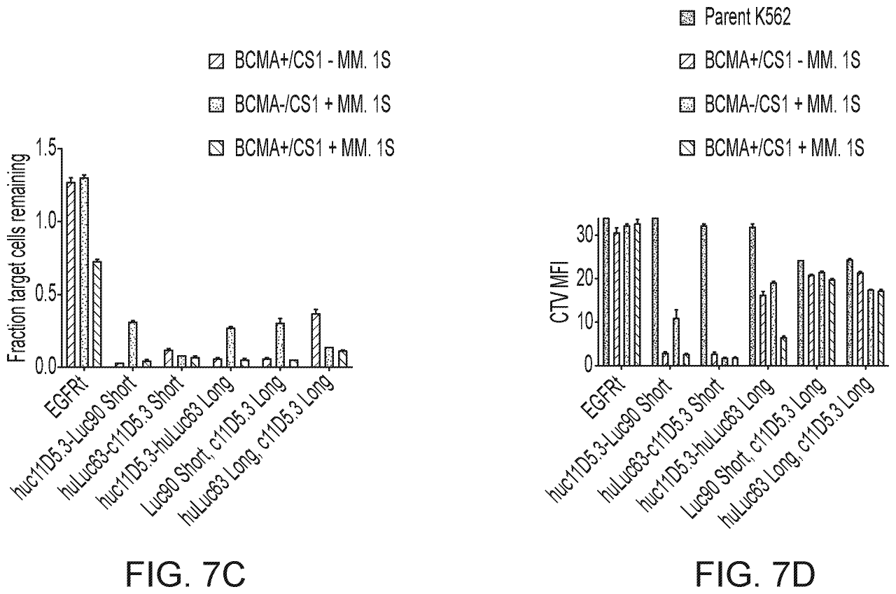

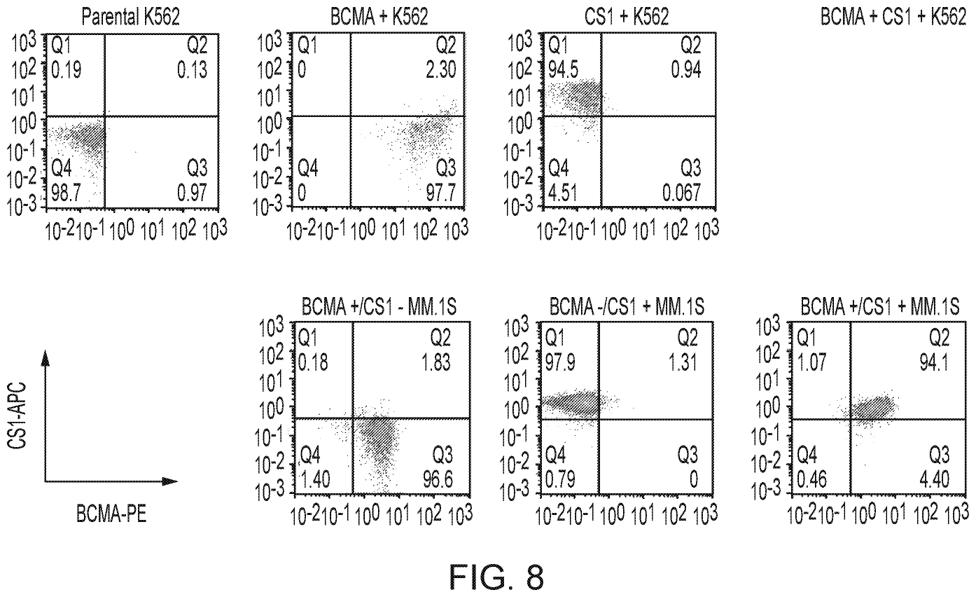

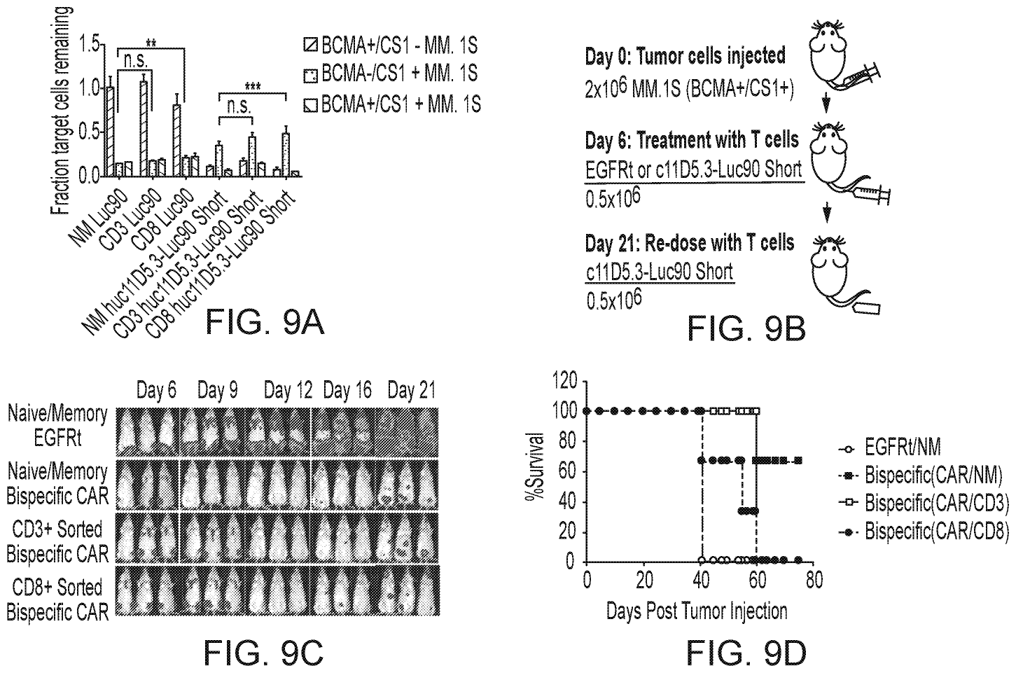

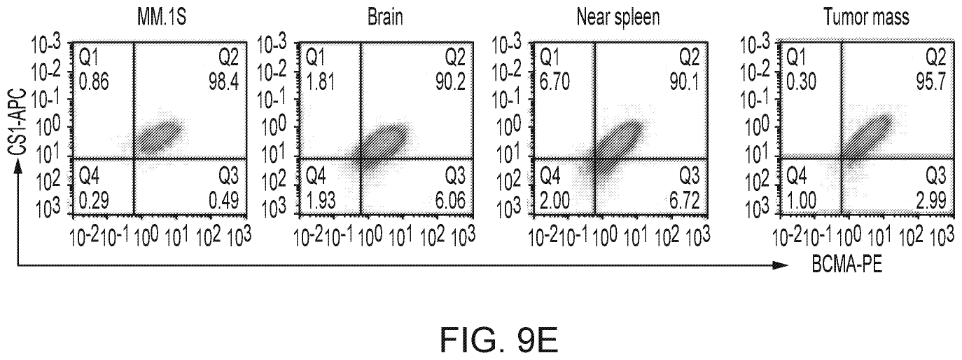

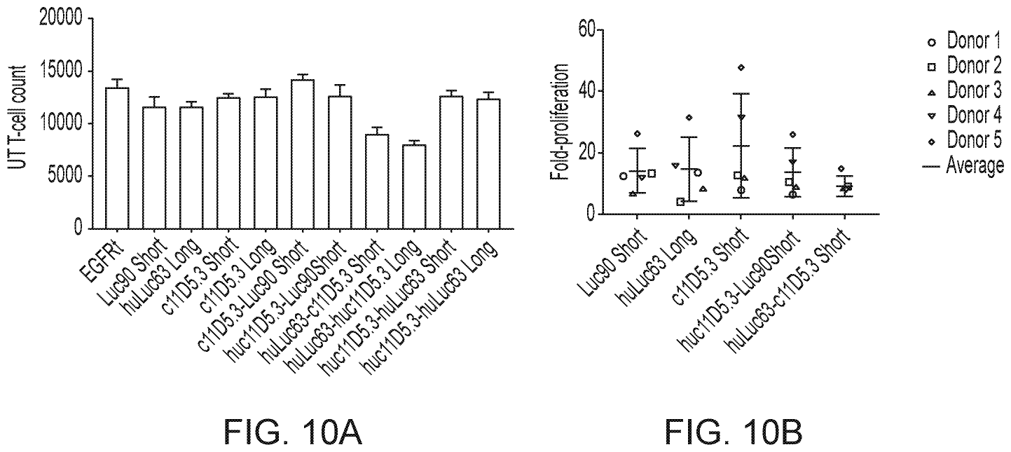

FIG. 3 provides expression data for different bispecific CARs in primary T cells. It shows effective expression of most bispecific constructs. FIG. 4. shows that particular bispecific constructs had favorable results with respect to cytotoxicity and T cell proliferation. Moreover, the bispecific constructs showed effective results with respect to both CS1 and BCMA targeting. In FIG. 5, c11D5.3 scFv shows superior function when separated from the cell membrane by a long extracellular spacer. FIG. 6 shows effective bispecific targeting and cell lysis by T cells expressing bispecific CARs. FIG. 7 shows BCMA/CS1 bispecific CAR-T cells outperform T cells co-expressing BCMA and CS1 CARs. Compared to bispecific CAR-T cells, dual CAR-T cells exhibit poor antigen-specific proliferation. Overall, the superiority of bispecific BCMA/CS1 CAR-expressing cells has been demonstrated as compared to T cells expressing single-targeted CARs or dual CARS (that express two different CAR molecules, one targeting BCMA and the other targeting CS1).

Materials and Methods

1. Plasmid Construction

Bispecific BCMA-CS1 CARs were constructed by isothermal assembly (1) of DNA fragments encoding the following components. BCMA-specific single-chain variable fragments (scFvs) were derived from either the c11D5.3 (2, 3) or the J.22-xi monoclonal antibody (mAb) (4). Truncated APRIL (dAPRIL) (5) was used as an alternative BCMA-binding domain. CS1-specific scFvs were derived from Luc90 or huLuc63 mAb (6, 7). Each CAR also contained an IgG4-based extracellular spacer, the CD28 transmembrane domain, and the cytoplasmic domains of 4-1BB and CD3 zeta. Amino acid sequences of all CAR components are shown in Table 1. All CARs were fused to a truncated epidermal growth factor receptor (EGFRt) via a T2A peptide to facilitate antibody staining of CAR-expressing cells (8).

2. Cell Line Generation and Maintenance

K562 cells were generated by retrovirally transducing parental K562 with BCMA and/or CS1 constructs. EGFP⁺, firefly luciferase (ffLuc)-expressing MM.1S cells were a generous gift from Dr. Xiuli Wang (City of Hope). BCMA⁻ or CS1⁻ MM.1S cells were generated by CRISPR/Cas9-mediated gene knockout. MM.1S cells (5×10⁶) were nucleofected with ribonucleoprotein (RNP), consisting of chemically synthesized gRNA (Synthego) targeting BCMA or CS1 complexed to purified Cas9 protein, using Ingenio Electroporation Solution (Mirus Bio) and the Amaxa Nucleofector 2B Device (Lonza). Four days after nucleofection, cells were surface-stained with BCMA-PE and CS1-APC antibodies (Biolegend) to verify antigen knockout. The cells were subsequently sorted for pure BCMA⁻ or CS1⁻ population by fluorescence-activated cell sorting using the FACSAria (II) at the UCLA Flow Cytometry Core Facility. K562 and MM.1S cells were cultured in complete T cell medium (RPMI-1640 (Lonza) with 10% heat-inactivated FBS (HI-FBS; Life Technologies)). Human embryonic kidney 293T cells (ATCC) were cultured in DMEM (VWR) supplemented with 10% HI-FBS.

3. Generation of CAR-Expressing Primary Human T Cells

CD8⁺ or CD25⁻/CD14⁻/CD62L⁺ naïve memory (NM) T cells were isolated from healthy donor whole blood obtained from the UCLA blood and platelet center. CD8+ cells were isolated using the RosetteSep Human CD8+ T Cell Enrichment Cocktail (StemCell Technologies) following manufacturer's protocols. Peripheral mononuclear blood cells (PBMCs) were isolated using Ficoll density gradient separation, and NM T cells were subsequently isolated from PBMCs using magnetism-activated cell sorting (Miltenyi) to first deplete CD25- and CD14-expressing cells and next enrich for CD62L⁺ cells. Isolated T cells were stimulated with CD3/CD28 T cell activation Dynabeads (Life Technologies) at a 1:3 bead:cell ratio. In initial screens, T cells were retrovirally transduced 48 and 72 hours post-stimulation. For the reduced CAR-T cell panel, T cells were lentivirally transduced 48 hours after stimulation at a multiplicity of infection of 1.5. All T cells were expanded in complete T cell medium and fed IL-2 (50 U/mL; Life Technologies) and IL-15 (1 ng/mL; Miltenyi) every 2-3 days. Dynabeads were removed 7 days post stimulation. CAR-T cells were evaluated without sorting.

4. Cytotoxicity Assay

Target cells (1×10⁴ cells) were seeded in a 96-well plate and coincubated with effector cells at an E:T ratio of 2:1 (150 μl total volume/well). Remaining target cells was quantified every 2 hours by fluorescence imaging of target cells using Incucyte ZOOM Live Cell Imaging System (Essen Bioscience).

5. Proliferation Assay

Effector cells were stained with CellTrace Violet (CTV; Thermo Fisher Scientific) and coincubated with 2.5×10⁴ target cells/well in a 96-well plate at an E:T ratio of 2:1, where effector cell seeding was based on CAR+ T cell count (150 μl total volume/well). After 120 hours, CTV-dilution of effector cells was quantified by flow cytometry using Macsquant VYB (Miltenyi).

6. Repeated Antigen Challenge

Target cells were seeded at 5×10⁵ cells/well in a 24-well plate and coincubated with effector cells at an E:T ratio of 1:1 or 1:2 (1.5 ml total volume/well). Remaining target cells were quantified by flow cytometry every 2 days. Fresh target cells were added to effector cells every 2 days after cell counting.

7. In Vivo Xenograft Studies in NOD/SCID/γ_{c}−/− (NSG) mice

[symbol]

All in vivo experiments were approved by the UCLA Institutional Animal Care and Use committee. Six- to eight-week-old male or female NSG mice were bred in house by the UCLA Department of Radiation and Oncology. EGFP⁺, ffLuc-expressing MM.1S cells (2×10⁵) were administered to NSG mice via tail-vein injection. Six days later, mice bearing engrafted tumors were treated with 0.5×10⁶ EGFRt-transduced or CAR⁺/EGFRt⁺ cells via tail-vein injection. Tumor progression was monitored by bioluminescence imaging using an IVIS Lumina III LT Imaging System (PerkinElmer).

### Example 2: Vertically Integrated Design of Bispecific CAR-T Cell Therapy Yields Superior Treatment for Heterogeneous Multiple Myeloma

Chimeric antigen receptor (CAR)-T cell therapy has shown remarkable clinical efficacy against B-cell malignancies as well as marked vulnerability to antigen escape and tumor relapse. It has become increasingly clear that achieving durable clinical efficacy with CAR-T cell therapy requires the improvement of multiple process parameters that extend far beyond the CAR molecule itself. Here, the inventors report a vertically integrated development process that systematically improves (1) CAR protein design, (2) cell manufacturing process, and (3) cell administration method, yielding bispecific CAR-T cells with robust activity against heterogenous multiple myeloma (MM) that is resistant to conventional CAR-T cell therapy targeting B-cell maturation antigen (BCMA). The inventors demonstrate that BCMA/CS1 bispecific CAR-T cells generated from naïve/memory T cells efficiently target both BCMA and CS1, and are functionally superior to T cells that co-express individual BCMA and CS1 CARs. Compared to single-input BCMA⁻ or CS1⁻ targeting CAR-T cells, BCMA/CS1 bispecific CAR-T cells significantly prolong the survival of animals bearing heterogenous MM tumors, and combination therapy with anti-PD-1 antibody further increases the anti-tumor response in vivo. Taken together, the BCMA/CS1 bispecific CAR presents a promising treatment approach to prevent antigen escape in CAR-T cell therapy against MM, and the vertically integrated design process can be used to develop robust cell-based therapy against novel disease targets.

A. Introduction

Multiple myeloma (MM) is the second-most common hematologic malignancy, with an estimated 32,110 new diagnoses expected in the US in 2019 (1). In recent years, immunomodulatory drugs and proteasome inhibitors such as thalidomide, lenalidomide, and bortezomib, which may be administered in conjunction with autologous stem-cell transplant, have dramatically improved survival of patients suffering from MM (2). However, MM remains an incurable disease despite these therapeutic options.

The adoptive transfer of CAR-T cells targeting B-cell maturation antigen (BCMA) has shown clinical efficacy against MM, achieving 80%-100% overall response rate across multiple clinical trials (3-7). However, BCMA is not uniformly expressed on MM cells, as evidenced by a recent study that screened 85 MM patients and found 33 to be BCMA negative (3). Furthermore, multiple cases of patient relapse involving tumor cells with downregulated BCMA expression have been reported (3, 4, 6), underscoring antigen escape as a significant obstacle in the treatment of MM with BCMA CAR-T cell therapy. In addition, a substantial fraction of patients treated with BCMA CAR-T cells eventually relapse even when BCMA expression is retained (3, 4, 6), suggesting a lack of durable effector function by the engineered T cells.

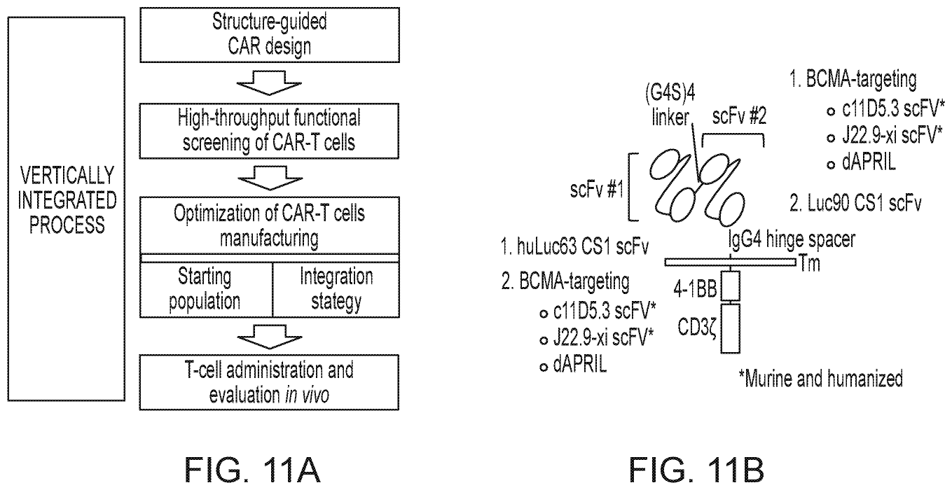

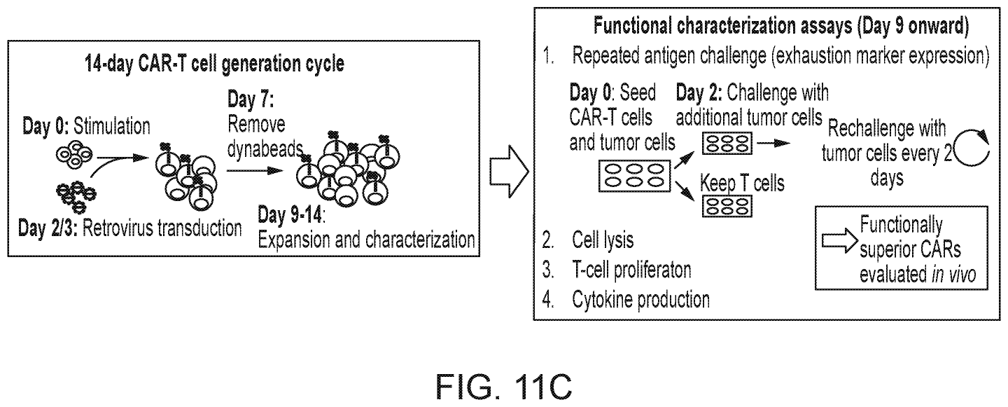

To address these challenges, the inventors set out to develop a new CAR-T cell treatment for MM exhibiting greater resistance to antigen escape and improved long-term effector function. As a living drug, CAR-T cells constitute a complex treatment modality involving multiple process parameters that extend well beyond the CAR molecule itself. Therefore, drawing an analogy to microeconomics, the inventors developed a vertically integrated design process that begins with structure-guided design and high-throughput functional screening of CAR variants, followed by systematic identification of improved cell manufacturing conditions including the choice of starting T cell population and the method of CAR transgene integration, and ending with the evaluation of long-term in vivo anti-tumor efficacy of CAR-T cell therapy alone and in combination with checkpoint inhibitor therapy (FIG. 11A).

Here, the inventors report the design of BCMA/CS1 OR-gate CAR-T cells that can efficiently target BCMA⁻ or CS1-expressing tumor cells while maintaining robust ex vivo expansion with minimal fratricidal side effects. The inventors show that BCMA/CS1 OR-gate CAR-T cells have superior CAR expression and anti-tumor functions compared to T cells co-expressing two separate CARs targeting BCMA and CS1. Furthermore, BCMA/CS1 OR-gate CAR-T cells are substantially more effective than single-input BCMA or CS1 CAR-T cells in controlling heterogenous MM tumor populations in vivo, resulting in significantly prolonged survival of tumor-bearing mice. The inventors further demonstrate that CAR-T cells generated from naïve/memory T cells are functionally superior to those generated from bulk CD3⁺ or CD8⁺ T cells, and that lentivirus-mediated CAR transgene integration surprisingly yields more robust T cells than CRISPR/Cas9-mediated CAR integration into the TRAC locus. Finally, the inventors demonstrate that combination therapy with anti-PD-1 antibody further enhances the in vivo anti-tumor efficacy of BCMA/CS1 OR-gate CAR-T cells against established MM, leading to effective and durable control of highly aggressive tumors.

B. Materials and Methods

1. Plasmid Construction

Single-chain bispecific BCMA-OR-CS1 CARs were constructed by isothermal assembly (19) of DNA fragments encoding the following components. BCMA-specific single-chain variable fragments (scFvs) were derived from either the c11D5.3 (20, 21) or the J.22-xi (22) monoclonal antibody (mAb), and dAPRIL23 was also evaluated as an alternative BCMA-binding domain. CS1-specific scFvs were derived from Luc90 or huLuc63 mAb (24, 25). Each CAR also contained an IgG4-based extracellular spacer, the CD28 transmembrane domain, and the cytoplasmic domains of 4-1BB and CD3ζ. Amino acid sequences of all CAR components are shown in Supplementary Table 1. All CARs were fused to a truncated epidermal growth factor receptor (EGFRt) via a T2A peptide to facilitate antibody staining of CAR-expressing cells (26). An N-terminal FLAG or HA tag was also added to each CAR to enable quantification of CAR surface expression.

2. Cell-Line Generation and Maintenance

Parental K562 cells were a gift from Dr. Michael C. Jensen (Seattle Children's Research Institute) received in 2011. Antigen-expressing K562 cells were generated by retrovirally transducing parental K562 with BCMA− and/or CS1-encoding constructs. EGFP+, firefly luciferase (ffLuc)-expressing MM.1S cells were a generous gift from Dr. Xiuli Wang (City of Hope). BCMA− or CS1− MM.1S cells were generated by CRISPR/Cas9-mediated gene knockout. MM.1S cells (5×10⁶) were nucleofected with ribonucleoprotein (RNP), consisting of chemically synthesized gRNA (Synthego) targeting BCMA or CS1 complexed to purified Cas9 protein, using Ingenio Electroporation Solution (Mirus Bio) and the Amaxa Nucleofector 2B Device (Lonza) following manufacturers' protocols. Four days after nucleofection, cells were surface-stained with BCMA-PE and CS1− APC antibodies (Biolegend) to verify antigen knockout. The cells were subsequently bulk-sorted for BCMA− or CS1− populations by fluorescence-activated cell sorting using a FACSAria (II) sorter at the UCLA Flow Cytometry Core Facility, and the sorted polyclonal population was expanded for use in in vitro and in vivo experiments. K562 and MM.1S cells were cultured in complete T cell medium (RPMI-1640 (Lonza) with 10% heat-inactivated FBS (HI-FBS; Life Technologies)). HEK293T cells (ATCC) were cultured in DMEM (VWR) supplemented with 10% HI-FBS.

3. Retrovirus Production

HEK 293T cells seeded in 10-cm dishes at 5.5×10⁶ cells in 9 mL of DMEM+10% HI-FBS+20 mM HEPES (DMEM-HEPES) were transfected by linear polyethylenimine (PEI). Sixteen hours post-transfection, cells were washed with 5 mL of 1×-phosphate buffered saline without magnesium and calcium (PBS) (Lonza) and supplemented with fresh DMEM-HEPES supplemented with 10 mM sodium butyrate (Sigma-Aldrich). After 8 hours, cells were washed with sterile PBS and then 8 mL of DMEM-HEPES was added. Viral supernatant was collected the following morning and cell debris was removed by filtering the viral supernatant through a 0.45 μM membrane (Corning). Six milliliters of fresh DMEM-HEPES was added to the cells following the first round of viral collection. After 24 hours, a second viral harvest was performed the following day, and virus harvested from first and second batches were combined and stored at −80° C. until further use.

4. Lentivirus Production

HEK 293T cells seeded in 10-cm dishes at 3.5×10⁶ cells in 9 mL DMEM+10% HI-FBS media were transfected by linear PEI. Sixteen hours post-transfection, cells were washed with PBS and supplemented with fresh media containing 60 mM sodium butyrate (Sigma-Aldrich). Viral supernatant was collected 24 hours and 48 hours after media change, and cell debris was removed from the supernatant by centrifugation at 450×g for 10 min at 4° C., followed by filtration through a 0.45 μM membrane (Corning). Viral supernatant collected 24 hours after media change was mixed with ¼ volume 40% polyethylene glycol 8000 (PEG) (Amresco) in 1×-PBS and rotated overnight at 4° C. PEG-treated virus was pelleted at 1,000×g for 20 min at 4° C., then resuspended in viral supernatant collected 48 hours after media change, and finally ultracentrifuged at 51,300×g for 1 hour and 35 minutes at 4° C. Pellets were resuspended in 200 L of serum-free RPMI-1640 and then incubated for 1 hour at 4° C. to allow complete dissolution. Virus was then stored at −80° C. for subsequent titer and use.

5. Adeno-Associated Virus Production

HEK 293T cells seeded in eighteen 10-cm dishes at 3×10⁶ cells in 9 mL of DMEM+10% HI-FBS media were transfected by linear PEI. After 72 hours, cells were harvested, pelleted at 1,000×g for 5 minutes at 4° C., then resuspended in 14.4 mL of 50 mM Tris+150 mM NaCl (pH 8.2). The cells were lysed by undergoing three freeze/thaw cycles, then incubated at 37° C. for 1 hour with benzonase (10 U/mL; EMD Millipore). The lysate was then centrifuged at 13,200×g for 10 min at room temperature. Supernatant was collected and stored at 4° C. until next step. The lysate supernatant was ultracentrifuged with iodixanol (OptiPrep; StemCell Technologies) density gradient solutions (54%, 40%, 25%, and 15% w/v) at 76,900×g for 18 hours at 4° C. Then, ⅘ of the 40% layer and % of the 54% layer were extracted from the polyallomer Quick-seal ultracentrifuge tube (Fisher) with an 18-gauge needle (Fisher) attached to a 10-mL syringe (VWR). The collected virus fraction was diluted in an equal volume of PBS+0.001% Tween-20, applied to an Amicon Ultra-15 (EMD Millipore, 10 kDa NMWL) column, and centrifuged at 4,000×g for 20 minutes at 4° C. The resulting virus fraction was diluted with PBS+0.001% Tween-20 and centrifuged until 500 L of the virus fraction remained in the column. Concentrated virus was stored at 4° C. for subsequent titer and use.

6. Generation of CAR-Expressing Primary Human T Cells

CD25−/CD14−/CD62L+naïve/memory (NM), CD8⁺, or bulk T cells were isolated from healthy donor whole blood obtained from the UCLA blood and platelet center. CD8+ cells were isolated using the RosetteSep Human CD8⁺ T Cell Enrichment Cocktail (StemCell Technologies) following manufacturer's protocols. Bulk T cells were isolated using RosetteSep Human T cell Enrichment Cocktail (StemCell Technologies). Peripheral mononuclear blood cells (PBMCs) were isolated using Ficoll density-gradient separation, and NM T cells were subsequently isolated from PBMCs using magnetism-activated cell sorting (Miltenyi) to first deplete CD25- and CD14-expressing cells and next enrich for CD62L⁺ cells. Isolated T cells were stimulated with CD3/CD28 T cell activation Dynabeads (Life Technologies) at a 1:3 bead:cell ratio. In initial screens, T cells were retrovirally transduced 48 and 72 hours post-stimulation. For the reduced CAR-T cell panel, T cells were lentivirally transduced 48 hours after stimulation at a multiplicity of infection (m.o.i.) of 1.5. For retrovirally and lentivirally transduced CAR-T cells, Dynabeads were removed 7 days post stimulation. For CAR-T cells with CAR integrated via homology-directed repair (HDR), Dynabeads were removed 3 days post stimulation, and T cells were nucleofected with RNP, consisting of a previously reported single-guide RNA targeting the 5′ end of exon 1 of T cell receptor a constant (TRAC) locus (27) complexed to purified Cas9 protein. Nucleofected cells were incubated at 37° C. for 10 minutes, and then transduced with adeno-associated virus (AAV) at a multiplicity of infection of 3×10⁵. All T cells were expanded in complete T cell medium and fed interleukin (IL)-2 (50 U/mL; Life Technologies) and IL-15 (1 ng/mL; Miltenyi) every 2-3 days. CAR-T cells were evaluated without further cell sorting.

7. Cytotoxicity Assay

Target cells (1×10⁴ cells) were seeded in a 96-well plate and coincubated with effector cells at an effector:target (E:T) ratio of 2:1 (150 μl total volume/well). Effector cell seeding was based on CAR⁺ T cell count. Remaining target cells was quantified every 2 hours by GFP fluorescence imaging of target cells using IncuCyte ZOOM Live Cell Imaging System (Essen Bioscience). The amount of green fluorescence at specific time points was normalized to fluorescence at time 0 to calculate the fraction of live tumor cells remaining. Kill rates were calculated by applying log-linear models with R 3.5.2 software.

8. Proliferation Assay

Effector cells were stained with CellTrace Violet (CTV; Thermo Fisher Scientific) and coincubated with 2.5×10⁴ target cells/well in a 96-well plate at an E:T ratio of 2:1, where effector-cell seeding was based on CAR⁺ T cell count (150 μl total volume/well). After 120 hours, CTV-dilution of effector cells was quantified by flow cytometry using a MACSQuant VYB instrument (Miltenyi).

9. Cytokine Production

Target cells were seeded at 5×10⁴ cells/well in a 96-well plate and coincubated with effector cells at an E:T ratio of 2:1 for 24 hours. Effector-cell seeding was based on CAR⁺ T cell count. Cytokine concentrations in the culture supernatant were measured using BD Cytometric Bead Array Human Th1/Th2 Cytokine Kit II (BD Biosciences). 10. Repeated antigen challenge

Target cells were seeded at 1.8-5×10⁵ cells/well in a 48- or 24-well plate and coincubated with effector cells at an E:T ratio of 1:1 or 1:2 (1-1.5 ml total volume/well). Effector cell seeding was based on CAR⁺ T cell count. Remaining target cells were quantified by flow cytometry every 2 days. Fresh target cells (1.8-5×10⁵ cells/well) were added to effector cells every 2 days after cell counting.

11. In Vivo Xenograft Studies in NOD/SCID/γ_{c}−/− (NSG) Mice

All in vivo experiments were approved by the UCLA Animal Research Committee. Six-to-eight-week-old male and female NSG mice were bred in house by the UCLA Department of Radiation and Oncology. EGFP⁺, ffLuc-expressing MM.1S cells (1.5×10⁶-2×10⁶) were administered to NSG mice via tail-vein injection. Upon confirmation of tumor engraftment (5-8 days post tumor cell injection), mice were treated with 0.5×10⁶-1.5×10⁶ EGFRt-transduced or CAR⁺/EGFRt⁺ cells via tail-vein injection. In some experiments, animals were redosed 8 days later with a second injection of 1.5×10⁶ T cells as noted in the text and figure captions. Tumor progression was monitored by bioluminescence imaging using an IVIS Lumina III LT Imaging System (PerkinElmer). For combination therapy with anti-PD-1, mice were treated with 200 g of anti-PD-1 (Ultra-LEAF, BioLegend) via intraperitoneal (i.p.) injection every 3-4 days starting one day before T cell injection.

12. Amplicon DNA Sequencing

Genomic DNA was isolated from 1×10⁶ tumor cells using DNeasy Blood & Tissue Kit (Qiagen). BCMA and CS1 loci amplicons, with Nextera transposase adapters (Illumina) flanking each target locus, were prepared via PCR with the isolated genomic DNA. Nextera indices (Illumina) were attached to the adapters to barcode each amplicon samples via PCR. After each PCR round, amplicons were purified using AMPure XP beads (Beckman Coulter). The barcoded amplicon samples were then sent to the UCLA Technology Center for Genomics & Bioinformatics for multiplex sequencing with 2×300 paired-end configuration in a single-lane flow cell of MiSeq instrument (Illumina). Fastq paired-end raw data were filtered, trimmed, and merged with DADA2 (version 1.12) on R 3.5.0 software. This work used computational and storage services associated with the Hoffman2 Shared Cluster provided by UCLA Institute for Digital Research and Education's Research Technology Group.

13. Statistical Analysis

Statistical significance of in vitro results was analyzed using two-tailed, unpaired, Student's t-test. Animal survival data were analyzed by log-rank analysis.

C. Results

1. Construction of Single-Chain Bispecific BCMA/CS1 CARs

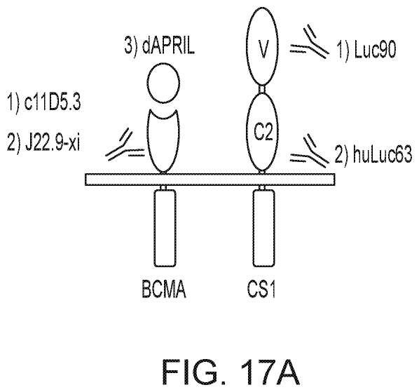

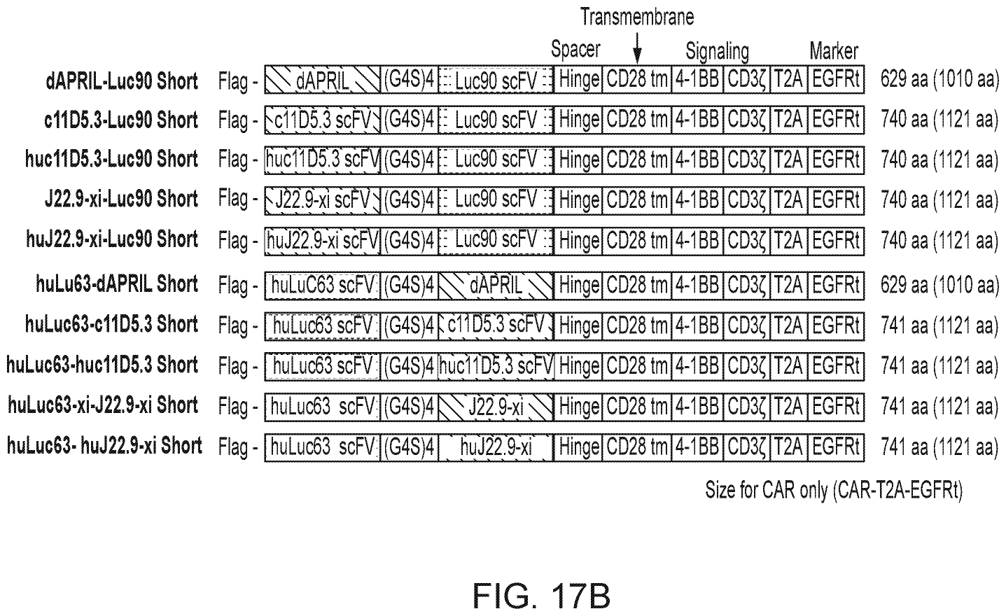

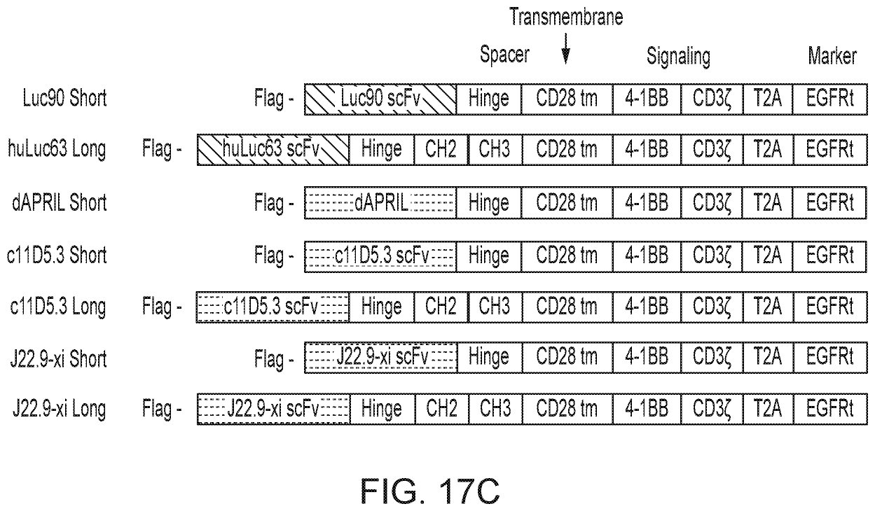

A panel of second-generation, 4-1BB-containing OR-gate CAR variants was constructed to evaluate multiple ligand-binding moieties, including three BCMA-recognition domains (dAPRIL and single-chain variable fragments (scFvs) derived from two BCMA− binding antibodies, c11D5.3 or J22.9-xi), each paired with one of two CS1-binding scFvs (Luc90 or huLuc63) (FIG. 11B). The inventors and others have shown that optimal CAR signaling requires the CAR's ligand-binding domain to be precisely positioned to create an immunological synapse of an appropriate dimension when bound to the target antigen (9, 28-30). Among the CS1-targeting antibodies, huLuc63 and Luc90 are known to bind the membrane-proximal C2 epitope and the membrane-distal V epitope of CS1, respectively (31) (FIG. 17A). Therefore, the inventors reasoned that the huLuc63-derived scFv should be placed at the membrane-distal position relative to the T cell membrane, paired with a BCMA-binding domain at the membrane-proximal position, such that the huLuc63 scFv can have sufficient extension to make proper contact with the C2 epitope close to the target cell surface. Conversely, the inventors fixed the Luc90 scFv at the membrane-proximal position for a second set of CARs (FIG. 17B). To increase potential clinical applicability, both murine and humanized versions of the BCMA-binding c11D5.3 and J22.9-xi scFvs were evaluated. All OR-gate CARs in this initial panel contained a short (12-amino acid) extracellular spacer. In total, 10 bispecific CARs plus 7 single-input CAR controls were constructed for the first round of screening (FIG. 11B, FIG. 17B,C).

2. Identifying Lead BCMA/CS1 OR-Gate CAR Candidates by Rapid Functional Testing

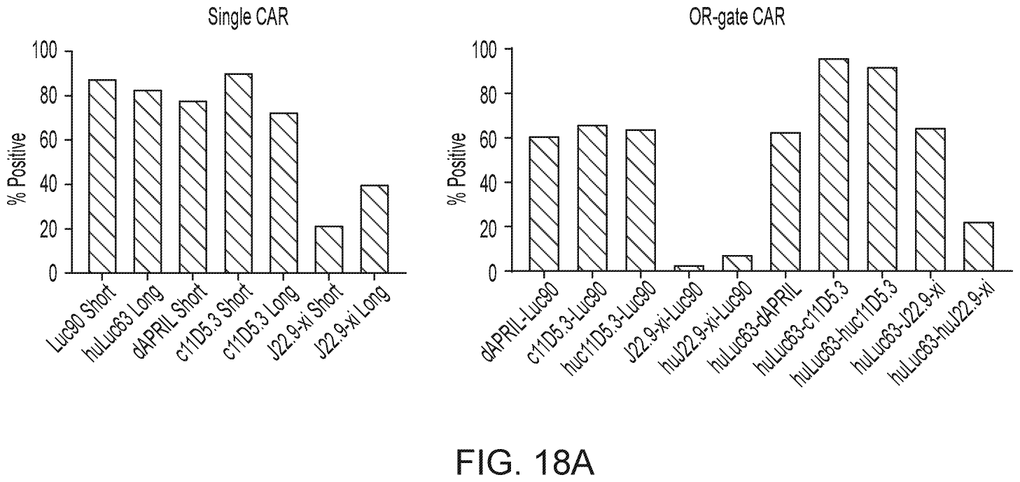

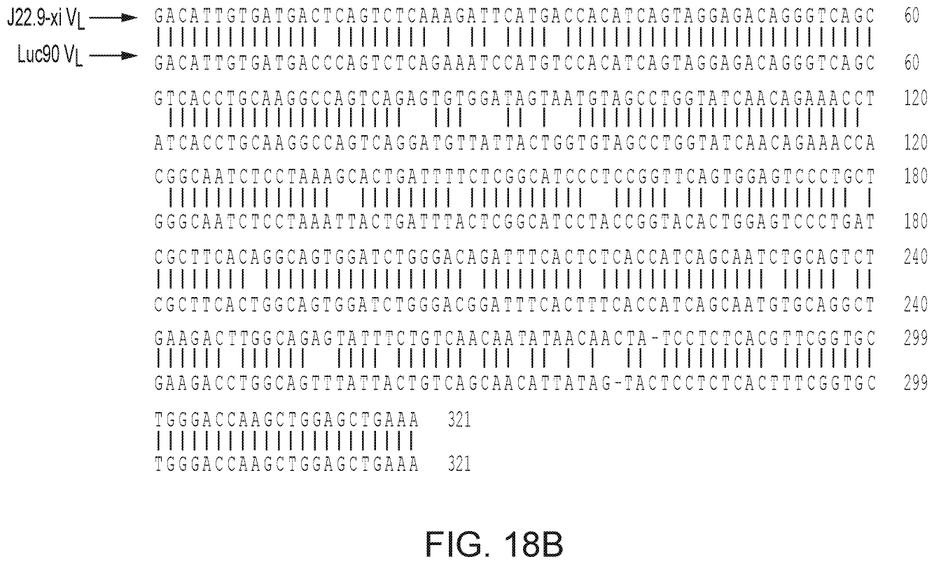

A methodology for high-throughput generation and screening of new CAR-T cells was developed to support the rapid evaluation of novel OR-gate CAR designs, with low-volume functional assays that enabled simultaneous comparison of up to 17 different T cell lines, all generated using cells from the same donor to ensure comparability (FIG. 18A).

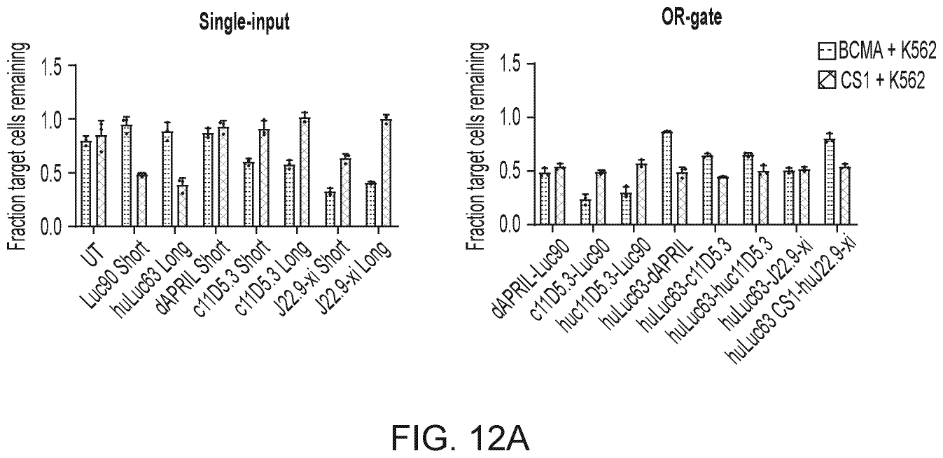

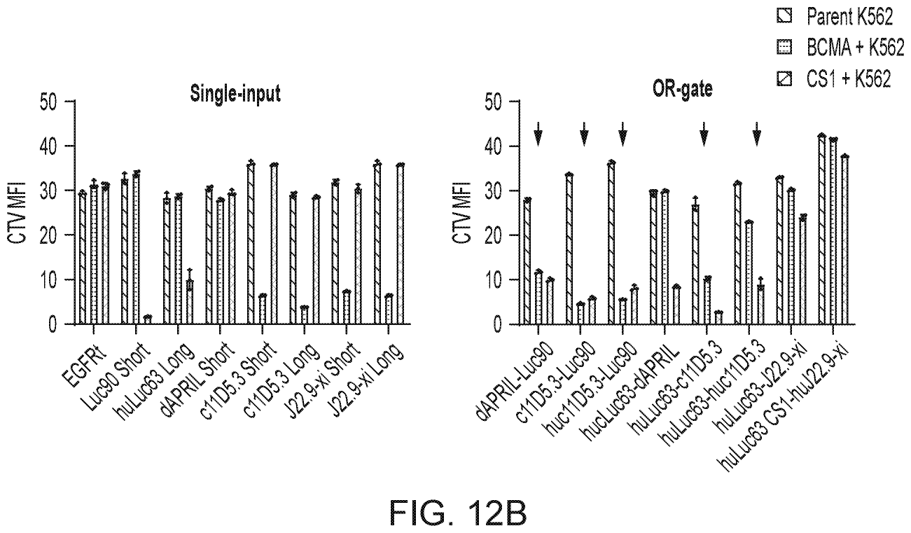

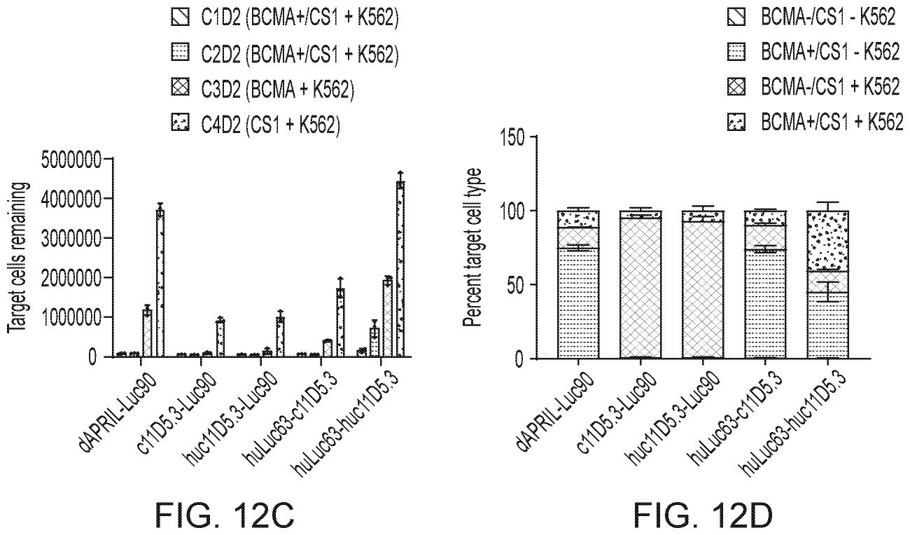

CAR surface expression staining revealed that receptors comprising Luc90 paired with either humanized or murine J22.9-xi scFv were poorly expressed on primary human T cells and thus eliminated from further consideration (FIG. 18A). Analysis of the DNA sequences for J22.9-xi and Luc90 revealed significant homology in the light chains of the two scFvs (FIG. 18B), which may have resulted in genomic instability of these constructs due to homologous recombination. Among the remaining 8 OR-gate candidates, c11D5.3-Luc90 and huc11D5.3-Luc90 CAR-T cells were the most effective against BCMA⁺ target cells based on both target cell lysis and antigen-stimulated T cell proliferation (FIG. 12A,B), whereas CS1 was best targeted by huLuc63-c11D5.3 (FIG. 12B).

The top five OR-gate CAR-T cell lines based on target cell lysis and T cell proliferation (FIG. 12B, marked by arrows) were subjected to repeated antigen challenge to evaluate their propensity for exhaustion. CAR-T cells were challenged with BCMA⁺/CS1⁺ K562 cells in the first two rounds, followed by BCMA⁺/CS1⁻ and BCMA⁻/CS1⁺ K562 cells in the third and fourth rounds, respectively. Here, c11D5.3-Luc90, huc1D5.3-Luc90, and huLuc63-c11D5.3 CAR-T cells continued to outperform other candidates by maintaining efficient target cell killing for three rounds of antigen challenge before succumbing to tumor outgrowth (FIG. 12C). Antigen expression patterns on surviving tumor cells confirmed that c11D5.3-Luc90 and huc11D5.3-Luc90 CAR-T cells showed superior targeting of BCMA+ tumor cells, resulting in a disproportionately large fraction of BCMA⁻/CS1⁺ K562 cells in the remaining tumor population (FIG. 12D). In contrast, huLuc63-c11D5.3 CAR-T cells had a higher proportion of BCMA⁺/CS1⁻ target cells remaining, indicating greater efficacy against CS1⁺ targets.

Surprisingly, all dAPRIL-based CARs failed to achieve efficient target cell lysis and T cell proliferation (FIG. 12A-C), and defective BCMA-targeting appeared to be the main cause based on the composition of residual tumor cells (FIG. 12D). Given these results, the dAPRIL-based designs evaluated in this panel were eliminated from further consideration.

3. Functional Tradeoff Between BCMA and CS1 Targeting by Single-Chain OR-Gate CARs

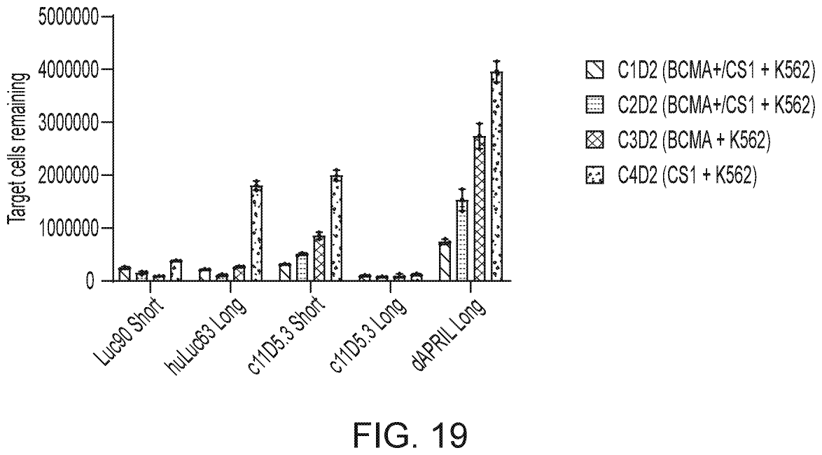

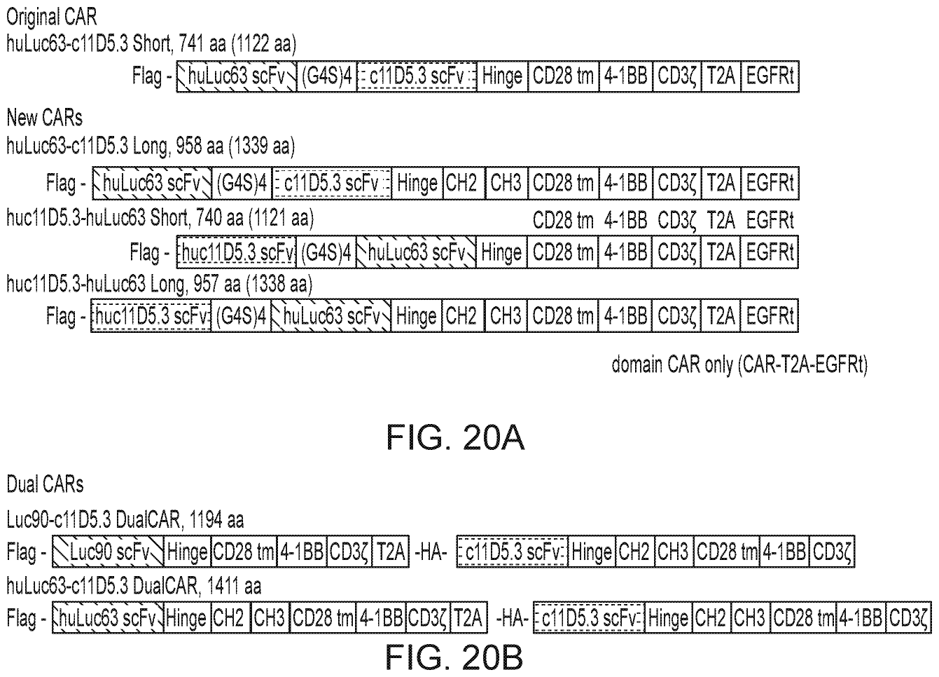

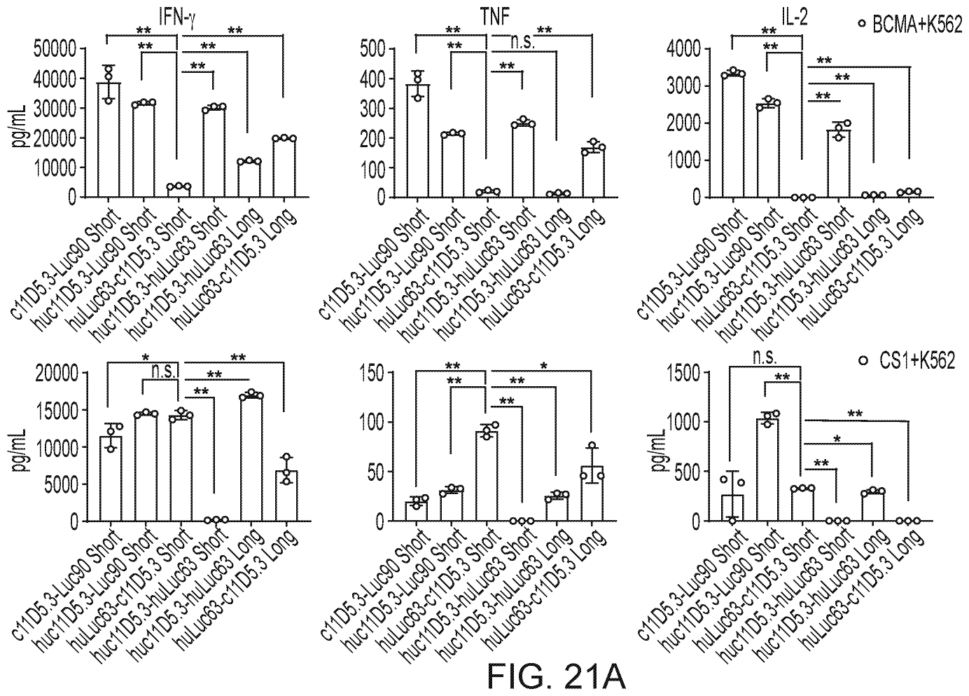

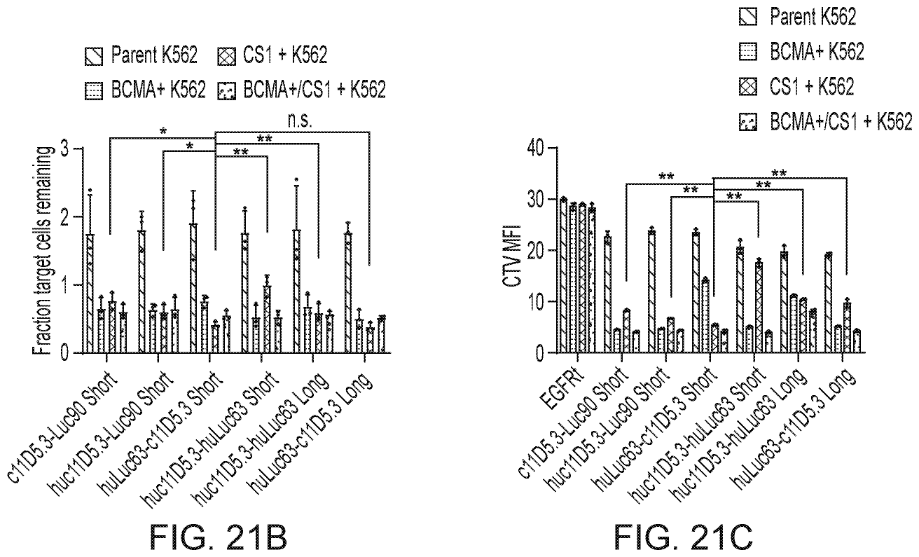

In the repeated antigen challenge assay, the inventors had observed that the single-input c11D5.3 BCMA CAR showed superior function when coupled to a long (229-amino acid) extracellular spacer (FIG. 19). This observation is unsurprising given that BCMA has a very short (36-amino acid) ectodomain, thus the BCMA CAR needs to extend farther out to reach the target antigen. However, as previously noted, the binding epitope for the CS1− targeting huLuc63 scFv is also expected to work best with a long spacer (FIG. 17A), thus raising the prospect of an unavoidable tradeoff between BCMA and CS1 targeting. Indeed, when the inventors evaluated the effect of lengthening of the extracellular spacer (from 12 to 229 amino acids) and/or changing the relative positioning of the two scFvs, they found the original huLuc63-c11D5.3 Short CAR design to possess the best balance of BCMA and CS1 targeting efficiency while requiring a relatively compact DNA footprint (FIGS. 20A and 21). Based on cumulative in vitro functional assay results, the inventors chose to focus on huc11D5.3-Luc90 Short and huLuc63-c11D5.3 Short as their final two candidates, each with a slight advantage against BCMA or CS1, respectively.

4. Functional Superiority of OR-Gate CARs Over DuaCARs

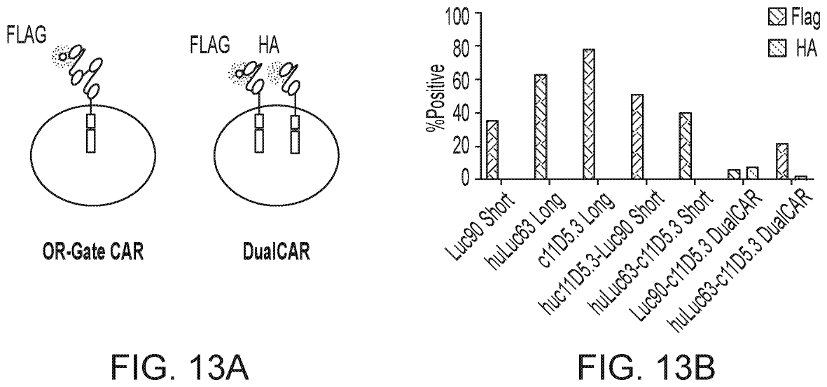

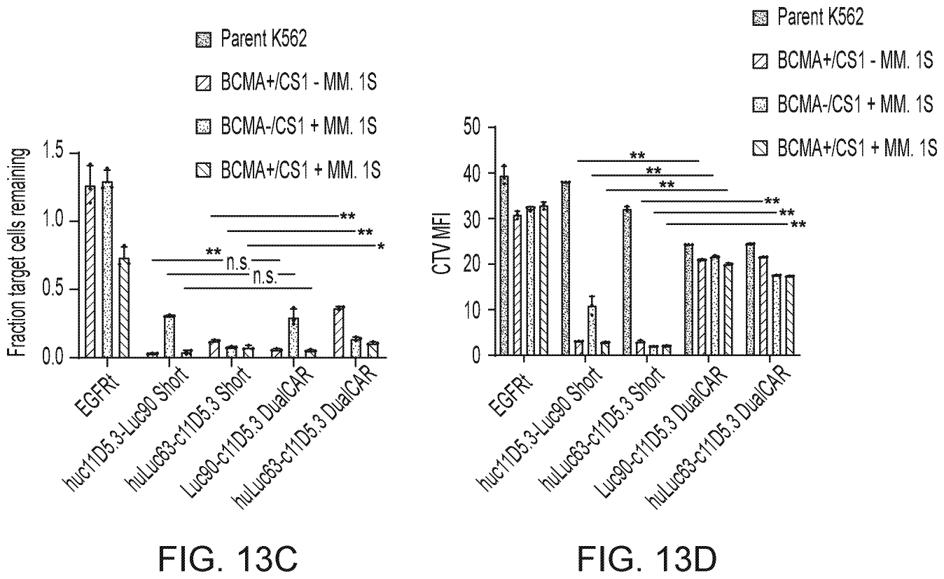

The fact that the single-chain bispecific CAR structure employed here could not be fully optimized for both BCMA and CS1 targeting due to overlapping and thus incompatible structural preferences for the two target epitopes raises the question of whether co-expressing two separate single-input CARs (“DualCAR” approach) would be a more effective way to achieve T cell bispecificity (FIG. 13A)(32). The inventors reasoned that the single-chain OR-gate CARs should be more efficiently integrated and expressed due to their compact size, thus yielding a more functional CAR-T cell product compared to the DualCAR approach. This hypothesis was experimentally verified when the inventors compared T cells expressing either an OR-gate CAR or the corresponding pairs of single-input CARs encoded in bicistronic cassettes connected by a 2A sequence (FIG. 20B). Flow-cytometry analysis revealed that the DualCAR-T cells had substantially lower CAR surface expression compared to OR-gate CAR-T cells (FIG. 13B). Furthermore, DualCAR-T cells exhibited slightly lower cytotoxicity against MM.1S myeloma cells (FIG. 13C) and significantly weaker T cell proliferation upon antigen stimulation (FIG. 13D) compared to the corresponding OR-gate CAR-T cells, even when the assay setup was normalized by CAR⁺ T cell count. These results indicate that OR-gate CAR-T cells are not only easier to manufacture due to higher transduction efficiency, but also functionally superior to DualCAR-expressing T cells when challenged with MM tumor cells.

5. Efficient Ex Vivo Expansion and Lack of Fratricide in OR-Gate CAR-T Cells

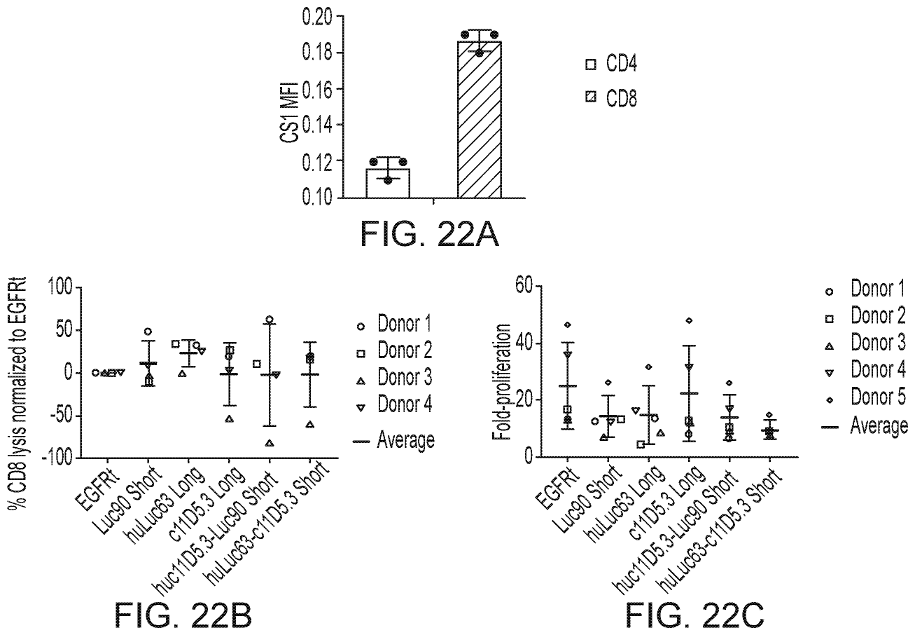

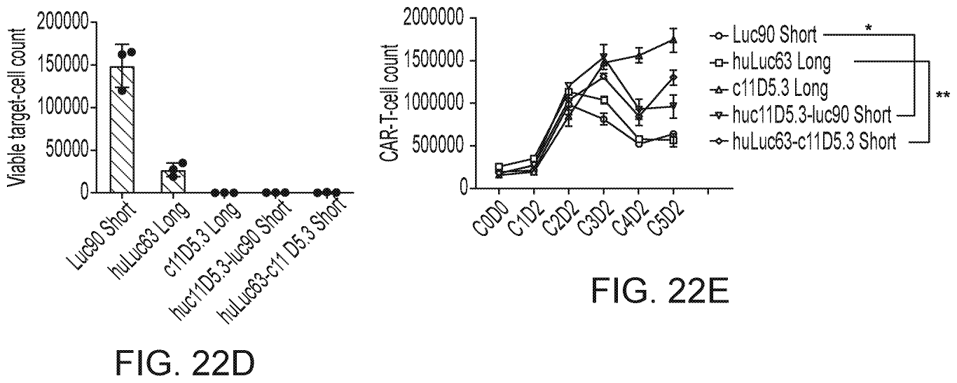

CS1 is highly expressed on myeloma cells but is also found on other hematopoietic cells, including CD8⁺ T cells (FIG. 22A) (16, 33). To evaluate the propensity for fratricide, OR-gate CAR-T cells were coincubated with donor-matched, untransduced, CellTrace Violet (CTV)-labeled CD8⁺ T cells, whose survival was quantified after a 24-hour coincubation. Results showed no significant difference in either the killing of bystander CD8⁺ T cells or ex vivo culture expansion by OR-gate CAR-T cells in comparison to single-input BCMA CAR-T cells (c11D5.3 Long) or mock-transduced T cells (FIG. 22B,C). Interestingly, OR-gate CAR-T cells showed superior performance compared to single-input CS1 CAR-T cells (Luc90 Short and huLuc63 Long) upon repeated antigen challenge (FIG. 22D,E). A likely explanation is that the OR-gate CAR-T cells have slightly weaker reaction to CS1⁺ target cells compared to the single-input CS1 CAR-T cells, striking a balance that enables robust tumor killing without inducing premature T cell exhaustion.

6. Identification of Naïve/Memory T Cells as Functionally Superior Subtype

Functional testing of the OR-gate CARs designed in this study were initially performed using bulk-sorted CD8⁺ T cells. However, it had been shown that administering a mixture of CD8⁺ and CD4⁺ T cells could improve performance over CD8⁺ T cells alone (34), and that T cells exhibiting a memory phenotype could improve CAR-T cell persistence and function in vivo (34-27). Therefore, the inventors next compared CD8-derived CAR-T cells against CAR-T cells derived from a naïve/memory (NM) starting population, which contains a mixture of both CD8⁺ and CD4⁺ T cells obtained by subjecting peripheral blood mononuclear cells (PBMCs) to CD14 and CD25 depletion followed by CD62L enrichment.

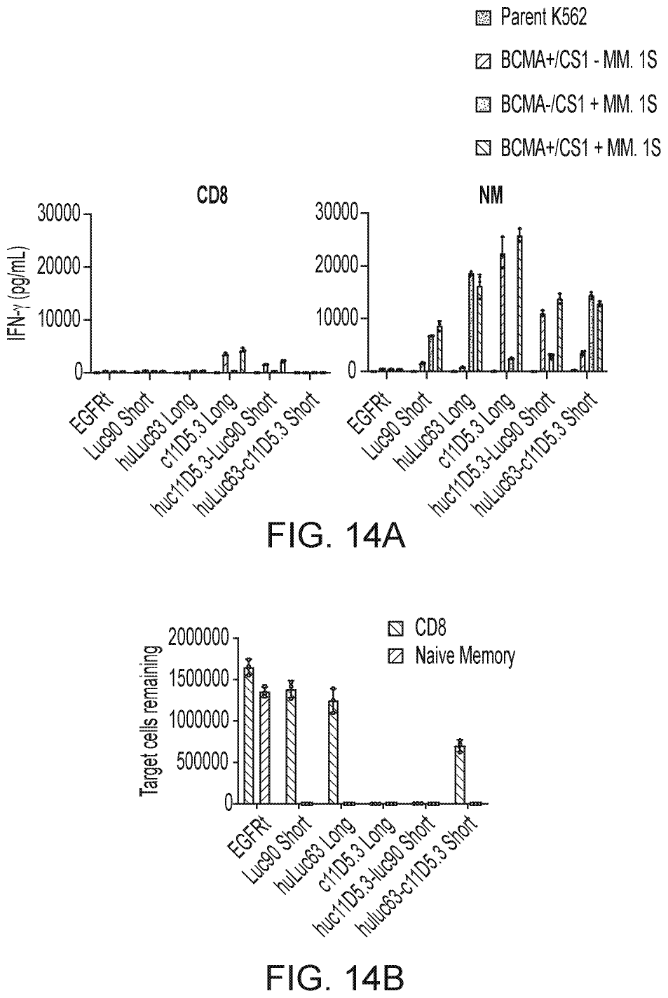

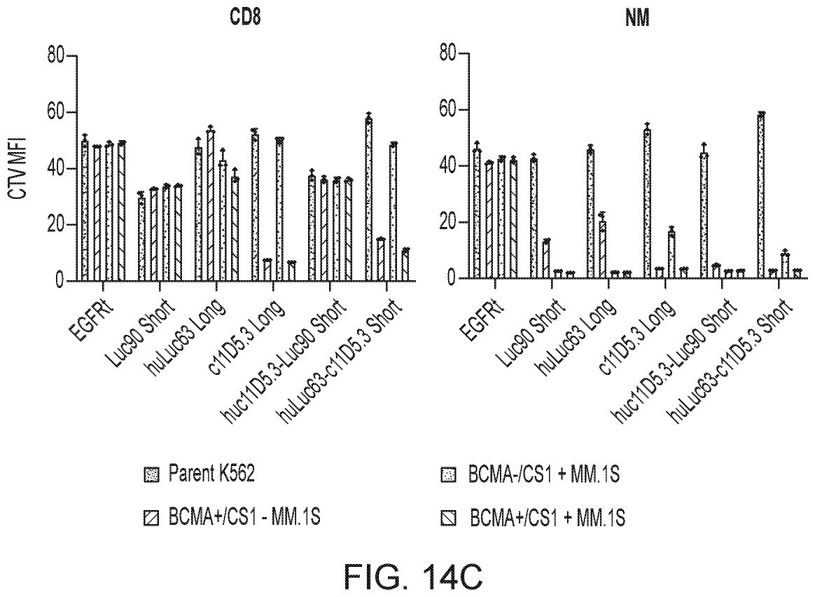

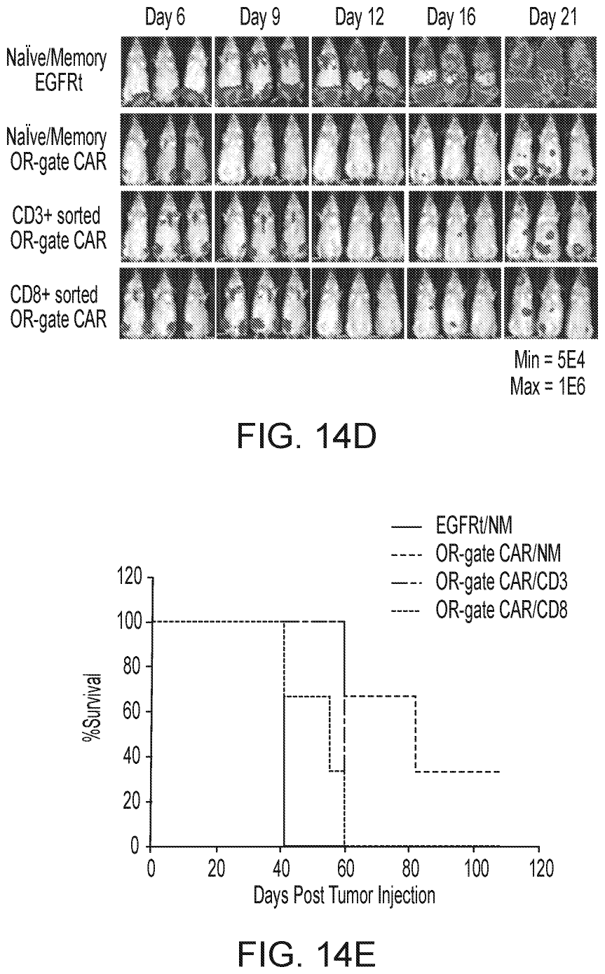

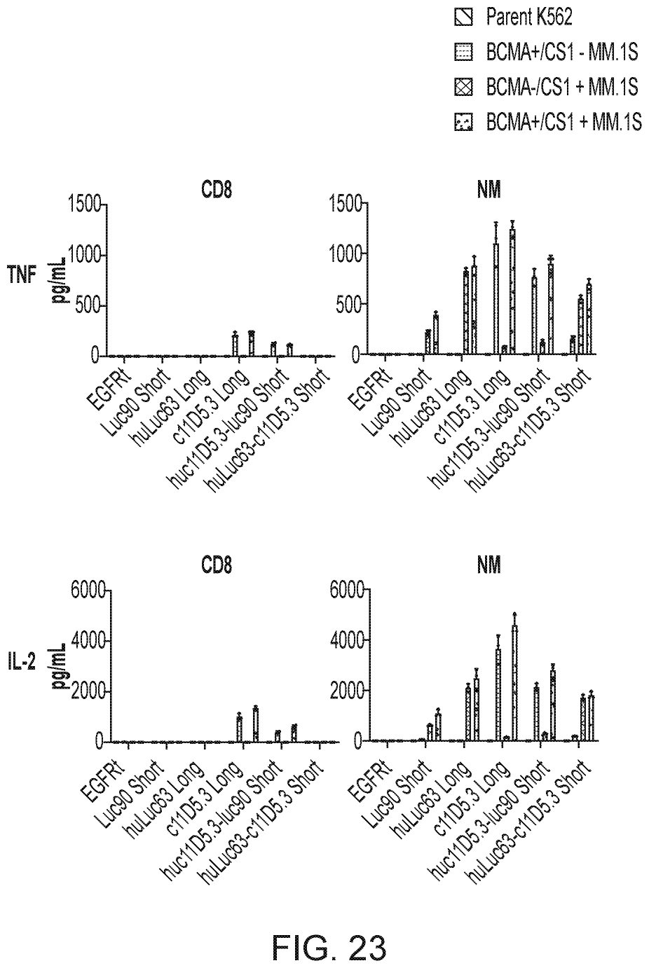

Across all CAR constructs, NM-derived CAR-T cells demonstrated substantially higher cytokine production, more sustained target cell killing upon repeated challenge, and greater T cell proliferation compared to CD8-derived CAR-T cells (FIG. 14A-C and FIG. 23). Given that NM-derived T cells contained CD4⁺ cells, and CD4⁺ T cells naturally have greater cytokine-production capacity than CD8⁺ T cells, it is possible the functional superiority observed above was simply due to the presence of CD4⁺ cells rather than the naïve/memory phenotype. Therefore, the inventors further compared NM-derived CAR-T cells with CAR-T cells generated from a CD3-sorted population in subsequent in vivo studies.

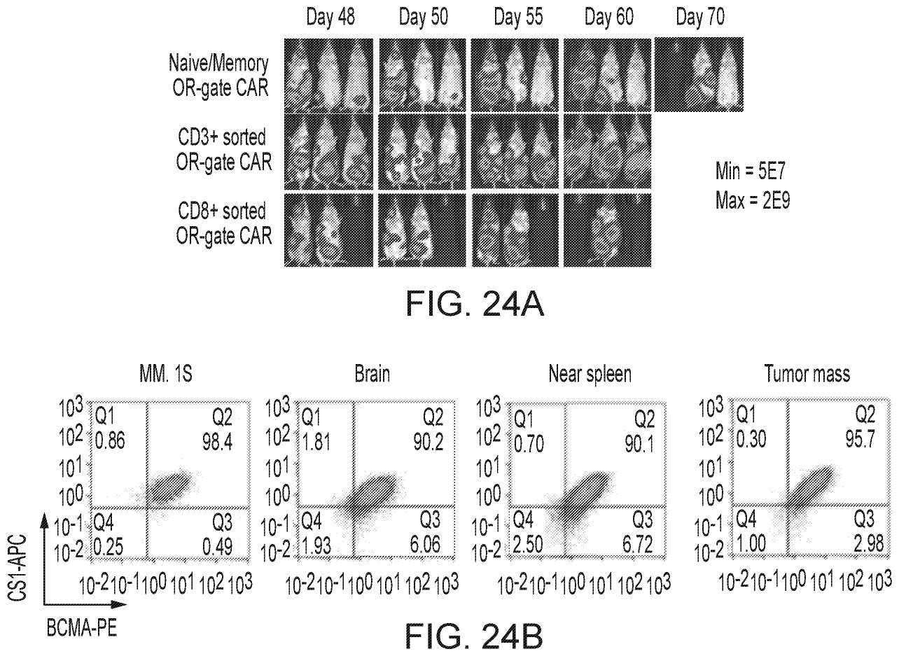

NSG mice bearing wildtype BCMA⁺/CS1⁺ MM.1S xenografts were treated with 0.5×10⁶ NM-derived, CD3-derived, or CD8-derived c11D5.3-Luc90 OR-gate CAR-T cells. All three treated groups achieved tumor clearance by day 12, but NM-derived OR-gate CAR-T cells cleared tumor cells more rapidly compared to the other two cell types (FIG. 14D). Animals were given a second dose of T cells on day 21 after tumor injection, when tumor relapse became apparent in all groups. One out of three mice in the NM-derived CAR-T cell group subsequently achieved complete tumor clearance (FIG. 24), and this group showed the highest overall median survival (FIG. 14E). Analysis of tumor cells recovered at the time of sacrifice indicated that the cells retained antigen expression (FIG. 24), thus the failure to eradicate tumors was not a result of spontaneous antigen escape and likely attributable to the tumor model's aggressiveness and the low T cell dose administered. Taken together, these results indicate that NM-derived BCMA/CS1 OR-gate CAR-T cells have superior anti-tumor functionality in vivo compared to CD3- or CD8-derived T cells. Therefore, subsequent studies were performed with NM-derived T cells.

7. Superior Performance of Bispecific CARs Compared to Single-Input BCMA and CS1 CARs In Vivo

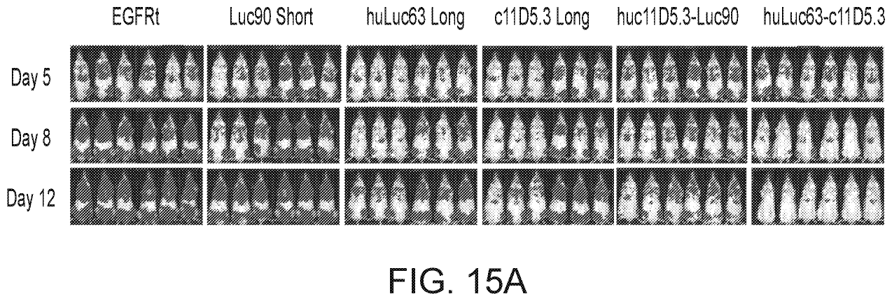

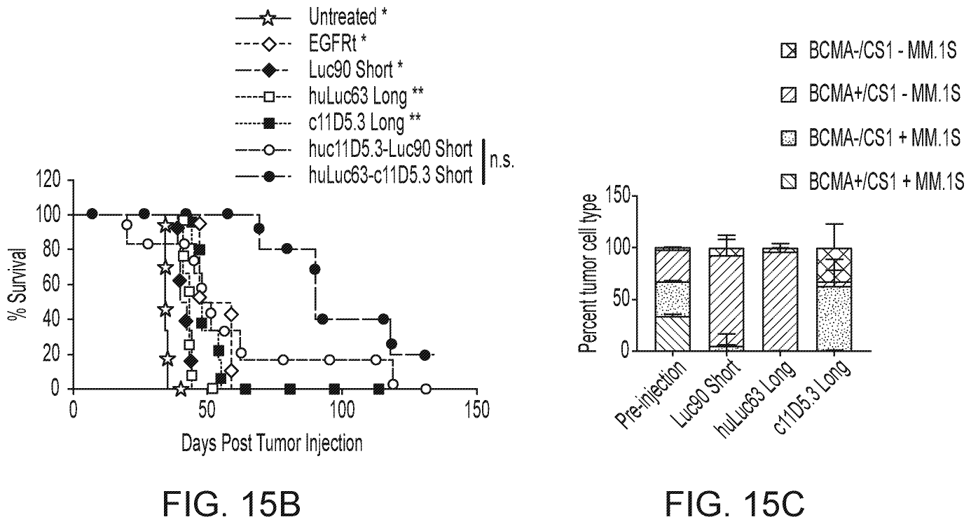

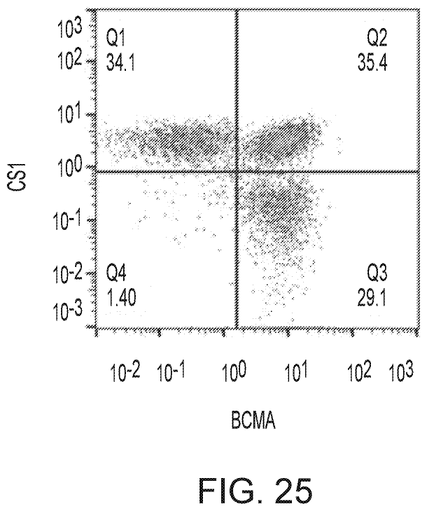

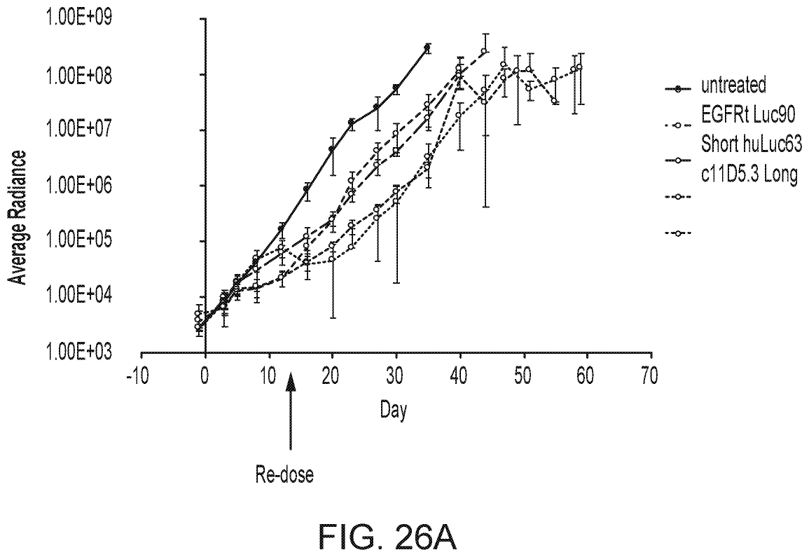

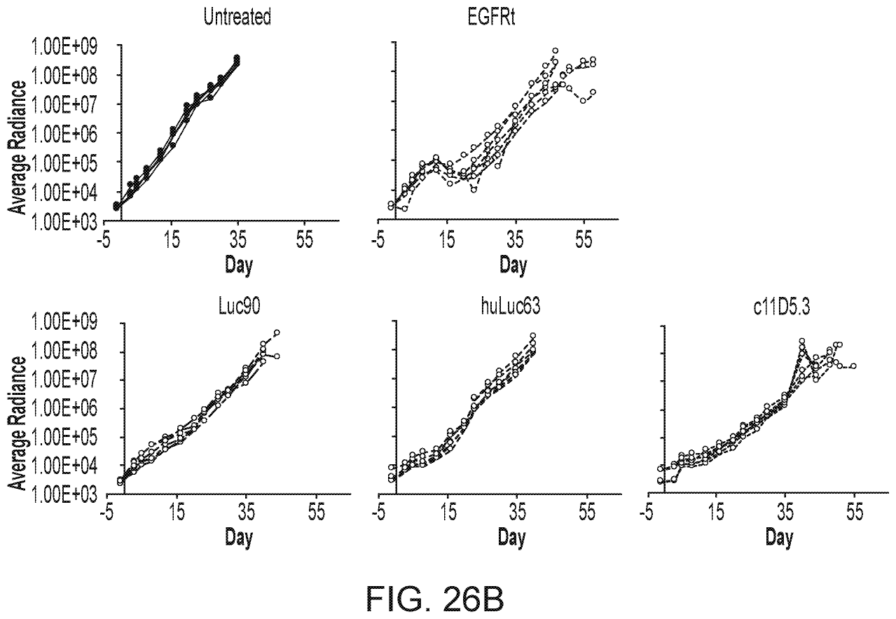

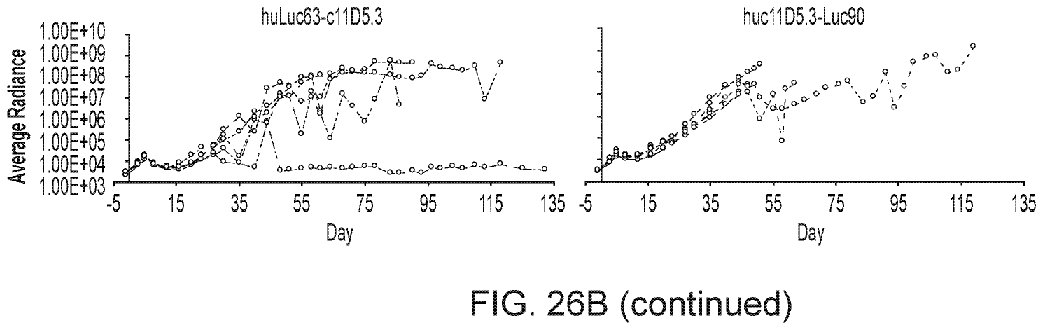

To evaluate the ability of OR-gate CAR-T cells to prevent antigen escape in vivo, NSG mice were engrafted with a mixed population of three firefly luciferase-expressing MM.1S cell lines, containing a 1:1:1 ratio of BCMA⁺/CS1⁺, BCMA⁺/CS1⁻, and BCMA⁻/CS1⁺ MM.1S cells (FIG. 25). Tumor-bearing mice were treated with single-input or OR-gate CAR-T cells on days 5 and 13 post tumor injection. Bioluminescence imaging revealed huLuc63-c11D5.3 Short CAR-T cells as the clear leader in anti-tumor activity, yielding near-complete tumor clearance by day 12 (FIG. 15A,B). Notably, animals treated with single-input BCMA or CS1 CAR-T cells fared no better than those treated with mock-transduced (EGFRt-expressing) T cells, whose anti-tumor efficacy was presumed to originate from allogeneic effects (38). In contrast, animals in the huLuc63-c11D5.3 Short CAR-T cell-treated groups showed significantly longer overall survival, with one animal achieving complete and durable tumor clearance through the 134-day study (FIG. 15A,B and FIG. 26).

MM.1S cells recovered from tumor-bearing animals at the time of animal sacrifice revealed an intriguing pattern of antigen expression. Although all MM.1S cells expressed at least one antigen at the time of tumor injection (FIG. 25), a substantial fraction of tumor cells recovered from animals treated with single-input BCMA CAR-T cells (c11D5.3) were BCMA⁻/CS1⁻ (FIG. 15C), suggesting some MM.1S cells may have spontaneously lost BCMA expression under selective pressure from BCMA CAR-T cells. This double-negative tumor population was not observed in significant numbers in untreated animals or animals treated with single-input CS1 CAR-T cells (Luc90 and huLuc63; FIG. 15C), underscoring BCMA's particular vulnerability to antigen escape when treated with single-input BCMA CAR-T cells.

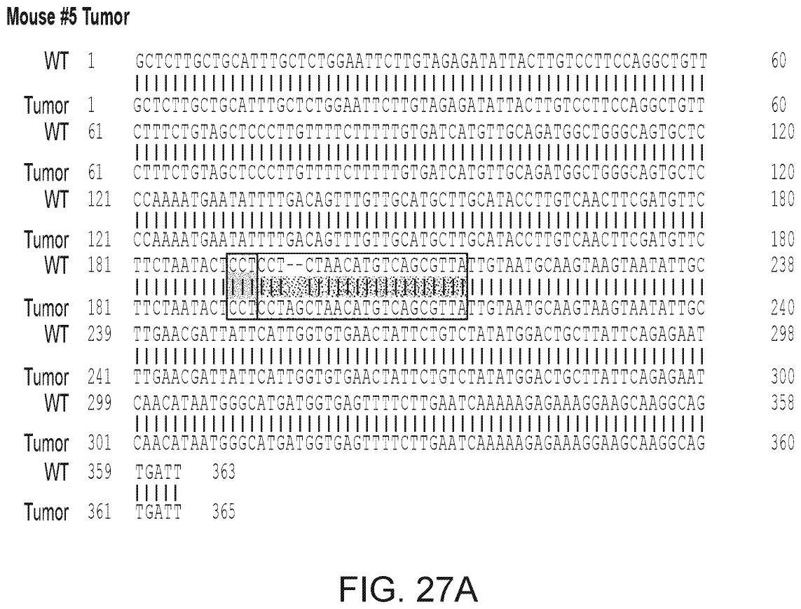

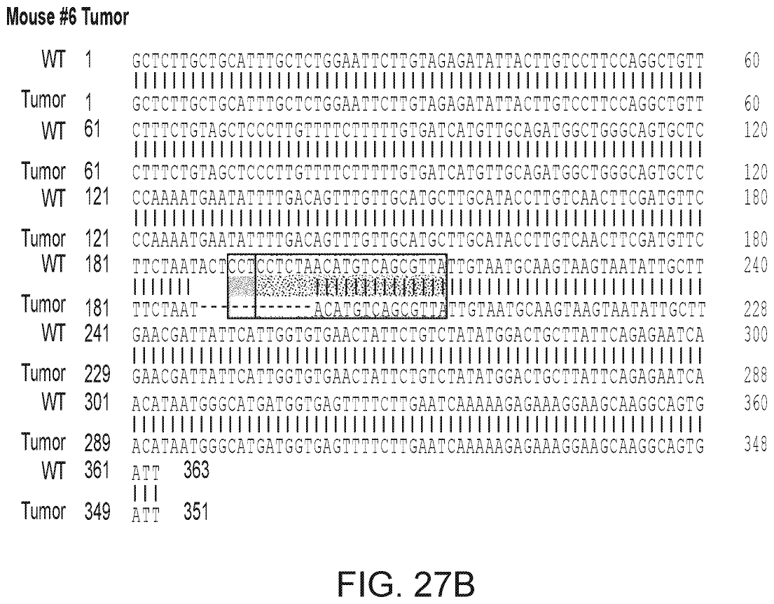

The inventors further observed that tumors remaining in OR-gate CAR-treated animals were mostly BCMA⁻/CS1⁺, with a minor population of double-negative tumors (FIG. 15D). Two predominantly BCMA⁻/CS1⁺ tumor samples recovered from two different animals in the huLuc63-c11D5.3 Short CAR-T cell-treated group were analyzed by amplicon sequencing, and results indicated that both tumors originated from cells that had been engineered by CRISPR/Cas9 editing to be BCMA⁻ prior to in vivo engraftment (FIG. 27). Three non-mutually exclusive possibilities could explain the particularly strong persistence of BCMA⁻/CS1⁺ tumor cells in animals treated with OR-gate CAR-T cells: (i) the OR-gate CAR-T cells were less effective against CS1 than against BCMA, (ii) the residual tumor cells have become unrecognizable or resistant to CAR-T cells, or (iii) the BCMA⁻/CS1⁺ tumor line has an inherent growth advantage over the wildtype and BCMA⁺/CS1⁻ MM.1S lines. The inventors empirically evaluated each possibility in turn.

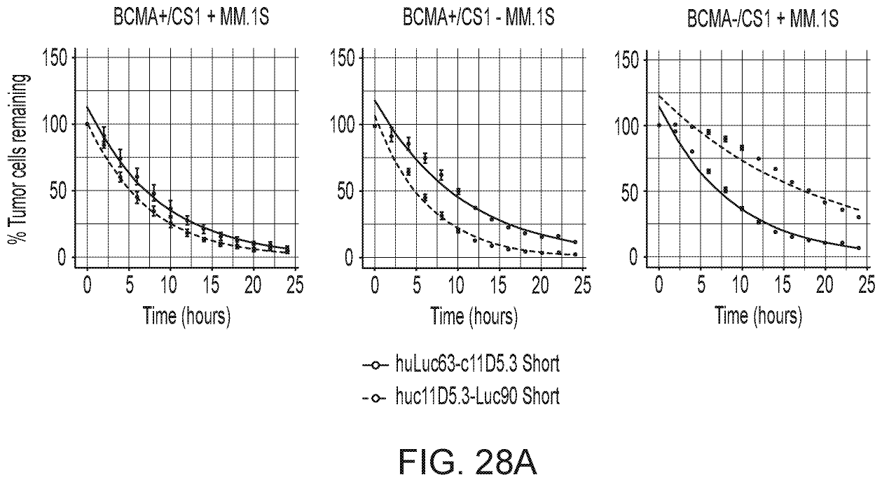

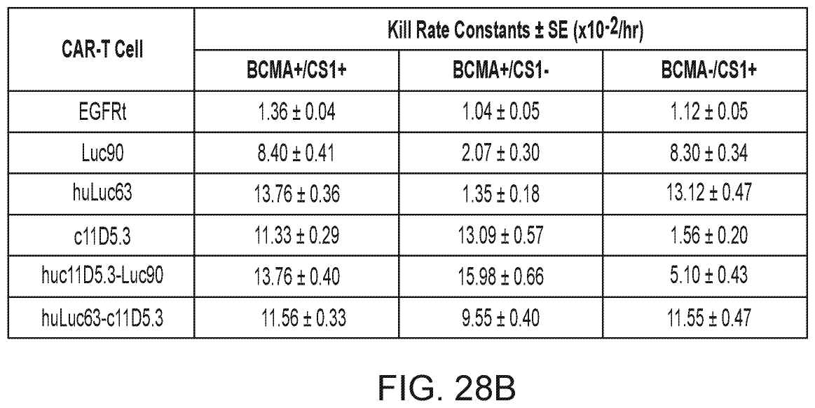

First, time-lapse imaging analysis (IncuCyte) was performed to quantify the kinetics of tumor-cell killing by OR-gate CAR-T cells in vitro. Results indicated huLuc63-c11D5.3 Short CAR-T cells killed CS1⁺ tumor cells more rapidly than BCMA⁺ targets (FIG. 28), which is consistent with previous observations (FIG. 12D) and argues against the hypothesis that OR-gate CAR-T cells were less effective against the CS1 antigen in general.

Second, tumor cells recovered from two different animals in the huLuc63-c11D5.3 Short CAR-T cell-treated group (same as those analyzed in FIG. 27) were re-challenged by huLuc63-c11D5.3 Short CAR-T cells ex vivo, and both tumor samples were efficiently eliminated by the CAR-T cells, indicating that the tumor cells remained recognizable and vulnerable to CAR-T cells (FIG. 2929A,B).

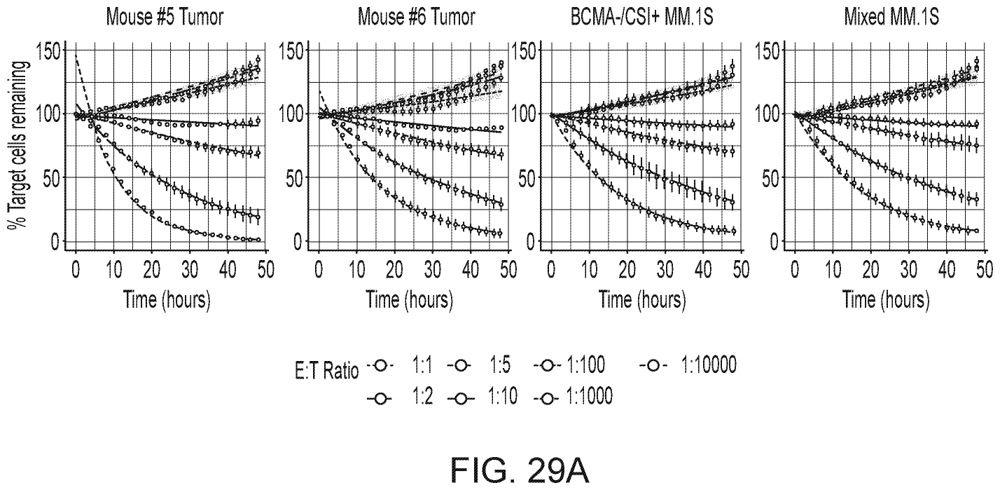

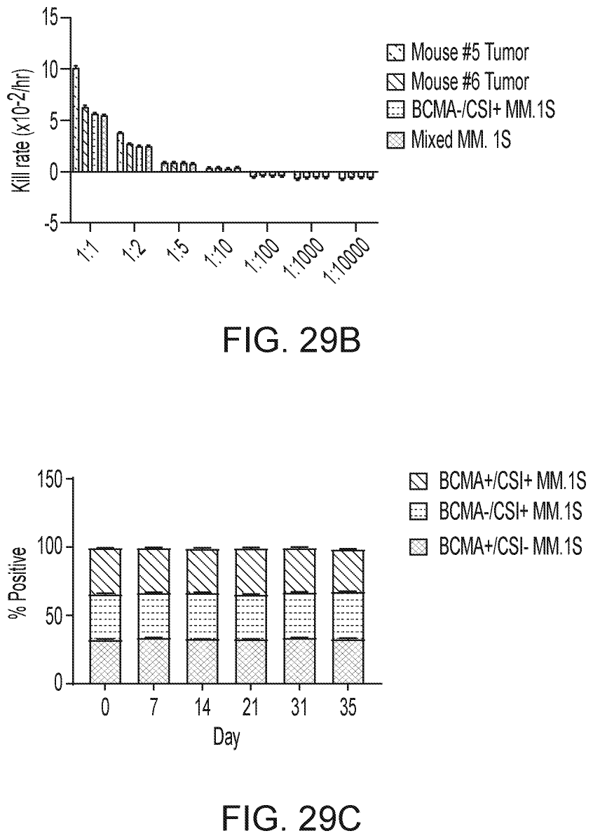

Third, wildtype, BCMA⁺/CS1⁻, and BCMA⁻/CS1⁺ MM.1S cells were co-cultured at 1:1:1 ratio in vitro, and no difference in their relative growth rate was observed over a 5-week period (FIG. 29C). However, tumors recovered from animals that were either untreated or treated with mock-transduced (EGFRt) T cells both showed an enrichment of BCMA⁻/CS1⁺ cell content despite the lack of selective pressure against either antigen (FIG. 15D), suggesting that the BCMA⁻/CS1⁺ cell line may have a growth advantage in the in vivo milieu that is not evident in cell culture. Interestingly, amplicon sequencing results from tumor samples indicate that the vast majority of cells in each tumor arose from a single clone of MM.1S, but different clones gave rise to the two tumors that were sequenced. This was evidenced by the fact that within each tumor, nearly all cells (99.5%-99.7%) contained the same BCMA mutation, but the two tumors contained two different BCMA mutations within the CRISPR-edited region. Therefore, an intriguing possibility is that BCMA⁻/CS1⁺ cells may have greater capacity to undergo clonal expansion in vivo compared to WT or BCMA⁺/CS1⁻ cells.

8. Lentivirally Modified Cells Outperform HDR-Modified Cells In Vitro

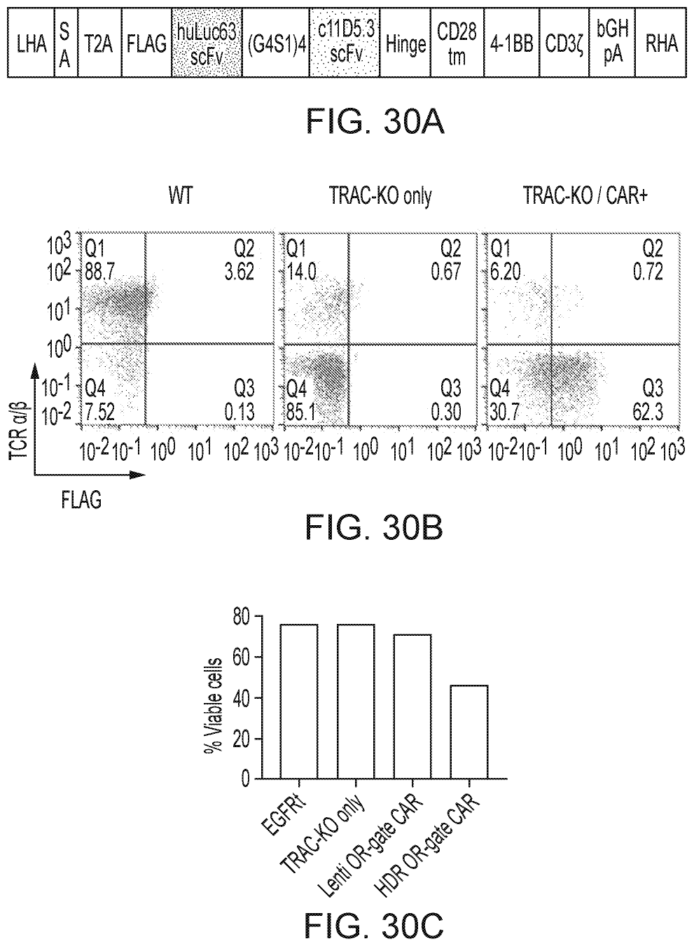

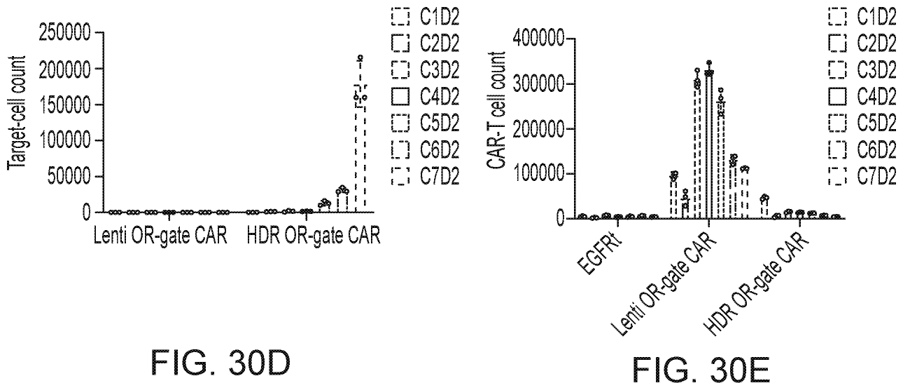

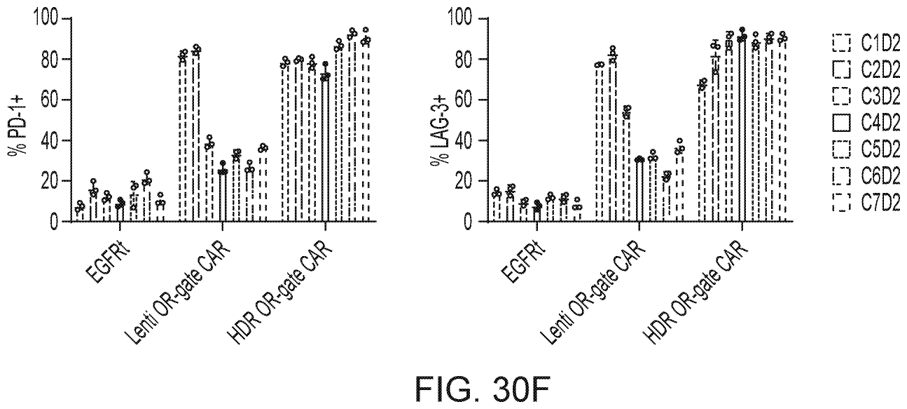

Although the in vivo study above demonstrated that BCMA/CS1 bispecific CAR-T cells can significantly prolong survival of animals bearing heterogenous MM xenografts, the majority of treated animals eventually succumbed to tumor growth, prompting us to evaluate alternative strategies to further bolster T cell function. It had been reported that CAR-T cells with the CAR integrated into the T cell receptor a constant (TRAC) locus via homology-directed repair (HDR) exhibit longer T cell persistence and less exhaustion upon antigen stimulation in vivo compared to retrovirally transduced CAR-T cells (27). The inventors thus integrated a FLAG-tagged huLuc63-c11D5.3 OR-gate CAR into the TRAC locus (FIG. 30A) and verified TRAC knock-out and CAR knock-in by surface antibody staining for TCR α/β chains and the FLAG-tag, respectively (FIG. 30B).

TRAC-knockout T cells showed comparable viability to that of lentivirally transduced cells, indicating CRISPR/Cas9-mediated editing through RNP nucleofection did not compromise cell viability (FIG. 30C). However, contrary to expectations, TRAC-knockout T cells that were further HDR-modified to express OR-gate CARs showed poor viability and inferior cytotoxicity upon repeated antigen challenge compared to lentivirally transduced OR-gate CAR-T cells (FIG. 30C,D). Furthermore, HDR-modified cells showed weaker antigen-stimulated T cell proliferation (FIG. 30E), as well as higher and more durable exhaustion-marker expression (FIG. 30F), compared to lentivirally transduced cells. Based on these results, lentiviral transduction was retained as the preferred method for CAR-T cell generation.

9. Combination Therapy with Anti-PD-1 Antibody Enhances In Vivo Anti-Tumor Efficacy

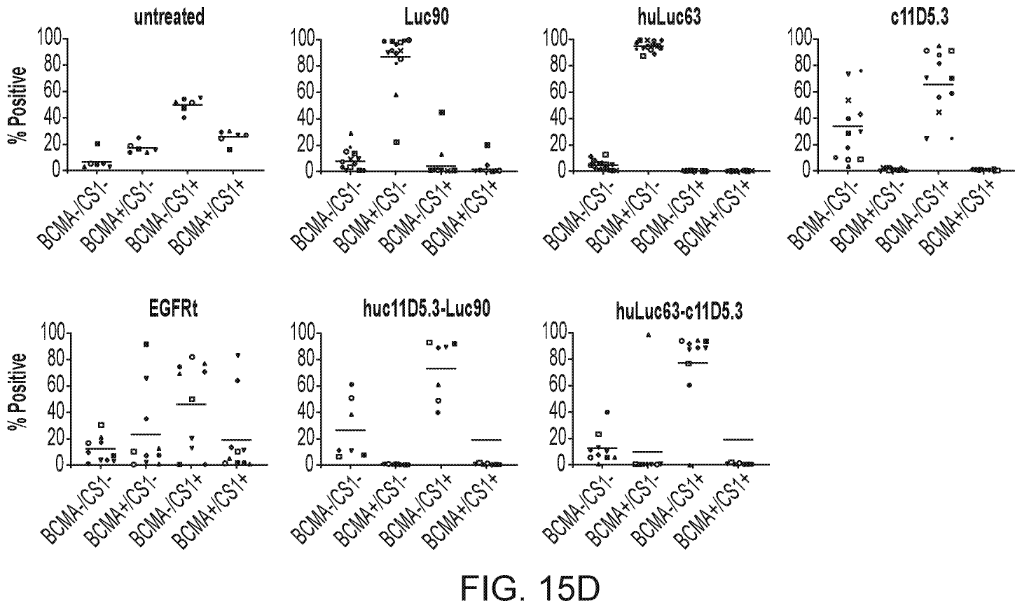

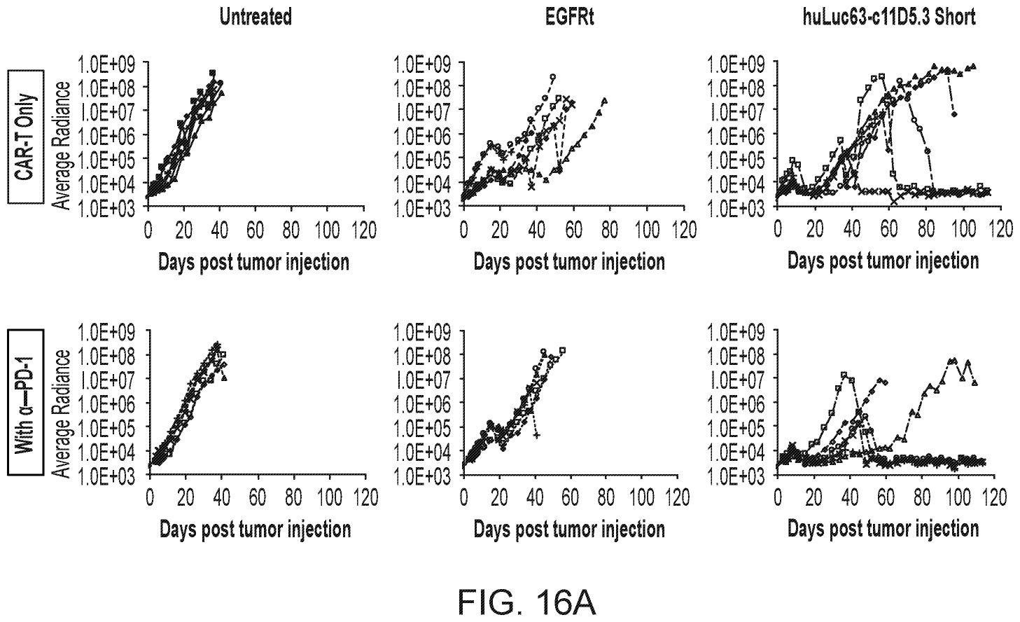

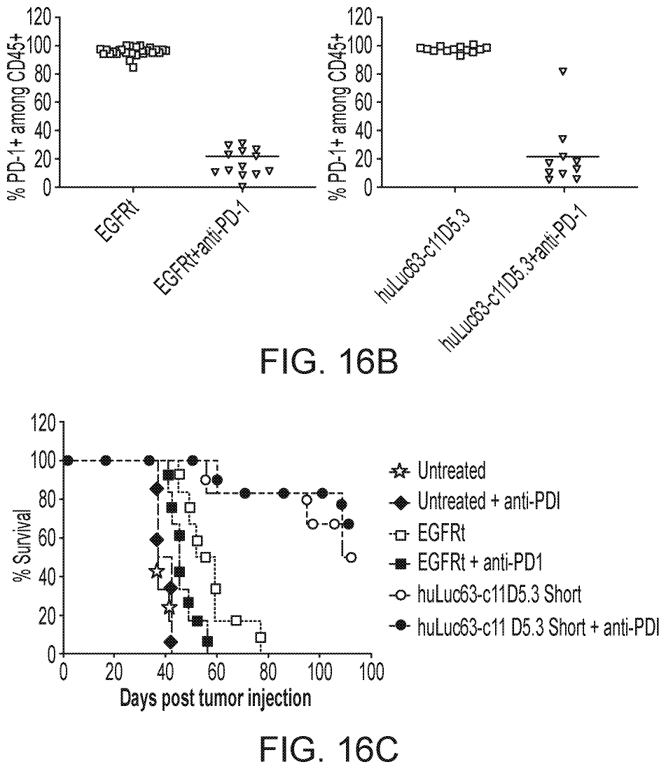

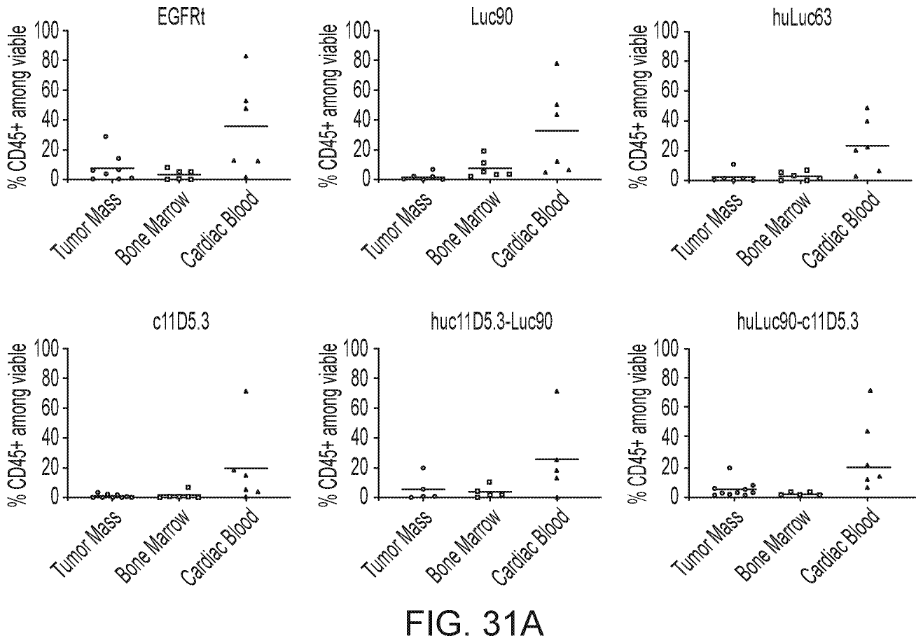

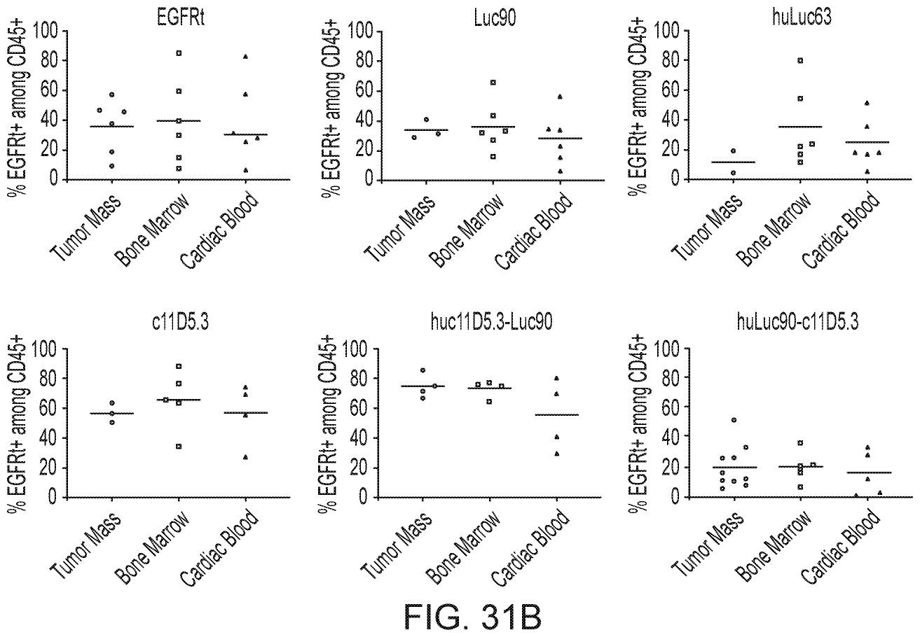

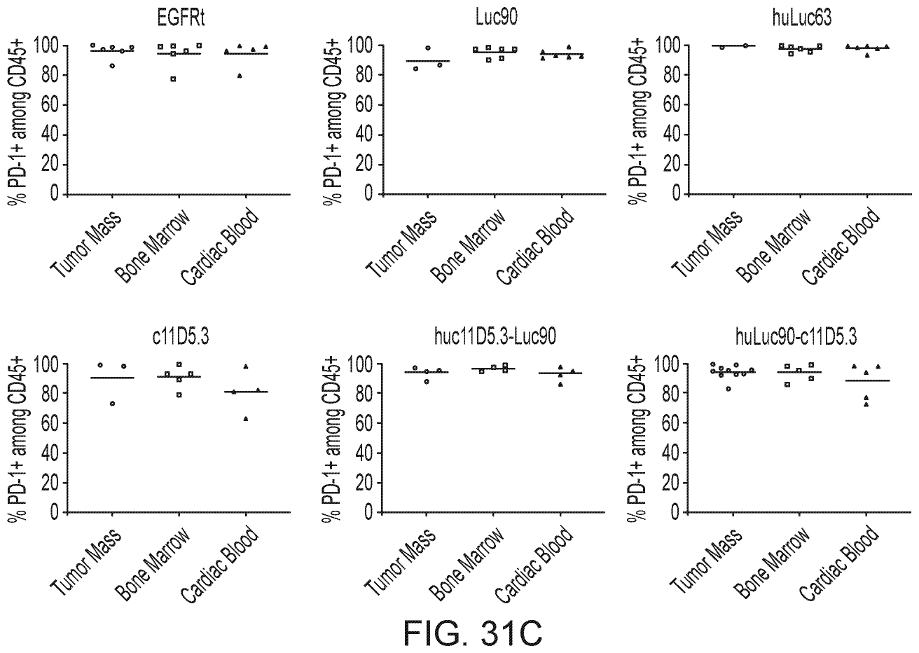

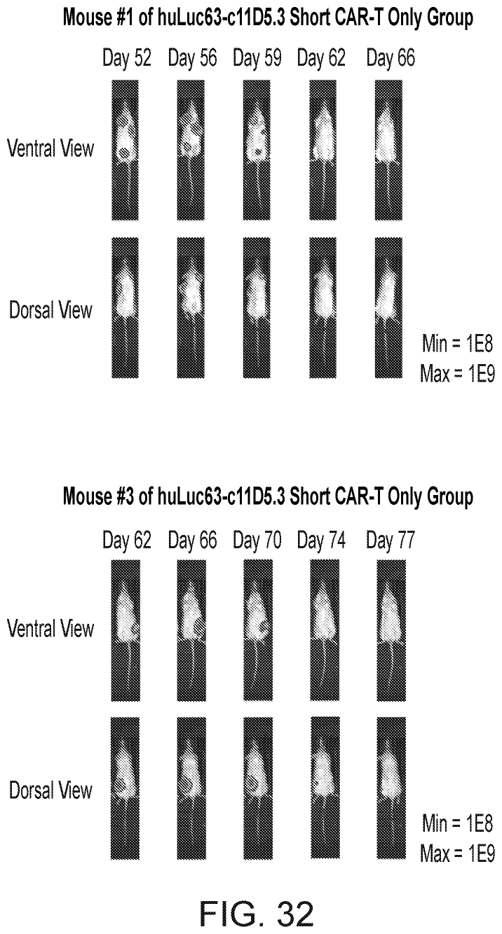

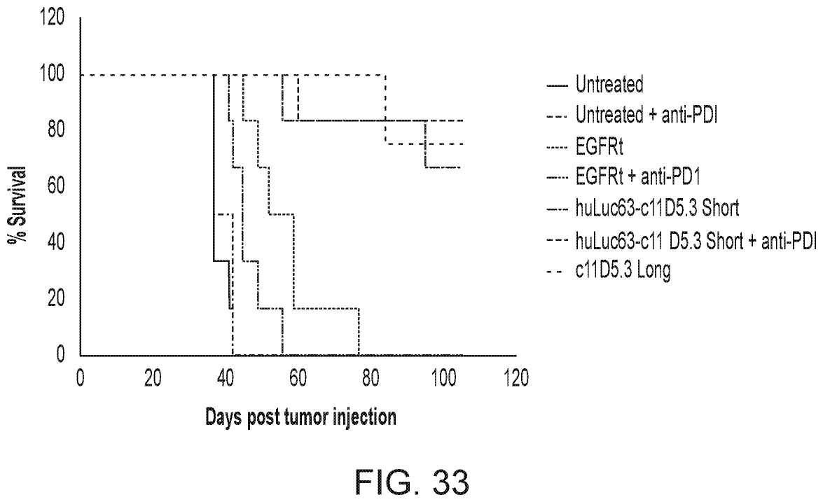

Tissue recovered at the time of animal sacrifice in the in vivo study shown in FIG. 15 revealed the presence of CAR-T cells, but they were generally present at low frequency and with high PD-1 expression (FIG. 31). This observation suggests combination therapy with checkpoint inhibitors may be an alternative method to improve treatment efficacy. Indeed, the inventors found that co-administration of anti-PD-1 antibody and the huLuc63-c11D5.3 OR-gate CAR-T cells led to significantly more effective tumor control compared to OR-gate CAR-T cells alone (FIG. 16A). By day 48 post T-cell injection (day 56 post tumor injection), 5/6 animals treated with OR-gate CAR-T cells plus anti-PD-1 exhibited complete tumor clearance or minimal residual tumor, compared to 1/6 animal in the group treated with CAR-T cells alone (FIG. 16A). The beneficial effect of checkpoint inhibition is dependent on the presence of CAR-T cells, as anti-PD-1 therapy alone or in combination with mock-transduced T cells did not confer anti-tumor capability and actually appeared to reduce the allogeneic effect exerted by mock-transduced T cells on engrafted tumors (FIG. 16A). T cells recovered at the time of sacrifice showed substantially lower PD-1 expression in animals treated with anti-PD-1, confirming checkpoint inhibition (FIG. 16B).

Interestingly, although CAR-T cell treatment alone failed to control initial tumor progression in most animals, two mice that had developed palpable solid tumors became tumor free 50 and 68 days after the second and final T-cell infusion, respectively (FIG. 6A and Fig. S16). This result highlights BCMA/CS1 OR-gate CAR-T cells' ability to eradicate established solid tumor masses even after a prolonged period in vivo. At the time of this writing (113 days post tumor injection), 50% of animals treated with OR-gate CAR-T cells alone are viable and have been tumor free for >4 weeks. In comparison, 67% of animals treated with OR-gate CAR-T cells plus anti-PD-1 are viable and have been tumor-free for >8 weeks (FIG. 16A,C).

D. Discussion

Following the success of CD19 CAR-T cell therapy for B-cell leukemia and lymphoma, the BCMA CAR is a leading candidate to receive the next FDA approval for adoptive T cell therapy for cancer. However, outcomes from recent clinical trials indicate that BCMA-targeted CAR-T cell therapy is vulnerable to antigen escape (3, 4, 6). To develop a more effective CAR for MM treatment, the inventors engineered single-chain bispecific (OR-gate) CARs that efficiently target not only BCMA but also CS1. Via high-throughput CAR construction and screening as well as improvements to the cell manufacturing process, the inventors generated BCMA/CS1 OR-gate CAR-T cells that can robustly eliminate heterogeneous MM cells in vitro and in vivo.

CS1 is expressed in more than 90% of patient MM samples and not expressed on non-hematological and essential tissues such as the stomach, lung, kidney, brain, and heart (16, 17, 33). As such, it is an ideal target to be paired with the more heterogeneously expressed but clinically validated BCMA for MM treatment. However, CS1 is expressed on natural killer (NK) cells, natural killer T (NKT) cells, CD8⁺ T cells, activated monocytes, and dendritic cells, albeit at much lower levels than on plasma cells (16, 33). CS1 expression on non-cancerous hematological cell types raises the question of potential off-tumor toxicities. However, the inventors noted that CS1-specific CAR-T cells showed slightly but not statistically significantly higher lytic activity against bystander CD8⁺ T cells, and they showed no defects in ex vivo expansion.

In principle, given CS1's nearly uniform expression on MM cells, a CS1 single-input CAR-T cell therapy may be adequate. However, the inventors observed that single-input CS1 CAR-T cells show signs of functional defect upon repeated antigen challenge in vitro (FIG. 22D,E) and are less potent in vivo compared to both single-input BCMA CAR-T cells and bispecific BCMA/CS1 OR-gate CAR-T cells (FIG. 15A,B). By combining both BCMA and CS1 in a single-chain bispecific CAR design, the inventors take advantage of both the uniform expression of CS1 and the strong anti-tumor output elicited by BCMA targeting to achieve more effective tumor control.

In addition to the OR-gate CAR, other strategies can be taken to demonstrate bispecific targeting including, DualCAR (co-expressing two full-length receptors on one cell) and CARpool (combining two single-input CAR-T cell products). Compared to the OR-gate CAR, the DualCAR requires a much larger genetic payload, which leads to poor transduction efficiency (42, 43) as well as reduced anti-tumor functions (FIG. 13), consistent with previous reports (10, 44). A CARpool strategy could avoid the issue of poor transduction efficiency, but it requires manufacturing two clinical products and sets up potential competition between the two engineered T cell populations. In contrast, the OR-gate CAR structure guarantees that all CAR-T cells are capable of recognizing both BCMA and CS1, thus every engineered T cell would be able to safeguard against the loss of either antigen.

The inventors' results indicate that rules dictating target epitope location and CAR structural requirements can indeed be rationally applied to the design of BCMA/CS1 OR-gate CARs. However, in the presence of conflicting preferences (e.g., when both target epitopes require their corresponding ligand-binding domains to be placed at the same position on a OR-gate CAR), empirical testing remains necessary, and some tradeoffs in relative targeting efficiency remain unavoidable. An alternative CAR architecture that was not explored in this study is the “loop CAR” structure in which the scFv for one antigen is inserted between the V_{L} and V_{H} domains of the scFv for the second antigen (44). A comparison of the inventors' r OR-gate CAR design, which utilizes two scFvs connected in tandem, with the loop CAR design may yield interesting insights into additional parameters that may be tuned in the CAR architecture. The low-volume, high-throughput functional assays developed for this study enabled head-to-head comparisons of up to 17 CAR-T cell lines derived from the same donor's cells across multiple effector outputs. This capability allowed us to identify differences across CAR designs that were only noticeable in some functional assays and not others, thus enabling the rapid and effective selection of lead candidates that exhibit superior anti-tumor function compared to single-input CS1 and BCMA CARs in vitro and in vivo.

In this study, the inventors demonstrated the use of naïve/memory T cells as the starting population yields functionally superior cell products compared to those generated from bulk CD3⁺ or CD8⁺ T cells (FIG. 14), highlighting the importance of cell-manufacturing parameters to eventual therapeutic efficacy. Furthermore, the inventors found that lentivirally transduced CAR-T cells exhibit greater anti-tumor activity compared to CAR-T cells generated through CRISPR/Cas9-mediated editing (FIG. 30). This finding was unexpected given the compelling data from a previous study demonstrating functional superiority of T cells that had undergone site-specific integration of the CAR transgene into the TRAC locus (27). Further exploration would be needed to determine whether the benefit of site-specific CAR transgene integration is limited to some CARs and, if so, whether the difference is determined by the antigen specificity of the CAR or more generalizable properties such as the size of the CAR construct (and thus the size of the HDR template).

The inventors' in vivo study demonstrated that BCMA/CS1 OR-gate CAR-T cells are uniquely capable of controlling heterogenous MM that proves resistant to single-input BCMA or CS1 CAR-T cell therapy (FIG. 15). The inventors further demonstrated that the co-administration of anti-PD-1 antibody with OR-gate CAR-T cells can lead to durable, tumor-free survival of animals that had been engrafted with highly aggressive MM xenografts (FIG. 16). The inventors' in vivo data revealed intriguing dynamics of MM evolution under selective pressure. Specifically, single-input BCMA CAR-T cells led to a substantial (mean 33%, range 4%-76%) fraction of residual BCMA⁻/CS1⁻ tumors in treated animals. In contrast, animals treated with single-input CS1 CAR-T cells showed few double-negative residual tumor cells (FIG. 15D). These results suggest that BCMA may be particularly susceptible to antigen escape under selective pressure from single-input BCMA CAR-T cell therapy, and underscores the utility of dual-antigen targeting for MM.

This work presents a rational approach for the engineering of BCMA/CS1 OR-gate CAR-T cells that can effectively target MM tumor and substantially reduce the probability of tumor antigen escape. The small genetic footprint of OR-gate CAR constructs facilitate the clinical manufacturing of T cell products, and OR-gate CAR-T cells' functional superiority over DualCAR-T cells provide a compelling advantage for clinical translation. Finally, the vertically integrated design process outlined in this study can be applied towards the engineering of novel CARs to expand the applications of adoptive T cell therapy to additional cancer types currently lacking effective treatment options.

E. Tables

Supplementary Table 1. Amino-Acid Sequences of CAR Components

BCMA-targeting domains

| | |
|---|---|
| c11D5.3 murine scFv (V_{L}-V_{H}) | SEQ ID NO: 22 |
| c11D5.3 human scFv (V_{L}-V_{H}) | SEQ ID NO: 25 |
| J22.9-xi murine scFv (V_{L}-V_{H}) | SEQ ID NO: 34 |
| J22.9-xi human scFv (V_{L}-V_{H}) | SEQ ID NO: 37 |
| dAPRIL | SEQ ID NO: 38 |

CS1-targeting domains

| Luc90 (V_{H}-V_{L}) | SEQ ID NO: 47 |
|---|---|
| huLuc63 (V_{H}-V_{L}) | SEQ ID NO: 56 |
| Extracellular spacer, transmembrane domain, and intracellular | |
| signaling domains | |
| IgG4 hinge- | SEQ ID NO: 73 |
| CH2(L235E, N297Q)- | SEQ ID NO: 74 |
| CH3 | SEQ ID NO: 75 |
| CD28tm- | SEQ ID NO: 76 |
| 4-1BB - | SEQ ID NO: 77 |
| Zeta | SEQ ID NO: 78 |

### Example 3: Efficacy of Newly Designed Single-Input BCMA-CAR

Bioluminescence imaging results indicate that animals treated with newly-designed single-input BCMA CAR-T cells (c11D5.3 Long) also showed complete tumor control that was comparable or better to animals treated with bispecific CAR-T cells. Two different but non-mutually exclusive explanations may account for this result. First, the animals used in the c11D5.3 Long treatment group showed slower tumor engraftment compared to animals used in all other treatment groups. Specifically, animals in the c11D5.3 Long treatment group took 5 days longer than those in the other test groups to reach the same tumor burden and were, accordingly, treated with T cells 5 days later than the animals in the other test groups. The fact that the tumor cells took longer to engraft points to lower viability or poorer health of the tumor cells at the time of injection, which could conceivably have led to weaker tumor progression at subsequent time points, independent of any anti-tumor effect exerted by CAR-T cells. The alternative explanation is that, as described in the manuscript, a tradeoff had to be made between BCMA and CS1 targeting when designing the bispecific CAR. Consequently, the “huLuc63-c11D5.3 Short” bispecific CAR was slightly weaker in BCMA targeting compared to the “c11D5.3 Long” single-input BCMA CAR. When the tumor is 100% BCMA⁺, as was the case in this particular in vivo study, the superior BCMA-targeting efficiency of the single-input BCMA CAR may account for the superior tumor control observed with this CAR-T cell treatment. Therefore, these results provide evidence of superior BCMA-targeting efficiency of the single-input BCMA CAR comprising the “long” spacer.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. All publications, references, patent publications and patent applications recited in this specification are herein specifically incorporated by reference for all purposes.

### REFERENCES

The following references and the publications referred to throughout the specification, to the extent that they provide exemplary procedural or other details supplementary to those set forth herein, are specifically incorporated herein by reference.

### EXAMPLE 1 REFERENCES

1. Gibson D G, Young L, Chuang R-Y, Venter J C, Hutchison C a, Smith H O. Enzymatic assembly of DNA molecules up to several hundred kilobases. Nat Methods. 2009; 6(5):343-345. doi:10.1038/nmeth.1318.
2. Carpenter R O, Evbuomwan M O, Pittaluga S, et al. B-cell maturation antigen is a promising target for adoptive T cell therapy of multiple myeloma. Clin Cancer Res. 2013; 19(8):2048-2060. doi:10.1158/1078-0432.CCR-12-2422.
3. WO 2016/014565 A2 (Brogdon et al.) 28 Jan. 2016.
4. Oden F, Marino S F, Brand J, et al. Potent anti-tumor response by targeting B cell maturation antigen (BCMA) in a mouse model of multiple myeloma. Mol Oncol. 2015; 9(7):1348-1358. doi:10.1016/j.molonc.2015.03.010.
5. Lee L S H, Draper B O, Chaplin N, et al. An APRIL Based Chimeric Antigen Receptor to Simultaneously Target BCMA and TACI in Multiple Myeloma (MM) Has Potent Activity in Vitro and in Vivo. Blood. 2016; 128(22). http://www.bloodjournal.org/content/128/22/379. Accessed Nov. 1, 2017.
6. Chu J, He S, Deng Y, et al. Genetic modification of T cells redirected toward CS1 enhances eradication of myeloma cells. Clin Cancer Res. 2014; 20(15):3989-4000. doi:10.1158/1078-0432.CCR-13-2510.
7. WO 2014/179759 A1 (Yu et al.) 6 Nov. 2014.
8. Wang X, Chang W C, Wong C W, et al. A transgene-encoded cell surface polypeptide for selection, in vivo tracking, and ablation of engineered cells. Blood. 2011; 118(5):1255-1263. doi:10.1182/blood-2011-02-337360.

### EXAMPLE 2 REFERENCES

1. American Cancer Society. Cancer facts & figures 2019. Atlanta Am. Cancer Soc. (2019).
2. Moreau, P., Attal, M. & Facon, T. Frontline therapy of multiple myeloma. Blood 125, 3076-3085 (2015).
3. Ali, S. A. et al. T cells expressing an anti-B-cell maturation antigen chimeric antigen receptor cause remissions of multiple myeloma. Blood 128, 1688-1701 (2016).
4. Brudno, J. et al. T cells genetically modified to express an anti-B-cell maturation antigen chimeric antigen receptor cause remissions of poor-prognosis relapsed multiple myeloma. J. Clin. Oncol. 130, (2018).
5. Berdeja, J. G., Lin, Y., Raje, N., Munshi, N., Siegel, D., Liedtke, M., Jagannath, S., Maus, M. V., Turka, A., Lam, L. P., Hege, K., Morgan, R. A., Quigley, M. T., & Kochenderfer, J. N. Durable clinical responses in heavily pretreated patients with relapsed/refractory multiple myeloma: Updated results from a multicenter study of bb2121 anti-bcma CAR T cell therapy. Blood 130, 740 (2017).
6. Cohen, A. D., Garfall, A. L., Stadtmauer, E. A., Lacey, S. F., Lancaster, E., Vogl, D. T., Weiss, B. M., Ambrose, D. E., Nelson, A. M., Chen, F., Plesa, G., Kulikovskaya, I., Gonzalez, V., Gupta, M., Young, R. M., Dengel, K., O'keefe, L., Le, S., Richards, M. C. Safety and efficacy of B-Cell maturation antigen (BCMA)-specific chimeric antigen receptor T Cells (CART-BCMA) with cyclophosphamide conditioning for refractory multiple myeloma (MM). Blood 130, 505 (2017).
7. Smith, E. L., Mailankody, S., Ghosh, A., Masakayan, R., Staehr, M., Purdon, T. J., Halton, E., Diamonte, C., Anant, P., Bernal, Y., Wang, X., Wang, Y., Garrett, S., Almo, S., Wang, P., Liu, H., Xu, Y., Mead, E., Park, J. H., Curran, K. J., Sauter, C. S., R. J. Development and evaluation of a human single chain variable fragment (scFv) derived bcma targeted CAR T cell vector leads to a high objective response rate in patients with advanced MM. Blood 130, 742 (2017).
8. Grada, Z. et al. TanCAR: A novel bispecific chimeric antigen receptor for cancer immunotherapy. Mol. Ther. Nucleic Acids 2, e105 (2013).
9. Zah, E., Lin, M.-Y., Silva-Benedict, A., Jensen, M. C. & Chen, Y. Y. T cells expressing CD19/CD20 bi-specific chimeric antigen receptors prevent antigen escape by malignant B cells. Cancer Immunol. Res. (2016). doi:10.1158/2326-6066.CIR-15-0231
10. Hegde, M. et al. Tandem CAR T cells targeting HER2 and IL13R α 2 mitigate tumor antigen escape. J. Clin. Invest. 126, 3036-3052 (2016).
11. Qin, H. et al. Novel CD19/CD22 cicistronic chimeric antigen receptors outperform single or bivalent CARs in eradicating CD19+CD22+, CD19−, and CD22− pre-B leukemia. Am. Soc. Hematol. 130 n, (2017).
12. Lee, L. et al. An APRIL-based chimeric antigen receptor for dual targeting of BCMA and TACI in multiple myeloma. Blood 131, 746-758 (2018).
13. Novak, A. J. et al. Expression of BCMA, TACI, and BAFF-R in multiple myeloma: A mechanism for growth and survival. Blood 103, 689-694 (2004).
14. Moreaux, J. et al. The level of TACI gene expression in myeloma cells is associated with a signature of microenvironment dependence versus a plasmablastic signature. Blood 106, 1021-1030 (2005).
15. Lee, L. et al. Evaluation of B cell maturation antigen as a target for antibody drug conjugate mediated cytotoxicity in multiple myeloma. Br. J. Haematol. 174, 911-922 (2016).
16. Hsi, E. D. et al. CS1, a potential new therapeutic antibody target for the treatment of multiple myeloma. Clin. Cancer Res. 14, 2775-2784 (2008).
17. Tai, Y.-T. et al. Anti-CS1 humanized monoclonal antibody HuLuc63 inhibits myeloma cell adhesion and induces aritibody-dependent cellular cytotoxicity in the bone marrow mitieu. Blood 112, 1329-1337 (2008).
18. Wang, X. et al. Lenalidomide enhances the function of CS1 chimeric antigen receptor-redirected T cells against multiple myeloma. Clin. Cancer Res. 24, 106-119 (2018).
19. Gibson, D. G. et al. Enzymatic assembly of DNA molecules up to several hundred kilobases. Nat. Methods 6, 343-345 (2009).
20. Carpenter, R. O. et al. B-cell maturation antigen is a promising target for adoptive T cell therapy of multiple myeloma. Clin. Cancer Res. 19, 2048-2060 (2013).
21. Brogdon, Jennifer; Choi, Eugene; Ebersbach, HIlmar; Glass, David; Huet, Heather; June, Carl H.; Mannick, Joan; Milone, Michael C.; Murphy, Leon; Plesa, Gabriela; Richardson, Celeste; Ruella, Marco; Singh, Reshma; Wang, Yongqiang; Wu, Q. WO2016014565_Treatment of cancer using humanized anti-BCMA CAR.pdf. (2016).
22. Oden, F. et al. Potent anti-tumor response by targeting B cell maturation antigen (BCMA) in a mouse model of multiple myeloma. Mol. Oncol. 9, 1348-1358 (2015).
23. Lee, L. S. H. et al. An APRIL based chimeric antigen receptor to simultaneously target BCMA and TACI in multiple myeloma (MM) has potent activity in vitro and in vivo. Blood 128, (2016).
24. Chu, J. et al. Genetic modification of T cells redirected toward CS1 enhances eradication of myeloma cells. Clin. Cancer Res. 20, 3989-4000 (2014).
25. Yu, Jianhua; Hofmeister, Craig; Chu, J. WO2014179759_CS1 CAR.pdf. (2014).
26. Wang, X. et al. A transgene-encoded cell surface polypeptide for selection, in vivo tracking, and ablation of engineered cells. Blood 118, 1255-1263 (2011).
27. Eyquem, J. et al. Targeting a CAR to the TRAC locus with CRISPR/Cas9 enhances tumour rejection. Nature 543, 113-117 (2017).
28. Hudecek, M. et al. Receptor affinity and extracellular domain modifications affect tumor recognition by ROR1-specific chimeric antigen receptor T cells. Clin. Cancer Res. 19, 3153-3164 (2013).
29. Hudecek, M. et al. The nonsignaling extracellular spacer domain of chimeric antigen receptors is decisive for in vivo antitumor activity. Cancer Immunol. Res. 3, 125-35 (2015).
30. James, J. R. & Vale, R. D. Biophysical mechanism of T cell receptor triggering in a reconstituted system. Nature 487, 64-69 (2012).
31. Williams, M., Tso, Y., Landolfi, N. F., Powers, D. B. & Liu, G. Therapeutic use of anti-CS1 antibodies U.S. Pat. No. 7,709,610B2. (2010).
32. Chen, K. H. et al. A compound chimeric antigen receptor strategy for targeting multiple myeloma. Leukemia 32, 402-412 (2018).
33. Bouchon, A., Cella, M., Grierson, H. L., Cohen, J. I. & Colonna, M. Cutting Edge: Activation of NK cell-mediated cytotoxicity by a SAP-independent receptor of the CD2 Family. J. Immunol. 167, 5517-5521 (2001).
34. Turtle, C. J. et al. CD19 CAR-T cells of defined CD4+: CD8+ composition in adult B cell ALL patients. J Clin Invest 1, 1-16 (2016).
35. Berger, C. et al. Adoptive transfer of effector CD8+ T cells derived from central memory cells establishes persistent T cell memory in primates. J. Clin. Invest. 118, 294-305 (2008).
36. Sommermeyer, D. et al. Chimeric antigen receptor-modified T cells derived from defined CD8+ and CD4+ subsets confer superior antitumor reactivity in vivo. Leukemia 30, 492-500 (2016).
37. Xu, Y. et al. Closely-related T-memory stem cells correlate with in-vivo expansion of CAR.CD19-T cells in patients and are preserved by IL-7 and IL-15. Blood 123, 3750-3759 (2014).
38. Frigault, M. J. et al. Identification of chimeric antigen receptors that mediate constitutive or inducible proliferation of T cells. Cancer Immunol. Res. 3, 356-67 (2015).
39. Magen, H. & Muchtar, E. Elotuzumab: the first approved monoclonal antibody for multiple myeloma treatment. Ther. Adv. Hematol. 7, 187-195 (2016).
40. Lonial, S. et al. Elotuzumab therapy for relapsed or refractory multiple myeloma. N. Engl. J. Med. 373, 621-631 (2015).
41. Zonder, J. A. et al. A phase 1, multicenter, open-label, dose escalation study of elotuzumab in patients with advanced multiple myeloma. Blood 120, 552-559 (2013).
42. Kumar, M., Keller, B., Makalou, N. & Sutton, R. E. Systematic determination of the packaging limit of lentiviral vectors. Hum. Gene Ther. 12, 1893-905 (2001).
43. Bos, T. J., De Bruyne, E., Van Lint, S., Heirman, C. & Vanderkerken, K. Large double copy vectors are functional but show a size-dependent decline in transduction efficiency. J. Biotechnol. 150, 37-40 (2010).
44. Qin, H. et al. Preclinical development of bivalent chimeric antigen receptors targeting Both CD19 and CD22. Mol. Ther. Oncolytics 11, 127-137 (2018).

What is claimed is:

1. A bispecific chimeric antigen receptor (CAR) comprising:
a) a bispecific extracellular binding domain comprising i) one or more BCMA-binding regions, and ii) one or more CS1-binding regions separated by one or more linkers;
b) a single transmembrane domain; and,
c) a single cytoplasmic region comprising a primary intracellular signaling domain.

2. The bispecific CAR of claim 1, wherein the bispecific extracellular binding domain a) comprises a BCMA/CS1 loop.

3. The bispecific CAR of claim 1, wherein the bispecific extracellular binding domain a) comprises i) a BCMA-binding region, that is a single-chain variable fragment (scFv) or a binding region of a proliferation-inducing ligand (dAPRIL) and ii) a CS1-specific scFv separated by a linker.

4. The bispecific CAR of any one of claims 1-3, wherein the CS1-specific scFv is membrane proximal.

5. The bispecific CAR of any of claims 1-3, wherein the BCMA-binding region is membrane proximal.

6. The bispecific CAR of any of claims 1-5, wherein the linker is 4-40 amino acids in length.

7. The bispecific CAR of claim 6, wherein the linker comprises (G4S)n, wherein n is 1, 2, 3, 4, 5, or 6, or the linker comprises, or consists of, the amino acid sequence: (EAAAK)n, wherein n is 1, 2, 3, 4, 5, or 6.

8. The bispecific CAR of claim 7, wherein the linker comprises G4S

9. The bispecific CAR of claim 8, wherein the linker is (G4S)₄.

10. The bispecific CAR of any of claims 1-9, wherein the BCMA-binding regions comprise either i) CDR1, CDR2, CDR3 of both the heavy and light chains from murine or humanized c11D5.3 antibody or murine or humanized J22.9-xi antibody or ii) the heavy and light chain variable regions from murine or humanized c11D5.3 or murine or humanized J229-xi antibody.

11. The bispecific CAR of any of claims 1-10, wherein the CS1-binding regions comprise CDR1, CDR2, CDR3 or the variable region from huLuc63 antibody or murine Luc90 antibody.

12. The bispecific CAR of claim 10, wherein the BCMA-binding regions comprise heavy-chain CDR1 (SEQ ID NO:14), CDR2 (SEQ ID NO:15), and CDR3 (SEQ ID NO:16) from anti-BCMA antibody c11D5.3.

13. The bispecific CAR of claim 10, wherein the BCMA-binding regions comprise light-chain CDR1 (SEQ ID NO:18), CDR2 (SEQ ID NO:19), and CDR3 (SEQ ID NO:20) from anti-BCMA antibody c11D5.3.

14. The bispecific CAR of claim 12 or 13, wherein the BCMA-binding regions comprise heavy-chain CDR1 (SEQ ID NO:14), CDR2 (SEQ ID NO:15), and CDR3 (SEQ ID NO:16) and light-chain CDR1 (SEQ ID NO:18), CDR2 (SEQ ID NO:19), and CDR3 (SEQ ID NO:20) from anti-BCMA antibody c11D5.3.

15. The bispecific CAR of claim 12, wherein the BCMA-binding regions comprise the heavy-chain variable region from murine anti-BCMA antibody c11D5.3 (SEQ ID NO:17).

16. The bispecific CAR of claim 13, wherein the BCMA-binding regions comprise the light-chain variable region from murine anti-BCMA antibody c11D5.3 (SEQ ID NO:21).

17. The bispecific CAR of claim 15 or 16, wherein the BCMA-binding regions comprise a variable region comprising SEQ ID NO:22.

18. The bispecific CAR of claim 12 or 14, wherein the BCMA-binding regions comprise a humanized heavy-chain variable region from anti-BCMA antibody c11D5.3.

19. The bispecific CAR of claim 18, wherein the humanized heavy-chain variable region comprises the amino acid sequence of SEQ ID NO:23.

20. The bispecific CAR of claim 13 or 14, wherein the BCMA-binding regions comprise a humanized light-chain variable region from anti-BCMA antibody c11D5.3.

21. The bispecific CAR of claim 20, wherein the humanized light-chain variable region comprises the amino acid sequence of SEQ ID NO:24.

22. The bispecific CAR of claim 19 or 21, wherein the BCMA-binding regions comprise a humanized variable heavy chain and variable light chain from anti-BCMA antibody c11D5.3.

23. The bispecific CAR of claim 22, wherein the BCMA-binding regions comprise the amino acid sequence of the variable regions of the humanized heavy and light chains of c11D5.3 (SEQ ID NO:25).

24. The bispecific CAR of any of claims 1-11, wherein the BCMA-binding regions comprise heavy-chain CDR1 (SEQ ID NO:26), CDR2 (SEQ ID NO:27), and CDR3 (SEQ ID NO:28) from anti-BCMA antibody J22.9-xi.

25. The bispecific CAR of claim 10, wherein the BCMA-binding regions comprise light-chain CDR1 (SEQ ID NO:30), CDR2 (SEQ ID NO:31), and CDR3 (SEQ ID NO:32) from anti-BCMA antibody J22.9-xi.

26. The bispecific CAR of claim 24 or 25, wherein the BCMA-binding regions comprise heavy-chain CDR1 (SEQ ID NO:26), CDR2 (SEQ ID NO:27), and CDR3 (SEQ ID NO:28) and light-chain CDR1 (SEQ ID NO:30), CDR2 (SEQ ID NO:31), and CDR3 (SEQ ID NO:32) from anti-BCMA antibody J22.9-xi.

27. The bispecific CAR of claim 24, wherein the BCMA-binding regions comprise the heavy-chain variable region from murine anti-BCMA antibody J22.9-xi (SEQ ID NO:29).

28. The bispecific CAR of claim 25, wherein the BCMA-binding regions comprise the light-chain variable region from murine anti-BCMA antibody J22.9-xi (SEQ ID NO:33).

29. The bispecific CAR of claim 27 or 28, wherein the BCMA-binding regions comprise a variable region comprising SEQ ID NO:34.

30. The bispecific CAR of claim 24 or 26, wherein the BCMA-binding regions comprise a humanized heavy-chain variable region from anti-BCMA antibody J22.9-xi.

31. The bispecific CAR of claim 30, wherein the humanized heavy-chain variable region comprises the amino acid sequence of SEQ ID NO:35.

32. The bispecific CAR of claim 25 or 26, wherein the BCMA-binding regions comprise a humanized light-chain variable region from anti-BCMA antibody J22.9-xi.

33. The bispecific CAR of claim 32, wherein the humanized light-chain variable region comprises the nucleic-acid sequence of SEQ ID NO:36.

34. The bispecific CAR of claim 31 or 33, wherein the BCMA-binding regions comprise a humanized variable heavy chain and variable light chain from anti-BCMA antibody J22.9-xi.

35. The bispecific CAR of claim 34, wherein the BCMA-binding regions comprise the amino acid sequence of SEQ ID NO:37.

36. The bispecific CAR of any of claims 1-11, wherein the BCMA-binding regions comprise a derivative APRIL fragment (dAPRIL).

37. The bispecific CAR of claim 36, wherein the dAPRIL fragment is at least 80% identical to SEQ ID NO:38.

38. The bispecific CAR of claim 36, wherein the dAPRIL fragment is at least 90% identical to SEQ ID NO:38.

39. The bispecific CAR of claim 38, wherein the dAPRIL fragment is at least 95% identical to SEQ ID NO:38.

40. The bispecific CAR of claim 39, wherein the dAPRIL fragment comprises SEQ ID NO:38.

41. The bispecific CAR of claim 11, wherein the CS1-binding regions comprise heavy-chain CDR1 (SEQ ID NO:39), CDR2 (SEQ ID NO:40), and CDR3 (SEQ ID NO:41) from murine anti-CS1 antibody Luc90.

42. The bispecific CAR of claim 41, wherein the CS1-binding regions comprise the heavy-chain variable region from anti-CS1 antibody Luc90 (SEQ ID NO:42).

43. The bispecific CAR of claim 11, wherein the CS1-binding regions comprise light-chain CDR1 (SEQ ID NO:43), CDR2 (SEQ ID NO:44), and CDR3 (SEQ ID NO:45) from anti-CS1 antibody Luc90.

44. The bispecific CAR of claim 42, wherein the CS1-binding regions comprise the light-chain variable region from anti-CS1 antibody Luc90 (SEQ ID NO:46).

45. The bispecific CAR of claim 41 or 43, wherein the CS1-binding regions comprise heavy-chain CDR1 (SEQ ID NO:39), CDR2 (SEQ ID NO:40), and CDR3 (SEQ ID NO:41) and light-chain CDR1 (SEQ ID NO:43), CDR2 (SEQ ID NO:44), and CDR3 (SEQ ID NO:45) from anti-CS1 antibody Luc90.

46. The bispecific CAR of claim 42 or 44 wherein the CS1-binding regions comprise a variable region comprising SEQ ID NO:47.

47. The bispecific CAR of claim 11, wherein the CS1-binding regions comprise heavy-chain CDR1 (SEQ ID NO:48), CDR2 (SEQ ID NO:49), and CDR3 (SEQ ID NO:50) from anti-CS1 antibody huLuc63.

48. The bispecific CAR of claim 11, wherein the CS1-binding regions comprise light-chain CDR1 (SEQ ID NO:52), CDR2 (SEQ ID NO:53), and CDR3 (SEQ ID NO:54) from anti-CS1 antibody huLuc63.

49. The bispecific CAR of claim 47 or 48, wherein the CS1-binding regions comprise heavy-chain CDR1 (SEQ ID NO:48), CDR2 (SEQ ID NO:49), and CDR3 (SEQ ID NO:50) and light-chain CDR1 (SEQ ID NO:52), CDR2 (SEQ ID NO:53), and CDR3 (SEQ ID NO:54) from anti-CS1 antibody huLuc63.

50. The bispecific CAR of claim 47, wherein the CS1-binding regions comprise the heavy-chain variable region from anti-CS1 antibody huLuc63 (SEQ ID NO:51).

51. The bispecific CAR of claim 48, wherein the CS1-binding regions comprise the light-chain variable region from anti-CS1 antibody huLuc63 (SEQ ID NO:55).

52. The bispecific CAR of claim 50 or 51 wherein the CS1-binding regions comprise a variable region comprising SEQ ID NO:56.

53. The bispecific CAR of claim 10 or 11, wherein the BCMA-binding regions comprise dAPRIL or CDR1, CDR2, and/or CDR3 of both the variable heavy and light chains or the heavy and light chain variable regions from c11D5.3 or J22.9-xi antibody; and the CS1-binding regions comprise CDR1, CDR2, and/or CDR3 of both the variable heavy and light chains or the heavy and light chain variable regions from huLuc63 or Luc90 antibody.

54. The bispecific CAR of any of claims 1-53, wherein the bispecific CAR further comprises an extracellular spacer.

55. The bispecific CAR of claim 54, wherein the extracellular spacer is between 8 and 1000 amino acids in length.

56. The bispecific CAR of claim 55, wherein the extracellular spacer is between 8 and 500 amino acids in length.

57. The bispecific CAR of claim 56, wherein the extracellular spacer is between 100-300 amino acids in length.

58. The bispecific CAR of claim 56, wherein the extracellular spacer has fewer than 100 amino acids.

59. The bispecific CAR of claim any of claims 54-58, wherein the extracellular spacer is an IgG4 hinge, a CD8a hinge, an IgG1 hinge, or a CD34 hinge.

60. The bispecific CAR of any of claims 54-55, wherein the extracellular spacer comprises an IgG4 hinge.

61. The bispecific CAR of any of claims 54-60, wherein the extracellular spacer comprises a CH1, CH2, and/or a CH3 region

62. The bispecific CAR of any of claims 1-61, wherein the transmembrane domain is an alpha or beta chain of the T cell receptor, CD28, CD3ε (epsilon), CD45, CD4, CD5, CD8, CD9, CD16, CD22, CD33, CD37, CD64, CD80, CD86, CD123, CD134, CD137 or CD154 transmembrane domain.

63. The bispecific CAR of claim 62, wherein the transmembrane domain is a CD28 transmembrane domain.

64. The bispecific CAR of any of claims 1-63, wherein the primary intracellular signaling domain is CD3ζ (zeta).

65. The bispecific CAR of any of claims 1-64, wherein the single cytoplasmic region further comprises one or more costimulatory domains.

66. The bispecific CAR of claim 65, wherein the single cytoplasmic region comprises two costimulatory domains.

67. The bispecific CAR of claim 65 or 66, wherein the one or more costimulatory domain(s) comprise 4-1BB (CD137), CD28, IL-15Rα, OX40, CD2, CD27, CDS, ICAM-1, LFA-1 (CD11a/CD18), and/or ICOS (CD278).

68. The bispecific CAR of claim 67, wherein the one or more costimulatory domains comprise 4-1BB.

69. A bispecific chimeric antigen receptor (CAR) comprising:
i) a bispecific extracellular binding domain comprising a BCMA single-chain variable fragment (scFv) and a CS1-specific scFv separated by a linker; wherein the BCMA-specific scFv comprises CDR1, CDR2, and CDR3 from the heavy and light chains of C11D5.3 or J22.9-xi antibodies and wherein the CS1-specific scFv comprises CDR1, CDR2, and CDR3 from the heavy and light chains of Luc90 or huLuc63 antibodies and wherein the linker comprises G4S;
ii) a hinge spacer between 8-300 amino acids in length;
iii) one CD28 transmembrane domain; and,
iv) one cytoplasmic region comprising 4-1BB co-stimulatory domain and CD3zeta intracellular signaling domain.

70. The bispecific chimeric antigen receptor (CAR) of claim 69, wherein the BCMA-specific scFv is membrane proximal.

71. The bispecific chimeric antigen receptor (CAR) of claim 69, wherein the BCMA-specific scFv is membrane distal.

72. A bispecific chimeric antigen receptor (CAR) comprising:
i) a bispecific extracellular binding domain comprising a BCMA-binding region comprising a dAPRIL fragment and a CS1-specific scFv separated by a linker; wherein the CS1-specific scFv comprises CDR1, CDR2, and CDR3 from the heavy and light variable chains of Luc90 or huLuc63 antibodies and wherein the linker comprises G4S;
ii) a hinge spacer between 8-300 amino acids in length;
iii) one CD28 transmembrane domain; and,
iv) one cytoplasmic region comprising 4-1BB co-stimulatory domain and CD3zeta intracellular signaling domain.

73. The bispecific CAR of claim 72, wherein the dAPRIL fragment is membrane proximal.

74. The bispecific CAR of claim 72, wherein the dAPRIL fragment is membrane distal.

75. The bispecific CAR of any of claims 72-74, wherein the dAPRIL fragment comprises SEQ ID NO:73.

76. A chimeric antigen receptor (CAR) comprising:
a) an extracellular binding domain comprising a BCMA-binding region and an extracellular spacer of SEQ ID NO:172 or 73;
b) a single transmembrane domain; and,
c) a single cytoplasmic region comprising a primary intracellular signaling domain.

77. The CAR of claim 76, wherein the BCMA-specific scFv comprises either i) CDR1, CDR2, CDR3 of both the heavy and light chains from murine or humanized c11D5.3 antibody or murine or humanized J22.9-xi antibody or ii) the heavy and light chain variable regions from murine or humanized c11D5.3 or murine or humanized J229-xi antibody.

78. The CAR of claim 77, wherein the BCMA-specific scFv comprises heavy-chain CDR1 (SEQ ID NO:14), CDR2 (SEQ ID NO:15), and CDR3 (SEQ ID NO:16) from anti-BCMA antibody c11D5.3.

79. The CAR of claim 77 or 78, wherein the BCMA-specific scFv comprises light-chain CDR1 (SEQ ID NO:18), CDR2 (SEQ ID NO:19), and CDR3 (SEQ ID NO:20) from anti-BCMA antibody c11D5.3.

80. The CAR of claim 77, wherein the BCMA-specific scFv comprises heavy-chain CDR1 (SEQ ID NO:14), CDR2 (SEQ ID NO:15), and CDR3 (SEQ ID NO:16) and light-chain CDR1 (SEQ ID NO:18), CDR2 (SEQ ID NO:19), and CDR3 (SEQ ID NO:20) from anti-BCMA antibody c11D5.3.

81. The CAR of claim 77, wherein the BCMA-specific scFv comprises the heavy-chain variable region from murine anti-BCMA antibody c11D5.3 (SEQ ID NO:17).

82. The CAR of claim 77 or 81, wherein the BCMA-specific scFv comprises the light-chain variable region from murine anti-BCMA antibody c11D5.3 (SEQ ID NO:21).

83. The CAR of claim 77, 81, or 82, wherein the BCMA-specific scFv comprises a variable region comprising SEQ ID NO:22.

84. The CAR of claim 77, wherein the BCMA-specific scFv comprises a humanized heavy-chain variable region from anti-BCMA antibody c11D5.3.

85. The CAR of claim 84, wherein the humanized heavy-chain variable region comprises the amino acid sequence of SEQ ID NO:23.

86. The CAR of claim 77, 84, or 85, wherein the BCMA-specific scFv comprises a humanized light-chain variable region from anti-BCMA antibody c11D5.3.

87. The CAR of claim 86, wherein the humanized light-chain variable region comprises the amino acid sequence of SEQ ID NO:24.

88. The CAR of any one of claims 84-87, wherein the BCMA-specific scFv comprises a humanized variable heavy chain and variable light chain from anti-BCMA antibody c11D5.3.

89. The CAR of claim 88, wherein the BCMA-specific scFv comprises the amino acid sequence of the variable regions of the humanized heavy and light chains of c11D5.3 (SEQ ID NO:25).

90. The CAR of claim 77, wherein the BCMA-specific scFv comprises heavy-chain CDR1 (SEQ ID NO:26), CDR2 (SEQ ID NO:27), and CDR3 (SEQ ID NO:28) from anti-BCMA antibody J22.9-xi.

91. The CAR of claim 77 or 90, wherein the BCMA-specific scFv comprises light-chain CDR1 (SEQ ID NO:30), CDR2 (SEQ ID NO:31), and CDR3 (SEQ ID NO:32) from anti-BCMA antibody J22.9-xi.

92. The CAR of claim 77, 90, or 91, wherein the BCMA-specific scFv comprises heavy-chain CDR1 (SEQ ID NO:26), CDR2 (SEQ ID NO:27), and CDR3 (SEQ ID NO:28) and light-chain CDR1 (SEQ ID NO:30), CDR2 (SEQ ID NO:31), and CDR3 (SEQ ID NO:32) from anti-BCMA antibody J22.9-xi.

93. The CAR of any one of claims 90-92, wherein the BCMA-specific scFv comprises the heavy-chain variable region from murine anti-BCMA antibody J22.9-xi (SEQ ID NO:29).

94. The CAR of any one of claims 90-93, wherein the BCMA-specific scFv comprises the light-chain variable region from murine anti-BCMA antibody J22.9-xi (SEQ ID NO:33).

95. The CAR of any one of claims 90-94, wherein the BCMA-specific scFv comprises a variable region comprising SEQ ID NO:34.

96. The CAR of claim 77, wherein the BCMA-specific scFv comprises a humanized heavy-chain variable region from anti-BCMA antibody J22.9-xi.

97. The CAR of claim 96, wherein the humanized heavy-chain variable region comprises the amino acid sequence of SEQ ID NO:35.

98. The CAR of claim 77, 96, or 97, wherein the BCMA-specific scFv comprises a humanized light-chain variable region from anti-BCMA antibody J22.9-xi.

99. The CAR of claim 98, wherein the humanized light-chain variable region comprises the nucleic-acid sequence of SEQ ID NO:36.

100. The CAR of any one of claims 96-99, wherein the BCMA-specific scFv comprises a humanized variable heavy chain and variable light chain from anti-BCMA antibody J22.9-xi.

101. The CAR of claim 100, wherein the BCMA-specific scFv comprises the amino acid sequence of SEQ ID NO:37.

102. The CAR of claim 76, wherein the BCMA-binding region comprises a derivative APRIL fragment (dAPRIL).

103. The CAR of claim 103, wherein the dAPRIL fragment is at least 80% identical to SEQ ID NO:38.

104. The CAR of any one of claims 76-103, wherein the transmembrane domain is an alpha or beta chain of the T cell receptor, CD28, CD3 epsilon, CD45, CD4, CD5, CD8, CD9, CD16, CD22, CD33, CD37, CD64, CD80, CD86, CD123, CD134, CD137 or CD154 transmembrane domain.

105. The CAR of claim 104, wherein the transmembrane domain is a CD28 transmembrane domain.

106. The CAR of any one of claims 76-105, wherein the primary intracellular signaling domain is CD3ζ (zeta).

107. The CAR of any one of claims 76-108, wherein the single cytoplasmic region further comprises one or more costimulatory domains.

108. The CAR of claim 107, wherein the single cytoplasmic region comprises two costimulatory domains.

109. The CAR of claim 107 or 108, wherein the one or more costimulatory domain(s) comprise 4-1BB (CD137), CD28, IL-15Rα, OX40, CD2, CD27, CDS, ICAM-1, LFA-1 (CD11a/CD18), and/or ICOS (CD278).

110. The CAR of claim 109, wherein the one or more costimulatory domains comprise 4-1BB.

111. A chimeric antigen receptor (CAR) comprising:
a) an extracellular binding domain comprising a BCMA scFv of SEQ ID NO:22 or 25 and an extracellular spacer of SEQ ID NO:172;
b) a single transmembrane domain of SEQ ID NO:76; and,
c) a cytoplasmic region comprising a costimulatory domain of SEQ ID NO:77 and a primary intracellular signaling domain of SEQ ID NO:78.

112. The CAR of anyone of claims 76-111, wherein the CAR is monospecific.

113. A nucleic acid comprising a sequence encoding a chimeric antigen receptor of any of claims 1-112.

114. The nucleic acid of claim 113, wherein the nucleic acid is an expression construct.

115. The nucleic acid of claim 114, wherein the expression construct is a viral vector.

116. The nucleic acid of claim 115, wherein the viral vector comprises a retroviral vector a vector derived from a retrovirus.

117. The nucleic acid of claim 116, wherein the viral vector is a lentiviral vector or a vector derived from a lentivirus.

118. A lentivirus vector comprising a sequence encoding the chimeric antigen receptor (CAR) of any one of claims 1-112.

119. A cell comprising the nucleic acid of any of claims 113-118.

120. The cell comprising the nucleic acid of claim 119, wherein the viral vector has integrated into the cell's genome.

121. A cell expressing a chimeric antigen receptor of any of claims 1-112.

122. The cell of any of claims 119-121, wherein the cell is a T cell, a natural killer (NK) cell, a natural killer T cell (NKT), an invariant natural killer T cell (iNKT), stem cell, lymphoid progenitor cell, peripheral blood mononuclear cell (PBMC), bone marrow cell, fetal liver cell, embryonic stem cell, cord blood cell, induced pluripotent stem cell (iPS cell).

123. The cell of claim 122, wherein the cell is a T cell or an NK cell.

124. The method of claim 123, wherein the T cell comprises a naïve memory T cell.

125. The method of claim 124, wherein the naïve memory T cell comprises a CD4+ or CD8+ T cell.

126. The method of any one of claims 123-125, wherein the T cell comprises a T cell from a population of CD14 depleted, CD25 depleted, and CD62L enriched PBMCs.

127. A population of cell comprising any of the cells of claims 119-126.

128. The population of cells of claim 127, wherein the population comprises 10³-10⁸ cells.

129. A composition comprising the population of cells of claim 127 or 128, wherein the composition is a pharmaceutically acceptable formulation.

130. A method of making a cell that expresses a chimeric antigen receptor comprising introducing into a cell the nucleic acid of any of claims 113-118.

131. The method of claim 130, wherein the cell is infected with a virus encoding the CAR.

132. The method of claim 131, wherein the virus comprises lentivirus or a lentiviral-derived virus or vector.

133. The method of any one of claims 130-132, wherein the cell is a T cell, a natural killer (NK) cell, a natural killer T cell (NKT), an invariant natural killer T cell (iNKT), stem cell, lymphoid progenitor cell, peripheral blood mononuclear cell (PBMC), bone marrow cell, fetal liver cell, embryonic stem cell, cord blood cell, induced pluripotent stem cell (iPS cell).

134. The method of claim 133, wherein the cell is a T cell or an NK cell.

135. The method of claim 134, wherein the T cell comprises a naïve memory T cell.

136. The method of claim 135, wherein the naïve memory T cell comprises a CD4+ or CD8+ T cell.

137. The method of any one of claims 134-136, wherein the T cell comprises a T cell from a population of CD14 depleted, CD25 depleted, and CD62L enriched PBMCs.

138. The method of any one of claims 134-137, wherein the cell is not yet a T cell or NK cell, the method further comprising culturing the cell under conditions that promote the differentiation of the cell into a T cell or an NK cell.

139. The method of any of claims 130-138, further comprising culturing the cell under conditions to expand the cell before and or after introducing the nucleic acid into the cell.

140. The method of claim 139, wherein the cell is cultured with serum-free medium.

141. A method of treating a patient with cancer comprising administering to the patient an effective amount of the composition of claim 129.

142. The method of claim 141, wherein the patient has a myeloma or lymphoma.

143. The method of claim 142, wherein the patient has multiple myeloma.

144. The method of claim 143, wherein the patient has relapsed multiple myeloma.

145. The method of any of claims 141-144, further comprising administering an additional therapy to the patient.

146. The method of claim 145, wherein the additional therapy comprises an immunotherapy.

147. The method of claim 146, wherein the immunotherapy comprises immune checkpoint inhibitor therapy.

148. The method of claim 147, wherein the immune checkpoint inhibitor therapy comprises a PD-1 inhibitor.

149. A method of treating a patient with multiple myeloma comprising administering to the patient a composition comprising a population of cells expressing a chimeric antigen receptor (CAR) comprising:
a) a bispecific extracellular binding domain comprising i) a dAPRIL fragment or a BCMA single-chain variable fragment (scFv) and ii) a CS1-specific scFv separated by a linker; wherein the BCMA-specific scFv comprises CDR1, CDR2, and CDR3 from the heavy and light chains of C11D5.3 or J22.9-xi antibodies and wherein the CS1-specific scFv comprises CDR1, CDR2, and CDR3 from the heavy and light chains of murine Luc90 or huLuc63 antibodies and wherein the linker comprises G4S;
b) a hinge spacer between 8-300 amino acids in length;
c) one CD28 transmembrane domain; and,
d) one cytoplasmic region comprising 4-1BB co-stimulatory domain and CD3zeta intracellular signaling domain.

150. The method of claim 149, wherein the cells are autologous.

151. A bispecific chimeric antigen receptor (CAR) comprising in order from the amino to carboxy end of the CAR:
i) a bispecific extracellular binding domain comprising a BCMA-specific scFv comprising SEQ ID NO:25; a (G4S)₄ linker; and a CS1-specific scFv comprising SEQ ID NO:56;
ii) a hinge spacer comprising a IgG4 hinge with CH2 and/or CH3 regions and wherein the hinge spacer is between 100-250 amino acids in length;
iii) one CD28 transmembrane domain; and,
iv) one cytoplasmic region comprising 4-1BB co-stimulatory domain and CD3zeta intracellular signaling domain.

152. A bispecific chimeric antigen receptor (CAR) comprising in order from the amino to carboxy end of the CAR:
i) a bispecific extracellular binding domain comprising a CS1-specific scFv comprising SEQ ID NO:56; a (G4S)₄ linker; and a BCMA-specific scFv comprising SEQ ID NO:25;
ii) a hinge spacer comprising a IgG4 hinge and wherein the spacer is between 4-50 amino acids in length;
iii) one CD28 transmembrane domain; and,
iv) one cytoplasmic region comprising 4-1BB co-stimulatory domain and CD3zeta intracellular signaling domain.

153. A bispecific chimeric antigen receptor (CAR) comprising in order from the amino to carboxy end of the CAR:
i) a bispecific extracellular binding domain comprising a BCMA-specific scFv comprising SEQ ID NO:25; a (G4S)₄ linker; and a CS1-specific scFv comprising SEQ ID NO:47;
ii) a hinge spacer comprising a IgG4 hinge and wherein the spacer is between 4-50 amino acids in length;
iii) one CD28 transmembrane domain; and,
iv) one cytoplasmic region comprising 4-1BB co-stimulatory domain and CD3zeta intracellular signaling domain.

154. A method of treating a patient with multiple myeloma comprising administering to the patient a composition comprising a population of cells expressing a bispecific chimeric antigen receptor (CAR) comprising in order from the amino to carboxy end of the CAR:
i) a bispecific extracellular binding domain comprising a BCMA-specific scFv comprising SEQ ID NO:25; a (G4S)₄ linker; and a CS1-specific scFv comprising SEQ ID NO:56;
ii) a hinge spacer comprising a IgG4 hinge, CH2, and CH3 region and wherein the spacer is between 200-250 amino acids in length;
iii) one CD28 transmembrane domain; and,
iv) one cytoplasmic region comprising 4-1BB co-stimulatory domain and CD3zeta intracellular signaling domain.

155. A method of treating a patient with multiple myeloma comprising administering to the patient a composition comprising a population of cells expressing a bispecific chimeric antigen receptor (CAR) comprising in order from the amino to carboxy end of the CAR:
i) a bispecific extracellular binding domain comprising a CS1-specific scFv comprising SEQ ID NO:56; a (G4S)₄ linker; and a and BCMA-specific scFv comprising SEQ ID NO:25;
ii) a hinge spacer comprising a IgG4 hinge and wherein the spacer is between 4-50 amino acids in length;
iii) one CD28 transmembrane domain; and,
iv) one cytoplasmic region comprising 4-1BB co-stimulatory domain and CD3zeta intracellular signaling domain.

156. A method of treating a patient with multiple myeloma comprising administering to the patient a composition comprising a population of cells expressing a bispecific chimeric antigen receptor (CAR) comprising in order from the amino to carboxy end of the CAR:
i) a bispecific extracellular binding domain comprising a BCMA-specific scFv comprising SEQ ID NO:25; a (G4S)₄ linker; and a CS1-specific scFv comprising SEQ ID NO:47;
ii) a hinge spacer comprising a IgG4 hinge and wherein the spacer is between 4-50 amino acids in length;
iii) one CD28 transmembrane domain; and,
iv) one cytoplasmic region comprising 4-1BB co-stimulatory domain and CD3zeta intracellular signaling domain.

157. A method of treating a patient with multiple myeloma comprising administering to the patient a composition comprising a population of cells expressing the bispecific chimeric antigen receptor (CAR) of any of claims 1-112 or 151-103.
